(19) United States
(12) Patent Application Publication
(10) Pub. No.: US 2023/0357447 A1
(43) Pub. Date: Nov. 9, 2023

(54) TUMOR ACTIVATED MULTISPECIFIC ANTIBODIES FOR TARGETING CD28 AND PD-L1 AND METHODS OF USE THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US)

(21) Appl. No.: 18/314,090

(22) Filed: May 8, 2023

Related U.S. Application Data
(63) Continuation of application No. PCT/US2023/066567, filed on May 3, 2023.
(60) Provisional application No. 63/338,115, filed on May 4, 2022.

(51) Int. Cl. C07K 16/46

(52) U.S. Cl. CPC C07K 16/468; C07K 2317/31; C07K 2317/55; C07K 2317/622; C07K 2317/565

(57) ABSTRACT

Provided herein are antibodies that selectively bind to CD28 and PD-L1, pharmaceutical compositions thereof, as well as nucleic acids, and methods of use, and methods for making and discovering the same.

### CROSS REFERENCE

The present application is a continuation of International Application No. PCT/US2023/066567, filed May 3, 2023, which claims the benefit of U.S. Provisional Application No. 63/338,115, filed on May 4, 2022, which is incorporated by reference in its entirety.

### SEQUENCE LISTING

The instant application contains a sequence listing which has been submitted electronically in XML format and is hereby incorporated by reference in its entirety. Said XML copy was created on Apr. 13, 2023, and is named 52426-741_301_SL.xml and is 578,045 bytes in size.

### SUMMARY

Disclosed herein are isolated multispecific antibodies according to the following formula: P₁-L₁-A₁-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20. In some embodiments, the multispecific antibody is according to the following formula: P₁-L₁-A₁-L-B-L₂-P₂ (Formula Ia) wherein P₂ comprises a peptide that binds to B and L₂ comprises a linking moiety that connects B to P₂ and is a substrate for a tumor specific protease. In some embodiments, P₁ comprises an amino acid sequence according to any one of SEQ ID NOs: 24-53, 128-148, and the amino acid sequences of Table 20. In some embodiments, P₁ comprises an amino acid sequence according to any one of SEQ ID NOs: 42-53 or an amino acid sequence that has 1, 2, or 3 amino acid mutations, substitutions, or deletions relative to any one of SEQ ID NOs: 42-53. In some embodiments, P₁ comprises an amino acid sequence according to any one of SEQ ID NOs: 42-53. In some embodiments, P₁ comprises an amino acid sequence according to any one of the amino acid sequences of Table 20 or an amino acid sequence that has 1, 2, or 3 amino acid mutations, substitutions, or deletions relative to any one of the amino acid sequences of Table 20. In some embodiments, P₁ comprises an amino acid sequence according to any one of the amino acid sequences of Table 20. In some embodiments, P₁ comprises an amino acid sequence according to any one of SEQ ID NOs: 128-147 or an amino acid sequence that has 1, 2, or 3 amino acid mutations, substitutions, or deletions relative to any one of SEQ ID NOs: 128-147. In some embodiments, P₁ comprises an amino acid sequence according to any one of SEQ ID NOs: 128-147. In some embodiments, P₁ comprises an amino acid sequence according to X₁-X₂-X₃-C-X₄-X₅-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y. In some embodiments, X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F. In some embodiments, P₁ comprises an amino acid sequence according to SEQ ID NO: 32 or an amino acid sequence that has 1, 2, or 3 amino acid mutations, substitutions, or deletions relative to SEQ ID NO: 32. In some embodiments, P₁ comprises an amino acid sequence according to SEQ ID NO: 32. In some embodiments, P₁ comprises an amino acid sequence according to SEQ ID NO: 138 or an amino acid sequence that has 1, 2, or 3 amino acid mutations, substitutions, or deletions relative to SEQ ID NO: 138. In some embodiments, P₁ comprises an amino acid sequence according to SEQ ID NO: 138. In some embodiments, P₁ impairs binding of A₁ to CD28. In some embodiments, P₁ is bound to A₁ through ionic interactions, electrostatic interactions, hydrophobic interactions, Pi-stacking interactions, and H-bonding interactions, or a combination thereof. In some embodiments, P₁ is bound to A₁ at or near an antigen binding site. In some embodiments, P₁ becomes unbound from A₁ when L₁ is cleaved by the tumor specific protease thereby exposing A₁ to CD28. In some embodiments, P₁ has less than 75% sequence identity to CD28. In some embodiments, P₁ has less than 80% sequence identity to CD28. In some embodiments, P₁ has less than 85% sequence identity to CD28. In some embodiments, P₁ has less than 90% sequence identity to CD28. In some embodiments, P₁ has less than 95% sequence identity to CD28. In some embodiments, P₁ comprises a de novo amino acid sequence that shares less than 10% sequence identity to CD28. In some embodiments, P₂ impairs binding of B to PD-L1. In some embodiments, P₂ is bound to B through ionic interactions, electrostatic interactions, hydrophobic interactions, Pi-stacking interactions, and H-bonding interactions, or a combination thereof. In some embodiments, P₂ is bound to B at or near an antigen binding site. In some embodiments, P₂ becomes unbound from B when L₂ is cleaved by the tumor specific protease thereby exposing B to the PD-L1. In some embodiments, P₂ has less than 70% sequence identity to the PD-L1. In some embodiments, P₂ has less than 75% sequence identity to the PD-L1. In some embodiments, P₂ has less than 80% sequence identity to the PD-L1. In some embodiments, P₂ has less than 85% sequence identity to the PD-L1. In some embodiments, P₂ has less than 90% sequence identity to the PD-L1. In some embodiments, P₂ has less than 95% sequence identity to the PD-L1. In some embodiments, P₂ comprises a de novo amino acid sequence that shares less than 10% sequence identity to the PD-L1. In some embodiments, P₂ comprises a peptide sequence of at least 5 amino acids in length. In some embodiments, P₂ comprises a peptide sequence of at least 6 amino acids in length. In some embodiments, P₂ comprises a peptide sequence of at least 10 amino acids in length. In some embodiments, P₂ comprises a peptide sequence of at least 10 amino acids in length and no more than 20 amino acids in length. In some embodiments, P₂ comprises a peptide sequence of at least 16 amino acids in length. In some embodiments, P₂ comprises a peptide sequence of no more than 40 amino acids in length. In some embodiments, P₁ or P₂ comprises at least two cysteine amino acid residues. In some embodiments, P₁ or P₂ comprises a cyclic peptide or a linear peptide. In some embodiments, P₁ or P₂ comprises a cyclic peptide. In some embodiments, P₁ or P₂ comprises a linear peptide. In some embodiments, P₁ or P₂ comprise a modified amino acid or non-natural amino acid, or a modified non-natural amino acid, or a combination thereof. In some embodiments, P₁ or P₂ does not comprise albumin or an albumin fragment. In some embodiments, P₁ or P₂ does not comprise an albumin binding domain. In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids. In some embodiments, L₁ or L₂ comprises a formula comprising (G2S)n, wherein n is an integer from 1 to 3 (SEQ ID NO: 228). In some embodiments, L₁ or L₂ comprises a formula comprising (G2S)n, wherein n is an integer of at least 1. In some embodiments, L₁ or L₂ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments, L₁ or L₂ comprises a urokinase cleavable amino acid sequence, a matriptase cleavable amino acid sequence, or a matrix metalloprotease cleavable amino acid sequence. In some embodiments, L₁ or L₂ comprises a sequence according to SEQ ID NOs: 18-19, 62-88. In some embodiments, L₁ is bound to N-terminus of A₁. In some embodiments, L₁ is bound to C-terminus of A₁. In some embodiments, L₂ is bound to N-terminus of B. In some embodiments, L₂ is bound to C-terminus of B. In some embodiments, the CD28 binding domain comprises a single chain variable fragment, a single domain antibody, a Fab, or a Fab′. In some embodiments, the CD28 binding domain comprises the single chain variable fragment. In some embodiments, the CD28 binding domain comprises the single domain antibody. In some embodiments, the CD28 binding domain comprises the Fab or the Fab′. In some embodiments, the PD-L1 binding domain comprises a single chain variable fragment, a single domain antibody, a Fab, or a Fab′. In some embodiments, the PD-L1 binding domain comprises the Fab or the Fab′. In some embodiments, the PD-L1 binding domain comprises the Fab or the Fab′ and the CD28 binding domain comprises the single chain variable fragment. In some embodiments, the PD-L1 binding domain that comprises the Fab or the Fab′ comprises a Fab heavy chain polypeptide comprising a Fab heavy chain variable domain and a Fab light chain polypeptide comprising a Fab light chain variable domain. In some embodiments, the CD28 binding domain that comprises the single chain variable fragment comprises a scFv heavy chain variable domain and a scFv light chain variable domain. In some embodiments, the linker connects the C-terminus of A₁ to an N-terminus of B. In some embodiments, the linker connects the N-terminus of A₁ to a C-terminus of B. In some embodiments, the linker connects the C-terminus of A₁ to the N-terminus of the Fab heavy chain polypeptide. In some embodiments, the linker connects the N-terminus of A₁ to the C-terminus of the Fab heavy chain polypeptide. In some embodiments, the linker connects the C-terminus of A₁ to the N-terminus of the Fab light chain polypeptide. In some embodiments, the linker connects the N-terminus of A₁ to the C-terminus of the Fab light chain polypeptide. In some embodiments, the linker connects the Fab light chain polypeptide to the scFv light chain variable domain. In some embodiments, the linker connects the Fab light chain polypeptide to the scFv heavy chain variable domain. In some embodiments, the linker connects the Fab heavy chain polypeptide to the scFv light chain variable domain. In some embodiments, the linker connects the Fab heavy chain polypeptide to the scFv heavy chain variable domain. In some embodiments, the linker connects the Fab light chain polypeptide to the N-terminus of the scFv light chain variable domain. In some embodiments, the linker connects the Fab light chain polypeptide to the C-terminus of the scFv light chain variable domain. In some embodiments, the linker connects the Fab light chain polypeptide to the N-terminus of the scFv heavy chain variable domain. In some embodiments, the linker connects the Fab light chain polypeptide to the C-terminus of the scFv heavy chain variable domain. In some embodiments, the linker connects the Fab heavy chain polypeptide to the N-terminus of the scFv light chain variable domain. In some embodiments, the linker connects the Fab heavy chain polypeptide to the C-terminus of the scFv light chain variable domain. In some embodiments, the linker connects the Fab heavy chain polypeptide to the N-terminus of the scFv heavy chain variable domain. In some embodiments, the linker connects the Fab heavy chain polypeptide to the C-terminus of the scFv heavy chain variable domain. In some embodiments, the linker is at least 5 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker is 15 amino acids in length. In some embodiments, the linker comprises (G2S)n, (GS)n, (GSGGS)n (SEQ ID NO: 58), (GGGS)n (SEQ ID NO: 59), (GGGGS)n (SEQ ID NO: 60), and (GSSGGS)n (SEQ ID NO: 61), wherein n is an integer of at least 1. In some embodiments, L comprises a formula comprising (G2S)n, wherein n is an integer from 1 to 3 (SEQ ID NO: 228). In some embodiments, the L comprises an amino acid sequence of SEQ ID NO: 18 (GGGGSGGGGSGGGGS) or SEQ ID NO: 19 (GGGGS). In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5 (KA); and LC-CDR3: SEQ ID NO: 6, and wherein the CDRs comprise from 0-2 amino acid modifications in at least one of said LC-CDR1, LC-CDR2, or LC-CDR3. In some embodiments, A₁ comprises complementarity determining regions (CDRs): LC-CDR1, LC-CDR2, and LC-CDR3, wherein the LC-CDR1, the LC-CDR2, and the LC-CDR3 of A₁ comprise: LC-CDR1: SEQ ID NO: 4; LC-CDR2: SEQ ID NO: 5 (KA); and LC-CDR3: SEQ ID NO: 6; wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 1; HC-CDR2: SEQ ID NO: 2; HC-CDR3: SEQ ID NO: 3. In some embodiments, the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12; and wherein said CDRs comprise from 0-2 amino acid modifications in at least one of said HC-CDR1, HC-CDR2, or HC-CDR3. In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14 (DA); and LC-CDR3: SEQ ID NO: 15; and wherein the CDRs comprise from 0-2 amino acid modifications in at least one of said LC-CDR1, LC-CDR2, or LC-CDR3. In some embodiments, B comprises complementarity determining region (CDRs): HC-CDR1, HC-CDR2, and HC-CDR3, wherein the HC-CDR1, the HC-CDR2, and the HC-CDR3 of B comprise: HC-CDR1: SEQ ID NO: 10; HC-CDR2: SEQ ID NO: 11; HC-CDR3: SEQ ID NO: 12; and wherein B comprises complementarity determining regions (CDRs): LC-CDR1, LC-CDR2, and LC-CDR3, wherein the LC-CDR1, the LC-CDR2, and the LC-CDR3 of B comprise: LC-CDR1: SEQ ID NO: 13; LC-CDR2: SEQ ID NO: 14 (DA); and LC-CDR3: SEQ ID NO: 15. In some embodiments, the scFv heavy chain variable domain comprises an amino acid sequence that has at least 80% sequence identity to the amino acid sequence according to SEQ ID NO: 7. In some embodiments, the scFv heavy chain variable domain comprises an amino acid sequence of at least 75 consecutive amino acid residues of SEQ ID NO: 7 In some embodiments, the scFv heavy chain variable domain comprises an amino acid sequence of at least 110 consecutive amino acid residues of SEQ ID NO: 7. In some embodiments, the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7. In some embodiments, the scFv heavy chain variable domain comprises an amino acid sequence according to SEQ ID NO: 7. In some embodiments, the scFv light chain variable domain comprises an amino acid sequence that has at least 80% sequence identity to the amino acid sequence according to SEQ ID NO: 8. In some embodiments, the scFv light chain variable domain comprises an amino acid sequence of at least 75 consecutive amino acid residues of SEQ ID NO: 8. In some embodiments, the scFv light chain variable domain comprises an amino acid sequence of at least 100 consecutive amino acid residues of SEQ ID NO: 8. In some embodiments, the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8. In some embodiments, the scFv light chain variable domain comprises an amino acid sequence according to SEQ ID NO: 8. In some embodiments, the scFv comprises an amino acid sequence that has at least 80% sequence identity to the amino acid sequence according to SEQ ID NO: 9. In some embodiments, the scFv comprises an amino acid sequence of at least 175 consecutive amino acid residues of SEQ ID NO: 9. In some embodiments, the scFv comprises an amino acid sequence of at least 210 consecutive amino acid residues of SEQ ID NO: 9. In some embodiments, the scFv comprises an amino acid sequence of at least 210 consecutive amino acid residues of SEQ ID NO: 9 and has at least 80% sequence identity to the at least 210 consecutive amino acid residues of SEQ ID NO: 9. In some embodiments, the scFv comprises an amino acid sequence according to SEQ ID NO: 9. In some embodiments, the Fab heavy chain polypeptide comprises an amino acid sequence that has at least 80% sequence identity to the amino acid sequence according to SEQ ID NO: 17. In some embodiments, the Fab heavy chain polypeptide comprises an amino acid sequence of at least 175 consecutive amino acid residues of SEQ ID NO: 17. In some embodiments, the Fab heavy chain polypeptide comprises an amino acid sequence of at least 215 consecutive amino acid residues of SEQ ID NO: 17. In some embodiments, the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17. In some embodiments, the Fab heavy chain polypeptide comprises an amino acid sequence according to SEQ ID NO: 17. In some embodiments, the Fab light chain polypeptide comprises an amino acid sequence that has at least 80% sequence identity to the amino acid sequence according to SEQ ID NO: 16. In some embodiments, the Fab light chain polypeptide comprises an amino acid sequence of at least 175 consecutive amino acid residues of SEQ ID NO: 16. In some embodiments, the Fab light chain polypeptide comprises an amino acid sequence of at least 200 consecutive amino acid residues of SEQ ID NO: 16. In some embodiments, the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16. In some embodiments, the Fab light chain polypeptide comprises an amino acid sequence according to SEQ ID NO: 16. In some embodiments, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20 and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21. In some embodiments, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO:21. In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO:22. In some embodiments, the multispecific antibody further comprises a half-life extending molecule (H₁). In some embodiments, H₁ is connected to P₁. In some embodiments, H₁ is connected to P₂. In some embodiments, H₁ does not block A₁ binding to CD28. In some embodiments, H₁ does not block B binding to PD-L1. In some embodiments, H₁ comprises a linking moiety (L₅) that connects H₁ to P₁ or H₁ to P₂. In some embodiments, the half-life extending molecule (H₁) does not have binding affinity to PD-L1. In some embodiments, the half-life extending molecule (H₁) does not have binding affinity to CD28. In some embodiments, the half-life extending molecule (H₁) does not shield the multispecific antibody from CD28. In some embodiments, H₁ comprises a sequence according to SEQ ID NOs: 54-57. In some embodiments, H₁ comprises an amino acid sequence that has repetitive sequence motifs. In some embodiments, H₁ comprises an amino acid sequence that has highly ordered secondary structure. In some embodiments, H₁ comprises a polymer. The isolated multispecific antibody of claim 148, wherein the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, sIgA, IgM or IgD. In some embodiments, the serum protein is albumin. In some embodiments, the polypeptide is an antibody. In some embodiments, the antibody comprises a single domain antibody, a single chain variable fragment, a Fab, or a Fab′. In some embodiments, the single domain antibody comprises a single domain antibody that binds to albumin. In some embodiments, the single domain antibody is a human or humanized antibody. In some embodiments, the single domain antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10E and SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3. In some embodiments, H₁ comprises an amino acid sequence according to SEQ ID NO: 57. In some embodiments, H₁ comprises an amino acid sequence that has at least 80% sequence identity to SEQ ID NO: 57. In some embodiments, H₁ comprises an amino acid sequence that has at least 85% sequence identity to SEQ ID NO: 57. In some embodiments, H₁ comprises an amino acid sequence that has at least 90% sequence identity to SEQ ID NO: 57. In some embodiments, H₁ comprises an amino acid sequence that has at least 95% sequence identity to SEQ ID NO: 57. In some embodiments, H₁ comprises an amino acid sequence that has at least 99% sequence identity to SEQ ID NO: 57. In some embodiments, H₁ comprise a modified amino acid or non-natural amino acid, or a modified non-natural amino acid, or a combination thereof. In some embodiments, the modified amino acid or a modified non-natural amino acid comprises a post-translational modification. In some embodiments, H₁ comprises a linking moiety (L₅) that connects H₁ to P₁ or P₂. In some embodiments, L₅ is a peptide sequence having at least 5 to no more than 50 amino acids. In some embodiments, L₅ is a peptide sequence having at least 10 to no more than 30 amino acids. In some embodiments, L₅ is a peptide sequence having at least 10 amino acids. In some embodiments, L₅ is a peptide sequence having at least 18 amino acids. In some embodiments, L₅ is a peptide sequence having at least 26 amino acids. In some embodiments, L₅ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the isolated multispecific antibody comprises an amino acid sequence that has at least 80% sequence identity to SEQ ID NOs: 149-170. In some embodiments, the isolated multispecific antibody comprises an amino acid sequence that has at least 85% sequence identity to SEQ ID NOs: 149-170. In some embodiments, the isolated multispecific antibody comprises an amino acid sequence that has at least 90% sequence identity to SEQ ID NOs: 149-170. In some embodiments, the isolated multispecific antibody comprises an amino acid sequence that has at least 95% sequence identity to SEQ ID NOs: 149-170. In some embodiments, the isolated multispecific antibody comprises an amino acid sequence that has at least 99% sequence identity to SEQ ID NOs: 149-170. In some embodiments, the isolated multispecific antibody comprises an amino acid sequence that has at least 95% sequence identity to SEQ ID NOs: 149 and 150. In some embodiments, the isolated multispecific antibody comprises an amino acid sequence that has at least 99% sequence identity to SEQ ID NOs: 149 and 150. In some embodiments, the isolated multispecific antibody comprises an amino acid sequence that has at least 95% sequence identity to SEQ ID NOs: 151 and 152. In some embodiments, the isolated multispecific antibody comprises an amino acid sequence that has at least 99% sequence identity to SEQ ID NOs: 151 and 152. In some embodiments, the isolated multispecific antibody comprises an amino acid sequence that has at least 95% sequence identity to SEQ ID NOs: 153 and 154. In some embodiments, the isolated multispecific antibody comprises an amino acid sequence that has at least 99% sequence identity to SEQ ID NOs: 153 and 154. In some embodiments, the isolated multispecific antibody comprises an amino acid sequence that has at least 95% sequence identity to SEQ ID NOs: 155 and 156. In some embodiments, the isolated multispecific antibody comprises an amino acid sequence that has at least 99% sequence identity to SEQ ID NOs: 155 and 156. In some embodiments, the isolated multispecific antibody comprises an amino acid sequence that has at least 95% sequence identity to SEQ ID NOs: 157 and 158. In some embodiments, the isolated multispecific antibody comprises an amino acid sequence that has at least 99% sequence identity to SEQ ID NOs: 157 and 158. In some embodiments, the isolated multispecific antibody comprises an amino acid sequence that has at least 95% sequence identity to SEQ ID NOs: 159 and 160. In some embodiments, the isolated multispecific antibody comprises an amino acid sequence that has at least 99% sequence identity to SEQ ID NOs: 159 and 160. In some embodiments, the isolated multispecific antibody comprises an amino acid sequence that has at least 95% sequence identity to SEQ ID NOs: 161 and 162. In some embodiments, the isolated multispecific antibody comprises an amino acid sequence that has at least 99% sequence identity to SEQ ID NOs: 161 and 162. In some embodiments, the isolated multispecific antibody comprises an amino acid sequence that has at least 95% sequence identity to SEQ ID NOs: 163 and 164. In some embodiments, the isolated multispecific antibody comprises an amino acid sequence that has at least 99% sequence identity to SEQ ID NOs: 163 and 164. In some embodiments, the isolated multispecific antibody comprises an amino acid sequence that has at least 95% sequence identity to SEQ ID NOs: 165 and 166. In some embodiments, the isolated multispecific antibody comprises an amino acid sequence that has at least 99% sequence identity to SEQ ID NOs: 165 and 166. In some embodiments, the isolated multispecific antibody comprises an amino acid sequence that has at least 95% sequence identity to SEQ ID NOs: 167 and 168. In some embodiments, the isolated multispecific antibody comprises an amino acid sequence that has at least 99% sequence identity to SEQ ID NOs: 167 and 168. In some embodiments, the isolated multispecific antibody comprises an amino acid sequence that has at least 95% sequence identity to SEQ ID NOs: 169 and 170. In some embodiments, the isolated multispecific antibody comprises an amino acid sequence that has at least 99% sequence identity to SEQ ID NOs: 169 and 170. In some embodiments, the isolated multispecific antibody comprises an amino acid sequence that has at least 95% sequence identity to SEQ ID NOs: 208 and 209. In some embodiments, the isolated multispecific antibody comprises an amino acid sequence that has at least 99% sequence identity to SEQ ID NOs: 208 and 209.

Disclosed herein are isolated recombinant nucleic acid molecules encoding a polypeptide of the isolated multispecific antibody of any one of the above embodiments.

Disclosed herein are pharmaceutical compositions comprising: (a) the isolated multispecific antibody of any one of the above embodiments; and (b) a pharmaceutically acceptable excipient.

Disclosed herein are pharmaceutical compositions comprising: (a) the isolated multispecific antibody of any one of the above embodiments, (b) an anti-cancer therapy, and (c) a pharmaceutically acceptable excipient. In some embodiments, the anti-cancer therapy comprises a small molecule, a cell-based therapy, or an antibody-based therapy. In some embodiments, the antibody-based therapy is a T cell engager. In some embodiments, the T cell engager comprises a formula according to: D₁-L₀-E₁ (Formula II), wherein D₁ comprises an effector cell binding domain that binds to an effector cell antigen, E₁ comprises a tumor antigen binding domain that binds to a tumor antigen, and L₀ comprises a linker that connects D₁ to E₁. In some embodiments, D₁ comprises a single chain variable fragment, a single domain antibody, a Fab fragment, or a Fab′. In some embodiments, D₁ comprises the single chain variable fragment. In some embodiments, E₁ comprises a single chain variable fragment, a single domain antibody, a Fab fragment, or a Fab′. In some embodiments, E₁ comprises the Fab fragment. In some embodiments, the effector cell antigen comprises CD3. In some embodiments, the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the effector cell binding domain comprises an amino acid sequence according to SEQ ID NOs: 89-101. In some embodiments, the tumor antigen comprises epidermal growth factor receptor (EGFR), prostate-specific membrane antigen (PSMA), or tumor-associated calcium signal transducer 2 (referred to herein after as TROP2). In some embodiments, the tumor antigen comprises EGFR. In some embodiments, the tumor antigen binding domain comprises an amino acid sequence according to SEQ ID NOs: 102-111. In some embodiments, the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 105; HC-CDR2: SEQ ID NO: 106; HC-CDR3: SEQ ID NO: 107; and wherein the LC-CDR1, the LC-CDR2, and the LC-CDR3 comprise: LC-CDR1: SEQ ID NO: 102; LC-CDR2: SEQ ID NO: 103 (YAS); and LC-CDR3: SEQ ID NO: 104. In some embodiments, the tumor antigen comprises EGFR, and the T cell engager comprises amino acid sequences with at least 95% sequence identity according to SEQ ID NOs: 181 and 182. In some embodiments, the tumor antigen comprises EGFR, and the T cell engager comprises amino acid sequences according to SEQ ID NOs: 181 and 182. In some embodiments, the tumor antigen comprises EGFR, and the T cell engager comprises amino acid sequences with at least 95% sequence identity according to SEQ ID NOs: 181 and 182. In some embodiments, the tumor antigen comprises EGFR, and the T cell engager comprises amino acid sequences according to SEQ ID NOs: 214 and 215. In some embodiments, the tumor antigen comprises TROP2. In some embodiments, the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 112; HC-CDR2: SEQ ID NO: 113; HC-CDR3: SEQ ID NO: 114; and wherein the LC-CDR1, the LC-CDR2, and the LC-CDR3 comprise: LC-CDR1: SEQ ID NO: 115; LC-CDR2: SEQ ID NO: 116 (SAS); and LC-CDR3: SEQ ID NO: 117. In some embodiments, the tumor antigen comprises TROP2, and the T cell engager comprises amino acid sequences with at least 95% sequence identity according to SEQ ID NOs: 187-192. In some embodiments, the tumor antigen comprises TROP2, and the T cell engager comprises amino acid sequences according to any one of SEQ ID NOs: 187-192. In some embodiments, the tumor antigen binding domain comprises an amino acid sequence according to SEQ ID NOs: 112-119. In some embodiments, the tumor antigen comprises PSMA. In some embodiments, the tumor antigen binding domain comprises an amino acid sequence according to SEQ ID NOs: 120-127. In some embodiments, the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 120; HC-CDR2: SEQ ID NO: 121; HC-CDR3: SEQ ID NO: 122; and wherein the LC-CDR1, the LC-CDR2, and the LC-CDR3 comprise: LC-CDR1: SEQ ID NO: 123; LC-CDR2: SEQ ID NO: 124 (EA); and LC-CDR3: SEQ ID NO: 125. In some embodiments, the tumor antigen comprises PSMA, and the T cell engager comprises amino acid sequences with at least 95% sequence identity according to SEQ ID NOs: 173 and 174. In some embodiments, the tumor antigen comprises PSMA, and the T cell engager comprises amino acid sequences according to SEQ ID NOs: 173 and 174. In some embodiments, the T cell engager molecule is selectively activated in tumor microenvironments. In some embodiments, the T cell engager is according to the following subformula: P₃-L₃-D₁-L₀-E₁ (Formula IIa) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease. In some embodiments, the T cell engager is according to the following subformula: D₁-L₀-E₁-L₄-P₄ (Formula IIb) wherein D₁ comprises the CD3 binding domain; E₁ comprises the tumor antigen binding domain; L₀ comprises the linker that connects D₁ to E₁; P₄ comprises a peptide that binds to E₁ and L₄ comprises a linking moiety that connects E₁ to P₄ and is a substrate for a tumor specific protease. In some embodiments, the T cell engager is according to the following subformula: P₃-L₃-D₁-L₀-E₁-L₄-P₄ (Formula IIc) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease; P₄ comprises a peptide that binds to E₁ and L₄ comprises a linking moiety that connects E₁ to P₄ and is a substrate for a tumor specific protease. In some embodiments, the T cell engager comprises H₁. In some embodiments, H₁ comprises a sequence according to SEQ ID NO: 54-57. In some embodiments, H₁ comprises a single domain antibody. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56. In some embodiments, L₃ or L₄ is a peptide sequence having at least 5 to no more than 50 amino acids. In some embodiments, L₃ or L₄ is a peptide sequence having at least 10 to no more than 30 amino acids. In some embodiments, L₃ or L₄ is a peptide sequence having at least 10 amino acids. In some embodiments, L₃ or L₄ is a peptide sequence having at least 18 amino acids. In some embodiments, L₃ or L₄ is a peptide sequence having at least 26 amino acids. In some embodiments, L₃ or L₄ comprises a formula comprising (G2S)n, wherein n is an integer from 1 to 3 (SEQ ID NO: 228). In some embodiments, L₃ or L₄ comprises a formula comprising (G2S)n, wherein n is an integer of at least 1. In some embodiments, L₃ or L₄ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments, L₃ or L₄ comprises a urokinase cleavable amino acid sequence, a matriptase cleavable amino acid sequence, or a matrix metalloprotease cleavable amino acid sequence. In some embodiments, L₃ or L₄ comprises a sequence according to SEQ ID NOs: 18-19, 62-88. In some embodiments, L₃ is bound to N-terminus of D₁. In some embodiments, L₃ is bound to C-terminus of D₁. In some embodiments, L₄ is bound to N-terminus of E₁. In some embodiments, L₄ is bound to C-terminus of E₁. In some embodiments, P₃ becomes unbound from D₁ when L₃ is cleaved by the tumor specific protease thereby exposing D₁ to CD3. In some embodiments, P₄ becomes unbound from E₁ when L₄ is cleaved by the tumor specific protease thereby exposing E₁ to the tumor antigen. In some embodiments, P₃ impairs binding of D₁ to CD3. In some embodiments, P₃ is bound to D₁ through ionic interactions, electrostatic interactions, hydrophobic interactions, Pi-stacking interactions, and H-bonding interactions, or a combination thereof. In some embodiments, P₃ is bound to D₁ at or near an antigen binding site. In some embodiments, P₃ becomes unbound from D₁ when L₃ is cleaved by the tumor specific protease thereby exposing D₁ to CD3. In some embodiments, P₃ has less than 70% sequence identity to CD3. In some embodiments, P₃ has less than 85% sequence identity to CD3. In some embodiments, P₃ has less than 90% sequence identity to CD3. In some embodiments, P₃ has less than 95% sequence identity to CD3. In some embodiments, P₃ has less than 98% sequence identity to CD3. In some embodiments, P₃ has less than 99% sequence identity to CD3. In some embodiments, P₃ comprises the amino acid sequence according to SEQ ID NOs: 177-180. In some embodiments, P₃ comprises a de novo amino acid sequence that shares less than 10% sequence identity to CD3. In some embodiments, P₄ impairs binding of E₁ to the tumor antigen. In some embodiments, P₄ is bound to E₁ through ionic interactions, electrostatic interactions, hydrophobic interactions, Pi-stacking interactions, and H-bonding interactions, or a combination thereof. In some embodiments, P₄ is bound to E₁ at or near an antigen binding site. In some embodiments, P₄ becomes unbound from E₁ when L₄ is cleaved by the tumor specific protease thereby exposing E₁ to the tumor antigen. In some embodiments, P₄ has less than 70% sequence identity to the tumor antigen. In some embodiments, P₄ has less than 80% sequence identity to the tumor antigen. In some embodiments, P₄ has less than 85% sequence identity to the tumor antigen. In some embodiments, P₄ has less than 90% sequence identity to the tumor antigen. In some embodiments, P₄ has less than 95% sequence identity to the tumor antigen. In some embodiments, P₄ comprises a de novo amino acid sequence that shares less than 10% sequence identity to the tumor antigen. In some embodiments, P₃ or P₄ comprises a peptide sequence of at least 5 amino acids in length. In some embodiments, P₃ or P₄ comprises a peptide sequence of at least 6 amino acids in length. In some embodiments, P₃ or P₄ comprises a peptide sequence of at least 10 amino acids in length. In some embodiments, P₃ or P₄ comprises a peptide sequence of at least 10 amino acids in length and no more than 20 amino acids in length. In some embodiments, P₃ or P₄ comprises a peptide sequence of at least 16 amino acids in length. In some embodiments, P₃ or P₄ comprises a peptide sequence of no more than 40 amino acids in length. In some embodiments, P₃ or P₄ comprises at least two cysteine amino acid residues. In some embodiments, P₃ or P₄ comprises a cyclic peptide or a linear peptide. In some embodiments, P₃ or P₄ comprises a cyclic peptide. In some embodiments, P₃ or P₄ comprises a linear peptide. In some embodiments, P₄ comprises the amino acid sequence according to SEQ ID NO: 185 or 186. In some embodiments, the tumor antigen comprises EGFR, and the T cell engager comprises the amino acid sequence of SEQ ID NOs: 183 and 184. In some embodiments, P₄ comprises the amino acid sequence according to SEQ ID NOs: 199-201. In some embodiments, the tumor antigen comprises TROP2, and the T cell engager comprises any one of amino acid sequences of SEQ ID NOs: 193-198. In some embodiments, the tumor antigen comprises PSMA, and the T cell engager comprises the amino acid sequence of SEQ ID NOs: 175 and 176.

Disclosed herein are isolated polypeptides or polypeptide complexes comprising a CD28 binding domain that is linked to a peptide that impairs binding of the CD28 binding domain to CD28 wherein the peptide comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20. In some embodiments, the peptide comprises an amino acid sequence according to any one of SEQ ID NOs: 24-53, 128-148, and the amino acid sequences of Table 20. In some embodiments, the peptide comprises an amino acid sequence according to any one of SEQ ID NOs: 42-53 or an amino acid sequence that has 1, 2, or 3 amino acid mutations, substitutions, or deletions relative to any one of SEQ ID NOs: 42-53. In some embodiments, the peptide comprises an amino acid sequence according to any one of SEQ ID NOs: 42-53. In some embodiments, the peptide comprises an amino acid sequence according to any one of the amino acid sequences of Table 20 or an amino acid sequence that has 1, 2, or 3 amino acid mutations, substitutions, or deletions relative to any one of the amino acid sequences of Table 20. In some embodiments, the peptide comprises an amino acid sequence according to any one of the amino acid sequences of Table 20. In some embodiments, the peptide comprises an amino acid sequence according to any one of SEQ ID NOs: 128-147 or an amino acid sequence that has 1, 2, or 3 amino acid mutations, substitutions, or deletions relative to any one of SEQ ID NOs: 128-147. In some embodiments, the peptide comprises an amino acid sequence according to any one of SEQ ID NOs: 128-147. In some embodiments, the peptide comprises an amino acid sequence according to X₁-X₂-X₃-C-X₄-X₅-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₁) is selected from E, V, L, D, and H; X₁₁ is selected from F, Y, and L; and X₁₂ is selected from N, A, F, S, and Y. In some embodiments, X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F. In some embodiments, the peptide comprises an amino acid sequence according to SEQ ID NO: 32 or an amino acid sequence that has 1, 2, or 3 amino acid mutations, substitutions, or deletions relative to SEQ ID NO: 32. In some embodiments, the peptide comprises an amino acid sequence according to SEQ ID NO: 32. In some embodiments, the peptide comprises an amino acid sequence according to SEQ ID NO: 138 or an amino acid sequence that has 1, 2, or 3 amino acid mutations, substitutions, or deletions relative to SEQ ID NO: 138. In some embodiments, the peptide comprises an amino acid sequence according to SEQ ID NO: 138. In some embodiments, the CD28 binding domain comprises a single chain variable fragment, a single domain antibody, a Fab, or a Fab′. In some embodiments, the CD28 binding domain comprises the single chain variable fragment and the single chain variable fragment comprises a scFv heavy chain variable domain and a scFv light chain variable domain. In some embodiments, the CD28 binding domain comprises the single domain antibody. The isolated polypeptide or polypeptide complex of claim 313, wherein the CD28 binding domain comprises the Fab or the Fab′. In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5 (KA); and LC-CDR3: SEQ ID NO: 6, and wherein the CDRs comprise from 0-2 amino acid modifications in at least one of said LC-CDR1, LC-CDR2, or LC-CDR3. In some embodiments, the scFv heavy chain variable domain comprises an amino acid sequence that has at least 80% sequence identity to the amino acid sequence according to SEQ ID NO: 7. In some embodiments, the scFv heavy chain variable domain comprises an amino acid sequence of at least 75 consecutive amino acid residues of SEQ ID NO: 7. In some embodiments, the scFv heavy chain variable domain comprises an amino acid sequence of at least 110 consecutive amino acid residues of SEQ ID NO: 7. In some embodiments, the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7. In some embodiments, the scFv heavy chain variable domain comprises an amino acid sequence according to SEQ ID NO: 7. In some embodiments, the scFv light chain variable domain comprises an amino acid sequence that has at least 80% sequence identity to the amino acid sequence according to SEQ ID NO: 8. In some embodiments, the scFv light chain variable domain comprises an amino acid sequence of at least 75 consecutive amino acid residues of SEQ ID NO: 8. In some embodiments, the scFv light chain variable domain comprises an amino acid sequence of at least 100 consecutive amino acid residues of SEQ ID NO: 8. In some embodiments, the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8. In some embodiments, the scFv light chain variable domain comprises an amino acid sequence according to SEQ ID NO: 8. In some embodiments, the scFv comprises an amino acid sequence that has at least 80% sequence identity to the amino acid sequence according to SEQ ID NO: 9. In some embodiments, the scFv comprises an amino acid sequence of at least 175 consecutive amino acid residues of SEQ ID NO: 9. In some embodiments, the scFv comprises an amino acid sequence of at least 210 consecutive amino acid residues of SEQ ID NO: 9. In some embodiments, the scFv comprises an amino acid sequence of at least 210 consecutive amino acid residues of SEQ ID NO: 9 and has at least 80% sequence identity to the at least 210 consecutive amino acid residues of SEQ ID NO: 9. In some embodiments, the scFv comprises an amino acid sequence according to SEQ ID NO: 9. In some embodiments, the CD28 binding domain is linked to the peptide through a linking moiety (L₁). In some embodiments, L₁ is a substrate for a tumor specific protease. In some embodiments, L₁ is a peptide sequence having at least 5 to no more than 50 amino acids. In some embodiments, L₁ is a peptide sequence having at least 10 to no more than 30 amino acids. In some embodiments, L₁ is a peptide sequence having at least 10 amino acids. In some embodiments, L₁ is a peptide sequence having at least 18 amino acids. In some embodiments, L₁ is a peptide sequence having at least 26 amino acids. In some embodiments, L₁ comprises a formula comprising (G₂S)ₙ, wherein n is an integer from 1 to 3 (SEQ ID NO: 228). In some embodiments, L₁ comprises a formula comprising (G₂S)ₙ, wherein n is an integer of at least 1. In some embodiments, L₁ comprises a formula selected from the group consisting of (G₂S)n, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments, L₁ comprises a urokinase cleavable amino acid sequence, a matriptase cleavable amino acid sequence, or a matrix metalloprotease cleavable amino acid sequence. In some embodiments, L₁ comprises a sequence according to SEQ ID NOs: 18-19, 62-88. In some embodiments, L₁ is bound to N-terminus of A₁. In some embodiments, L₁ is bound to C-terminus of A₁. In some embodiments, P₁ becomes unbound from A₁ when L₁ is cleaved by the tumor specific protease thereby exposing A₁ to CD28. In some embodiments, L₁ comprise a modified amino acid or non-natural amino acid, or a modified non-natural amino acid, or a combination thereof. In some embodiments, the modified amino acid or a modified non-natural amino acid comprises a post-translational modification. In some embodiments, the isolated polypeptide or polypeptide complex further comprises a half-life extending molecule (H₁). In some embodiments, H₁ is connected to the peptide. In some embodiments, H₁ does not block the CD28 binding domain to CD28. In some embodiments, H₁ comprises a linking moiety (L₅) that connects H₁ to the peptide. In some embodiments, the half-life extending molecule (H₁) does not have binding affinity to CD28. In some embodiments, the half-life extending molecule (H₁) does not shield the isolated polypeptide or polypeptide complex from CD28. In some embodiments, H₁ comprises a sequence according to SEQ ID NOs: 54-57. In some embodiments, H₁ comprises an amino acid sequence that has repetitive sequence motifs. In some embodiments, H₁ comprises an amino acid sequence that has highly ordered secondary structure. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, sIgA, IgM or IgD. In some embodiments, the serum protein is albumin. In some embodiments, the polypeptide is an antibody. In some embodiments, the antibody comprises a single domain antibody, a single chain variable fragment, a Fab, or a Fab′. In some embodiments, the single domain antibody comprises a single domain antibody that binds to albumin. In some embodiments, the single domain antibody is a human or humanized antibody. In some embodiments, the single domain antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10E and SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3. In some embodiments, H₁ comprises an amino acid sequence according to SEQ ID NO: 57. In some embodiments, H₁ comprises an amino acid sequence that has at least 80% sequence identity to SEQ ID NO: 57. In some embodiments, H₁ comprises an amino acid sequence that has at least 85% sequence identity to SEQ ID NO: 57. In some embodiments, H₁ comprises an amino acid sequence that has at least 90% sequence identity to SEQ ID NO: 57. In some embodiments, H₁ comprises an amino acid sequence that has at least 95% sequence identity to SEQ ID NO: 57. In some embodiments, H₁ comprises an amino acid sequence that has at least 99% sequence identity to SEQ ID NO: 57. In some embodiments, H₁ comprise a modified amino acid or non-natural amino acid, or a modified non-natural amino acid, or a combination thereof. In some embodiments, the modified amino acid or a modified non-natural amino acid comprises a post-translational modification. In some embodiments, H₁ comprises a linking moiety (L₅) that connects H₁ to P₁ or P₂. In some embodiments, L₅ is a peptide sequence having at least 5 to no more than 50 amino acids. In some embodiments, L₅ is a peptide sequence having at least 10 to no more than 30 amino acids. In some embodiments, L₅ is a peptide sequence having at least 10 amino acids. In some embodiments, L₅ is a peptide sequence having at least 18 amino acids. In some embodiments, L₅ is a peptide sequence having at least 26 amino acids. In some embodiments, L₅ comprises a formula selected from the group consisting of (G₂S)n, (GS)n, (GSGGS)n (SEQ ID NO: 58), (GGGS)n (SEQ ID NO: 59), (GGGGS)n (SEQ ID NO: 60), and (GSSGGS)n (SEQ ID NO: 61), wherein n is an integer of at least 1.

Disclosed herein are methods of treating cancer in a subject in need thereof comprising administering to the subject the multispecific antibody of any one of the above embodiments. In some embodiments, the multispecific antibody induces T cell mediated cytotoxicity of tumor cells. In some embodiments, the cancer is a hematological malignancy. In some embodiments, the cancer is leukemia or lymphoma. In some embodiments, the cancer is lymphoma, and wherein the lymphoma is B-cell lymphoma. In some embodiments, the cancer is a solid tumor. In some embodiments, the solid tumor expresses PD-L1. In some embodiments, the solid tumor is sarcoma, breast cancer, lung cancer, or carcinoma. In some embodiments, the solid tumor is lung cancer, and wherein the lung cancer is non-small cell lung cancer. In some embodiments, the multispecific antibody is administered in combination with an anti-cancer therapy. In some embodiments, the multispecific antibody and the anti-cancer therapy are administered in the same pharmaceutical composition. In some embodiments, the multispecific antibody and the anti-cancer therapy are administered as separate pharmaceutical compositions. In some embodiments, the subject is refractory to checkpoint inhibitor therapy. In some embodiments, the subject has relapsed from checkpoint inhibitor therapy. In some embodiments, the anti-cancer therapy comprises a small molecule, a cell-based therapy, or an antibody-based therapy. In some embodiments, the antibody-based therapy is a T cell engager. In some embodiments, the T cell engager comprises a formula according to: D₁-L₀-E₁ (Formula II), wherein D₁ comprises an effector cell binding domain that binds to an effector cell antigen, E₁ comprises a tumor antigen binding domain that binds to a tumor antigen, and L₀ comprises a linker that connects D₁ to E₁. In some embodiments, D₁ comprises a single chain variable fragment, a single domain antibody, a Fab fragment, or a Fab′. In some embodiments, D₁ comprises the single chain variable fragment. In some embodiments, E₁ comprises a single chain variable fragment, a single domain antibody, a Fab fragment, or a Fab′. In some embodiments, E₁ comprises the Fab fragment. In some embodiments, the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the effector cell binding domain comprises an amino acid sequence according to SEQ ID NOs: 89-101. In some embodiments, the tumor antigen comprises epidermal growth factor receptor (EGFR), prostate-specific membrane antigen (PSMA), or tumor-associated calcium signal transducer 2 (referred to herein after as TROP2). In some embodiments, the tumor antigen comprises EGFR. In some embodiments, the tumor antigen binding domain comprises an amino acid sequence according to SEQ ID NOs: 102-111. In some embodiments, the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 105; HC-CDR2: SEQ ID NO: 106; HC-CDR3: SEQ ID NO: 107; and wherein the LC-CDR1, the LC-CDR2, and the LC-CDR3 comprise: LC-CDR1: SEQ ID NO: 102; LC-CDR2: SEQ ID NO: 103 (YAS); and LC-CDR3: SEQ ID NO: 104. In some embodiments, the tumor antigen comprises EGFR, and the T cell engager comprises amino acid sequences with at least 95% sequence identity according to SEQ ID NOs: 181 and 182. In some embodiments, the tumor antigen comprises EGFR, and the T cell engager comprises amino acid sequences according to SEQ ID NOs: 181 and 182. In some embodiments, the tumor antigen comprises EGFR, and the T cell engager comprises amino acid sequences with at least 95% sequence identity according to SEQ ID NOs: 181 and 182. In some embodiments, the tumor antigen comprises EGFR, and the T cell engager comprises amino acid sequences according to SEQ ID NOs: 214 and 215. In some embodiments, the cancer is colorectal cancer (CRC), squamous cell carcinoma of the head and Neck (SCCHN), non-small cell lung cancer (NSCLC), prostate cancer, breast cancer, colon/rectum cancer, head and neck cancer, esophagogastric cancer, liver cancer, glioblastoma, cervical cancer, ovarian cancer, bladder cancer, kidney cancer, or pancreatic cancer. In some embodiments, the tumor antigen comprises TROP2. In some embodiments, the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 112; HC-CDR2: SEQ ID NO: 113; HC-CDR3: SEQ ID NO: 114; and wherein the LC-CDR1, the LC-CDR2, and the LC-CDR3 comprise: LC-CDR1: SEQ ID NO: 115; LC-CDR2: SEQ ID NO: 116 (SAS); and LC-CDR3: SEQ ID NO: 117. In some embodiments, the tumor antigen comprises TROP2, and the T cell engager comprises amino acid sequences with at least 95% sequence identity according to SEQ ID NOs: 187-192. In some embodiments, the tumor antigen comprises TROP2, and the T cell engager comprises amino acid sequences according to any one of SEQ ID NOs: 187-192. In some embodiments, the tumor antigen binding domain comprises an amino acid sequence according to SEQ ID NOs: 112-119. The method of claim 416, wherein the cancer is the cancer is lung, breast (e.g. HER2+; ER/PR+; TNBC), cervical, ovarian, colorectal, pancreatic, gastric, triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer. In some embodiments, the tumor antigen comprises PSMA. In some embodiments, the tumor antigen binding domain comprises an amino acid sequence according to SEQ ID NOs: 120-127. In some embodiments, the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 120; HC-CDR2: SEQ ID NO: 121; HC-CDR3: SEQ ID NO: 122; and wherein the LC-CDR1, the LC-CDR2, and the LC-CDR3 comprise: LC-CDR1: SEQ ID NO: 123; LC-CDR2: SEQ ID NO: 124 (EA); and LC-CDR3: SEQ ID NO: 125. In some embodiments, the tumor antigen comprises PSMA, and the T cell engager comprises amino acid sequences with at least 95% sequence identity according to SEQ ID NOs: 173 and 174. In some embodiments, the tumor antigen comprises PSMA, and the T cell engager comprises amino acid sequences according to SEQ ID NOs: 173 and 174. In some embodiments, the cancer is cancer is lung, breast (e.g. HER2+; ER/PR+; TNBC), cervical, ovarian, colorectal, pancreatic or gastric. In some embodiments, the T cell engager molecule is selectively activated in tumor microenvironments.

In some embodiments, the T cell engager is according to the following subformula: P₃-L₃-D₁-L₀-E₁ (Formula IIa) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease. In some embodiments, the T cell engager is according to the following subformula: D₁-L₀-E₁-L₄-P₄ (Formula IIb) wherein D₁ comprises the CD3 binding domain; E₁ comprises the tumor antigen binding domain; L₀ comprises the linker that connects D₁ to E₁; P₄ comprises a peptide that binds to E₁ and L₄ comprises a linking moiety that connects E₁ to P₄ and is a substrate for a tumor specific protease. In some embodiments, the T cell engager is according to the following subformula: P₃-L₃-D₁-L₀-E₁-L₄-P₄ (Formula IIc) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease; P₄ comprises a peptide that binds to E₁ and L₄ comprises a linking moiety that connects E₁ to P₄ and is a substrate for a tumor specific protease. In some embodiments, the T cell engager comprises H₁. In some embodiments, H₁ comprises a sequence according to SEQ ID NO: 54-57. In some embodiments, H₁ comprises a single domain antibody. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56. In some embodiments, L₃ or L₄ is a peptide sequence having at least 5 to no more than 50 amino acids. In some embodiments, L₃ or L₄ is a peptide sequence having at least 10 to no more than 30 amino acids. In some embodiments, L₃ or L₄ is a peptide sequence having at least 10 amino acids. In some embodiments, L₃ or L₄ is a peptide sequence having at least 18 amino acids. In some embodiments, L₃ or L₄ is a peptide sequence having at least 26 amino acids. In some embodiments, L₃ or L₄ comprises a formula comprising (G₂S)ₙ, wherein n is an integer from 1 to 3 (SEQ ID NO: 228). In some embodiments, L₃ or L₄ comprises a formula comprising (G₂S)ₙ, wherein n is an integer of at least 1. In some embodiments, L₃ or L₄ comprises a formula selected from the group consisting of (G₂S)ₙ, (GS)ₙ, (GSGGS)ₙ (SEQ ID NO: 58), (GGGS)ₙ (SEQ ID NO: 59), (GGGGS). (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments, L₃ or L₄ comprises a urokinase cleavable amino acid sequence, a matriptase cleavable amino acid sequence, or a matrix metalloprotease cleavable amino acid sequence. In some embodiments, L₃ or L₄ comprises a sequence according to SEQ ID NOs: 18-19, 62-88. In some embodiments, L₃ is bound to N-terminus of D₁. In some embodiments, L₃ is bound to C-terminus of D₁. In some embodiments, L₄ is bound to N-terminus of E₁. In some embodiments, L₄ is bound to C-terminus of E₁. In some embodiments, P₃ becomes unbound from D₁ when L₃ is cleaved by the tumor specific protease thereby exposing D₁ to CD3. In some embodiments, P₄ becomes unbound from E₁ when L₄ is cleaved by the tumor specific protease thereby exposing E₁ to the tumor antigen. In some embodiments, P₃ impairs binding of D₁ to CD3. In some embodiments, P₃ is bound to D₁ through ionic interactions, electrostatic interactions, hydrophobic interactions, Pi-stacking interactions, and H-bonding interactions, or a combination thereof. In some embodiments, P₃ is bound to D₁ at or near an antigen binding site. In some embodiments, P₃ becomes unbound from D₁ when L₃ is cleaved by the tumor specific protease thereby exposing D₁ to CD3. In some embodiments, P₃ has less than 70% sequence identity to CD3. In some embodiments, P₃ has less than 85% sequence identity to CD3. In some embodiments, P₃ has less than 90% sequence identity to CD3. In some embodiments, P₃ has less than 95% sequence identity to CD3. In some embodiments, P₃ has less than 98% sequence identity to CD3. In some embodiments, P₃ has less than 99% sequence identity to CD3. In some embodiments, P₃ comprises the amino acid sequence according to SEQ ID NOs: 177-180. In some embodiments, P₃ comprises a de novo amino acid sequence that shares less than 10% sequence identity to CD3. In some embodiments, P₄ impairs binding of E₁ to the tumor antigen. In some embodiments, P₄ is bound to E₁ through ionic interactions, electrostatic interactions, hydrophobic interactions, Pi-stacking interactions, and H-bonding interactions, or a combination thereof. In some embodiments, P₄ is bound to E₁ at or near an antigen binding site. In some embodiments, P₄ becomes unbound from E₁ when L₄ is cleaved by the tumor specific protease thereby exposing E₁ to the tumor antigen. In some embodiments, P₄ has less than 70% sequence identity to the tumor antigen. In some embodiments, P₄ has less than 80% sequence identity to the tumor antigen. In some embodiments, P₄ has less than 85% sequence identity to the tumor antigen. In some embodiments, P₄ has less than 90% sequence identity to the tumor antigen. In some embodiments, P₄ has less than 95% sequence identity to the tumor antigen. In some embodiments, P₄ comprises a de novo amino acid sequence that shares less than 10% sequence identity to the tumor antigen. In some embodiments, P₃ or P₄ comprises a peptide sequence of at least 5 amino acids in length. In some embodiments, P₃ or P₄ comprises a peptide sequence of at least 6 amino acids in length. In some embodiments, P₃ or P₄ comprises a peptide sequence of at least 10 amino acids in length. In some embodiments, P₃ or P₄ comprises a peptide sequence of at least 10 amino acids in length and no more than 20 amino acids in length. In some embodiments, P₃ or P₄ comprises a peptide sequence of at least 16 amino acids in length. In some embodiments, P₃ or P₄ comprises a peptide sequence of no more than 40 amino acids in length. In some embodiments, P₃ or P₄ comprises at least two cysteine amino acid residues. In some embodiments, P₃ or P₄ comprises a cyclic peptide or a linear peptide. In some embodiments, P₃ or P₄ comprises a cyclic peptide. In some embodiments, P₃ or P₄ comprises a linear peptide. In some embodiments, P₄ comprises the amino acid sequence according to SEQ ID NO: 185 or 186. In some embodiments, the tumor antigen comprises EGFR, and the T cell engager comprises the amino acid sequence of SEQ ID NOs: 183 and 184. In some embodiments, P₄ comprises the amino acid sequence according to SEQ ID NOs: 199-201. In some embodiments, the tumor antigen comprises TROP2, and the T cell engager comprises any one of amino acid sequences of SEQ ID NOs: 193-198. In some embodiments, the tumor antigen comprises PSMA, and the T cell engager comprises the amino acid sequence of SEQ ID NOs: 175 and 176.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B illustrate exemplary schemas of anti-PDL1×CD28 multispecific antibodies. FIG. 1A illustrates “Vh” format of the antibody Fab-scFv format. FIG. 1B illustrates “V1” format of the Fab-scFv antibody format.

FIG. 2 illustrates a schematic for identifying peptides that can be attached to the anti-PD-L1 and anti-CD28 multispecific antibodies for selective activation in tumor microenvironments. The schematic illustrates a directed evolution and phage display technology to identify peptides that block antigen recognition by antigen binding domains.

FIG. 3A illustrates anti-CD28 scFv binding to peptides measured by ELISA.

FIG. 3B illustrates Ab-12 binding to peptides measured by ELISA.

FIG. 3C illustrates anti-CD28 scFv binding to peptides measured by ELISA.

FIG. 3D illustrates Ab-12 binding to peptides measured by ELISA.

FIGS. 3E-3F illustrate that peptides inhibit anti-CD28 scFv from binding to CD28 antigen as measured by ELISA.

FIG. 3G illustrates that peptides inhibit Ab-12 from binding CD28 antigen as measured by ELISA.

FIGS. 4A-4D illustrate kinetic binding of anti-CD28 scFv or Ab-12 to Peptide-9 and Peptide-12 by Octet.

FIGS. 5A-5B illustrate binding of anti-CD28 scFv to Ala scan peptides of Peptide-9.

FIGS. 6A-6B illustrate inhibition of anti-CD28 scFv by Ala scan peptides of Peptide-9.

FIG. 7 illustrates the core sequence motif of optimized anti-CD28 scFv Peptide-9 sequences generated using WebLogo 3.7.4.

FIGS. 8A-8C illustrate peptides that inhibit the anti-CD28 scFv from binding the CD28 antigen measured by ELISA.

FIGS. 9A-9C illustrate peptides that inhibit Ab-12 from binding the CD28 antigen by ELISA.

FIGS. 10A-10U illustrate kinetic binding of anti-CD28 scFv binding to peptides as measured by Octet.

FIG. 11A illustrates binding of Ab-12 and an anti-PD-L1 Fab 1 (SEQ ID NOs: 16 and 17) to PD-L1 as measured by ELISA.

FIG. 11B illustrates binding of Ab-12 and an anti-CD28 scFv (SEQ ID NO: 9) to CD28 as measured by ELISA.

FIG. 11C illustrates binding of Ab-12 and Ab-13 to PD-L1 as measured by ELISA.

FIG. 11D illustrates binding of Ab-12 and Ab-13 to CD28 as measured by ELISA.

FIG. 11E illustrates binding of Ab-1, Ab-2, Ab-3, Ab-4, Ab-5, Ab-6, and Ab-12 to PD-L1 as measured by ELISA. In some circumstances, the antibodies are incubated with MTSP1.

FIG. 11F illustrates binding of Ab-12, Ab-1, Ab-2, Ab-3, Ab-4, Ab-5, Ab-6, and Ab-7 to CD28 as measured by ELISA. In some circumstances, the antibodies are incubated with MTSP1.

FIG. 11G illustrates binding of Ab-12, Ab-1, Ab-2, Ab-5, and Ab-6 to PD-L1 as measured by ELISA. In some circumstances, the antibodies are incubated with MMP9.

FIG. 1111 illustrates binding of Ab-12, Ab-1, Ab-2, Ab-5, and Ab-6 to CD28 as measured by ELISA. In some circumstances, the antibodies are incubated with MMP9.

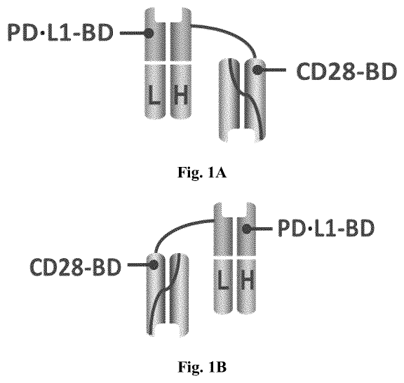

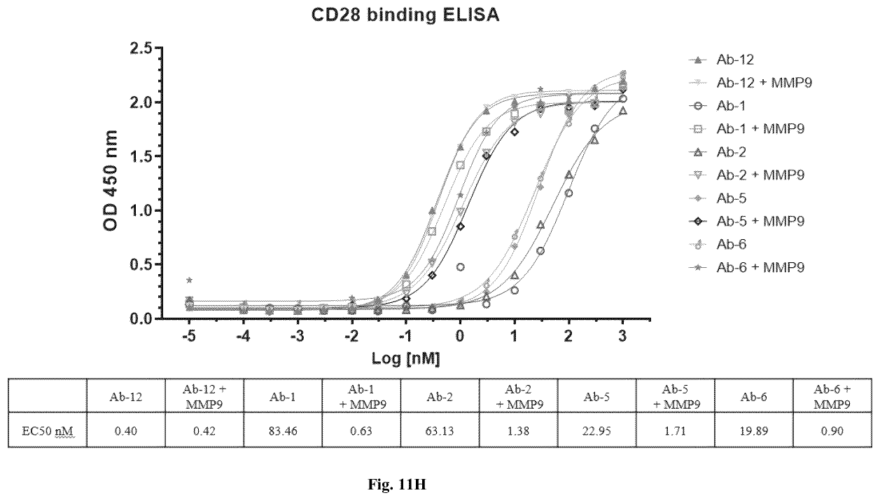

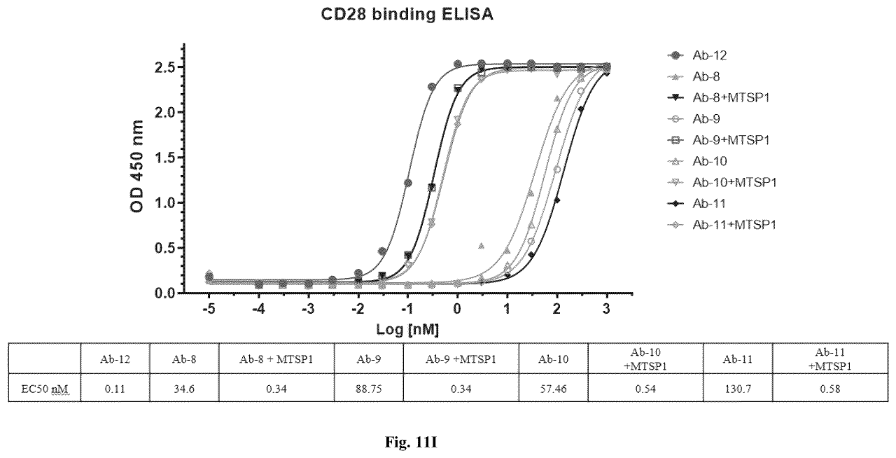

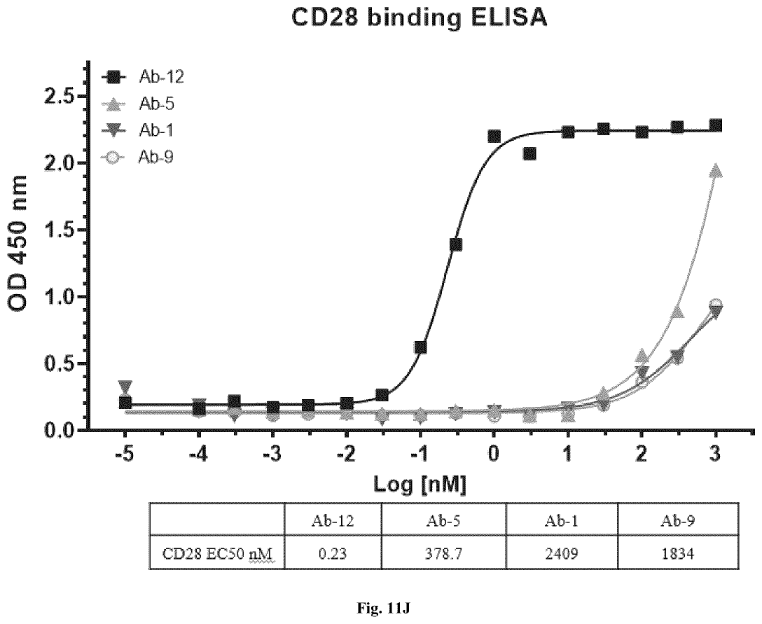

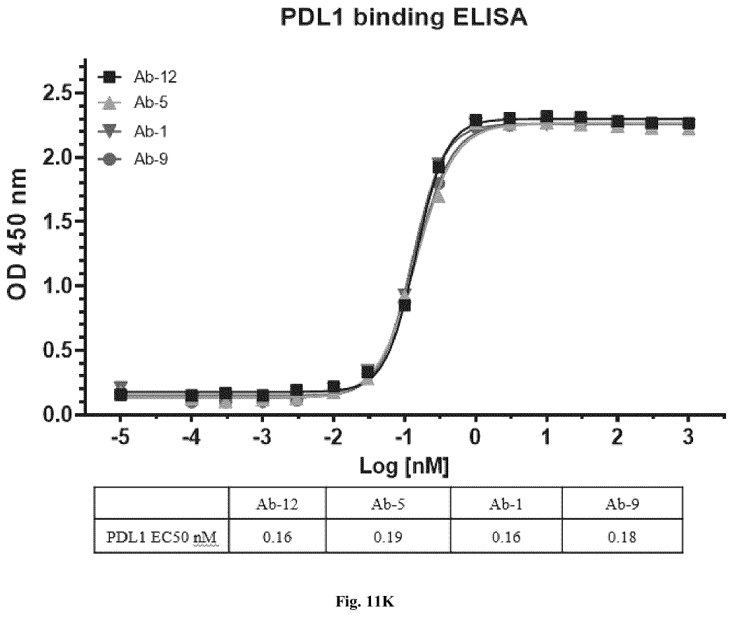

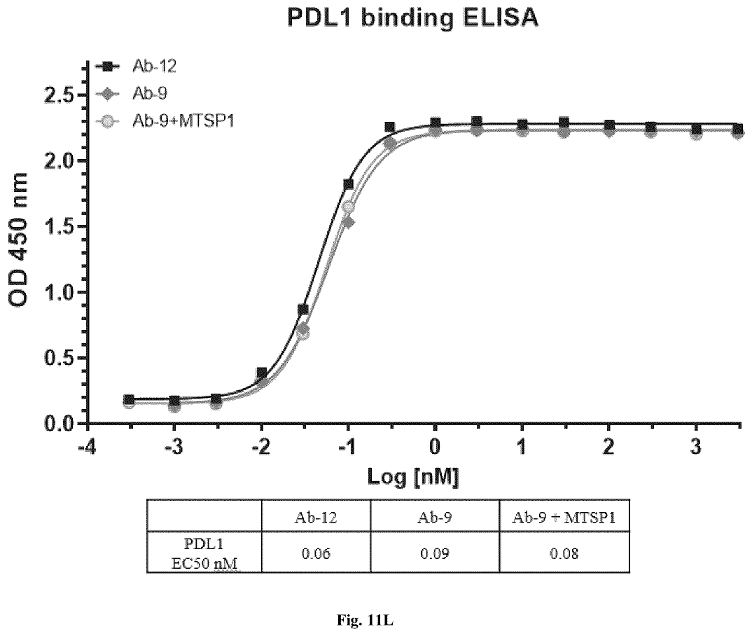

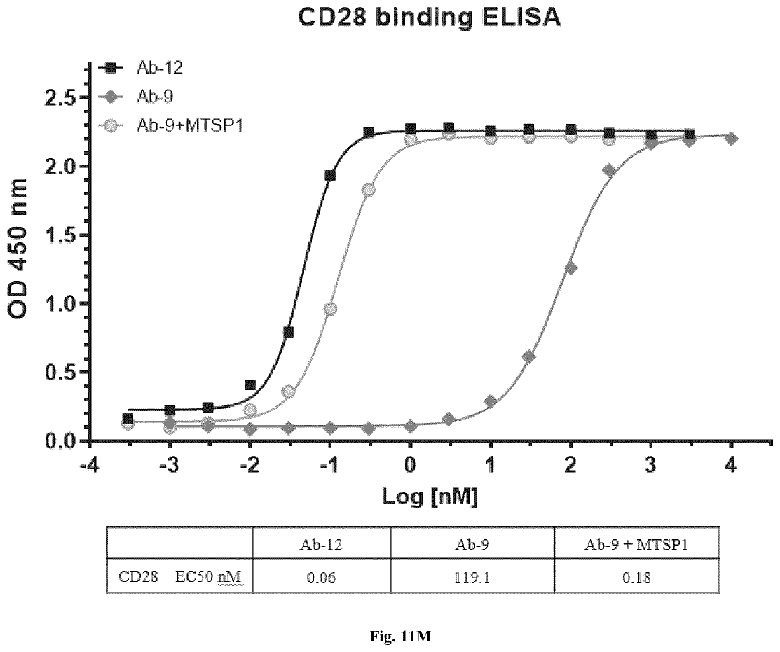

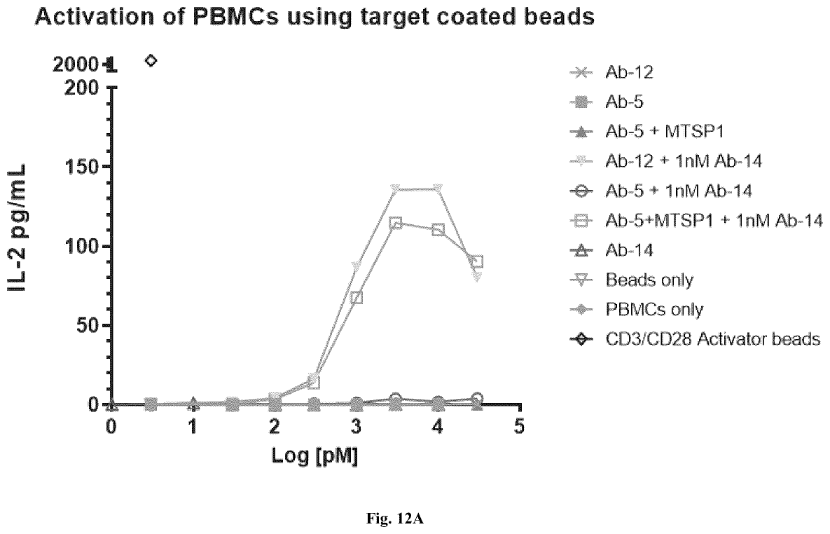

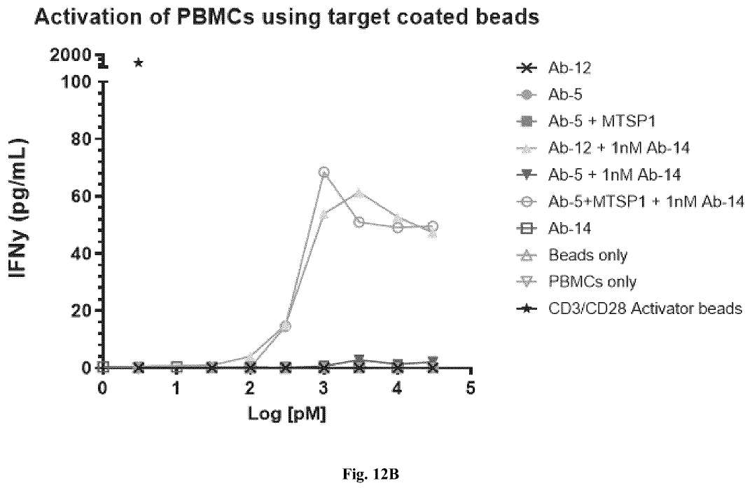

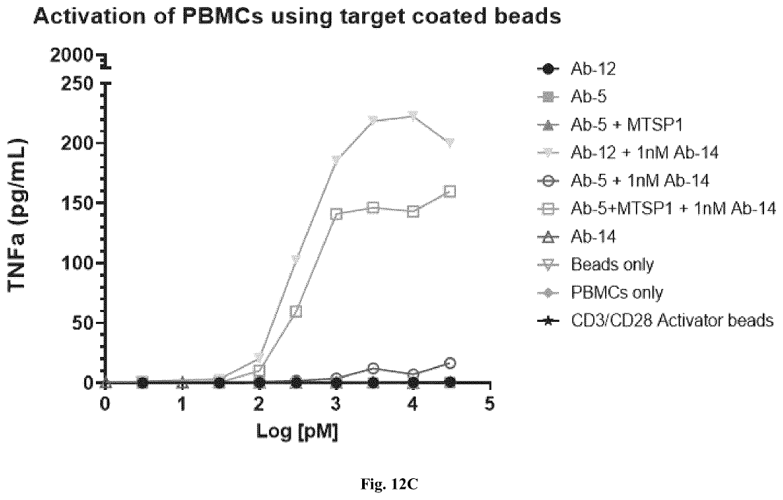

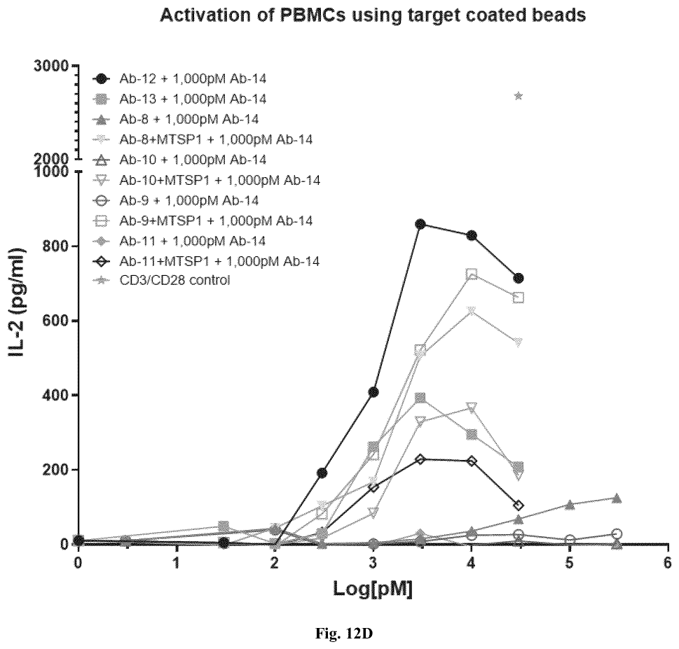

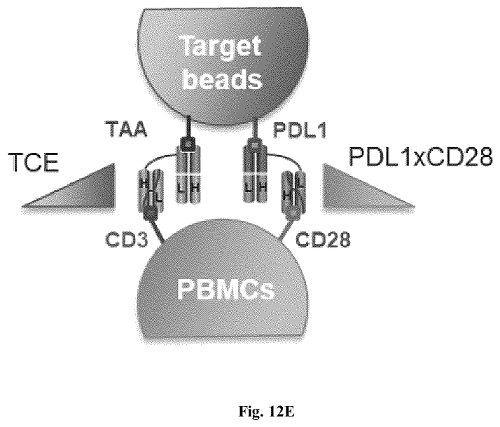

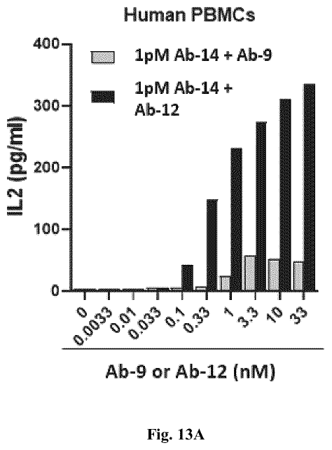

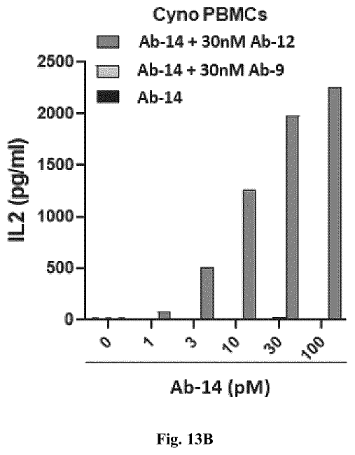

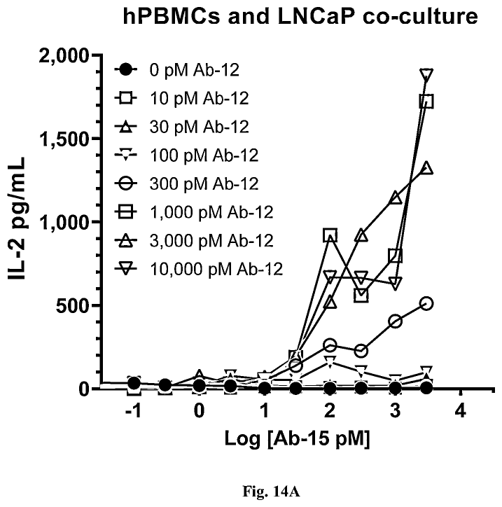

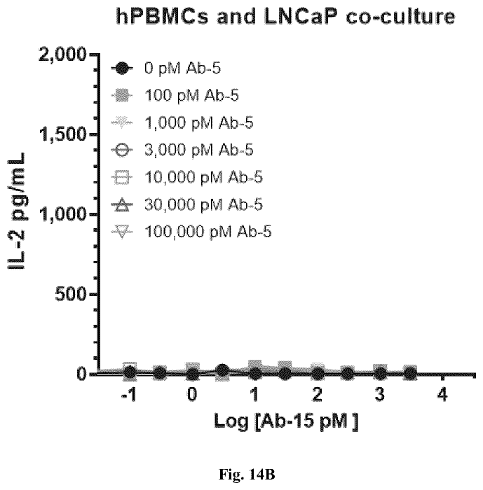

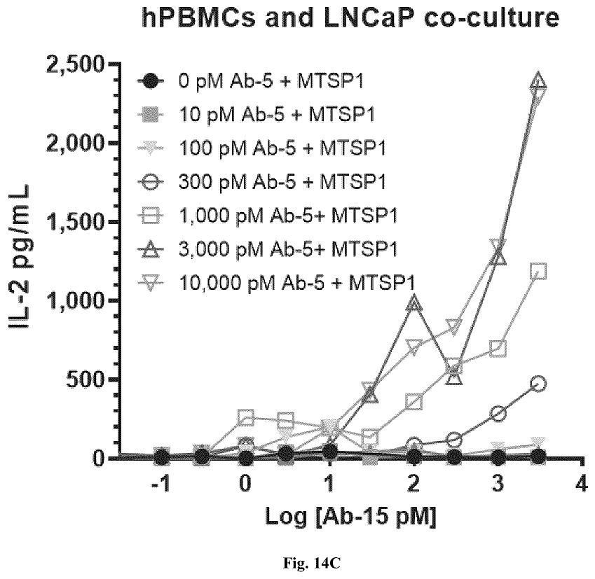

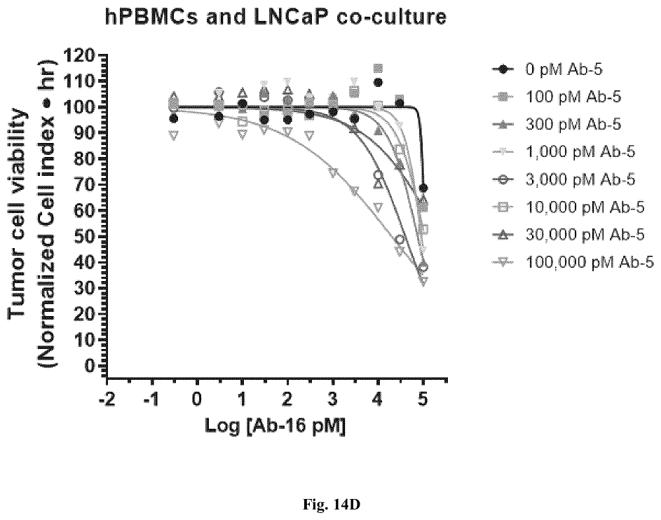

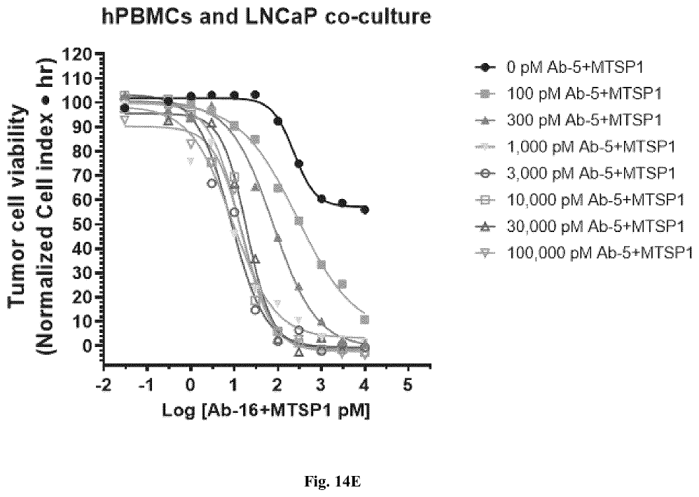

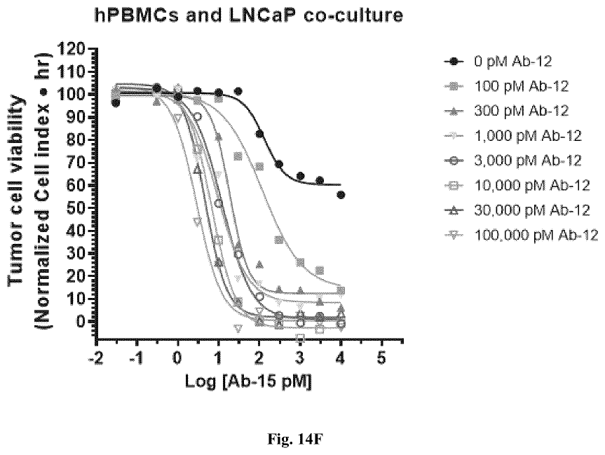

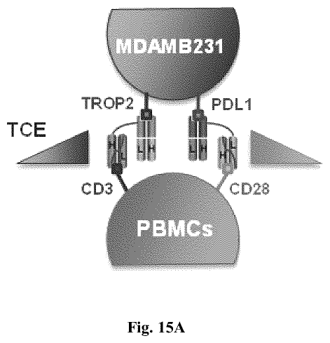

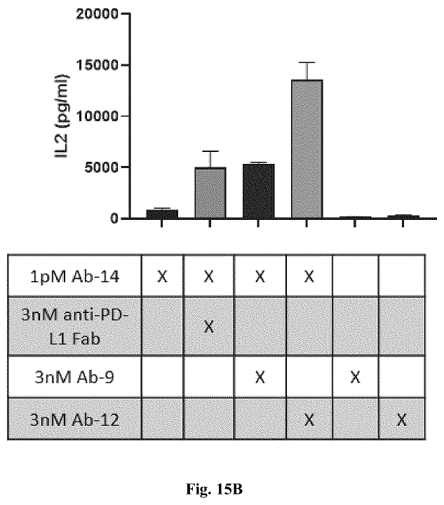

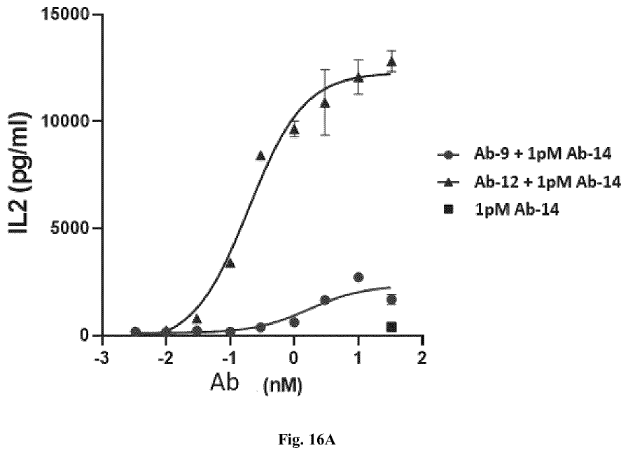

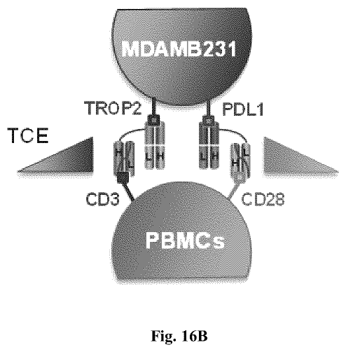

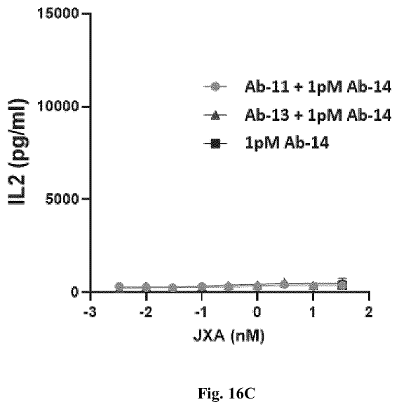

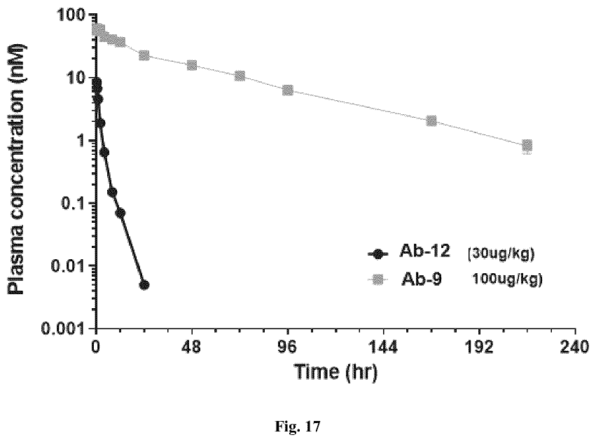

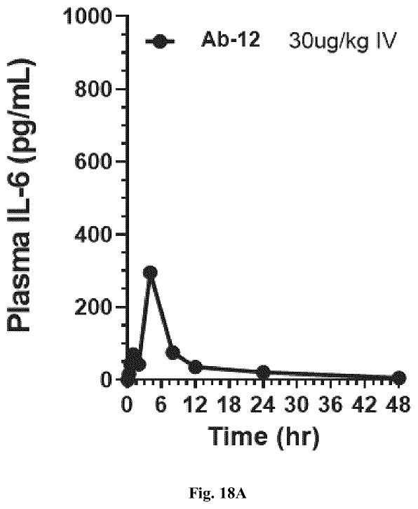

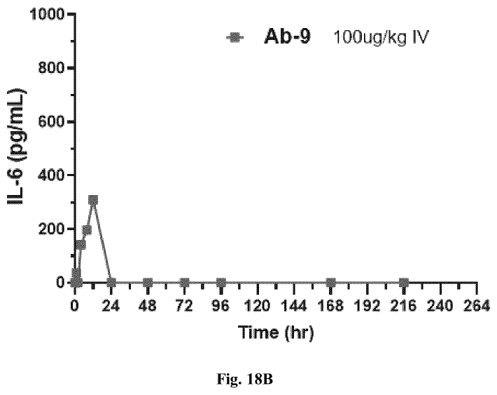

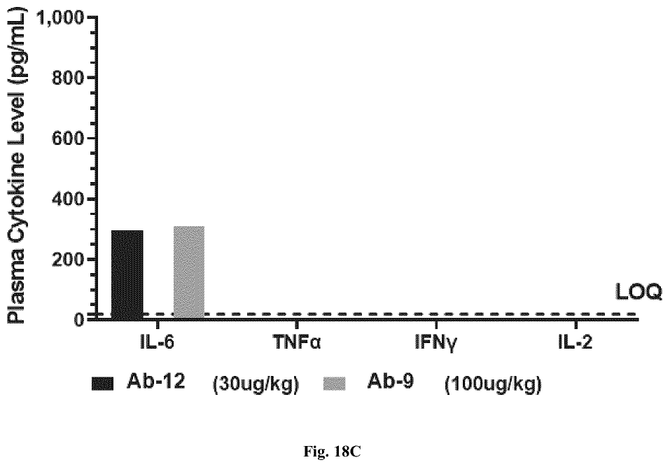

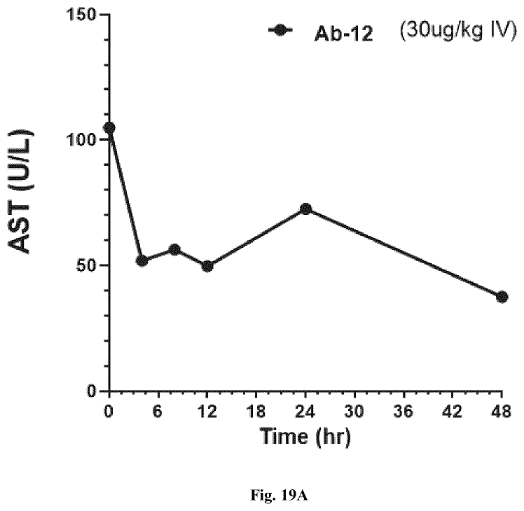

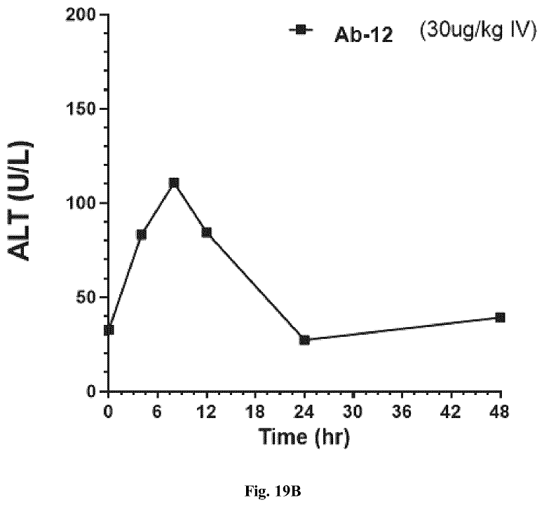

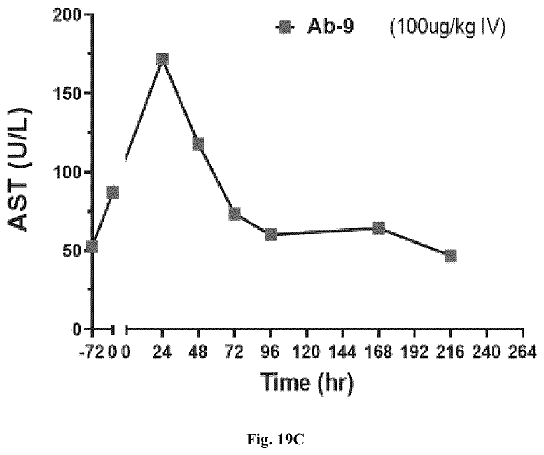

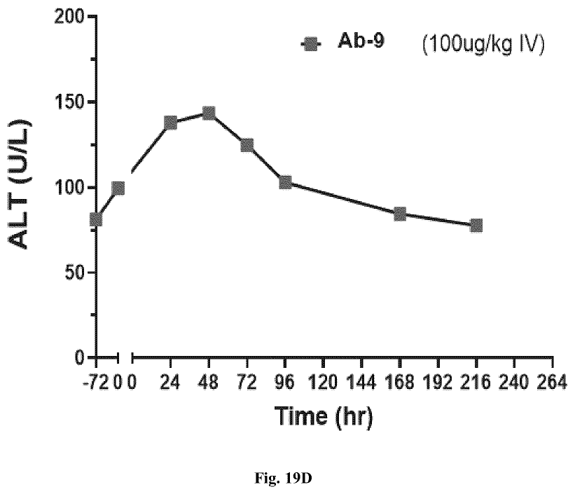

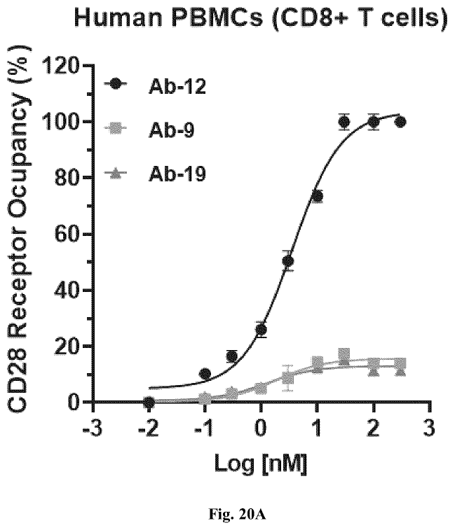

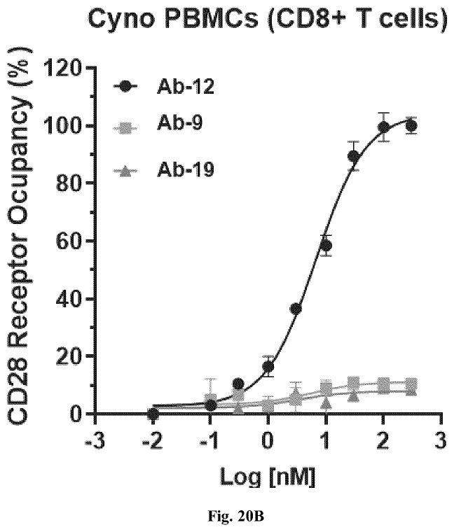

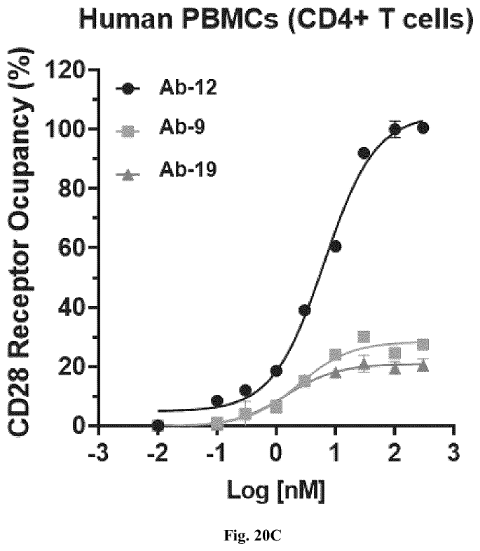

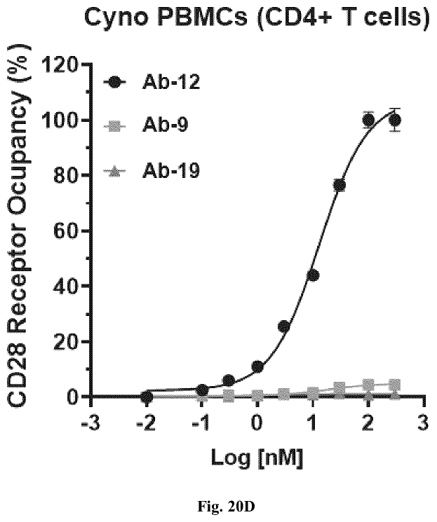

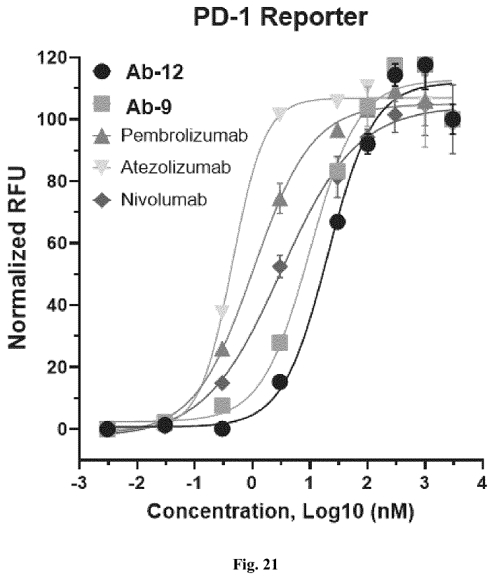

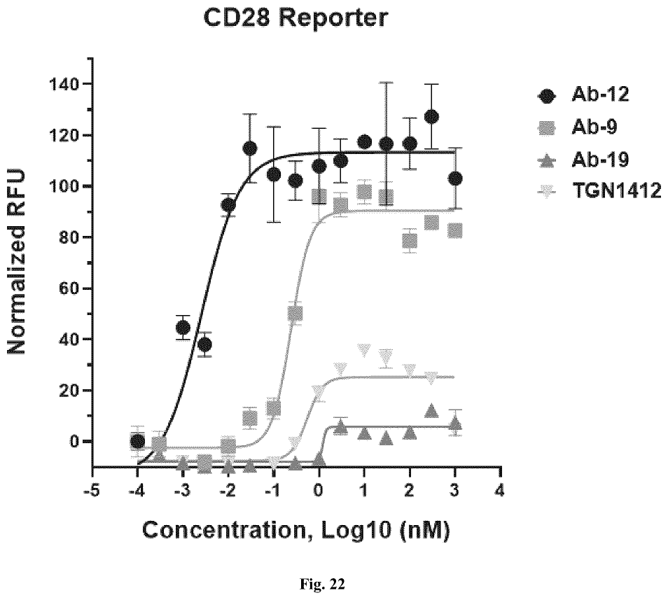

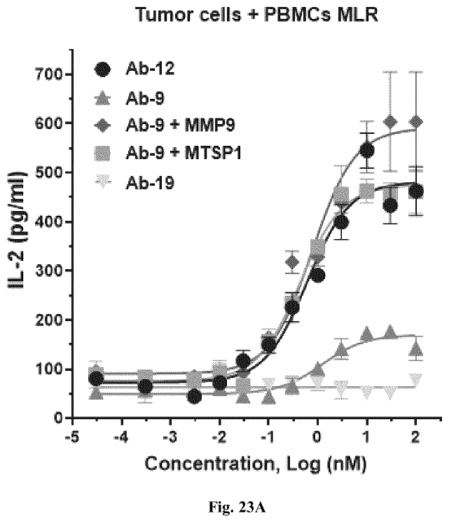

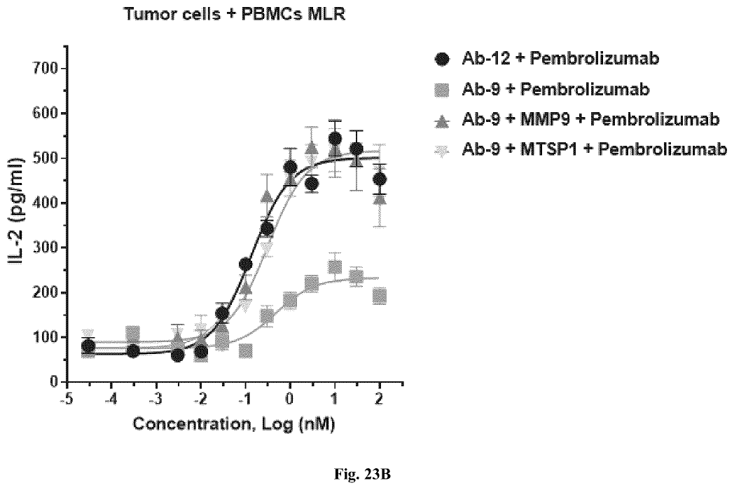

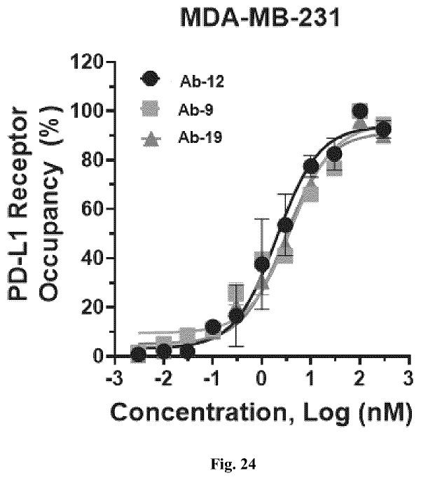

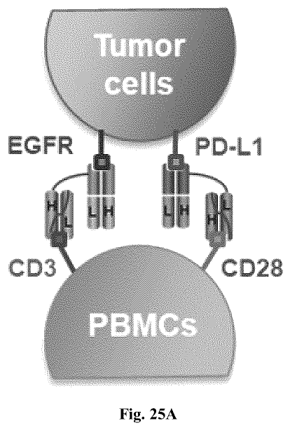

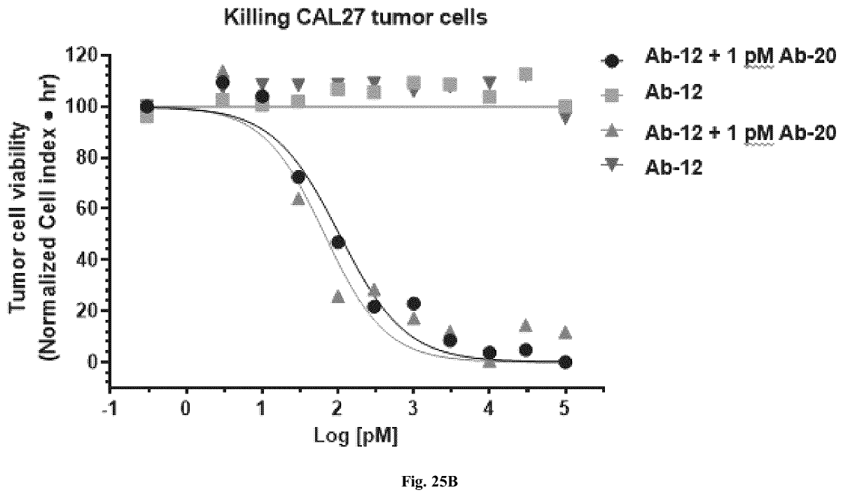

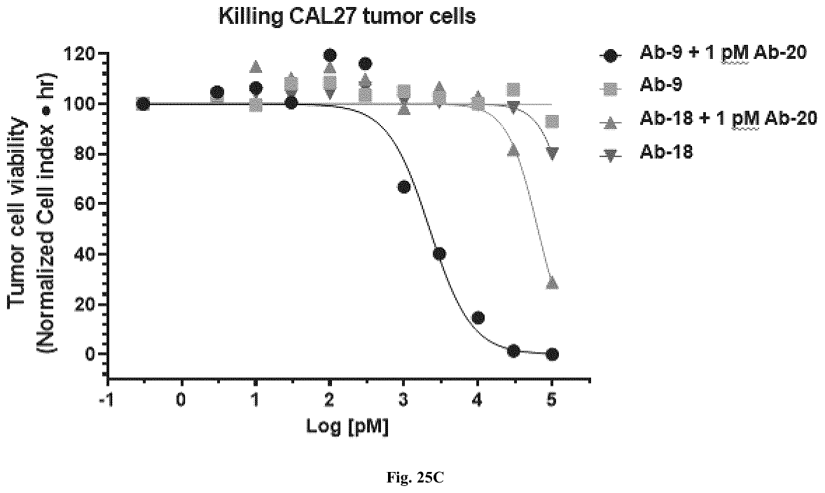

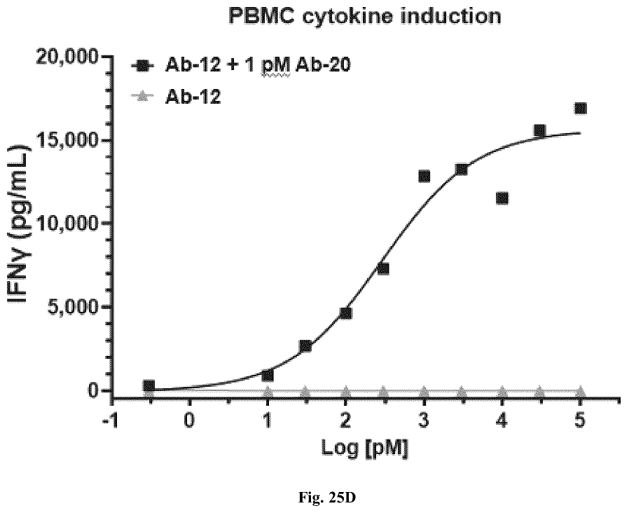

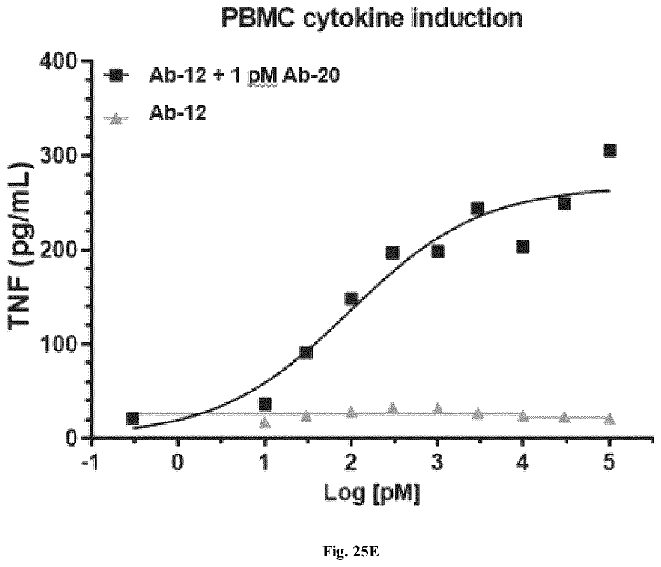

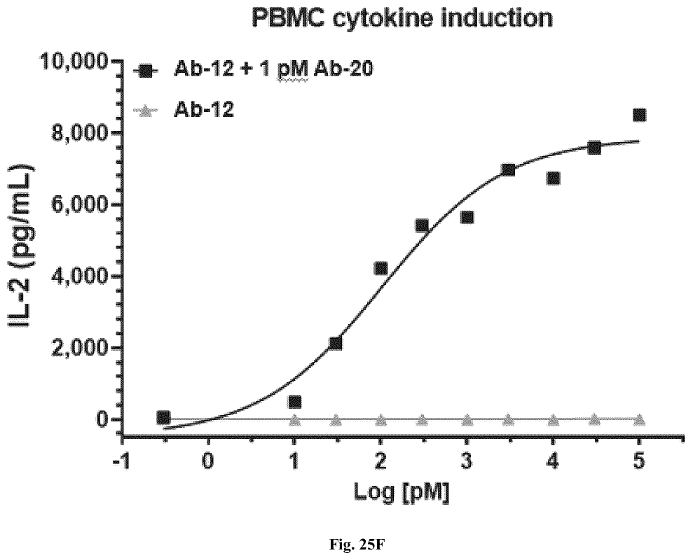

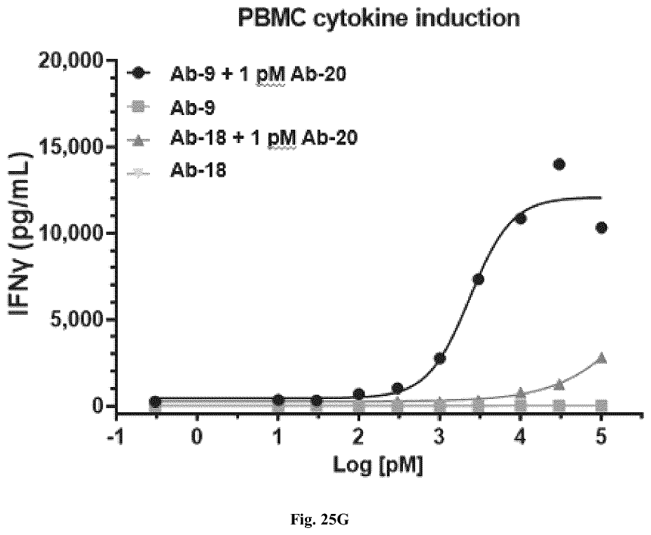

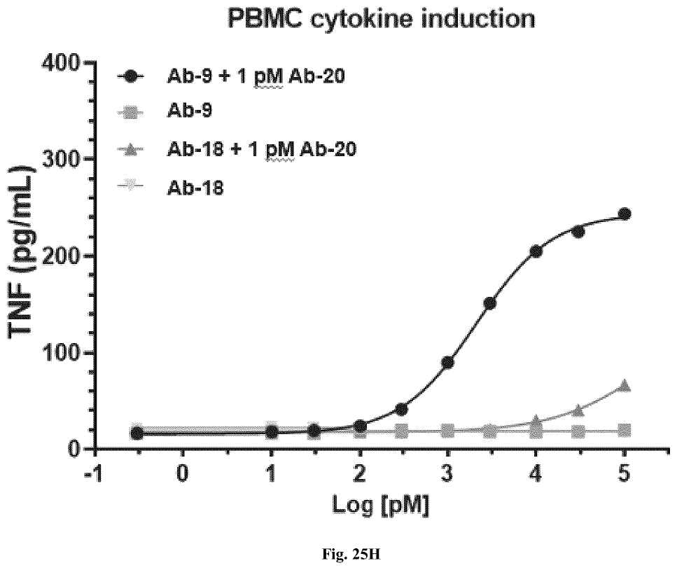

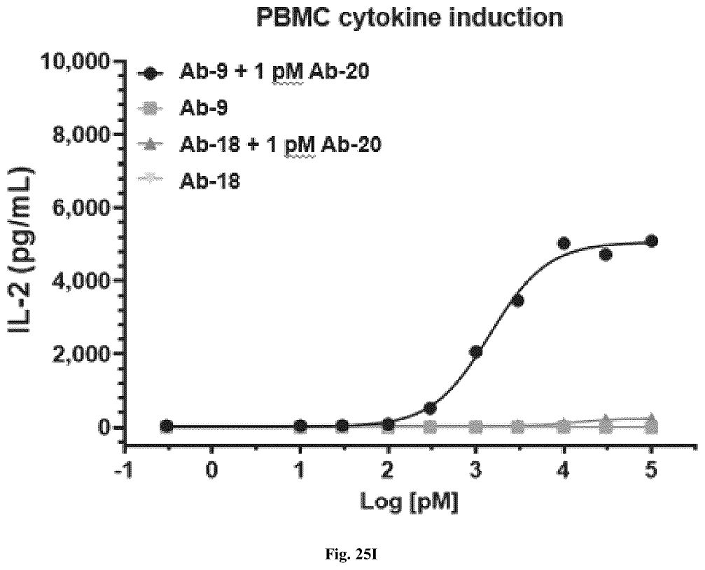

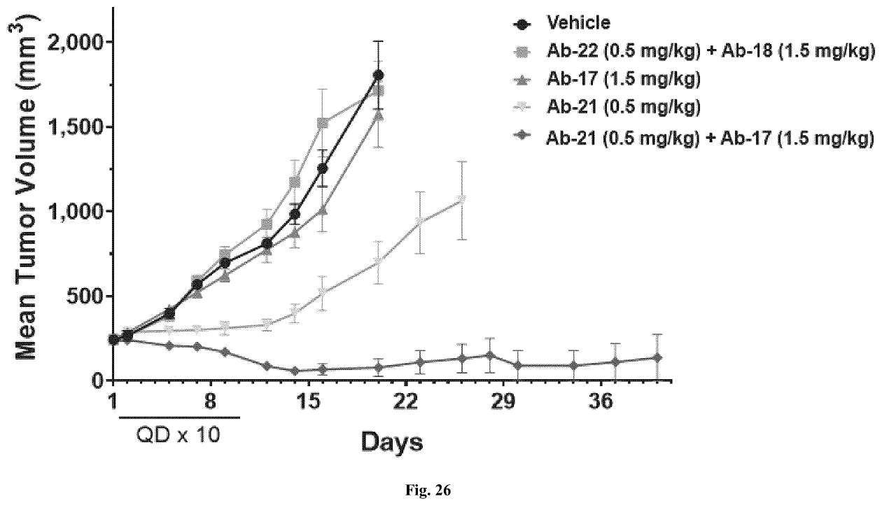

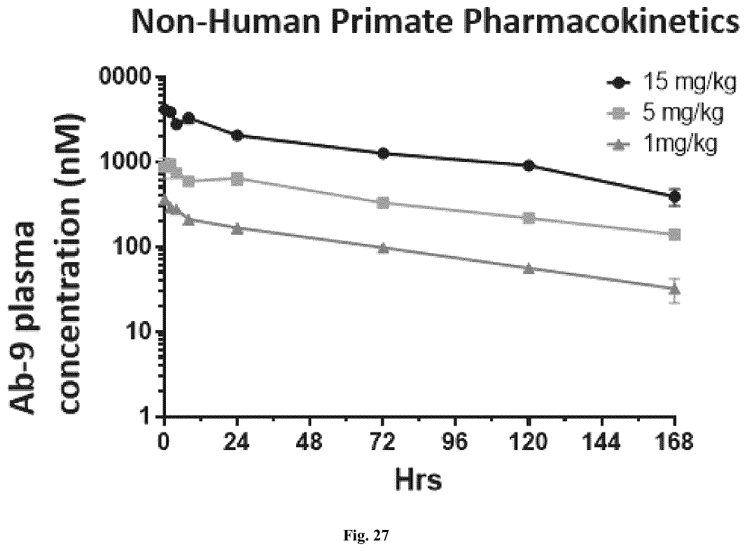

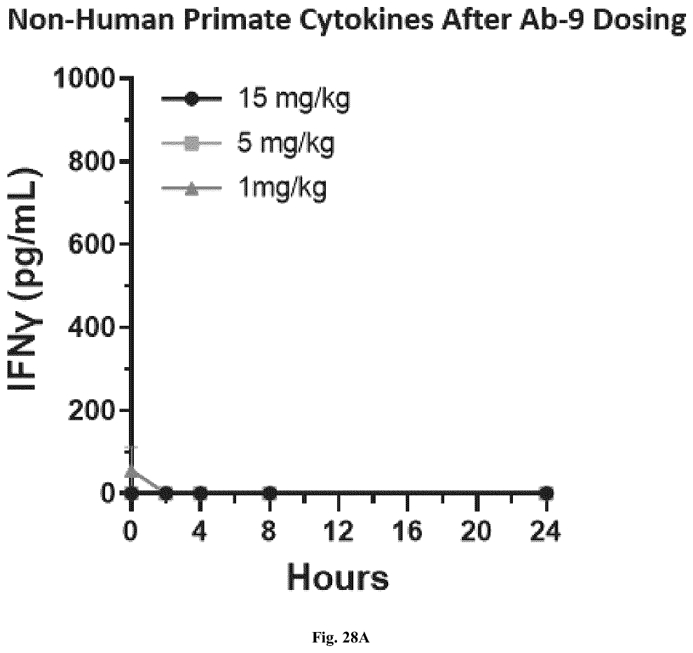

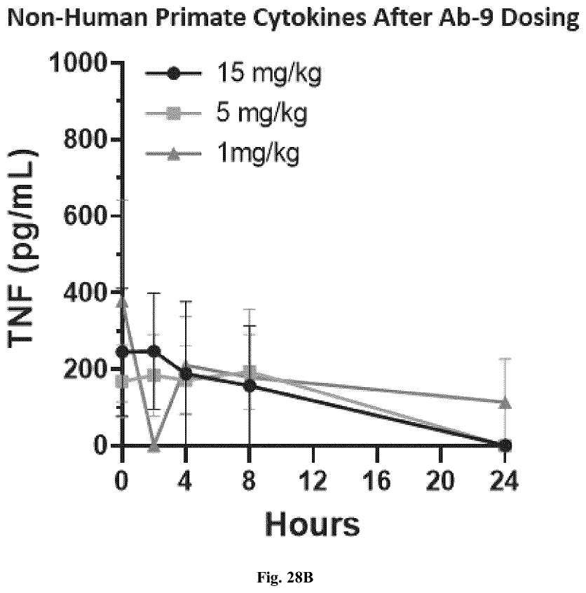

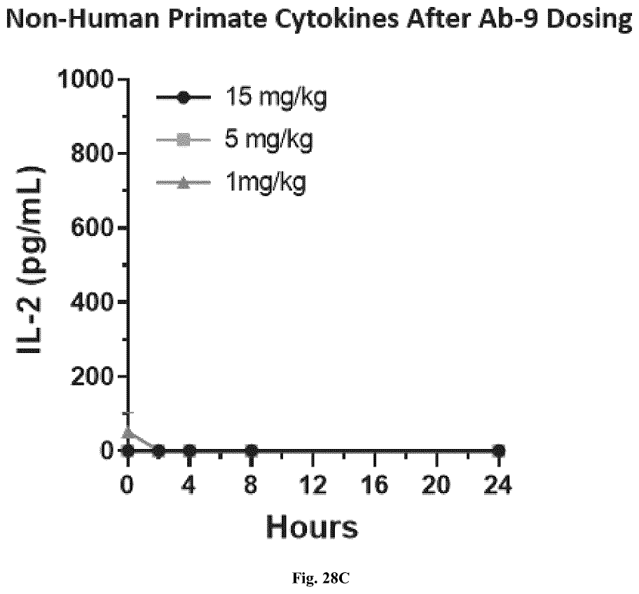

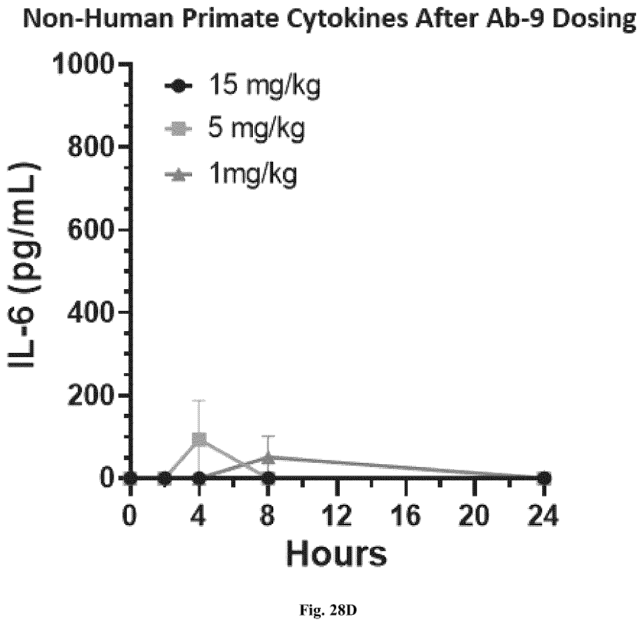

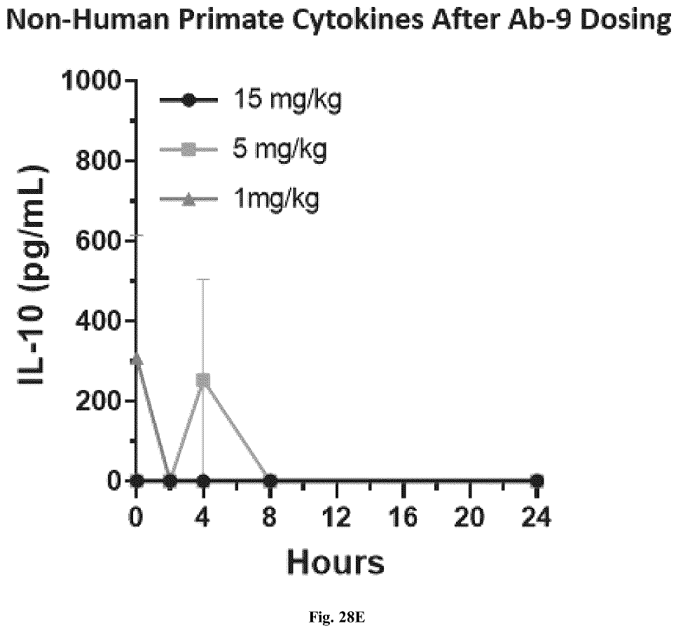

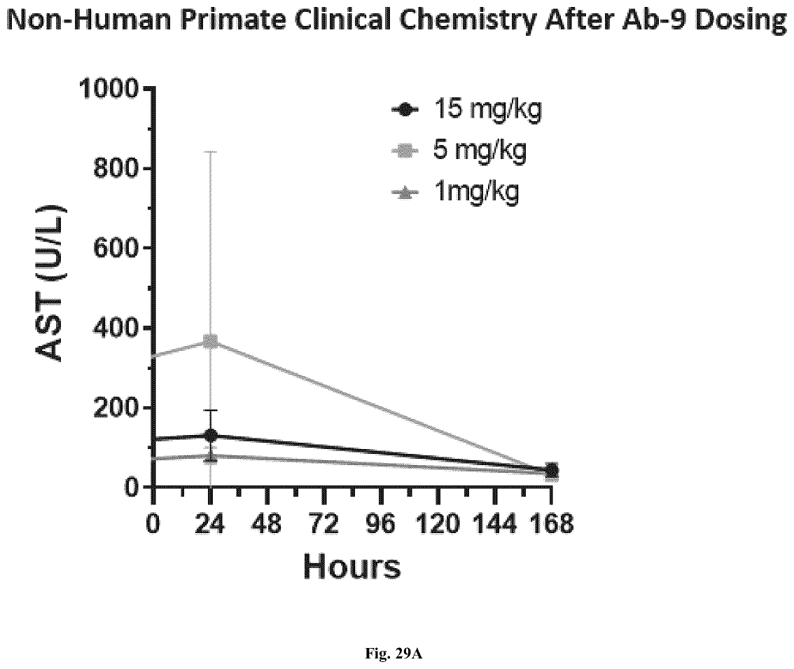

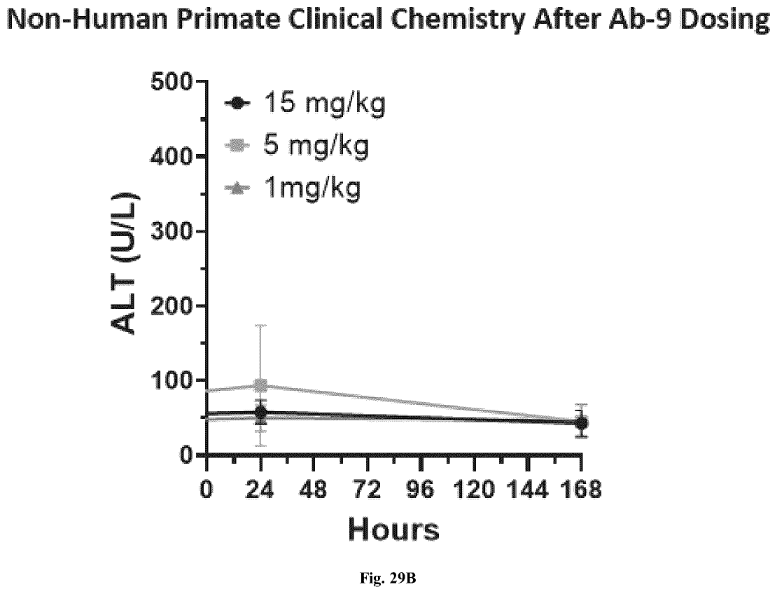

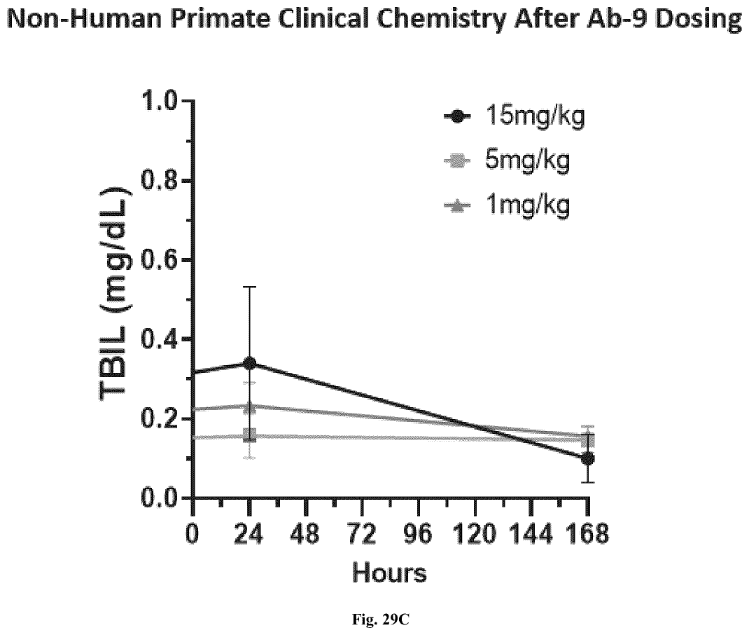

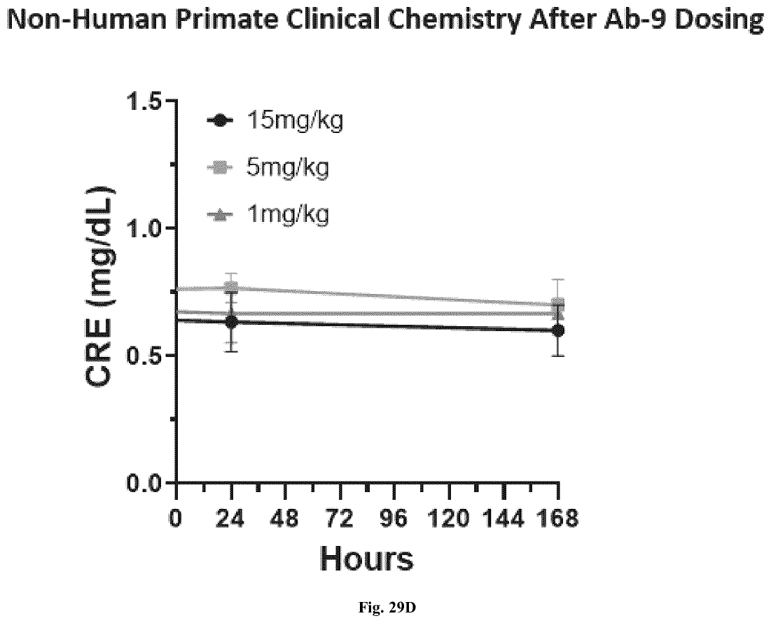

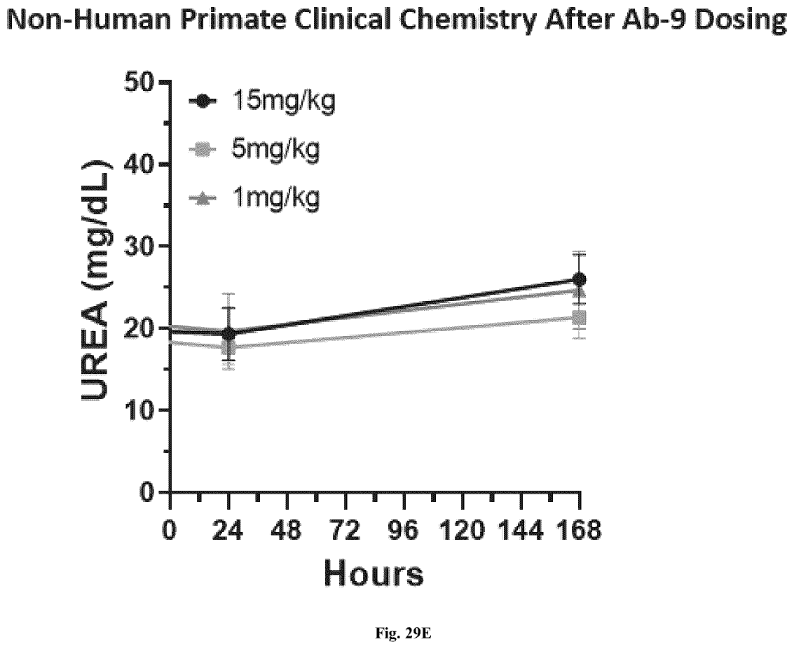

FIG. 11I illustrates binding of Ab-12, Ab-8, Ab-9, Ab-10, and Ab-11 to CD28 as measured by ELISA. In some circumstances, the antibodies are incubated with MTSP1.

FIG. 11J illustrates binding of Ab-12, Ab-5, Ab-1, and Ab-9 to CD28 as measured by ELISA.

FIG. 11K illustrates binding of Ab-12, Ab-5, Ab-1, and Ab-9 to PD-L1 as measured by ELISA.

FIG. 11L illustrates binding of Ab-12, Ab-9, and Ab-9+MTSP1 to PD-L1 as measured by ELISA.

FIG. 11M illustrates binding of Ab-12, Ab-9, and Ab-9+MTSP1 to CD28 as measured by ELISA.

FIGS. 12A-12D illustrate immune cell activation as measured by cytokine release after co-culture of target coated beads coated with TROP2 and PD-L1 and PBMCs and administration of antibody constructs that target CD28 and PD-L1 and an anti-TROP2×CD3 T cell engager (Ab-14).

FIG. 12E illustrates a cartoon configuration of an antibody construct that targets CD28 and PD-L1 that is administered in combination with a T cell engager (TCE) that targets a tumor associated antigen (TAA) such as TROP2 and CD3 of T cell.

FIG. 13A-13B illustrate immune cell activation as measured via IL-2 release after co-culture of targeted coated beads and human PBMCs (FIG. 13A) or cyno PSMCs (FIG. 13B). Beads are treated with biotinylated PD-L1 and soluble biotinylated TROP2 and antibody constructs that target CD28 and PD-L1 were administered as a single agent or in combination.

FIGS. 14A-14C illustrate results of an in vitro PBMC activation assay using the LNCaP PD-L1 positive tumor cell line in which various antibody constructs that target CD28 and PD-L1 and are co-administered with Ab-15 in the presence of human PBMCs. In vitro PBMC activation measured by cytokine release is synergized when various antibody constructs that target CD28 and PD-L1 are combined with an anti-PSMA×CD3 T cell engager (Ab-15).

FIGS. 14D-14F illustrate results of an in vitro tumor cell killing assay using the LNCaP PDL1 positive tumor cell line in the presence of human PBMCs. In vitro tumor cell killing is enhanced when various antibody constructs that target CD28 and PD-L1 are combined with an anti-PSMA×CD3 T cell engager (Ab-15) or masked PSMA×CD3 T cell engager (Ab-16). The tumor cell killing is mask dependent, where cleavage by MTSP1 that removes the mask results in enhanced tumor cell killing.

FIG. 15A illustrates a cartoon configuration of a multispecific antibody that targets CD28 and PD-L1 that is administered in combination with a T cell engager that targets a tumor associated antigen (TAA) such as TROP2 and CD3 of T cell.

FIG. 15B illustrates immune cell activation measured via IL-2 induction after co-culture PBMCs with MDAMB231 tumor cells and indicated antibodies.

FIGS. 16A and 16C illustrate immune cell activation measured via IL-2 induction after co-culture PBMCs with MDAMB231 tumor cells and indicated antibodies. FIG. 16B illustrates a cartoon configuration of a multispecific antibody that targets CD28 and PD-L1 that is administered in combination with a T cell engager that targets a tumor associated antigen (TAA) such as TROP2 and CD3 of T cell.

FIG. 17 illustrates pharmacokinetics of Ab-12 and Ab-9 in cynomolgus monkey after a single IV bolus injection.

FIGS. 18A-18C illustrate cytokine release in cynomolgus monkey after a single IV bolus injection of Ab-12 and Ab-9.

FIGS. 19A-19D illustrate serum liver enzymes in cynomolgus monkey after a single IV bolus injection of Ab-12 and Ab-9.

FIGS. 20A-20D illustrate binding results of Ab-12 (a non-masked antibody that binds to PD-L1 and CD28 in Vh format), Ab-9 (an antibody that binds to PD-L1 and CD28 in a cleavable masked Vh format), and Ab-19 (an antibody that binds to PD-L1 and CD28 in a non-cleavable masked Vh format) to human or Cyno PBMCs by flow cytometry.

FIG. 21 illustrates results of a PD-1 reporter assay for Ab-12, Ab-9, Pembrolizumab, Atezolizumab, and Nivolumab.

FIG. 22 illustrates results of the CD28 reporter assay of Ab-12, Ab-9, Ab-19, and TGN1412.

FIG. 23A illustrates results of in vitro IL-2 induction of Ab-12, Ab-9, and Ab-19 from human PBMC and tumor cell mixed lymphocyte reactions. Cleaved Ab-9 using MTSP1 and MMP9 is also shown. FIG. 23B illustrates results of Ab-12 in combination with Pembrolizumab, Ab-9 in combination with Pembrolizumab, MMP9 cleaved Ab-9 in combination with Pembrolizumab, and MTSP1 cleaved Ab-9 in combination with Pembrolizumab.

FIG. 24 illustrates results of Ab-12, Ab-9, and Ab-19 binding to PD-L1 on PD-L1-expressing MDA MB231 tumor cell line.

FIG. 25A illustrates a cartoon configuration of a multispecific antibody that targets CD28 and PD-L1 that is administered in combination with a T cell engager that targets a tumor associated antigen (TAA) such as EGFR and CD3 of T cell.

FIG. 25B-25C illustrate tumor cell killing of CAL27 tumor cells by Ab-12, Ab-9, Ab-18 alone or in combination with 1 pM of Ab-20, an EGFR T cell engager. Results of the plots are also summarized in Table 28.

FIG. 25D-25F illustrate cytokine induction (IFNγ, TNF, and IL-2) from human PBMCs co-cultures with Cal27 tumor cells in the presence of titrated Ab-12 or titrated Ab-12 in combination with 1 pM of Ab-20 in human serum supplemented medium.

FIG. 25G-25I illustrate cytokine induction (IFNγ, TNF, and IL-2) from human PBMCs co-cultures with Cal27 tumor cells in the presence of titrated Ab-9 or titrated Ab-9 in combination with 1 pM of Ab-20 and also titrated Ab-18 or titrated Ab-18 in combination with 1 pM of Ab-20 in human serum supplemented medium.

FIG. 26 illustrates in vivo tumor growth kinetics (mean tumor volume) of MDAMB231 in immunocompromised mice after treatment with Ab-22 in combination with Ab-18, or treatment with Ab-21 and Ab-17 in combination, or treatment with Ab-17 alone, or treatment with Ab-21 alone.

FIG. 27 illustrates non-human primate pharmacokinetics for dosing at 15 mg/kg, 5 mg/kg, and 1 mg/kg of Ab-9.

FIG. 28A-28E illustrate cytokine release (IFNγ, TNF, IL-2, IL-6, and IL-10) in non-human primates after administration of 15 mg/kg, 5 mg/kg, and 1 mg/kg of Ab-9.

FIG. 29A-29E illustrate non-human primate clinical chemistry results (AST, ALT, TBIL, CRE, urea) for dosing at 15 mg/kg, 5 mg/kg, and 1 mg/kg of Ab-9.

### DETAILED DESCRIPTION

Bispecific antibodies for redirecting T cells for mediating cancer cell killing have shown promise in both pre-clinical and in clinical studies. Efficient T cell activation has been obtained with single chain variable fragments (scFv), notably the Bispecific T-cell Engagers (BiTEs) format, in which one scFv targets a tumor cell antigen, and the other scFv targets an epitope such as CD3 that is involved in T cell activation. One such example of a BiTE is blinatumomab that targets CD19 and CD3 which has been approved in Europe and the United States for treatment of chemotherapy-resistant CD19+B cell acute lymphoblastic leukemia. Despite advances with T cell engagers such as blinatumomab some patients respond poorly to treatment even if the patient expresses the tumor antigen for reasons that are not entirely understood.

Strategies for increasing T cell cytotoxicity of T cell engagers have been explored through co-administration with a second antibody that targets the co-inhibitory immune checkpoint programmed death-ligand 1 (PD-L1) and/or CD28. CD28 is a protein expressed on T cells that provide co-stimulatory signals required for T cell activation and survival. It is known that stimulatory signaling through CD28 in combinations with BiTEs increase T cell-induced tumor cell cytotoxicity. However, central to obtaining T cell mediated cytotoxicity of tumor cells in prior studies required the presence of a BiTE that has a tumor binding domain, such as an anti-CD19 antibody, and a CD3 binding domain, while single agent administration of an anti-CD28 and anti-PD-L1 in a scFv-scFv format was found to not induce T cell mediated cytotoxicity against tumor cells.

Activation of T cells is a highly regulated process that typically requires two signaling events for full functionality: the first signal is initiated upon binding of the MHC-antigen complex, which helps distinguish “self” from “non-self” to the T cell receptor (TCR) and the second signal through activation of a costimulatory receptor. While the first recognition signal activates a T cell and triggers T cell mediated toxicity of the recognized cell, if the T cell does not receive a second costimulatory signal it can lead to T cell tolerance whereby the T cells continue to recognize the tumor antigen but do not mount an immune response against the tumor cell. The second costimulatory signal prevents T cell tolerance, and further activates the T cell to enhance T cell cytotoxicity towards the targeted cell.

Multispecific antibodies comprising a CD28 binding domain and PD-L1 binding domain as described herein are designed to act both as an antagonist of PD-L1 and a conditional agonist of C28. While CD28 agonism has shown some clinical promise, the efficacy seen with this approach has been limited due to dose-limiting toxicities that result from systemic activation of CD28. The multispecific antibodies comprising a CD28 binding domain and PD-L1 binding domain, described herein, are designed to conditionally agonize CD28 only in the presence of PD-L1, which is often overexpressed by tumors to avoid T cell mediated killing. In addition, engagement of PD-L1 is designed to block PD-1 binding and provide checkpoint inhibition. This combination provides a mechanism of action that enhances anti-tumor responses and limits the systemic toxicity of CD28 agonism. Studies of multispecific antibodies described herein demonstrate a lack of systemic immune system activation, as evidenced by the lack of cytokine release. Despite unprecedented clinical response rates, most patients fail to respond to therapies targeting PD-1 and PD-L1, which is due in part because T cells require costimulation for full functionality. As such, checkpoint inhibition alone is likely insufficient to fully enable the immune system to attack a tumor. Further benefit can be derived by the addition of the multispecific antibodies as described herein.

Disclosed herein are antibodies that bind specifically to PD-L1 and CD28 which are able to induce T cell mediated cytotoxicity of tumor cells as a single agent or in combination with a T cell engager. Significantly, such antibodies that target PD-L1 and CD28 are able to induce T cell mediated cytotoxicity of tumor cells as a single agent, even when not administered with a second agent that specifically targets a tumor cell antigen. Such antibodies that bind specifically to PD-L1 and CD28 are not in a scFv-scFv format.

Disclosed herein are isolated multispecific antibodies according to the following formula: P₁-L₁-A1-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

Disclosed herein are isolated multispecific antibodies comprising the following formula: P₁-L₁-A₁-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

Disclosed herein are isolated multispecific antibodies comprising the following formula: P₁-L₁-A₁-L-B (Formula I) wherein A₁ is a CD28 binding domain; B is a PD-L1 binding domain; L is a linker that connects A₁ to B; P₁ is a peptide that binds to A₁ and L₁ is a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

Disclosed herein are isolated multispecific antibodies according to the following formula: P₁-L₁-A₁-L-B (Formula I) wherein A₁ is a CD28 binding domain; B is a PD-L1 binding domain; L is a linker that connects A₁ to B; P₁ is a peptide that binds to A₁ and L₁ is a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

In some embodiments, the multispecific antibody is according to the following formula: P₁-L₁-A₁-L-B-L₂-P₂ (Formula Ia) wherein P₂ comprises a peptide that binds to B and L₂ comprises a linking moiety that connects B to P₂ and is a substrate for a tumor specific protease.

In some embodiments, the multispecific antibody comprises the following formula: P₁-L₁-A₁-L-B-L₂-P₂ (Formula Ia) wherein P₂ comprises a peptide that binds to B and L₂ comprises a linking moiety that connects B to P₂ and is a substrate for a tumor specific protease.

In some embodiments, the multispecific antibody comprises the following formula: P₁-L₁-A₁-L-B-L₂-P₂ (Formula Ia) wherein P₂ is a peptide that binds to B and L₂ is a linking moiety that connects B to P₂ and is a substrate for a tumor specific protease.

In some embodiments, the multispecific antibody is according to the following formula: P₁-L₁-A₁-L-B-L₂-P₂ (Formula Ia) wherein P₂ is a peptide that binds to B and L₂ is a linking moiety that connects B to P₂ and is a substrate for a tumor specific protease.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

In some instances, the CDRs of an antibody are determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “Fab” refers to a protein that contains the constant domain of the light chain and the first constant domain (CH1) of the heavy chain Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

The term “multispecific” means that the antibody is able to specifically bind to two or more distinct antigenic determinants for example two or more binding sites each formed by a pair of an antibody heavy chain variable domain (VH) and an antibody light chain variable domain (VL), or in the case of a single domain antibody a single variable domain, binding to different antigens.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

Peptide (P₁) or (P₂)

In some embodiments, P₁ comprises an amino acid sequence according to any one of SEQ ID NOs: 24-53, 128-148, and the amino acid sequences of Table 20.

In some embodiments, P₁ comprises an amino acid sequence according to any one of SEQ ID NOs: 42-53 or an amino acid sequence that has 1, 2, or 3 amino acid mutations, substitutions, or deletions relative to any one of SEQ ID NOs: 42-53. In some embodiments, P₁ comprises an amino acid sequence according to any one of SEQ ID NOs: 42-53.

In some embodiments, P₁ comprises an amino acid sequence according to any one of the amino acid sequences of Table 20 or an amino acid sequence that has 1, 2, or 3 amino acid mutations, substitutions, or deletions relative to any one of the amino acid sequences of Table 20. In some embodiments, P₁ comprises an amino acid sequence according to any one of the amino acid sequences of Table 20.

In some embodiments, P₁ comprises an amino acid sequence according to any one of SEQ ID NOs: 128-147 or an amino acid sequence that has 1, 2, or 3 amino acid mutations, substitutions, or deletions relative to any one of SEQ ID NOs: 128-147. In some embodiments, P₁ comprises an amino acid sequence according to any one of SEQ ID NOs: 128-147.

In some embodiments, P₁ comprises an amino acid sequence according to X₁-X₂-X₃-C-X₄-X₅-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y. In some embodiments, X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F.

In some embodiments, P₁ comprises an amino acid sequence according to SEQ ID NO: 32 or an amino acid sequence that has 1, 2, or 3 amino acid mutations, substitutions, or deletions relative to SEQ ID NO: 32. In some embodiments, P₁ comprises an amino acid sequence according to SEQ ID NO: 32.

In some embodiments, P₁ comprises an amino acid sequence according to SEQ ID NO: 138 or an amino acid sequence that has 1, 2, or 3 amino acid mutations, substitutions, or deletions relative to SEQ ID NO: 138. In some embodiments, P₁ comprises an amino acid sequence according to SEQ ID NO: 138.

In some embodiments, P₁ impairs binding of A₁ to CD28. In some embodiments, P₁ is bound to A₁ through ionic interactions, electrostatic interactions, hydrophobic interactions, Pi-stacking interactions, and H-bonding interactions, or a combination thereof. In some embodiments, P₁ is bound to A₁ at or near an antigen binding site. In some embodiments, P₁ becomes unbound from A₁ when L₁ is cleaved by the tumor specific protease thereby exposing A₁ to CD28. In some embodiments, P₁ has less than 75% sequence identity to CD28. In some embodiments, P₁ has less than 80% sequence identity to CD28. In some embodiments, P₁ has less than 85% sequence identity to CD28. In some embodiments, P₁ has less than 90% sequence identity to CD28. In some embodiments, P₁ has less than 95% sequence identity to CD28. In some embodiments, P₁ comprises a de novo amino acid sequence that shares less than 10% sequence identity to CD28.

In some embodiments, P₂ impairs binding of B to PD-L1. In some embodiments, P₂ is bound to B through ionic interactions, electrostatic interactions, hydrophobic interactions, Pi-stacking interactions, and H-bonding interactions, or a combination thereof.

In some embodiments, P₂ is bound to B at or near an antigen binding site. In some embodiments, P₂ becomes unbound from B when L₂ is cleaved by the tumor specific protease thereby exposing B to the PD-L1. In some embodiments, P₂ has less than 70% sequence identity to the PD-L1. In some embodiments, P₂ has less than 75% sequence identity to the PD-L1. In some embodiments, P₂ has less than 80% sequence identity to the PD-L. In some embodiments, P₂ has less than 85% sequence identity to the PD-L1. In some embodiments, P₂ has less than 90% sequence identity to the PD-L1. In some embodiments, P₂ has less than 95% sequence identity to the PD-L1. In some embodiments, P₂ comprises a de novo amino acid sequence that shares less than 10% sequence identity to the PD-L1. In some embodiments, P₂ comprises a peptide sequence of at least 5 amino acids in length. In some embodiments, P₂ comprises a peptide sequence of at least 6 amino acids in length. In some embodiments, P₂ comprises a peptide sequence of at least 10 amino acids in length. In some embodiments, P₂ comprises a peptide sequence of at least 10 amino acids in length and no more than 20 amino acids in length. In some embodiments, P₂ comprises a peptide sequence of at least 16 amino acids in length. In some embodiments, P₂ comprises a peptide sequence of no more than 40 amino acids in length.

In some embodiments, P₁ or P₂ comprises at least two cysteine amino acid residues. In some embodiments, P₁ or P₂ comprises a cyclic peptide or a linear peptide. In some embodiments, P₁ or P₂ comprises a cyclic peptide. In some embodiments, P₁ or P₂ comprises a linear peptide. In some embodiments, P₁ or P₂ comprise a modified amino acid or non-natural amino acid, or a modified non-natural amino acid, or a combination thereof. In some embodiments, P₁ or P₂ does not comprise albumin or an albumin fragment. In some embodiments, P₁ or P₂ does not comprise an albumin binding domain.

TABLE 1
P₁ Sequences

| Construct | Amino Acid Sequence | |
|---|---|---|
| Description | (N to C) | SEQ ID NO: |
| Peptide-1 | YWYCSPSIVRCVLV | 24 |
| Peptide-2 | LICKSGSILILCAQ | 25 |
| Peptide-3 | SPCGLFQWMEICEF | 26 |
| Peptide-4 | SFCGLFLDLWICEF | 27 |
| Peptide-5 | DNCYVIWGFEWQCR | 28 |
| Peptide-6 | VNCMRVHRTLTWCV | 29 |
| Peptide-7 | FCTPREWSFLNFVC | 30 |
| Peptide-8 | CFAYLWIDSWIRVC | 31 |
| Peptide-9 | MDWCPRERWVDCFF | 32 |
| Peptide-10 | GQHCATSMWRYCMF | 33 |
| Peptide-11 | WICDKSGSIMLCRA | 34 |
| Peptide-12 | GYCHYWGDMVMWCG | 35 |
| Peptide-13 | DNCHYIWGFEWQCG | 36 |
| Peptide-14 | IDCIMVHMVKPWCF | 37 |
| Peptide-15 | NCQPWYWNMFAFGC | 38 |
| Peptide-16 | NCQPWYWNMIAFGC | 39 |
| Peptide-17 | GCFTWSQRTFPFTC | 40 |
| Peptide-18 | CFYAEYYDQVYSFC | 41 |
| Peptide-19 | ADWCPRERWVDCFF | 42 |
| Peptide-20 | MAWCPRERWVDCFF | 43 |
| Peptide-21 | MDACPRERWVDCFF | 44 |
| Peptide-22 | MDWCARERWVDCFF | 45 |
| Peptide-23 | MDWCPAERWVDCFF | 46 |
| Peptide-24 | MDWCPRARWVDCFF | 47 |
| Peptide-25 | MDWCPREAWVDCFF | 48 |
| Peptide-26 | MDWCPRERAVDCFF | 49 |
| Peptide-27 | MDWCPRERWADCFF | 50 |
| Peptide-28 | MDWCPRERWVACFF | 51 |
| Peptide-29 | MDWCPRERWVDCAF | 52 |
| Peptide-30 | MDWCPRERWVDCFA | 53 |
| Peptide-31 | MDWCPIDLWNECFF | 128 |
| Peptide-32 | MDWCPIHLWHVCFN | 129 |
| Peptide-33 | MDWCPIYLWSECFN | 130 |
| Peptide-34 | MNWCPKDIWYLCFN | 131 |
| Peptide-35 | MDWCPLHMWHECFS | 132 |
| Peptide-36 | MDWCPLYLWNECFN | 133 |
| Peptide-37 | MDWCPRDLWDLCFA | 134 |
| Peptide-38 | MDWCPRDLWHECFA | 135 |
| Peptide-39 | MDWCPRDLWHLCFS | 136 |
| Peptide-40 | MDWCPRDLWSECFF | 137 |
| Peptide-41 | MDWCPRDLWVHCFA | 138 |
| Peptide-42 | MDWCPRDMWDECFA | 139 |
| Peptide-43 | MDWCPRDMWSECFA | 140 |
| Peptide-44 | MDWCPRDMWSVCFS | 141 |
| Peptide-45 | MDWCPRFMWDECFN | 142 |
| Peptide-46 | MDWCPRHMWNYCFA | 143 |
| Peptide-47 | MDWCPRSLWHECFA | 144 |
| Peptide-48 | MDWCPRYLWHVCFA | 145 |
| Peptide-49 | MHWCPVDLWYLCYN | 146 |
| Peptide-50 | MDWCPVHLWSVCFA | 147 |
| Peptide-51 | MDWCPRERWVDCFF | 148 |

Linking Moiety (L₁ or L₂)

In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or Leis a peptide sequence having at least 18 amino acids. In some embodiments, L₁ or L₂ is a peptide sequence having at least 26 amino acids. In some embodiments, L₁ or L₂ comprises a formula comprising (G₂S)ₙ, wherein n is an integer from 1 to 3 (SEQ ID NO: 228). In some embodiments, L₁ or L₂ comprises a formula comprising (G₂S)ₙ, wherein n is an integer of at least 1. In some embodiments, L₁ or L₂ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments, L₁ or L₂ comprises a urokinase cleavable amino acid sequence, a matriptase cleavable amino acid sequence, or a matrix metalloprotease cleavable amino acid sequence. In some embodiments, L₁ or L₂ comprises a sequence according to SEQ ID NOs: 18-19, 62-88. In some embodiments, L₁ is bound to N-terminus of A₁. In some embodiments, L₁ is bound to C-terminus of A₁. In some embodiments, L₂ is bound to N-terminus of B. In some embodiments, L₂ is bound to C-terminus of B.

TABLE 2
L₁ or L₂

| | Construct | Amino Acid Sequence | SEQ ID |
|---|---|---|---|
| | Description | (N to C) | NO: |
| CD28 | Linker 1 | GGGGSGGGGSGGGGS | 18 |
| | Linker 2 | GGGGS | 19 |
| | Linker 3 | GGGGSGGGS | 62 |
| | Cleavable | GGGGSGGGLSGRSDAGSPLGL | 63 |
| | linker | AGSGGGS | |
| | Linker 4 | GGGGSLSGRSDNHGSSGT | 64 |
| | Linker 5 | GGGGSSGGSGGSGLSGRSDNH | 65 |
| | | GSSGT | |
| | Linker 6 | ASGRSDNH | 66 |
| | Linker 7 | LAGRSDNH | 67 |
| | Linker 8 | ISSGLASGRSDNH | 68 |
| | Linker 9 | ISSGLLAGRSDNH | 69 |
| | Linker 10 | LSGRSDNH | 70 |
| | Linker 11 | ISSGLLSGRSDNP | 71 |
| | Linker 12 | ISSGLLSGRSDNH | 72 |
| | Linker 13 | LSGRSDNHSPLGLAGS | 73 |
| | Linker 14 | SPLGLAGSLSGRSDNH | 74 |
| | Linker 15 | SPLGLSGRSDNH | 75 |
| | Linker 16 | LAGRSDNHSPLGLAGS | 76 |
| | Linker 17 | LSGRSDNHVPLSLKMG | 77 |
| | Linker 18 | LSGRSDNHVPLSLSMG | 78 |
| | Linker 19 | GSSGGSGGSGGSGISSGLLSGR | 79 |
| | | SDNHGSSGT | |
| | Linker 20 | GSSGGSGGSGGISSGLLSGRSD | 80 |
| | | NHGGGS | |
| | Linker 21 | ASGRSDNH | 81 |
| | Linker 22 | LAGRSDNH | 82 |
| | Linker 23 | ISSGLASGRSDNH | 83 |
| | Linker 24 | LSGRSDAG | 84 |
| | Linker 25 | ISSGLLSGRSDAG | 85 |
| | Linker 26 | AAGLLAPPGGLSGRSDAG | 86 |
| | Linker 27 | SPLGLSGRSDAG | 87 |
| | Linker 28 | LSGRSDAGSPLGLAG | 88 |

### Binding Domain (A₁), PD-L1 Binding Domain (B), and Linker (L)

In some embodiments, the CD28 binding domain comprises a single chain variable fragment, a single domain antibody, a Fab, or a Fab′. In some embodiments, the CD28 binding domain comprises the single chain variable fragment. In some embodiments, the CD28 binding domain comprises the single domain antibody. In some embodiments, the CD28 binding domain comprises the Fab or the Fab′. In some embodiments, the PD-L1 binding domain comprises a single chain variable fragment, a single domain antibody, a Fab, or a Fab′. In some embodiments, the PD-L1 binding domain comprises the Fab or the Fab′. In some embodiments, the PD-L1 binding domain comprises the Fab or the Fab′ and the CD28 binding domain comprises the single chain variable fragment. In some embodiments, the PD-L1 binding domain that comprises the Fab or the Fab′ comprises a Fab heavy chain polypeptide comprising a Fab heavy chain variable domain and a Fab light chain polypeptide comprising a Fab light chain variable domain. In some embodiments, the CD28 binding domain that comprises the single chain variable fragment comprises a scFv heavy chain variable domain and a scFv light chain variable domain. In some embodiments, the linker connects the C-terminus of A₁ to an N-terminus of B. In some embodiments, the linker connects the N-terminus of A₁ to a C-terminus of B. In some embodiments, the linker connects the C-terminus of A₁ to the N-terminus of the Fab heavy chain polypeptide. In some embodiments, the linker connects the N-terminus of A₁ to the C-terminus of the Fab heavy chain polypeptide. In some embodiments, the linker connects the C-terminus of A₁ to the N-terminus of the Fab light chain polypeptide. In some embodiments, the linker connects the N-terminus of A₁ to the C-terminus of the Fab light chain polypeptide. In some embodiments, the linker connects the Fab light chain polypeptide to the scFv light chain variable domain. In some embodiments, the linker connects the Fab light chain polypeptide to the scFv heavy chain variable domain. In some embodiments, the linker connects the Fab heavy chain polypeptide to the scFv light chain variable domain. In some embodiments, the linker connects the Fab heavy chain polypeptide to the scFv heavy chain variable domain. In some embodiments, the linker connects the Fab light chain polypeptide to the N-terminus of the scFv light chain variable domain. In some embodiments, the linker connects the Fab light chain polypeptide to the C-terminus of the scFv light chain variable domain. In some embodiments, the linker connects the Fab light chain polypeptide to the N-terminus of the scFv heavy chain variable domain. In some embodiments, the linker connects the Fab light chain polypeptide to the C-terminus of the scFv heavy chain variable domain. In some embodiments, the linker connects the Fab heavy chain polypeptide to the N-terminus of the scFv light chain variable domain. In some embodiments, the linker connects the Fab heavy chain polypeptide to the C-terminus of the scFv light chain variable domain. In some embodiments, the linker connects the Fab heavy chain polypeptide to the N-terminus of the scFv heavy chain variable domain. In some embodiments, the linker connects the Fab heavy chain polypeptide to the C-terminus of the scFv heavy chain variable domain.

In some embodiments, the linker is at least 5 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker is 15 amino acids in length. In some embodiments, the linker comprises (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, L comprises a formula comprising (G₂S)ₙ, wherein n is an integer from 1 to 3 (SEQ ID NO: 228). In some embodiments, the L comprises an amino acid sequence of SEQ ID NO: 18 (GGGGSGGGGSGGGGS) or SEQ ID NO: 19 (GGGGS).

TABLE 3
Linker sequences

| Construct | Amino Acid Sequence | SEQ ID |
|---|---|---|
| Description | (N to C) | NO: |
| Linker 1 | GGGGSGGGGGSGGGGSGGGGS | 18 |
| Linker 2 | GGGGS | 19 |

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5 (KA); and LC-CDR3: SEQ ID NO: 6, and wherein the CDRs comprise from 0-2 amino acid modifications in at least one of said LC-CDR1, LC-CDR2, or LC-CDR3. In some embodiments, A₁ comprises complementarity determining regions (CDRs): LC-CDR1, LC-CDR2, and LC-CDR3, wherein the LC-CDR1, the LC-CDR2, and the LC-CDR3 of A, comprise: LC-CDR1: SEQ ID NO: 4; LC-CDR2: SEQ ID NO: 5 (KA); and LC-CDR3: SEQ ID NO: 6; wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 1; HC-CDR2: SEQ ID NO: 2; HC-CDR3: SEQ ID NO: 3.

TABLE 4
anti-CD28 heavy chain polypeptide complementarity
determining regions (CDR)s as determined by IMGT
definition.

| | Amino Acid Sequence | SEQ ID |
|---|---|---|
| Construct Description | (N to C) | NO: |
| anti-CD28: HC: CDR1 | GYTFTSYY | 1 |
| anti-CD28: HC: CDR2 | IYPGNVNT | 2 |
| anti-CD28: HC: CDR3 | TRSHYGLDWNFDV | 3 |

TABLE 5
anti-CD28 light chain polypeptide complementarity
determining regions (CDR)s as determined by IMGT
definition.

| | Amino Acid Sequence | SEQ ID |
|---|---|---|
| Construct Description | (N to C) | NO: |
| anti-CD28: LC: CDR1 | QNIYVW | 4 |
| anti-CD28: LC: CDR2 | KA | 5 |
| anti-CD28: LC: CDR3 | QQGQTYPYT | 6 |

In some embodiments, the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12; and wherein said CDRs comprise from 0-2 amino acid modifications in at least one of said HC-CDR1, HC-CDR2, or HC-CDR3. In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14 (DA); and LC-CDR3: SEQ ID NO: 15; and wherein the CDRs comprise from 0-2 amino acid modifications in at least one of said LC-CDR1, LC-CDR2, or LC-CDR3. In some embodiments, B comprises complementarity determining region (CDRs): HC-CDR1, HC-CDR2, and HC-CDR3, wherein the HC-CDR1, the HC-CDR2, and the HC-CDR3 of B comprise: HC-CDR1: SEQ ID NO: 10; HC-CDR2: SEQ ID NO: 11; HC-CDR3: SEQ ID NO: 12; and wherein B comprises complementarity determining regions (CDRs): LC-CDR1, LC-CDR2, and LC-CDR3, wherein the LC-CDR1, the LC-CDR2, and the LC-CDR3 of B comprise: LC-CDR1: SEQ ID NO: 13; LC-CDR2: SEQ ID NO: 14 (DA); and LC-CDR3: SEQ ID NO: 15.

TABLE 6
anti-PD-L1 heavy chain polypeptide complementarity
determining regions (CDR)s as determined by IMGT
definition.

| Construct | Amino Acid Sequence | SEQ ID |
|---|---|---|
| Description | (N to C) | NO: |
| anti-PD-L1 Fab 1: | GDTFSTYA | 10 |
| HC: CDR1 | | |
| anti-PD-L1 Fab 1: | IIPIFGKA | 11 |
| HC: CDR2 | | |
| anti-PD-L1 Fab 1: | ARKFHFVSGSPFGMDV | 12 |
| HC: CDR3 | | |

TABLE 7
anti-PD-L1 light chain polypeptide complementarity
determining regions (CDR)s as determined by IMGT
definition.

| Construct | Amino Acid Sequence | SEQ ID |
|---|---|---|
| Description | (N to C) | NO: |
| anti-PD-L1 Fab 1: | QSVSSY | 13 |
| LC: CDR1 | | |
| anti-PD-L1 Fab 1: | DA | 14 |
| LC: CDR2 | | |
| anti-PD-L1 Fab 1: | QQRSNWPT | 15 |
| LC: CDR3 | | |

In some embodiments, the scFv heavy chain variable domain comprises an amino acid sequence that has at least 80% sequence identity to the amino acid sequence according to SEQ ID NO: 7. In some embodiments, the scFv heavy chain variable domain comprises an amino acid sequence of at least 75 consecutive amino acid residues of SEQ ID NO: 7 In some embodiments, the scFv heavy chain variable domain comprises an amino acid sequence of at least 110 consecutive amino acid residues of SEQ ID NO: 7. In some embodiments, the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7. In some embodiments, the scFv heavy chain variable domain comprises an amino acid sequence according to SEQ ID NO: 7.

In some embodiments, the scFv light chain variable domain comprises an amino acid sequence that has at least 80% sequence identity to the amino acid sequence according to SEQ ID NO: 8. In some embodiments, the scFv light chain variable domain comprises an amino acid sequence of at least 75 consecutive amino acid residues of SEQ ID NO: 8. In some embodiments, the scFv light chain variable domain comprises an amino acid sequence of at least 100 consecutive amino acid residues of SEQ ID NO: 8. In some embodiments, the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8. In some embodiments, the scFv light chain variable domain comprises an amino acid sequence according to SEQ ID NO: 8.

In some embodiments, the scFv comprises an amino acid sequence that has at least 80% sequence identity to the amino acid sequence according to SEQ ID NO: 9. In some embodiments, the scFv comprises an amino acid sequence of at least 175 consecutive amino acid residues of SEQ ID NO: 9. In some embodiments, the scFv comprises an amino acid sequence of at least 210 consecutive amino acid residues of SEQ ID NO: 9. In some embodiments, the scFv comprises an amino acid sequence of at least 210 consecutive amino acid residues of SEQ ID NO: 9 and has at least 80% sequence identity to the at least 210 consecutive amino acid residues of SEQ ID NO: 9. In some embodiments, the scFv comprises an amino acid sequence according to SEQ ID NO: 9.

TABLE 8
anti-CD28 light chain variable domain, heavy chain variable
domain sequences, and fulll ength sequence. CDR sequences are
underlined and were determined using IMGT definition.

| | Amino Acid Sequence | SEQ ID |
|---|---|---|
| Construct Description | (N to C) | NO: |
| anti-CD28: HC | QVQLVQSGAEVKKPGASVKV | 7 |
| | SCKASGYTFTSYYIHWVRQAP | |
| | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSS | |
| anti-CD28: LC | DIQMTQSPSSLSASVGDRVTIT | 8 |
| | CHASQNIYVWLNWYQQKPG | |
| | KAPKLLIYKASNLHTGVPSRFS | |
| | GSGSGTDFTLTISSLQPEDFAT | |
| | YYCQQGQTYPYTFGGGTKVE | |
| | IK | |
| Anti-CD28 scFv | QVQLVQSGAEVKKPGASVKV | 9 |
| (VH-linker 1-VL) | SCKASGYTFTSYYIHWVRQAP | |
| | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIK | |

In some embodiments, the Fab heavy chain polypeptide comprises an amino acid sequence that has at least 80% sequence identity to the amino acid sequence according to SEQ ID NO: 17. In some embodiments, the Fab heavy chain polypeptide comprises an amino acid sequence of at least 175 consecutive amino acid residues of SEQ ID NO: 17. In some embodiments, the Fab heavy chain polypeptide comprises an amino acid sequence of at least 215 consecutive amino acid residues of SEQ ID NO: 17. In some embodiments, the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17. In some embodiments, the Fab heavy chain polypeptide comprises an amino acid sequence according to SEQ ID NO: 17.

In some embodiments, the Fab light chain polypeptide comprises an amino acid sequence that has at least 80% sequence identity to the amino acid sequence according to SEQ ID NO: 16. In some embodiments, the Fab light chain polypeptide comprises an amino acid sequence of at least 175 consecutive amino acid residues of SEQ ID NO: 16. In some embodiments, the Fab light chain polypeptide comprises an amino acid sequence of at least 200 consecutive amino acid residues of SEQ ID NO: 16. In some embodiments, the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16. In some embodiments, the Fab light chain polypeptide comprises an amino acid sequence according to SEQ ID NO: 16.

TABLE 9
anti-PD-L1 Fab light chain polypeptide and Fab heavy chain polypeptide
sequences. CDR sequences are underlined and were determined using
IMGT definition

| Construct | Amino Acid Sequence | SEQ ID |
|---|---|---|
| Description | (N to C) | NO: |
| anti-PD-L1 | EIVLTQSPATLSLSPGERATLSCRASQSVSSYLAWYQQKPGQ | 16 |
| Fab 1: LC | APRLLIYDASNRATGIPARFSGSGSGTDFTLTISSLEPEDFAVY | |
| | YCQQRSNWPTFGQGTKVEIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| anti-PD-L1 | QVQLVQSGAEVKKPGSSVKVSCKTSGDTFSTYAISWVRQAP | 17 |
| Fab 1: HC | GQGLEWMGGIIPIFGKAHYAQKFQGRVTITADESTSTAYMEL | |
| | SSLRSEDTAVYFCARKFHFVSGSPFGMDVWGQGTTVTVSSA | |
| | STKGPSVFPLAPSSKSTSGGTAALGCLVKDYFPEPVTVSWNS | |
| | GALTSGVHTFPAVLQSSGLYSLSSVVTVPSSSLGTQTYICNV | |
| | NHKPSNTKVDKKVEPKSC | |

In some embodiments, the linker connects the Fab heavy chain polypeptide to the C-terminus of the scFv light chain variable domain and wherein, the Fab light chain polypeptide comprises an amino acid sequence that has at least 80% sequence identity to the amino acid sequence according to SEQ ID NO: 20 and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21. In some embodiments, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO:21. In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

TABLE 10
Antibodies that Bind to CD28 and PD-L1

| Construct | Amino Acid Sequence | SEQ ID |
|---|---|---|
| Description | (N to C) | NO: |
| Ab-12 LC | EIVLTQSPATLSLSPGERATLSCRASQSVSS | 20 |
| PDL1xCD28 | YLAWYQQKPGQAPRLLIYDASNRATGIPA | |
| non-masked | RFSGSGSGTDFTLTISSLEPEDFAVYYCQQ | |
| (Vh) | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-12 HC | QVQLVQSGAEVKKPGASVKVSCKASGYT | 21 |
| PDL1xCD28 | FTSYYIHWVRQAPGQGLEWIGSIYPGNVN | |
| non-masked | TNYNEKFKDRATLTVDTSISTAYMELSRL | |
| (Vh) | RSDDTAVYFCTRSHYGLDWNFDVWGQG | |
| Anti-CD28 | TTVTVSSGGGGSGGGGSGGGGSDIQMTQ | |
| scFv-Linker 2- | SPSSLSASVGDRVTITCHASQNIYVWLNW | |
| anti-PDL1 | YQQKPGKAPKLLIYKASNLHTGVPSRFSG | |
| Fab HC | SGSGTDFTLTISSLQPEDFATYYCQQGQTY | |
| | PYTFGGGTKVEIKGGGGSQVQLVQSGAE | |
| | VKKPGSSVKVSCKTSGDTFSTYAISWVRQ | |
| | APGQGLEWMGGIIPIFGKAHYAQKFQGRV | |
| | TITADESTSTAYMELSSLRSEDTAVYFCAR | |
| | KFHFVSGSPFGMDVWGQGTTVTVSSAST | |
| | KGPSVFPLAPSSKSTSGGTAALGCLVKDY | |
| | FPEPVTVSWNSGALTSGVHTFPAVLQSSG | |
| | LYSLSSVVTVPSSSLGTQTYICNVNHKPSN | |
| | TKVDKKVEPKSC | |
| Ab-13 LC | QVQLVQSGAEVKKPGASVKVSCKASGYT | 22 |
| PDL1xCD28 | FTSYYIHWVRQAPGQGLEWIGSIYPGNVN | |
| non-masked | TNYNEKFKDRATLTVDTSISTAYMELSRL | |
| (VL) | RSDDTAVYFCTRSHYGLDWNFDVWGQG | |
| | TTVTVSSGGGGSGGGGSGGGGSDIQMTQ | |
| | SPSSLSASVGDRVTITCHASQNIYVWLNW | |
| | YQQKPGKAPKLLIYKASNLHTGVPSRFSG | |
| | SGSGTDFTLTISSLQPEDFATYYCQQGQTY | |
| | PYTFGGGTKVEIKGGGGSEIVLTQSPATLS | |
| | LSPGERATLSCRASQSVSSYLAWYQQKPG | |
| | QAPRLLIYDASNRATGIPARFSGSGSGTDF | |
| | TLTISSLEPEDFAVYYCQQRSNWPTFGQG | |
| | TKVEIKRTVAAPSVFIFPPSDEQLKSGTAS | |
| | VVCLLNNFYPREAKVQWKVDNALQSGNS | |
| | QESVTEQDSKDSTYSLSSTLTLSKADYEK | |
| | HKVYACEVTHQGLSSPVTKSFNRGEC | |
| Ab-13 HC | QVQLVQSGAEVKKPGSSVKVSCKTSGDT | 23 |
| PDL1xCD28 | FSTYAISWVRQAPGQGLEWMGGIIPIFGK | |
| non-masked | AHYAQKFQGRVTITADESTSTAYMELSSL | |
| (VL) | RSEDTAVYFCARKFHFVSGSPFGMDVWG | |
| | QGTTVTVSSASTKGPSVFPLAPSSKSTSGG | |
| | TAALGCLVKDYFPEPVTVSWNSGALTSG | |
| | VHTFPAVLQSSGLYSLSSVVTVPSSSLGTQ | |
| | TYICNVNHKPSNTKVDKKVEPKSC | |
| JXA2618 LC | EIVLTQSPATLSLSPGERATLSCRASQSVSS | 210 |
| PDL1xCD28 | YLAWYQQKPGQAPRLLIYDASNRATGIPA | |
| masked; non- | RFSGSGSGTDFTLTISSLEPEDFAVYYCQQ | |
| cleavable (Vh) | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| JXA2618 HC | EVQLVESGGGLVQPGNSLRLSCAASGFTF | 211 |
| PDL1xCD28 | SKFGMSWVRQAPGKGLEWVSSISGSGRD | |
| masked; non- | TLYADSVKGRFTISRDNAKTTLYLQMNSL | |
| cleavable (Vh) | RPEDTAVYYCTIGGSLSVSSQGTLVTVSSG | |
| | GGGSGGGSGGMDWCPRDLWVHCFAGGG | |
| | GSGGGSGGGGSGGASSGAGGSGGGSQVQ | |
| | LVQSGAEVKKPGASVKVSCKASGYTFTS | |
| | YYIHWVRQAPGQGLEWIGSIYPGNVNTN | |
| | YNEKFKDRATLTVDTSISTAYMELSRLRS | |
| | DDTAVYFCTRSHYGLDWNFDVWGQGTT | |
| | VTVSSGGGGSGGGGSGGGGSDIQMTQSPS | |
| | SLSASVGDRVTITCHASQNIYVWLNWYQ | |
| | QKPGKAPKLLIYKASNLHTGVPSRFSGSG | |
| | SGTDFTLTISSLQPEDFATYYCQQGQTYPY | |
| | TFGGGTKVEIKGGGGSQVQLVQSGAEVK | |
| | KPGSSVKVSCKTSGDTFSTYAISWVRQAP | |
| | GQGLEWMGGIIPIFGKAHYAQKFQGRVTI | |
| | TADESTSTAYMELSSLRSEDTAVYFCARK | |
| | FHFVSGSPFGMDVWGQGTTVTVSSASTK | |
| | GPSVFPLAPSSKSTSGGTAALGCLVKDYFP | |
| | EPVTVSWNSGALTSGVHTFPAVLQSSGLY | |
| | SLSSVVTVPSSSLGTQTYICNVNHKPSNTK | |
| | VDKKVEPKSC | |
| JXA3777 LC | EIVLTQSPATLSLSPGERATLSCRASQSVSS | 212 |
| PDL1xCD28 | YLAWYQQKPGQAPRLLIYDASNRATGIPA | |
| masked; non- | RFSGSGSGTDFTLTISSLEPEDFAVYYCQQ | |
| cleavable (Vh) | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| JXA3777 HC | EVQLVESGGGLVQPGGSLRLSCAASGSTF | 213 |
| PDL1xCD28 | YTAVMGWVRQAPGKGLEWVAAIRWTAL | |
| masked; non- | TTSYADSVKGRFTISRDGAKTTLYLQMNS | |
| cleavable (Vh) | LRPEDTAVYYCAARGTLGLFTTADSYDY | |
| | WGQGTLVTVSSGGGGSGGGSGGMDWCP | |
| | RDLWVHCFAGGGGSGGGSGGGGSGGASS | |
| | GAGGSGGGSQVQLVQSGAEVKKPGASVK | |
| | VSCKASGYTFTSYYIHWVRQAPGQGLEWI | |
| | GSIYPGNVNTNYNEKFKDRATLTVDTSIST | |
| | AYMELSRLRSDDTAVYFCTRSHYGLDWN | |
| | FDVWGQGTTVTVSSGGGGSGGGGSGGG | |
| | GSDIQMTQSPSSLSASVGDRVTITCHASQN | |
| | IYVWLNWYQQKPGKAPKLLIYKASNLHT | |
| | GVPSRFSGSGSGTDFTLTISSLQPEDFATY | |
| | YCQQGQTYPYTFGGGTKVEIKGGGGSQV | |
| | QLVQSGAEVKKPGSSVKVSCKTSGDTFST | |
| | YAISWVRQAPGQGLEWMGGIIPIFGKAHY | |
| | AQKFQGRVTITADESTSTAYMELSSLRSE | |
| | DTAVYFCARKFHFVSGSPFGMDVWGQGT | |
| | TVTVSSASTKGPSVFPLAPSSKSTSGGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPKSC | |

### Half-Life Extending Molecule (H₁)

In some embodiments, the multispecific antibody further comprises a half-life extending molecule (H₁). In some embodiments, H₁ is connected to P₁. In some embodiments, H₁ is connected to P₂. In some embodiments, H₁ does not block A₁ binding to CD28. In some embodiments, H₁ does not block B binding to PD-L1. H₁ comprises a linking moiety (L₅) that connects H₁ to P₁ or H₁ to P₂. In some embodiments, the half-life extending molecule (H₁) does not have binding affinity to PD-L1. In some embodiments, the half-life extending molecule (H₁) does not have binding affinity to CD28. In some embodiments, the half-life extending molecule (H₁) does not shield the multispecific antibody from CD28. In some embodiments, H₁ comprises a sequence according to SEQ ID NOs: 54-57. In some embodiments, H₁ comprises an amino acid sequence that has repetitive sequence motifs. In some embodiments, H₁ comprises an amino acid sequence that has highly ordered secondary structure. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, sIgA, IgM or IgD. In some embodiments, the serum protein is albumin. In some embodiments, the polypeptide is an antibody. In some embodiments, the antibody comprises a single domain antibody, a single chain variable fragment, a Fab, or a Fab′. In some embodiments, the single domain antibody comprises a single domain antibody that binds to albumin. In some embodiments, the single domain antibody is a human or humanized antibody. In some embodiments, the single domain antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10E and SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3. In some embodiments, H₁ comprises an amino acid sequence according to SEQ ID NO: 57. In some embodiments, H₁ comprises an amino acid sequence that has at least 80% sequence identity to SEQ ID NO: 57. In some embodiments, H₁ comprises an amino acid sequence that has at least 85% sequence identity to SEQ ID NO: 57. In some embodiments, H₁ comprises an amino acid sequence that has at least 90% sequence identity to SEQ ID NO: 57. In some embodiments, H₁ comprises an amino acid sequence that has at least 95% sequence identity to SEQ ID NO: 57. In some embodiments, H₁ comprises an amino acid sequence that has at least 99% sequence identity to SEQ ID NO: 57. In some embodiments, H₁ comprise a modified amino acid or non-natural amino acid, or a modified non-natural amino acid, or a combination thereof. In some embodiments, the modified amino acid or a modified non-natural amino acid comprises a post-translational modification. In some embodiments, H₁ comprises a linking moiety (L₅) that connects H₁ to P₁ or P₂. In some embodiments, L₅ is a peptide sequence having at least 5 to no more than 50 amino acids. In some embodiments, L₅ is a peptide sequence having at least 10 to no more than 30 amino acids. In some embodiments, L₅ is a peptide sequence having at least 10 amino acids. In some embodiments, L₅ is a peptide sequence having at least 18 amino acids. In some embodiments, L₅ is a peptide sequence having at least 26 amino acids. In some embodiments, L₅ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 204, HC-CDR2: SEQ ID NO: 205, and HC-CDR3: SEQ ID NO: 206; and wherein the CDRs comprise from 0-2 amino acid modifications in at least one of the HC-CDR1, HC-CDR2, or HC-CDR3. In some embodiments, H₁ comprises an amino acid sequence according to SEQ ID NO: 207. In some embodiments, H₁ comprises an amino acid sequence that has at least 80% sequence identity to SEQ ID NO: 207. In some embodiments, H₁ comprises an amino acid sequence that has at least 85% sequence identity to SEQ ID NO: 207. In some embodiments, H₁ comprises an amino acid sequence that has at least 90% sequence identity to SEQ ID NO: 207. In some embodiments, H₁ comprises an amino acid sequence that has at least 95% sequence identity to SEQ ID NO: 207. In some embodiments, H₁ comprises an amino acid sequence that has at least 99% sequence identity to SEQ ID NO: 207. In some embodiments, H₁ comprise a modified amino acid or non-natural amino acid, or a modified non-natural amino acid, or a combination thereof. In some embodiments, the modified amino acid or a modified non-natural amino acid comprises a post-translational modification. In some embodiments, H₁ comprises a linking moiety (L₅) that connects H₁ to P₁ or P₂. In some embodiments, L₅ is a peptide sequence having at least 5 to no more than 50 amino acids. In some embodiments, L₅ is a peptide sequence having at least 10 to no more than 30 amino acids. In some embodiments, L₅ is a peptide sequence having at least 10 amino acids. In some embodiments, L₅ is a peptide sequence having at least 18 amino acids. In some embodiments, L₅ is a peptide sequence having at least 26 amino acids. In some embodiments, L₅ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

TABLE 11
H1 Sequences

| | Amino Acid Sequence | |
|---|---|---|
| Construct Description | (N to C) | SEQ ID NO: |
| Anti-Albumin: CDR-H1 | GSTFYTAV | 54 |
| Anti-Albumin: CDR-H2 | IRWTALTT | 55 |
| Anti-Albumin: CDR-H3 | AARGTLGLFTTADSYDY | 56 |
| Anti-albumin (HE-1) | EVQLVESGGGLVQPGGSLRLSCAASGSTF | 57 |
| | YTAVMGWVRQAPGKGLEWVAAIRWTA | |
| | LTTSYADSVKGRFTISRDGAKTTLYLQM | |
| | NSLRPEDTAVYYCAARGTLGLFTTADSY | |
| | DYWGQGTLVTVSS | |
| Anti-Albumin: CDR-H1 | GFTFSKFG | 204 |
| Anti-Albumin: CDR-H2 | ISGSGRDT | 205 |
| Anti-Albumin: CDR-H3 | TIGGSLSV | 206 |
| Anti-albumin (HE-3) | EVQLVESGGGLVQPGNSLRLSCAASGFT | 207 |
| | FSKFGMSWVRQAPGKGLEWVSSISGSGR | |
| | DTLYADSVKGRFTISRDNAKTTLYLQMN | |
| | SLRPEDTAVYYCTIGGSLSVSSQGTLVTV | |
| | SS | |

Tumor Activated Multispecific Antibodies that Bind to CD28 and PD-L1

In some embodiments, the isolated multispecific antibody comprises an amino acid sequence that has at least 80% sequence identity to any one of SEQ ID NOs: 149-170. In some embodiments, the isolated multispecific antibody comprises an amino acid sequence that has at least 85% sequence identity to any one of SEQ ID NOs: 149-170. In some embodiments, the isolated multispecific antibody comprises an amino acid sequence that has at least 90% sequence identity to any one of SEQ ID NOs: 149-170. In some embodiments, the isolated multispecific antibody comprises an amino acid sequence that has at least 95% sequence identity to any one of SEQ ID NOs: 149-170. In some embodiments, the isolated multispecific antibody comprises an amino acid sequence that has at least 99% sequence identity to any one of SEQ ID NOs: 149-170. In some embodiments, the isolated multispecific antibody comprises an amino acid sequence of any one of SEQ ID NOs: 149-170.

In some embodiments, the isolated multispecific antibody comprises an amino acid sequence that has at least 95% sequence identity to SEQ ID NOs: 149 and 150. In some embodiments, the isolated multispecific antibody comprises an amino acid sequence that has at least 99% sequence identity to SEQ ID NOs: 149 and 150.

In some embodiments, the isolated multispecific antibody comprises an amino acid sequence that has at least 95% sequence identity to SEQ ID NOs: 151 and 152. In some embodiments, the isolated multispecific antibody comprises an amino acid sequence that has at least 99% sequence identity to SEQ ID NOs: 151 and 152.

In some embodiments, the isolated multispecific antibody comprises an amino acid sequence that has at least 95% sequence identity to SEQ ID NOs: 153 and 154. In some embodiments, the isolated multispecific antibody comprises an amino acid sequence that has at least 99% sequence identity to SEQ ID NOs: 153 and 154.

In some embodiments, the isolated multispecific antibody comprises an amino acid sequence that has at least 95% sequence identity to SEQ ID NOs: 155 and 156. In some embodiments, the isolated multispecific antibody comprises an amino acid sequence that has at least 99% sequence identity to SEQ ID NOs: 155 and 156.

In some embodiments, the isolated multispecific antibody comprises an amino acid sequence that has at least 95% sequence identity to SEQ ID NOs: 157 and 158. In some embodiments, the isolated multispecific antibody comprises an amino acid sequence that has at least 99% sequence identity to SEQ ID NOs: 157 and 158.

In some embodiments, the isolated multispecific antibody comprises an amino acid sequence that has at least 95% sequence identity to SEQ ID NOs: 159 and 160. In some embodiments, the isolated multispecific antibody comprises an amino acid sequence that has at least 99% sequence identity to SEQ ID NOs: 159 and 160.

In some embodiments, the isolated multispecific antibody comprises an amino acid sequence that has at least 95% sequence identity to SEQ ID NOs: 161 and 162. In some embodiments, the isolated multispecific antibody comprises an amino acid sequence that has at least 99% sequence identity to SEQ ID NOs: 161 and 162.

In some embodiments, the isolated multispecific antibody comprises an amino acid sequence that has at least 95% sequence identity to SEQ ID NOs: 163 and 164. In some embodiments, the isolated multispecific antibody comprises an amino acid sequence that has at least 99% sequence identity to SEQ ID NOs: 163 and 164.

In some embodiments, the isolated multispecific antibody comprises an amino acid sequence that has at least 95% sequence identity to SEQ ID NOs: 165 and 166. In some embodiments, the isolated multispecific antibody comprises an amino acid sequence that has at least 99% sequence identity to SEQ ID NOs: 165 and 166.

In some embodiments, the isolated multispecific antibody comprises an amino acid sequence that has at least 95% sequence identity to SEQ ID NOs: 167 and 168. In some embodiments, the isolated multispecific antibody comprises an amino acid sequence that has at least 99% sequence identity to SEQ ID NOs: 167 and 168.

In some embodiments, the isolated multispecific antibody comprises an amino acid sequence that has at least 95% sequence identity to SEQ ID NOs: 169 and 170. In some embodiments, the isolated multispecific antibody comprises an amino acid sequence that has at least 99% sequence identity to SEQ ID NOs: 169 and 170.

In some embodiments, the isolated multispecific antibody comprises an amino acid sequence that has at least 95% sequence identity to SEQ ID NOs: 208 and 209. In some embodiments, the isolated multispecific antibody comprises an amino acid sequence that has at least 99% sequence identity to SEQ ID NOs: 208 and 209.

TABLE 12
Tumor Activated Multispecific Antibody Sequences that Bind to
CD28 and PD-L1.

| Construct | Amino Acid Sequence | SEQ ID |
|---|---|---|
| Description | (N to C) | NO: |
| Ab-1 LC | EIVLTQSPATLSLSPGERATLSCRASQSVSS | 149 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-1 HC | EVQLVESGGGLVQPGNSLRLSCAASGFTF | 150 |
| | SKFGMSWVRQAPGKGLEWVSSISGSGRD | |
| | TLYADSVKGRFTISRDNAKTTLYLQMNSL | |
| | RPEDTAVYYCTIGGSLSVSSQGTLVTVSSG | |
| | GGGSGGGSGGMDWCPRERWVDCFFGGG | |
| | GSGGGLSGRSDAGSPLGLAGSGGGSQVQ | |
| | LVQSGAEVKKPGASVKVSCKASGYTFTS | |
| | YYIHWVRQAPGQGLEWIGSIYPGNVNTN | |
| | YNEKFKDRATLTVDTSISTAYMELSRLRS | |
| | DDTAVYFCTRSHYGLDWNFDVWGQGTT | |
| | VTVSSGGGGSGGGGSGGGGSDIQMTQSPS | |
| | SLSASVGDRVTITCHASQNIYVWLNWYQ | |
| | QKPGKAPKLLIYKASNLHTGVPSRFSGSG | |
| | SGTDFTLTISSLQPEDFATYYCQQGQTYPY | |
| | TFGGGTKVEIKGGGGSQVQLVQSGAEVK | |
| | KPGSSVKVSCKTSGDTFSTYAISWVRQAP | |
| | GQGLEWMGGIIPIFGKAHYAQKFQGRVTI | |
| | TADESTSTAYMELSSLRSEDTAVYFCARK | |
| | FHFVSGSPFGMDVWGQGTTVTVSSASTK | |
| | GPSVFPLAPSSKSTSGGTAALGCLVKDYFP | |
| | EPVTVSWNSGALTSGVHTFPAVLQSSGLY | |
| | SLSSVVTVPSSSLGTQTYICNVNHKPSNTK | |
| | VDKKVEPKSC | |
| Ab-2 LC | EIVLTQSPATLSLSPGERATLSCRASQSVSS | 151 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-2 HC | EVQLVESGGGLVQPGNSLRLSCAASGFTF | 152 |
| | SKFGMSWVRQAPGKGLEWVSSISGSGRD | |
| | TLYADSVKGRFTISRDNAKTTLYLQMNSL | |
| | RPEDTAVYYCTIGGSLSVSSQGTLVTVSSG | |
| | GGGSGGGSGGMDWCPRERWVDCFFGSS | |
| | GGSAAGLLAPPGGLSGRSDAGGGGSQVQ | |
| | LVQSGAEVKKPGASVKVSCKASGYTFTS | |
| | YYIHWVRQAPGQGLEWIGSIYPGNVNTN | |
| | YNEKFKDRATLTVDTSISTAYMELSRLRS | |
| | DDTAVYFCTRSHYGLDWNFDVWGQGTT | |
| | VTVSSGGGGSGGGGSGGGGSDIQMTQSPS | |
| | SLSASVGDRVTITCHASQNIYVWLNWYQ | |
| | QKPGKAPKLLIYKASNLHTGVPSRFSGSG | |
| | SGTDFTLTISSLQPEDFATYYCQQGQTYPY | |
| | TFGGGTKVEIKGGGGSQVQLVQSGAEVK | |
| | KPGSSVKVSCKTSGDTFSTYAISWVRQAP | |
| | GQGLEWMGGIIPIFGKAHYAQKFQGRVTI | |
| | TADESTSTAYMELSSLRSEDTAVYFCARK | |
| | FHFVSGSPFGMDVWGQGTTVTVSSASTK | |
| | GPSVFPLAPSSKSTSGGTAALGCLVKDYFP | |
| | EPVTVSWNSGALTSGVHTFPAVLQSSGLY | |
| | SLSSVVTVPSSSLGTQTYICNVNHKPSNTK | |
| | VDKKVEPKSC | |
| Ab-3 LC | EIVLTQSPATLSLSPGERATLSCRASQSVSS | 153 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-3 HC | EVQLVESGGGLVQPGNSLRLSCAASGFTF | 154 |
| | SKFGMSWVRQAPGKGLEWVSSISGSGRD | |
| | TLYADSVKGRFTISRDNAKTTLYLQMNSL | |
| | RPEDTAVYYCTIGGSLSVSSQGTLVTVSSG | |
| | GGGSGGGSGGMDWCPRERWVDCFFGGG | |
| | GSGGGSGGGGSGGASSGAGGSGGGSQVQ | |
| | LVQSGAEVKKPGASVKVSCKASGYTFTS | |
| | YYIHWVRQAPGQGLEWIGSIYPGNVNTN | |
| | YNEKFKDRATLTVDTSISTAYMELSRLRS | |
| | DDTAVYFCTRSHYGLDWNFDVWGQGTT | |
| | VTVSSGGGGSGGGGSGGGGSDIQMTQSPS | |
| | SLSASVGDRVTITCHASQNIYVWLNWYQ | |
| | QKPGKAPKLLIYKASNLHTGVPSRFSGSG | |
| | SGTDFTLTISSLQPEDFATYYCQQGQTYPY | |
| | TFGGGTKVEIKGGGGSQVQLVQSGAEVK | |
| | KPGSSVKVSCKTSGDTFSTYAISWVRQAP | |
| | GQGLEWMGGIIPIFGKAHYAQKFQGRVTI | |
| | TADESTSTAYMELSSLRSEDTAVYFCARK | |
| | FHFVSGSPFGMDVWGQGTTVTVSSASTK | |
| | GPSVFPLAPSSKSTSGGTAALGCLVKDYFP | |
| | EPVTVSWNSGALTSGVHTFPAVLQSSGLY | |
| | SLSSVVTVPSSSLGTQTYICNVNHKPSNTK | |
| | VDKKVEPKSC | |
| Ab-4 LC | EIVLTQSPATLSLSPGERATLSCRASQSVSS | 155 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-4 HC | EVQLVESGGGLVQPGNSLRLSCAASGFTF | 156 |
| | SKFGMSWVRQAPGKGLEWVSSISGSGRD | |
| | TLYADSVKGRFTISRDNAKTTLYLQMNSL | |
| | RPEDTAVYYCTIGGSLSVSSQGTLVTVSSG | |
| | GGGSGGGSGGGGSGSSGGASSGGSGGGG | |
| | SGGGSGGGGSGGASSGAGGSGGGSQVQL | |
| | VQSGAEVKKPGASVKVSCKASGYTFTSY | |
| | YIHWVRQAPGQGLEWIGSIYPGNVNTNY | |
| | NEKFKDRATLTVDTSISTAYMELSRLRSD | |
| | DTAVYFCTRSHYGLDWNFDVWGQGTTV | |
| | TVSSGGGGSGGGGSGGGGSDIQMTQSPSS | |
| | LSASVGDRVTITCHASQNIYVWLNWYQQ | |
| | KPGKAPKLLIYKASNLHTGVPSRFSGSGS | |
| | GTDFTLTISSLQPEDFATYYCQQGQTYPYT | |
| | FGGGTKVEIKGGGGSQVQLVQSGAEVKK | |
| | PGSSVKVSCKTSGDTFSTYAISWVRQAPG | |
| | QGLEWMGGIIPIFGKAHYAQKFQGRVTIT | |
| | ADESTSTAYMELSSLRSEDTAVYFCARKF | |
| | HFVSGSPFGMDVWGQGTTVTVSSASTKG | |
| | PSVFPLAPSSKSTSGGTAALGCLVKDYFPE | |
| | PVTVSWNSGALTSGVHTFPAVLQSSGLYS | |
| | LSSVVTVPSSSLGTQTYICNVNHKPSNTKV | |
| | DKKVEPKSC | |
| Ab-5 LC | EIVLTQSPATLSLSPGERATLSCRASQSVSS | 157 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-5 HC | EVQLVESGGGLVQPGGSLRLSCAASGSTF | 158 |
| | YTAVMGWVRQAPGKGLEWVAAIRWTAL | |
| | TTSYADSVKGRFTISRDGAKTTLYLQMNS | |
| | LRPEDTAVYYCAARGTLGLFTTADSYDY | |
| | WGQGTLVTVSSGGGGSGGGSGGMDWCP | |
| | RERWVDCFFGGGGSGGGLSGRSDAGSPL | |
| | GLAGSGGGSQVQLVQSGAEVKKPGASVK | |
| | VSCKASGYTFTSYYIHWVRQAPGQGLEWI | |
| | GSIYPGNVNTNYNEKFKDRATLTVDTSIST | |
| | AYMELSRLRSDDTAVYFCTRSHYGLDWN | |
| | FDVWGQGTTVTVSSGGGGSGGGGSGGG | |
| | GSDIQMTQSPSSLSASVGDRVTITCHASQN | |
| | IYVWLNWYQQKPGKAPKLLIYKASNLHT | |
| | GVPSRFSGSGSGTDFTLTISSLQPEDFATY | |
| | YCQQGQTYPYTFGGGTKVEIKGGGGSQV | |
| | QLVQSGAEVKKPGSSVKVSCKTSGDTFST | |
| | YAISWVRQAPGQGLEWMGGIIPIFGKAHY | |
| | AQKFQGRVTITADESTSTAYMELSSLRSE | |
| | DTAVYFCARKFHFVSGSPFGMDVWGQGT | |
| | TVTVSSASTKGPSVFPLAPSSKSTSGGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPKSC | |
| Ab-6 LC | EIVLTQSPATLSLSPGERATLSCRASQSVSS | 159 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-6 HC | EVQLVESGGGLVQPGGSLRLSCAASGSTF | 160 |
| | YTAVMGWVRQAPGKGLEWVAAIRWTAL | |
| | TTSYADSVKGRFTISRDGAKTTLYLQMNS | |
| | LRPEDTAVYYCAARGTLGLFTTADSYDY | |
| | WGQGTLVTVSSGGGGSGGGSGGMDWCP | |
| | RERWVDCFFGSSGGSAAGLLAPPGGLSGR | |
| | SDAGGGGSQVQLVQSGAEVKKPGASVKV | |
| | SCKASGYTFTSYYIHWVRQAPGQGLEWIG | |
| | SIYPGNVNTNYNEKFKDRATLTVDTSISTA | |
| | YMELSRLRSDDTAVYFCTRSHYGLDWNF | |
| | DVWGQGTTVTVSSGGGGSGGGGSGGGG | |
| | SDIQMTQSPSSLSASVGDRVTITCHASQNI | |
| | YVWLNWYQQKPGKAPKLLIYKASNLHTG | |
| | VPSRFSGSGSGTDFTLTISSLQPEDFATYY | |
| | CQQGQTYPYTFGGGTKVEIKGGGGSQVQ | |
| | LVQSGAEVKKPGSSVKVSCKTSGDTFSTY | |
| | AISWVRQAPGQGLEWMGGIIPIFGKAHYA | |
| | QKFQGRVTITADESTSTAYMELSSLRSEDT | |
| | AVYFCARKFHFVSGSPFGMDVWGQGTTV | |
| | TVSSASTKGPSVFPLAPSSKSTSGGTAALG | |
| | CLVKDYFPEPVTVSWNSGALTSGVHTFPA | |
| | VLQSSGLYSLSSVVTVPSSSLGTQTYICNV | |
| | NHKPSNTKVDKKVEPKSC | |
| Ab-7 LC | EIVLTQSPATLSLSPGERATLSCRASQSVSS | 161 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-7 HC | EVQLVESGGGLVQPGGSLRLSCAASGSTF | 162 |
| | YTAVMGWVRQAPGKGLEWVAAIRWTAL | |
| | TTSYADSVKGRFTISRDGAKTTLYLQMNS | |
| | LRPEDTAVYYCAARGTLGLFTTADSYDY | |
| | WGQGTLVTVSSGGGGSGGGSGGMDWCP | |
| | RERWVDCFFGGGGSGGGSGGGGSGGASS | |
| | GAGGSGGGSQVQLVQSGAEVKKPGASVK | |
| | VSCKASGYTFTSYYIHWVRQAPGQGLEWI | |
| | GSIYPGNVNTNYNEKFKDRATLTVDTSIST | |
| | AYMELSRLRSDDTAVYFCTRSHYGLDWN | |
| | FDVWGQGTTVTVSSGGGGSGGGGSGGG | |
| | GSDIQMTQSPSSLSASVGDRVTITCHASQN | |
| | IYVWLNWYQQKPGKAPKLLIYKASNLHT | |
| | GVPSRFSGSGSGTDFTLTISSLQPEDFATY | |
| | YCQQGQTYPYTFGGGTKVEIKGGGGSQV | |
| | QLVQSGAEVKKPGSSVKVSCKTSGDTFST | |
| | YAISWVRQAPGQGLEWMGGIIPIFGKAHY | |
| | AQKFQGRVTITADESTSTAYMELSSLRSE | |
| | DTAVYFCARKFHFVSGSPFGMDVWGQGT | |
| | TVTVSSASTKGPSVFPLAPSSKSTSGGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPKSC | |
| Ab-8 LC | EIVLTQSPATLSLSPGERATLSCRASQSVSS | 163 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-8 HC | EVQLVESGGGLVQPGGSLRLSCAASGSTF | 164 |
| | YTAVMGWVRQAPGKGLEWVAAIRWTAL | |
| | TTSYADSVKGRFTISRDGAKTTLYLQMNS | |
| | LRPEDTAVYYCAARGTLGLFTTADSYDY | |
| | WGQGTLVTVSSGGGGSGGGSGGMDWCPI | |
| | YLWSECFNGSSGGSGGLSGRSDAGSPLGL | |
| | AGSGGGSQVQLVQSGAEVKKPGASVKVS | |
| | CKASGYTFTSYYIHWVRQAPGQGLEWIGS | |
| | IYPGNVNTNYNEKFKDRATLTVDTSISTA | |
| | YMELSRLRSDDTAVYFCTRSHYGLDWNF | |
| | DVWGQGTTVTVSSGGGGSGGGGSGGGG | |
| | SDIQMTQSPSSLSASVGDRVTITCHASQNI | |
| | YVWLNWYQQKPGKAPKLLIYKASNLHTG | |
| | VPSRFSGSGSGTDFTLTISSLQPEDFATYY | |
| | CQQGQTYPYTFGGGTKVEIKGGGGSQVQ | |
| | LVQSGAEVKKPGSSVKVSCKTSGDTFSTY | |
| | AISWVRQAPGQGLEWMGGIIPIFGKAHYA | |
| | QKFQGRVTITADESTSTAYMELSSLRSEDT | |
| | AVYFCARKFHFVSGSPFGMDVWGQGTTV | |
| | TVSSASTKGPSVFPLAPSSKSTSGGTAALG | |
| | CLVKDYFPEPVTVSWNSGALTSGVHTFPA | |
| | VLQSSGLYSLSSVVTVPSSSLGTQTYICNV | |
| | NHKPSNTKVDKKVEPKSC | |
| Ab-9 LC | EIVLTQSPATLSLSPGERATLSCRASQSVSS | 165 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| Ab-9 HC | EVQLVESGGGLVQPGGSLRLSCAASGSTF | 166 |
| | YTAVMGWVRQAPGKGLEWVAAIRWTAL | |
| | TTSYADSVKGRFTISRDGAKTTLYLQMNS | |
| | LRPEDTAVYYCAARGTLGLFTTADSYDY | |
| | WGQGTLVTVSSGGGGSGGGSGGMDWCP | |
| | RDLWVHCFAGSSGGSGGLSGRSDAGSPL | |
| | GLAGSGGGSQVQLVQSGAEVKKPGASVK | |
| | VSCKASGYTFTSYYIHWVRQAPGQGLEWI | |
| | GSIYPGNVNTNYNEKFKDRATLTVDTSIST | |
| | AYMELSRLRSDDTAVYFCTRSHYGLDWN | |
| | FDVWGQGTTVTVSSGGGGSGGGGSGGG | |
| | GSDIQMTQSPSSLSASVGDRVTITCHASQN | |
| | IYVWLNWYQQKPGKAPKLLIYKASNLHT | |
| | GVPSRFSGSGSGTDFTLTISSLQPEDFATY | |
| | YCQQGQTYPYTFGGGTKVEIKGGGGSQV | |
| | QLVQSGAEVKKPGSSVKVSCKTSGDTFST | |
| | YAISWVRQAPGQGLEWMGGIIPIFGKAHY | |
| | AQKFQGRVTITADESTSTAYMELSSLRSE | |
| | DTAVYFCARKFHFVSGSPFGMDVWGQGT | |
| | TVTVSSASTKGPSVFPLAPSSKSTSGGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPKSC | |
| Ab-10 LC | EVQLVESGGGLVQPGGSLRLSCAASGSTF | 167 |
| | YTAVMGWVRQAPGKGLEWVAAIRWTAL | |
| | TTSYADSVKGRFTISRDGAKTTLYLQMNS | |
| | LRPEDTAVYYCAARGTLGLFTTADSYDY | |
| | WGQGTLVTVSSGGGGSGGGSGGMDWCPI | |
| | YLWSECFNGSSGGSGGLSGRSDAGSPLGL | |
| | AGSGGGSQVQLVQSGAEVKKPGASVKVS | |
| | CKASGYTFTSYYIHWVRQAPGQGLEWIGS | |
| | IYPGNVNTNYNEKFKDRATLTVDTSISTA | |
| | YMELSRLRSDDTAVYFCTRSHYGLDWNF | |
| | DVWGQGTTVTVSSGGGGSGGGGSGGGG | |
| | SDIQMTQSPSSLSASVGDRVTITCHASQNI | |
| | YVWLNWYQQKPGKAPKLLIYKASNLHTG | |
| | VPSRFSGSGSGTDFTLTISSLQPEDFATYY | |
| | CQQGQTYPYTFGGGTKVEIKGGGGSEIVL | |
| | TQSPATLSLSPGERATLSCRASQSVSSYLA | |
| | WYQQKPGQAPRLLIYDASNRATGIPARFS | |
| | GSGSGTDFTLTISSLEPEDFAVYYCQQRSN | |
| | WPTFGQGTKVEIKRTVAAPSVFIFPPSDEQ | |
| | LKSGTASVVCLLNNFYPREAKVQWKVDN | |
| | ALQSGNSQESVTEQDSKDSTYSLSSTLTLS | |
| | KADYEKHKVYACEVTHQGLSSPVTKSFN | |
| | RGEC | |
| Ab-10 HC | QVQLVQSGAEVKKPGSSVKVSCKTSGDT | 168 |
| | FSTYAISWVRQAPGQGLEWMGGIIPIFGK | |
| | AHYAQKFQGRVTITADESTSTAYMELSSL | |
| | RSEDTAVYFCARKFHFVSGSPFGMDVWG | |
| | QGTTVTVSSASTKGPSVFPLAPSSKSTSGG | |
| | TAALGCLVKDYFPEPVTVSWNSGALTSG | |
| | VHTFPAVLQSSGLYSLSSVVTVPSSSLGTQ | |
| | TYICNVNHKPSNTKVDKKVEPKSC | |
| Ab-11 LC | EVQLVESGGGLVQPGGSLRLSCAASGSTF | 169 |
| | YTAVMGWVRQAPGKGLEWVAAIRWTAL | |
| | TTSYADSVKGRFTISRDGAKTTLYLQMNS | |
| | LRPEDTAVYYCAARGTLGLFTTADSYDY | |
| | WGQGTLVTVSSGGGGSGGGSGGMDWCP | |
| | RDLWVHCFAGSSGGSGGLSGRSDAGSPL | |
| | GLAGSGGGSQVQLVQSGAEVKKPGASVK | |
| | VSCKASGYTFTSYYIHWVRQAPGQGLEWI | |
| | GSIYPGNVNTNYNEKFKDRATLTVDTSIST | |
| | AYMELSRLRSDDTAVYFCTRSHYGLDWN | |
| | FDVWGQGTTVTVSSGGGGSGGGGSGGG | |
| | GSDIQMTQSPSSLSASVGDRVTITCHASQN | |
| | IYVWLNWYQQKPGKAPKLLIYKASNLHT | |
| | GVPSRFSGSGSGTDFTLTISSLQPEDFATY | |
| | YCQQGQTYPYTFGGGTKVEIKGGGGSEIV | |
| | LTQSPATLSLSPGERATLSCRASQSVSSYL | |
| | AWYQQKPGQAPRLLIYDASNRATGIPARF | |
| | SGSGSGTDFTLTISSLEPEDFAVYYCQQRS | |
| | NWPTFGQGTKVEIKRTVAAPSVFIFPPSDE | |
| | QLKSGTASVVCLLNNFYPREAKVQWKVD | |
| | NALQSGNSQESVTEQDSKDSTYSLSSTLTL | |
| | SKADYEKHKVYACEVTHQGLSSPVTKSF | |
| | NRGEC | |
| Ab-11 HC | QVQLVQSGAEVKKPGSSVKVSCKTSGDT | 170 |
| | FSTYAISWVRQAPGQGLEWMGGIIPIFGK | |
| | AHYAQKFQGRVTITADESTSTAYMELSSL | |
| | RSEDTAVYFCARKFHFVSGSPFGMDVWG | |
| | QGTTVTVSSASTKGPSVFPLAPSSKSTSGG | |
| | TAALGCLVKDYFPEPVTVSWNSGALTSG | |
| | VHTFPAVLQSSGLYSLSSVVTVPSSSLGTQ | |
| | TYICNVNHKPSNTKVDKKVEPKSC | |
| JXA 2616 LC | EIVLTQSPATLSLSPGERATLSCRASQSVSS | 208 |
| | YLAWYQQKPGQAPRLLIYDASNRATGIPA | |
| | RFSGSGSGTDFTLTISSLEPEDFAVYYCQQ | |
| | RSNWPTFGQGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVT | |
| | KSFNRGEC | |
| JXA 2616 HC | EVQLVESGGGLVQPGNSLRLSCAASGFTF | 209 |
| | SKFGMSWVRQAPGKGLEWVSSISGSGRD | |
| | TLYADSVKGRFTISRDNAKTTLYLQMNSL | |
| | RPEDTAVYYCTIGGSLSVSSQGTLVTVSSG | |
| | GGGSGGGSGGMDWCPRDLWVHCFAGSS | |
| | GGSGGLSGRSDAGSPLGLAGSGGGSQVQ | |
| | LVQSGAEVKKPGASVKVSCKASGYTFTS | |
| | YYIHWVRQAPGQGLEWIGSIYPGNVNTN | |
| | YNEKFKDRATLTVDTSISTAYMELSRLRS | |
| | DDTAVYFCTRSHYGLDWNFDVWGQGTT | |
| | VTVSSGGGGSGGGGSGGGGSDIQMTQSPS | |
| | SLSASVGDRVTITCHASQNIYVWLNWYQ | |
| | QKPGKAPKLLIYKASNLHTGVPSRFSGSG | |
| | SGTDFTLTISSLQPEDFATYYCQQGQTYPY | |
| | TFGGGTKVEIKGGGGSQVQLVQSGAEVK | |
| | KPGSSVKVSCKTSGDTFSTYAISWVRQAP | |
| | GQGLEWMGGIIPIFGKAHYAQKFQGRVTI | |
| | TADESTSTAYMELSSLRSEDTAVYFCARK | |
| | FHFVSGSPFGMDVWGQGTTVTVSSASTK | |
| | GPSVFPLAPSSKSTSGGTAALGCLVKDYFP | |
| | EPVTVSWNSGALTSGVHTFPAVLQSSGLY | |
| | SLSSVVTVPSSSLGTQTYICNVNHKPSNTK | |
| | VDKKVEPKSC | |

Polynucleotides Encoding Tumor Activated Multispecific Antibodies that Bind to CD28 and PD-L1

Disclosed herein, in some embodiments, are isolated recombinant nucleic acid molecules encoding the multispecific antibodies disclosed herein.

Disclosed herein, in some embodiments, are isolated recombinant nucleic acid molecules encoding isolated multispecific antibodies according to the following formula: P₁-L₁-A₁-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

Disclosed herein, in some embodiments, are isolated recombinant nucleic acid molecules encoding isolated multispecific antibodies comprising the following formula: P₁-L₁-A₁-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

Disclosed herein, in some embodiments, are isolated recombinant nucleic acid molecules encoding isolated multispecific antibodies comprising the following formula: P₁-L₁-A₁-L-B (Formula I) wherein A₁ is a CD28 binding domain; B is a PD-L1 binding domain; L is a linker that connects A₁ to B; P₁ is a peptide that binds to A₁ and L₁ is a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

Disclosed herein, in some embodiments, are isolated recombinant nucleic acid molecules encoding isolated multispecific antibodies according to the following formula: P₁-L₁-A₁-L-B (Formula I) wherein A₁ is a CD28 binding domain; B is a PD-L1 binding domain; L is a linker that connects A₁ to B; P₁ is a peptide that binds to A₁ and L₁ is a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

In some embodiments, are isolated recombinant nucleic acid molecules encoding isolated multispecific antibodies according to the following formula: P₁-L₁-A₁-L-B-L₂-P₂ (Formula Ia) wherein P₂ comprises a peptide that binds to B and L₂ comprises a linking moiety that connects B to P₂ and is a substrate for a tumor specific protease.

In some embodiments, are isolated recombinant nucleic acid molecules encoding isolated multispecific antibodies comprises the following formula: P₁-L₁-A₁-L-B-L₂-P₂ (Formula Ia) wherein P₂ comprises a peptide that binds to B and L₂ comprises a linking moiety that connects B to P₂ and is a substrate for a tumor specific protease.

In some embodiments, are isolated recombinant nucleic acid molecules encoding isolated multispecific antibodies comprises the following formula: P₁-L₁-A₁-L-B-L₂-P₂ (Formula Ia) wherein P₂ is a peptide that binds to B and L₂ is a linking moiety that connects B to P₂ and is a substrate for a tumor specific protease.

In some embodiments, are isolated recombinant nucleic acid molecules encoding isolated multispecific antibodies according to the following formula: P₁-L₁-A₁-L-B-L₂-P₂ (Formula Ia) wherein P₂ is a peptide that binds to B and L₂ is a linking moiety that connects B to P₂ and is a substrate for a tumor specific protease.

Disclosed herein, in some embodiments, are isolated recombinant nucleic acid molecules encoding an isolated multispecific antibody that comprises an amino acid sequence that has at least 80% sequence identity to any one of SEQ ID NOs: 149-170. In some embodiments, are isolated recombinant nucleic acid molecules encoding an isolated multispecific antibody that comprises an amino acid sequence that has at least 85% sequence identity to any one of SEQ ID NOs: 149-170. In some embodiments, are isolated recombinant nucleic acid molecules encoding an isolated multispecific antibody that comprises an amino acid sequence that has at least 90% sequence identity to any one of SEQ ID NOs: 149-170. In some embodiments, are isolated recombinant nucleic acid molecules encoding an isolated multispecific antibody that comprises an amino acid sequence that has at least 95% sequence identity to any one of SEQ ID NOs: 149-170. In some embodiments, are isolated recombinant nucleic acid molecules encoding an isolated multispecific antibody that comprises an amino acid sequence that has at least 99% sequence identity to any one of SEQ ID NOs: 149-170. In some embodiments, are isolated recombinant nucleic acid molecules encoding an isolated multispecific antibody that comprises an amino acid sequence of any one of SEQ ID NOs: 149-170.

In some embodiments, the isolated multispecific antibody comprises an amino acid sequence that has at least 95% sequence identity to SEQ ID NOs: 149 and 150. In some embodiments, are isolated recombinant nucleic acid molecules encoding an isolated multispecific antibody that comprises an amino acid sequence that has at least 99% sequence identity to SEQ ID NOs: 149 and 150.

In some embodiments, are isolated recombinant nucleic acid molecules encoding an isolated multispecific antibody that comprises an amino acid sequence that has at least 95% sequence identity to SEQ ID NOs: 151 and 152. In some embodiments, are isolated recombinant nucleic acid molecules encoding an isolated multispecific antibody that comprises an amino acid sequence that has at least 99% sequence identity to SEQ ID NOs: 151 and 152.

In some embodiments, are isolated recombinant nucleic acid molecules encoding an isolated multispecific antibody that comprises an amino acid sequence that has at least 95% sequence identity to SEQ ID NOs: 153 and 154. In some embodiments, are isolated recombinant nucleic acid molecules encoding an isolated multispecific antibody that comprises an amino acid sequence that has at least 99% sequence identity to SEQ ID NOs: 153 and 154.

In some embodiments, are isolated recombinant nucleic acid molecules encoding an isolated multispecific antibody that comprises an amino acid sequence that has at least 95% sequence identity to SEQ ID NOs: 155 and 156. In some embodiments, are isolated recombinant nucleic acid molecules encoding an isolated multispecific antibody that comprises an amino acid sequence that has at least 99% sequence identity to SEQ ID NOs: 155 and 156.

In some embodiments, are isolated recombinant nucleic acid molecules encoding an isolated multispecific antibody that comprises an amino acid sequence that has at least 95% sequence identity to SEQ ID NOs: 157 and 158. In some embodiments, are isolated recombinant nucleic acid molecules encoding an isolated multispecific antibody that comprises an amino acid sequence that has at least 99% sequence identity to SEQ ID NOs: 157 and 158.

In some embodiments, are isolated recombinant nucleic acid molecules encoding an isolated multispecific antibody that comprises an amino acid sequence that has at least 95% sequence identity to SEQ ID NOs: 159 and 160. In some embodiments, are isolated recombinant nucleic acid molecules encoding an isolated multispecific antibody that comprises an amino acid sequence that has at least 99% sequence identity to SEQ ID NOs: 159 and 160.

In some embodiments, are isolated recombinant nucleic acid molecules encoding an isolated multispecific antibody that comprises an amino acid sequence that has at least 95% sequence identity to SEQ ID NOs: 161 and 162. In some embodiments, are isolated recombinant nucleic acid molecules encoding an isolated multispecific antibody that comprises an amino acid sequence that has at least 99% sequence identity to SEQ ID NOs: 161 and 162.

In some embodiments, are isolated recombinant nucleic acid molecules encoding an isolated multispecific antibody that comprises an amino acid sequence that has at least 95% sequence identity to SEQ ID NOs: 163 and 164. In some embodiments, are isolated recombinant nucleic acid molecules encoding an isolated multispecific antibody that comprises an amino acid sequence that has at least 99% sequence identity to SEQ ID NOs: 163 and 164.

In some embodiments, are isolated recombinant nucleic acid molecules encoding an isolated multispecific antibody that comprises an amino acid sequence that has at least 95% sequence identity to SEQ ID NOs: 165 and 166. In some embodiments, are isolated recombinant nucleic acid molecules encoding an isolated multispecific antibody that comprises an amino acid sequence that has at least 99% sequence identity to SEQ ID NOs: 165 and 166.

In some embodiments, are isolated recombinant nucleic acid molecules encoding an isolated multispecific antibody that comprises an amino acid sequence that has at least 95% sequence identity to SEQ ID NOs: 167 and 168. In some embodiments, are isolated recombinant nucleic acid molecules encoding an isolated multispecific antibody that comprises an amino acid sequence that has at least 99% sequence identity to SEQ ID NOs: 167 and 168.

In some embodiments, are isolated recombinant nucleic acid molecules encoding an isolated multispecific antibody that comprises an amino acid sequence that has at least 95% sequence identity to SEQ ID NOs: 169 and 170. In some embodiments, are isolated recombinant nucleic acid molecules encoding an isolated multispecific antibody that comprises an amino acid sequence that has at least 99% sequence identity to SEQ ID NOs: 169 and 170.

In some embodiments, are isolated recombinant nucleic acid molecules encoding an isolated multispecific antibody that comprises an amino acid sequence that has at least 95% sequence identity to SEQ ID NOs: 208 and 209. In some embodiments, are isolated recombinant nucleic acid molecules encoding an isolated multispecific antibody that comprises an amino acid sequence that has at least 99% sequence identity to SEQ ID NOs: 208 and 209.

### Pharmaceutical Compositions

Disclosed herein, in some embodiments, are pharmaceutical compositions comprising: (a) multispecific antibodies as disclosed herein; and (b) a pharmaceutically acceptable excipient.

In some embodiments, the pharmaceutical composition comprises (a) isolated multispecific antibodies according to the following formula: P₁-L₁-A₁-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20; and (b) a pharmaceutically acceptable excipient.

In some embodiments, the pharmaceutical composition comprises (a) isolated multispecific antibodies comprising the following formula: P₁-L₁-A₁-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20; and (b) a pharmaceutically acceptable excipient.

In some embodiments, the pharmaceutical composition comprises (a) isolated multispecific antibodies comprising the following formula: P₁-L₁-A₁-L-B (Formula I) wherein A₁ is a CD28 binding domain; B is a PD-L1 binding domain; L is a linker that connects A₁ to B; P₁ is a peptide that binds to A₁ and L₁ is a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20; and (b) a pharmaceutically acceptable excipient.

In some embodiments, the pharmaceutical composition comprises (a) isolated multispecific antibodies according to the following formula: P₁-L₁-A₁-L-B (Formula I) wherein A₁ is a CD28 binding domain; B is a PD-L1 binding domain; L is a linker that connects A₁ to B; P₁ is a peptide that binds to A₁ and L₁ is a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20; and (b) a pharmaceutically acceptable excipient.

In some embodiments, the pharmaceutical composition comprises (a) isolated multispecific antibodies according to the following formula: P₁-L₁-A₁-L-B-L₂-P₂ (Formula Ia) wherein P₂ comprises a peptide that binds to B and L₂ comprises a linking moiety that connects B to P₂ and is a substrate for a tumor specific protease; and (b) a pharmaceutically acceptable excipient.

In some embodiments, the pharmaceutical composition comprises (a) isolated multispecific antibodies comprising the following formula: P₁-L₁-A₁-L-B-L₂-P₂ (Formula Ia) wherein P₂ comprises a peptide that binds to B and L₂ comprises a linking moiety that connects B to P₂ and is a substrate for a tumor specific protease; and (b) a pharmaceutically acceptable excipient.

In some embodiments, the pharmaceutical composition comprises (a) isolated multispecific antibodies comprising the following formula: P₁-L₁-A₁-L-B-L₂-P₂ (Formula Ia) wherein P₂ is a peptide that binds to B and L₂ is a linking moiety that connects B to P₂ and is a substrate for a tumor specific protease; and (b) a pharmaceutically acceptable excipient.

In some embodiments, the pharmaceutical composition comprises (a) isolated multispecific antibodies according to the following formula: P₁-L₁-A₁-L-B-L₂-P₂ (Formula Ia) wherein P₂ is a peptide that binds to B and L₂ is a linking moiety that connects B to P₂ and is a substrate for a tumor specific protease; and (b) a pharmaceutically acceptable excipient.

In some embodiments, the pharmaceutical composition comprises (a) an isolated multispecific antibody that comprises an amino acid sequence that has at least 80% sequence identity to any one of SEQ ID NOs: 149-170; and (b) a pharmaceutically acceptable excipient.

In some embodiments, the pharmaceutical composition comprises (a) an isolated multispecific antibody that comprises an amino acid sequence that has at least 85% sequence identity to any one of SEQ ID NOs: 149-170; and (b) a pharmaceutically acceptable excipient. In some embodiments, the pharmaceutical composition comprises (a) an isolated multispecific antibody that comprises an amino acid sequence that has at least 90% sequence identity to any one of SEQ ID NOs: 149-170; and (b) a pharmaceutically acceptable excipient. In some embodiments, the pharmaceutical composition comprises (a) an isolated multispecific antibody that comprises an amino acid sequence that has at least 95% sequence identity to any one of SEQ ID NOs: 149-170; and (b) a pharmaceutically acceptable excipient. In some embodiments, the pharmaceutical composition comprises (a) an isolated multispecific antibody that comprises an amino acid sequence that has at least 99% sequence identity to any one of SEQ ID NOs: 149-170; and (b) a pharmaceutically acceptable excipient. In some embodiments, the pharmaceutical composition comprises (a) an isolated multispecific antibody that comprises an amino acid sequence of any one of SEQ ID NOs: 149-170; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 149 and 150; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 149 and 150; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 151 and 152; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 151 and 152; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 153 and 154; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 153 and 154; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 155 and 156; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 155 and 156; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 157 and 158; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 157 and 158; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 159 and 160; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 159 and 160; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 161 and 162; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 161 and 162; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 163 and 164; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 163 and 164; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 165 and 166; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 165 and 166; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 167 and 168; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 167 and 168; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 169 and 170; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 169 and 170; and (b) a pharmaceutically acceptable excipient.

In some embodiments, the pharmaceutical composition comprises (a) an isolated multispecific antibody that comprises an amino acid sequence that has at least 95% sequence identity to SEQ ID NOs: 208 and 209; and (b) a pharmaceutically acceptable excipient. In some embodiments, the pharmaceutical composition comprises (a) an isolated multispecific antibody that comprises an amino acid sequence that has at least 99% sequence identity to SEQ ID NOs: 208 and 209; and (b) a pharmaceutically acceptable excipient.

Disclosed herein, are pharmaceutical compositions comprising: (a) the isolated multispecific antibodies described herein, (b) an anti-cancer therapy, and (c) a pharmaceutically acceptable excipient. In some embodiments, the anti-cancer therapy comprises a small molecule, a cell-based therapy, or an antibody-based therapy.

In some embodiments, the antibody-based therapy is a T cell engager. In some embodiments, the T cell engager comprises a formula according to: D₁-L₀-E₁ (Formula II), wherein D₁ comprises an effector cell binding domain that binds to an effector cell antigen, E₁ comprises a tumor antigen binding domain that binds to a tumor antigen, and L₀ comprises a linker that connects D₁ to E₁. In some embodiments, D₁ comprises a single chain variable fragment, a single domain antibody, a Fab fragment, or a Fab′. In some embodiments, D₁ comprises the single chain variable fragment. In some embodiments, E₁ comprises a single chain variable fragment, a single domain antibody, a Fab fragment, or a Fab′. In some embodiments, E₁ comprises the Fab fragment. In some embodiments, the effector cell antigen comprises CD3. In some embodiments, the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the effector cell binding domain comprises an amino acid sequence according to SEQ ID NOs: 89-101.

TABLE 13
Effector cell binding domain amino acid sequences

| | Amino Acid Sequence | SEQ ID |
|---|---|---|
| Construct Description | (N to C) | NO: |
| SP34.185 CD3: HC: CDR1 | GFTFNKYA | 89 |
| SP34.185 CD3: HC: CDR2 | IRSKYNNYAT | 90 |
| SP34.185 CD3: HC: CDR3 | VRHGNFGNSYISYWAY | 91 |
| SP34.185 CD3: LC: CDR1 | TGAVTSGNY | 92 |
| SP34.185 CD3: LC: CDR2 | GTK | 93 |
| SP34.185 CD3: LC: CDR3 | VLWYSNRWV | 94 |
| SP34.194 CD3: HC: CDR1 | GFTFNTYA | 95 |
| SP34.194 CD3: HC: CDR2 | IRSKYNNYAT | 90 |
| SP34.194 CD3: HC: CDR3 | VRHGNFGNSYVSWFAY | 96 |
| SP34.194 CD3: LC: CDR1 | TGAVTTSNY | 97 |
| SP34.194 CD3: LC: CDR2 | GT | 98 |
| SP34.194 CD3: LC: CDR3 | ALWYSNLWV | 99 |
| SP34.185 scFv | EVQLVESGGGLVQPGGSLKLS | 100 |
| (VH-linker 1-VL) | CAASGFTFNKYAMNWVRQA | |
| | PGKGLEWVARIRSKYNNYAT | |
| | YYADSVKDRFTISRDDSKNTA | |
| | YLQMNNLKTEDTAVYYCVRH | |
| | GNFGNSYISYWAYWGQGTLV | |
| | TVSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKL | |
| | TVL | |
| SP34.194 scFv | QTVVTQEPSLTVSPGGTVTLT | 101 |
| (VL-linker 1-VH) | CRSSTGAVTTSNYANWVQQK | |
| | PGQAPRGLIGGTNKRAPGTPA | |
| | RFSGSLLGGKAALTLSGVQPE | |
| | DEAEYYCALWYSNLWVFGG | |
| | GTKLTVLGGGGSGGGGSGGG | |
| | GSEVQLVESGGGLVQPGGSLK | |
| | LSCAASGFTFNTYAMNWVRQ | |
| | APGKGLEWVARIRSKYNNYA | |
| | TYYADSVKDRFTISRDDSKNT | |
| | AYLQMNNLKTEDTAVYYCVR | |
| | HGNFGNSYVSWFAYWGQGT | |
| | LVTVSS | |
| SP34.185 Peptide mask (P3) | GSQCLGPEWEVCPY | 177 |
| SP34.185 Peptide mask (P3) | VYCGPEFDESVGCM | 178 |
| SP34.185 Peptide mask (P3) | VYCGPEFDESVGCA | 179 |
| SP34.185 Peptide mask (P3) | YLCGPDGDETLACY | 180 |

In some embodiments, the tumor antigen comprises epidermal growth factor receptor (EGFR), prostate-specific membrane antigen (PSMA), or tumor-associated calcium signal transducer 2 (referred to herein after as TROP2). In some embodiments, the tumor antigen comprises EGFR. In some embodiments, the tumor antigen binding domain comprises an amino acid sequence according to SEQ ID NOs: 102-111. In some embodiments, the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 105; HC-CDR2: SEQ ID NO: 106; HC-CDR3: SEQ ID NO: 107; and wherein the LC-CDR1, the LC-CDR2, and the LC-CDR3 comprise: LC-CDR1: SEQ ID NO: 102; LC-CDR2: SEQ ID NO: 103 (YAS); and LC-CDR3: SEQ ID NO: 104. In some embodiments, the tumor antigen comprises EGFR, and the T cell engager comprises amino acid sequences with at least 95% sequence identity according to SEQ ID NOs: 181 and 182. In some embodiments, the tumor antigen comprises EGFR, and the T cell engager comprises amino acid sequences according to SEQ ID NOs: 181 and 182. In some embodiments, the tumor antigen comprises EGFR, and the T cell engager comprises amino acid sequences with at least 95% sequence identity according to SEQ ID NOs: 214 and 215. In some embodiments, the tumor antigen comprises EGFR, and the T cell engager comprises amino acid sequences according to SEQ ID NOs: 214 and 215.

TABLE 14
Tumor antigen binding domain amino acid sequences-anti-EGFR

| Construct | Amino Acid Sequence | |
|---|---|---|
| Description | (N to C) | SEQ ID NO: |
| EGFR: LC: CDR1 | QSIGTN | 102 |
| EGFR: LC: CDR2 | YAS | 103 |
| EGFR: LC: CDR3 | QQNNNWPTT | 104 |
| EGFR: HC: CDR1 | GFSLTNYG | 105 |
| EGFR: HC: CDR2 | IWSGGNT | 106 |
| EGFR: HC: CDR3 | ARALTYYDYEFAY | 107 |
| EGFR Fab LC v1 | QILLTQSPVILSVSPGERVSFSCRASQ | 108 |
| | SIGTNIHWYQQRTNGSPRLLIKYAS | |
| | ESISGIPSRFSGSGSGTDFTLSINSVES | |
| | EDIADYYCQQNNNWPTTFGAGTKL | |
| | ELKRTVAAPSVFIFPPSDEQLKSGTA | |
| | SVVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR Fab LC v2 | DILLTQSPVILSVSPGERVSFSCRASQ | 109 |
| | SIGTNIHWYQQRTNGSPRLLIKYAS | |
| | ESISGIPSRFSGSGSGTDFTLSINSVES | |
| | EDIADYYCQQNNNWPTTFGAGTKL | |
| | ELKRTVAAPSVFIFPPSDEQLKSGTA | |
| | SVVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR Fab HC | QVQLKQSGPGLVQPSQSLSITCTVS | 110 |
| | GFSLTNYGVHWVRQSPGKGLEWL | |
| | GVIWSGGNTDYNTPFTSRLSINKDN | |
| | SKSQVFFKMNSLQSNDTAIYYCARA | |
| | LTYYDYEFAYWGQGTLVTVSAAST | |
| | KGPSVFPLAPSSKSTSGGTAALGCL | |
| | VKDYFPEPVTVSWNSGALTSGVHTF | |
| | PAVLQSSGLYSLSSVVTVPSSSLGTQ | |
| | TYICNVNHKPSNTKVDKKVEPKSC | |
| EGFR Fab HC | QVQLKQSGPGLVQPSQSLSITCTVS | 111 |
| (N88Q) | GFSLTNYGVHWVRQSPGKGLEWL | |
| | GVIWSGGNTDYNTPFTSRLSINKDN | |
| | SKSQVFFKMNSLQSQDTAIYYCARA | |
| | LTYYDYEFAYWGQGTLVTVSAAST | |
| | KGPSVFPLAPSSKSTSGGTAALGCL | |
| | VKDYFPEPVTVSWNSGALTSGVHTF | |
| | PAVLQSSGLYSLSSVVTVPSSSLGTQ | |
| | TYICNVNHKPSNTKVDKKVEPKSC | |
| EGFR TCE-1 LC, | QILLTQSPVILSVSPGERVSFSCRASQ | 181 |
| | SIGTNIHWYQQRTNGSPRLLIKYASE | |
| | SISGIPSRFSGSGSGTDFTLSINSVESE | |
| | DIADYYCQQNNNWPTTFGAGTKLE | |
| | LKRTVAAPSVFIFPPSDEQLKSGTAS | |
| | VVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR TCE-1 HC | EVQLVESGGGLVQPGGSLKLSCAAS | 182 |
| | GFTFNKYAMNWVRQAPGKGLEWV | |
| | ARIRSKYNNYATYYADSVKDRFTIS | |
| | RDDSKNTAYLQMNNLKTEDTAVY | |
| | YCVRHGNFGNSYISYWAYWGQGTL | |
| | VTVSSGGGGSGGGGSGGGGSQTVV | |
| | TQEPSLTVSPGGTVTLTCGSSTGAV | |
| | TSGNYPNWVQQKPGQAPRGLIGGT | |
| | KFLAPGTPARFSGSLLGGKAALTLS | |
| | GVQPEDEAEYYCVLWYSNRWVFG | |
| | GGTKLTVLGGGGSQVQLKQSGPGL | |
| | VQPSQSLSITCTVSGFSLTNYGVHW | |
| | VRQSPGKGLEWLGVIWSGGNTDYN | |
| | TPFTSRLSINKDNSKSQVFFKMNSLQ | |
| | SQDTAIYYCARALTYYDYEFAYWG | |
| | QGTLVTVSAASTKGPSVFPLAPSSKS | |
| | TSGGTAALGCLVKDYFPEPVTVSW | |
| | NSGALTSGVHTFPAVLQSSGLYSLS | |
| | SVVTVPSSSLGTQTYICNVNHKPSN | |
| | TKVDKKVEPKSC | |
| EGFR TCE-2 LC | QILLTQSPVILSVSPGERVSFSCRASQ | 214 |
| JXA2212 | SIGTNIHWYQQRTNGSPRLLIKYASE | |
| | SISGIPSRFSGSGSGTDFTLSINSVESE | |
| | DIADYYCQQNNNWPTTFGAGTKLE | |
| | LKRTVAAPSVFIFPPSDEQLKSGTAS | |
| | VVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR TCE-2 HC | EVQLVESGGGLVQPGGSLKLSCAAS | 215 |
| JXA2212 | GFTFNKYAMNWVRQAPGKGLEWV | |
| | ARIRSKYNNYATYYADSVKDRFTIS | |
| | RDDSKNTAYLQMNNLKTEDTAVY | |
| | YCVRHGNFGNSYISYWAYWGQGTL | |
| | VTVSSGGGGSGGGGSGGGGSQTVV | |
| | TQEPSLTVSPGGTVTLTCGSSTGAV | |
| | TSGNYPNWVQQKPGQAPRGLIGGT | |
| | KFLAPGTPARFSGSLLGGKAALTLS | |
| | GVQPEDEAEYYCVLWYSNRWVFG | |
| | GGTKLTVLGGGGSQVQLKQSGPGL | |
| | VQPSQSLSITCTVSGFSLTNYGVHW | |
| | VRQSPGKGLEWLGVIWSGGNTDYN | |
| | TPFTSRLSINKDNSKSQVFFKMNSLQ | |
| | SQDTAIYYCARALTYYDYEFAYWG | |
| | QGTLVTVSAASTKGPSVFPLAPSSKS | |
| | TSGGTAALGCLVKDYFPEPVTVSW | |
| | NSGALTSGVHTFPAVLQSSGLYSLS | |
| | SVVTVPSSSLGTQTYICNVNHKPSN | |
| | TKVDKKVEPKSC | |
| EGFR TRACTr-1 LC, | GGPCRSHIDVAKPICVGGGGSGGLS | 183 |
| | GRSDAGSPLGLAGSGGSDILLTQSP | |
| | VILSVSPGERVSFSCRASQSIGTNIH | |
| | WYQQRTNGSPRLLIKYASESISGIPS | |
| | RFSGSGSGTDFTLSINSVESEDIADY | |
| | YCQQNNNWPTTFGAGTKLELKRTV | |
| | AAPSVFIFPPSDEQLKSGTASVVCLL | |
| | NNFYPREAKVQWKVDNALQSGNS | |
| | QESVTEQDSKDSTYSLSSTLTLSKA | |
| | DYEKHKVYACEVTHQGLSSPVTKS | |
| | FNRGEC | |
| EGFR TRACTr-1 HC, | EVQLVESGGGLVQPGGSLRLSCAAS | 184 |
| | GSTFYTAVMGWVRQAPGKGLEWV | |
| | AAIRWTALTTSYADSVKGRFTISRD | |
| | GAKTTLYLQMNSLRPEDTAVYYCA | |
| | ARGTLGLFTTADSYDYWGQGTLVT | |
| | VSSGGGGSGGGSGGVYCGPEFDES | |
| | VGCMGGGGSGGGLSGRSDAGSPLG | |
| | LAGSGGGSEVQLVESGGGLVQPGG | |
| | SLKLSCAASGFTFNKYAMNWVRQA | |
| | PGKGLEWVARIRSKYNNYATYYAD | |
| | SVKDRFTISRDDSKNTAYLQMNNL | |
| | KTEDTAVYYCVRHGNFGNSYISYW | |
| | AYWGQGTLVTVSSGGGGSGGGGSG | |
| | GGGSQTVVTQEPSLTVSPGGTVTLT | |
| | CGSSTGAVTSGNYPNWVQQKPGQA | |
| | PRGLIGGTKFLAPGTPARFSGSLLGG | |
| | KAALTLSGVQPEDEAEYYCVLWYS | |
| | NRWVFGGGTKLTVLGGGGSQVQL | |
| | KQSGPGLVQPSQSLSITCTVSGFSLT | |
| | NYGVHWVRQSPGKGLEWLGVIWS | |
| | GGNTDYNTPFTSRLSINKDNSKSQV | |
| | FFKMNSLQSNDTAIYYCARALTYY | |
| | DYEFAYWGQGTLVTVSAASTKGPS | |
| | VFPLAPSSKSTSGGTAALGCLVKDY | |
| | FPEPVTVSWNSGALTSGVHTFPAVL | |
| | QSSGLYSLSSVVTVPSSSLGTQTYIC | |
| | NVNHKPSNTKVDKKVEPKSC | |
| EGFR TRACTr-1 | PCRSHIDVAKPICV | 185 |
| Peptide Mask (P₄) | | |
| EGFR TRACTr-2 | PCLFHFDPAKPICS | 186 |
| Peptide Mask (P₄), | | |

In some embodiments, the tumor antigen comprises TROP2. In some embodiments, the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 112; HC-CDR2: SEQ ID NO: 113; HC-CDR3: SEQ ID NO: 114; and wherein the LC-CDR1, the LC-CDR2, and the LC-CDR3 comprise: LC-CDR1: SEQ ID NO: 115; LC-CDR2: SEQ ID NO: 116 (SAS); and LC-CDR3: SEQ ID NO: 117. In some embodiments, the tumor antigen comprises TROP2, and the T cell engager comprises amino acid sequences with at least 95% sequence identity according to SEQ ID NOs: 187-192. In some embodiments, the tumor antigen comprises TROP2, and the T cell engager comprises amino acid sequences according to any one of SEQ ID NOs: 187-192. In some embodiments, the tumor antigen binding domain comprises an amino acid sequence according to SEQ ID NOs: 112-119.

TABLE 15
Tumor antigen binding domain amino acid sequences-anti-TROP2

| | Amino Acid Sequence | SEQ ID |
|---|---|---|
| Construct Description | (N to C) | NO: |
| TROP2: HC: CDR1 | GYTFTNYG | 112 |
| TROP2: HC: CDR2 | INTYTGEP | 113 |
| TROP2: HC: CDR3 | ARGGFGSSYWYFDV | 114 |
| TROP2: LC: CDR1 | QDVSIA | 115 |
| TROP2: LC: CDR2 | SAS | 116 |
| TROP2: LC: CDR3 | QQHYITPLT | 117 |
| TROP2 Fab LC | DIQLTQSPSSLSASVGDRVSITC | 118 |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 Fab HC | QVQLQQSGSELKKPGASVKVS | 119 |
| | CKASGYTFTNYGMNWVKQA | |
| | PGQGLKWMGWINTYTGEPTY | |
| | TDDFKGRFAFSLDTSVSTAYL | |
| | QISSLKADDTAVYFCARGGFG | |
| | SSYWYFDVWGQGSLVTVSSA | |
| | STKGPSVFPLAPSSKSTSGGTA | |
| | ALGCLVKDYFPEPVTVSWNSG | |
| | ALTSGVHTFPAVLQSSGLYSLS | |
| | SVVTVPSSSLGTQTYICNVNH | |
| | KPSNTKVDKKVEPKSC | |
| TROP2 TCE-1 LC | DIQLTQSPSSLSASVGDRVSITC | 187 |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TROP2 TCE-1 HC | EVQLVESGGGLVQPGGSLKLS | 188 |
| | CAASGFTFNKYAMNWVRQAP | |
| | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSQVQLQQSGSELKKP | |
| | GASVKVSCKASGYTFTNYGM | |
| | NWVKQAPGQGLKWMGWINT | |
| | YTGEPTYTDDFKGRFAFSLDT | |
| | SVSTAYLQISSLKADDTAVYF | |
| | CARGGFGSSYWYFDVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TROP2 TCE-2 LC | DIQLTQSPSSLSASVGDRVSITC | 189 |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TROP2 TCE-2 HC | EVQLVESGGGLVQPGGSLKLS | 190 |
| | CAASGFTFNKYAMNWVRQAP | |
| | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSQVQLQQSGSELKKP | |
| | GASVKVSCKASGYTFTNYGM | |
| | NWVKQAPGQGLKWMGWINT | |
| | YTGEPTYTDDFKGRFAFSLDT | |
| | SVSTAYLQISSLKADDTAVYF | |
| | CARGGFGSSYWYADVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TROP2 TCE-3 LC | DIQLTQSPSSLSASVGDRVSITC | 191 |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TROP2 TCE-3 HC | EVQLVESGGGLVQPGGSLKLS | 192 |
| | CAASGFTFNKYAMNWVRQAP | |
| | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSQVQLQQSGSELKKP | |
| | GASVKVSCKASGYTFTNYGM | |
| | NWVKQAPGQGLKWMGWINT | |
| | YTGEPTYTDDFKGRFAFSLDT | |
| | SVSTAYLQISSLKADDTAVYF | |
| | CARGGFGSSYWYFAVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TROP2 TRACTr-1 LC | GGIDFCMLYNWPICAGGGGGS | 193 |
| | GGLSGRSDAGSPLGLAGSGGS | |
| | DIQLTQSPSSLSASVGDRVSITC | |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TROP2 TRACTr-1 HC | EVQLVESGGGLVQPGGSLRLS | 194 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYFDVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-2 LC | GGVDFCGLYHWPICYQGGGG | 195 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-2 HC | EVQLVESGGGLVQPGGSLRLS | 196 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYFAVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-3 LC | GGVDFCALYHWPICYQGGGG | 197 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-3 HC | EVQLVESGGGLVQPGGSLRLS | 198 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYADVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-4 LC | GGIDFCMLYNWPICAGGGGGS | 216 |
| JXA2388 | GGLSGRSDAGSPLGLAGSGGS | |
| | DIQLTQSPSSLSASVGDRVSITC | |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TROP2 TRACTr-4 HC | EVQLVESGGGLVQPGNSLRLS | 217 |
| JXA2388 | CAASGFTFSKFGMSWVRQAP | |
| | GKGLEWVSSISGSGRDTLYAD | |
| | SVKGRFTISRDNAKTTLYLQM | |
| | NSLRPEDTAVYYCTIGGSLSVS | |
| | SQGTLVTVSSGGGGSGGGSGG | |
| | VYCGPEFDESVGCMGGGGSG | |
| | GGLSGRSDAGSPLGLAGSGGG | |
| | SEVQLVESGGGLVQPGGSLKL | |
| | SCAASGFTFNKYAMNWVRQA | |
| | PGKGLEWVARIRSKYNNYAT | |
| | YYADSVKDRFTISRDDSKNTA | |
| | YLQMNNLKTEDTAVYYCVRH | |
| | GNFGNSYISYWAYWGQGTLV | |
| | TVSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSQVQLQQSGSELKKP | |
| | GASVKVSCKASGYTFTNYGM | |
| | NWVKQAPGQGLKWMGWINT | |
| | YTGEPTYTDDFKGRFAFSLDT | |
| | SVSTAYLQISSLKADDTAVYF | |
| | CARGGFGSSYWYFDVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TROP2 TRACTr-5 LC | GGIDFCMLYNWPICAGGGGGS | 218 |
| JXA2389 | GGGGGSGGGGSGGASSGAGG | |
| | SGGSDIQLTQSPSSLSASVGDR | |
| | VSITCKASQDVSIAVAWYQQK | |
| | PGKAPKLLIYSASYRYTGVPD | |
| | RFSGSGSGTDFTLTISSLQPEDF | |
| | AVYYCQQHYITPLTFGAGTKV | |
| | EIKRTVAAPSVFIFPPSDEQLKS | |
| | GTASVVCLLNNFYPREAKVQ | |
| | WKVDNALQSGNSQESVTEQD | |
| | SKDSTYSLSSTLTLSKADYEKH | |
| | KVYACEVTHQGLSSPVTKSFN | |
| | RGEC | |
| TROP2 TRACTr-5 HC | EVQLVESGGGLVQPGNSLRLS | 219 |
| JXA2389 | CAASGFTFSKFGMSWVRQAP | |
| | GKGLEWVSSISGSGRDTLYAD | |
| | SVKGRFTISRDNAKTTLYLQM | |
| | NSLRPEDTAVYYCTIGGSLSVS | |
| | SQGTLVTVSSGGGGSGGGSGG | |
| | VYCGPEFDESVGCMGGGGSG | |
| | GGSGGGGSGGASSGAGGSGG | |
| | GSEVQLVESGGGLVQPGGSLK | |
| | LSCAASGFTFNKYAMNWVRQ | |
| | APGKGLEWVARIRSKYNNYA | |
| | TYYADSVKDRFTISRDDSKNT | |
| | AYLQMNNLKTEDTAVYYCVR | |
| | HGNFGNSYISYWAYWGQGTL | |
| | VTVSSGGGGSGGGGSGGGGS | |
| | QTVVTQEPSLTVSPGGTVTLT | |
| | CGSSTGAVTSGNYPNWVQQK | |
| | PGQAPRGLIGGTKFLAPGTPAR | |
| | FSGSLLGGKAALTLSGVQPED | |
| | EAEYYCVLWYSNRWVFGGGT | |
| | KLTVLGGGGSQVQLQQSGSEL | |
| | KKPGASVKVSCKASGYTFTNY | |
| | GMNWVKQAPGQGLKWMGWI | |
| | NTYTGEPTYTDDFKGRFAFSL | |
| | DTSVSTAYLQISSLKADDTAV | |
| | YFCARGGFGSSYWYFDVWGQ | |
| | GSLVTVSSASTKGPSVFPLAPS | |
| | SKSTSGGTAALGCLVKDYFPE | |
| | PVTVSWNSGALTSGVHTFPAV | |
| | LQSSGLYSLSSVVTVPSSSLGT | |
| | QTYICNVNHKPSNTKVDKKVE | |
| | PKSC | |
| TROP2 TRACTr-1 Peptide | IDFCMLYNWPICA | 199 |
| Mask (P₄) | | |
| TROP2 TRACTr-2 Peptide | VDFCGLYHWPICYQ | 200 |
| Mask (P₄) | | |
| TROP2 TRACTr-2 Peptide | VDFCALYHWPICYQ | 201 |
| Mask (P₄) | | |

In some embodiments, the tumor antigen comprises PSMA. In some embodiments, the tumor antigen binding domain comprises an amino acid sequence according to SEQ ID NOs: 120-127.

In some embodiments, the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 120; HC-CDR2: SEQ ID NO: 121; HC-CDR3: SEQ ID NO: 122; and wherein the LC-CDR1, the LC-CDR2, and the LC-CDR3 comprise: LC-CDR1: SEQ ID NO: 123; LC-CDR2: SEQ ID NO: 124 (EA); and LC-CDR3: SEQ ID NO: 125. In some embodiments, the tumor antigen comprises PSMA, and the T cell engager comprises amino acid sequences with at least 95% sequence identity according to SEQ ID NOs: 173 and 174. In some embodiments, the tumor antigen comprises PSMA, and the T cell engager comprises amino acid sequences according to SEQ ID NOs: 173 and 174.

TABLE 16
Tumor antigen binding domain amino acid sequences-anti-PSMA

| | Amino Acid Sequence | SEQ ID |
|---|---|---|
| Construct Description | (N to C) | NO: |
| PSMA: HC: CDR1 | GFAFSRYG | 120 |
| PSMA: HC: CDR2 | IWYDGSNK | 121 |
| PSMA: HC: CDR3 | ARGGDFLYYYYYGMDV | 122 |
| PSMA: LC: CDR1 | QGISNY | 123 |
| PSMA: LC: CDR2 | EA | 124 |
| PSMA: LC: CDR3 | QNYNSAPFT | 125 |
| 006 PSMA Fab LC | DIQMTQSPSSLSASVGDRVTIT | 126 |
| | CRASQGISNYLAWYQQKTGK | |
| | VPKFLIYEASTLQSGVPSRFSG | |
| | GGSGTDFTLTISSLQPEDVATY | |
| | YCQNYNSAPFTFGPGTKVDIK | |
| | RTVAAPSVFIFPPSDEQLKSGT | |
| | ASVVCLLNNFYPREAKVQWK | |
| | VDNALQSGNSQESVTEQDSKD | |
| | STYSLSSTLTLSKADYEKHKV | |
| | YACEVTHQGLSSPVTKSFNRG | |
| | EC | |
| 006 PSMA Fab HC | QVQLVESGGGVVQPGRSLRLS | 127 |
| | CAASGFAFSRYGMHWVRQAP | |
| | GKGLEWVAVIWYDGSNKYY | |
| | ADSVKGRFTISRDNSKNTQYL | |
| | QMNSLRAEDTAVYYCARGGD | |
| | FLYYYYYGMDVWGQGTTVT | |
| | VSSASTKGPSVFPLAPSSKSTS | |
| | GGTAALGCLVKDYFPEPVTVS | |
| | WNSGALTSGVHTFPAVLQSSG | |
| | LYSLSSVVTVPSSSLGTQTYIC | |
| | NVNHKPSNTKVDKKVEPKSC | |
| PSMA TCE-1 LC | EVQLVESGGGLVQPGGSLKLS | 173 |
| | CAASGFTFNKYAMNWVRQAP | |
| | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSDIQMTQSPSSLSAS | |
| | VGDRVTITCRASQGISNYLAW | |
| | YQQKTGKVPKFLIYEASTLQS | |
| | GVPSRFSGGGSGTDFTLTISSL | |
| | QPEDVATYYCQNYNSAPFTFG | |
| | PGTKVDIKRTVAAPSVFIFPPS | |
| | DEQLKSGTASVVCLLNNFYPR | |
| | EAKVQWKVDNALQSGNSQES | |
| | VTEQDSKDSTYSLSSTLTLSKA | |
| | DYEKHKVYACEVTHQGLSSP | |
| | VTKSFNRGEC | |
| PSMA TCE-1 HC | QVQLVESGGGVVQPGRSLRLS | 174 |
| | CAASGFAFSRYGMHWVRQAP | |
| | GKGLEWVAVIWYDGSNKYYA | |
| | DSVKGRFTISRDNSKNTQYLQ | |
| | MNSLRAEDTAVYYCARGGDF | |
| | LYYYYYGMDVWGQGTTVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |
| PSMA TRACTr-1 LC | EVQLVESGGGLVQPGGSLRLS | 175 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSDIQMTQSPSSLSASVGDRVT | |
| | ITCRASQGISNYLAWYQQKTG | |
| | KVPKFLIYEASTLQSGVPSRFS | |
| | GGGSGTDFTLTISSLQPEDVAT | |
| | YYCQNYNSAPFTFGPGTKVDI | |
| | KRTVAAPSVFIFPPSDEQLKSG | |
| | TASVVCLLNNFYPREAKVQW | |
| | KVDNALQSGNSQESVTEQDSK | |
| | DSTYSLSSTLTLSKADYEKHK | |
| | VYACEVTHQGLSSPVTKSFNR | |
| | GEC | |
| PSMA TRACTr-1 HC | QVQLVESGGGVVQPGRSLRLS | 176 |
| | CAASGFAFSRYGMHWVRQAP | |
| | GKGLEWVAVIWYDGSNKYYA | |
| | DSVKGRFTISRDNSKNTQYLQ | |
| | MNSLRAEDTAVYYCARGGDF | |
| | LYYYYYGMDVWGQGTTVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |

In some embodiments, the T cell engager molecule is selectively activated in tumor microenvironments. In some embodiments, the T cell engager is according to the following subformula: P₃-L₃-D₁-L₀-E₁ (Formula IIa) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease.

In some embodiments, the T cell engager is according to the following subformula: D₁-L₀-E₁-L₄-P₄ (Formula IIb) wherein D₁ comprises the CD3 binding domain; E₁ comprises the tumor antigen binding domain; L₀ comprises the linker that connects D₁ to E₁; P₄ comprises a peptide that binds to E₁ and L₄ comprises a linking moiety that connects E₁ to P₄ and is a substrate for a tumor specific protease.

In some embodiments, the T cell engager is according to the following subformula: P₃-L₃-D₁-L₀-E₁-L₄-P₄ (Formula IIc) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease; P₄ comprises a peptide that binds to E₁ and L₄ comprises a linking moiety that connects E₁ to P₄ and is a substrate for a tumor specific protease.

In some embodiments, the T cell engager comprises H₁. In some embodiments, H₁ comprises a sequence according to SEQ ID NO: 54-57. In some embodiments, H₁ comprises a single domain antibody.

In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56. In some embodiments, L₃ or L₄ is a peptide sequence having at least 5 to no more than 50 amino acids. In some embodiments, L₃ or L₄ is a peptide sequence having at least 10 to no more than 30 amino acids. In some embodiments, L₃ or L₄ is a peptide sequence having at least 10 amino acids. In some embodiments, L₃ or L₄ is a peptide sequence having at least 18 amino acids. In some embodiments, L₃ or L₄ is a peptide sequence having at least 26 amino acids. In some embodiments, L₃ or L₄ comprises a formula comprising (G₂S)ₙ, wherein n is an integer from 1 to 3 (SEQ ID NO: 228). In some embodiments, L₃ or L₄ comprises a formula comprising (G₂S)ₙ, wherein n is an integer of at least 1. In some embodiments, L₃ or L₄ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease.

In some embodiments, L₃ or L₄ comprises a urokinase cleavable amino acid sequence, a matriptase cleavable amino acid sequence, or a matrix metalloprotease cleavable amino acid sequence. In some embodiments, L₃ or L₄ comprises a sequence according to SEQ ID NOs: 18-19, 62-88. In some embodiments, L₃ is bound to N-terminus of D₁. In some embodiments, L₃ is bound to C-terminus of D₁. In some embodiments, L₄ is bound to N-terminus of E₁. In some embodiments, L₄ is bound to C-terminus of E₁.

In some embodiments, P₃ becomes unbound from D₁ when L₃ is cleaved by the tumor specific protease thereby exposing D₁ to CD3. In some embodiments, P₄ becomes unbound from E₁ when L₄ is cleaved by the tumor specific protease thereby exposing E₁ to the tumor antigen. In some embodiments, P₃ impairs binding of D₁ to CD3. In some embodiments, P₃ is bound to D₁ through ionic interactions, electrostatic interactions, hydrophobic interactions, Pi-stacking interactions, and H-bonding interactions, or a combination thereof. In some embodiments, P₃ is bound to D₁ at or near an antigen binding site. In some embodiments, P₃ becomes unbound from D₁ when L₃ is cleaved by the tumor specific protease thereby exposing D₁ to CD3. In some embodiments, P₃ has less than 70% sequence identity to CD3. In some embodiments, P₃ has less than 85% sequence identity to CD3. In some embodiments, P₃ has less than 90% sequence identity to CD3. In some embodiments, P₃ has less than 95% sequence identity to CD3. In some embodiments, P₃ has less than 98% sequence identity to CD3. In some embodiments, P₃ has less than 99% sequence identity to CD3. In some embodiments, P₃ comprises the amino acid sequence according to SEQ ID NOs: 177-180. In some embodiments, P₃ comprises a de novo amino acid sequence that shares less than 10% sequence identity to CD3. In some embodiments, P₄ impairs binding of E₁ to the tumor antigen. In some embodiments, P₄ is bound to E₁ through ionic interactions, electrostatic interactions, hydrophobic interactions, Pi-stacking interactions, and H-bonding interactions, or a combination thereof. In some embodiments, P₄ is bound to E₁ at or near an antigen binding site. In some embodiments, P₄ becomes unbound from E₁ when L₄ is cleaved by the tumor specific protease thereby exposing E₁ to the tumor antigen. In some embodiments, P₄ has less than 70% sequence identity to the tumor antigen. In some embodiments, P₄ has less than 80% sequence identity to the tumor antigen. In some embodiments, P₄ has less than 85% sequence identity to the tumor antigen. In some embodiments, P₄ has less than 90% sequence identity to the tumor antigen. In some embodiments, P₄ has less than 95% sequence identity to the tumor antigen. In some embodiments, P₄ comprises a de novo amino acid sequence that shares less than 10% sequence identity to the tumor antigen. In some embodiments, P₃ or P₄ comprises a peptide sequence of at least 5 amino acids in length. In some embodiments, P₃ or P₄ comprises a peptide sequence of at least 6 amino acids in length. In some embodiments, P₃ or P₄ comprises a peptide sequence of at least 10 amino acids in length. In some embodiments, P₃ or P₄ comprises a peptide sequence of at least 10 amino acids in length and no more than 20 amino acids in length. In some embodiments, P₃ or P₄ comprises a peptide sequence of at least 16 amino acids in length. In some embodiments, P₃ or P₄ comprises a peptide sequence of no more than 40 amino acids in length. In some embodiments, P₃ or P₄ comprises at least two cysteine amino acid residues. In some embodiments, P₃ or P₄ comprises a cyclic peptide or a linear peptide. In some embodiments, P₃ or P₄ comprises a cyclic peptide. In some embodiments, P₃ or P₄ comprises a linear peptide. In some embodiments, P₄ comprises the amino acid sequence according to SEQ ID NO: 185 or 186.

In some embodiments, the tumor antigen comprises EGFR, and the T cell engager comprises the amino acid sequence of SEQ ID NOs: 183 and 184. In some embodiments, P₄ comprises the amino acid sequence according to SEQ ID NOs: 199-201. In some embodiments, the tumor antigen comprises TROP2, and the T cell engager comprises any one of amino acid sequences of SEQ ID NOs: 193-198. In some embodiments, the tumor antigen comprises PSMA, and the T cell engager comprises the amino acid sequence of SEQ ID NOs: 175 and 176.

In some embodiments, the multispecific antibody further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the multispecific antibody as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

CD28 Binding Domains Linked to a Peptide that Impairs Binding to CD28

Disclosed herein are isolated polypeptide or polypeptide complex comprising a CD28 binding domain that is linked to a peptide that impairs binding of the CD28 binding domain to CD28 wherein the peptide comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20. In some embodiments, the peptide comprises an amino acid sequence according to any one of SEQ ID NOs: 24-53, 128-148, and the amino acid sequences of Table 20. In some embodiments, the peptide comprises an amino acid sequence according to any one of SEQ ID NOs: 42-53 or an amino acid sequence that has 1, 2, or 3 amino acid mutations, substitutions, or deletions relative to any one of SEQ ID NOs: 42-53. In some embodiments, the peptide comprises an amino acid sequence according to any one of SEQ ID NOs: 42-53. In some embodiments, the peptide comprises an amino acid sequence according to any one of the amino acid sequences of Table 20 or an amino acid sequence that has 1, 2, or 3 amino acid mutations, substitutions, or deletions relative to any one of the amino acid sequences of Table 20. In some embodiments, the peptide comprises an amino acid sequence according to any one of the amino acid sequences of Table 20. In some embodiments, the peptide comprises an amino acid sequence according to any one of SEQ ID NOs: 128-147 or an amino acid sequence that has 1, 2, or 3 amino acid mutations, substitutions, or deletions relative to any one of SEQ ID NOs: 128-147. In some embodiments, the peptide comprises an amino acid sequence according to any one of SEQ ID NOs: 128-147.

In some embodiments, the peptide comprises an amino acid sequence according to X₁-X₂-X₃-C-X₄-X₅-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₁, is selected from E, V, L, D, and H; X₁₁ is selected from F, Y, and L; and X₁₂ is selected from N, A, F, S, and Y. In some embodiments, X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F. In some embodiments, the peptide comprises an amino acid sequence according to SEQ ID NO: 32 or an amino acid sequence that has 1, 2, or 3 amino acid mutations, substitutions, or deletions relative to SEQ ID NO: 32. In some embodiments, the peptide comprises an amino acid sequence according to SEQ ID NO: 32. In some embodiments, the peptide comprises an amino acid sequence according to SEQ ID NO: 138 or an amino acid sequence that has 1, 2, or 3 amino acid mutations, substitutions, or deletions relative to SEQ ID NO: 138. In some embodiments, the peptide comprises an amino acid sequence according to SEQ ID NO: 138.

In some embodiments, the CD28 binding domain comprises a single chain variable fragment, a single domain antibody, a Fab, or a Fab′. In some embodiments, the CD28 binding domain comprises the single chain variable fragment. In some embodiments, the CD28 binding domain comprises the single domain antibody. In some embodiments, the CD28 binding domain comprises the Fab or the Fab′. In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5 (KA); and LC-CDR3: SEQ ID NO: 6, and wherein the CDRs comprise from 0-2 amino acid modifications in at least one of said LC-CDR1, LC-CDR2, or LC-CDR3. In some embodiments, the scFv heavy chain variable domain comprises an amino acid sequence that has at least 80% sequence identity to the amino acid sequence according to SEQ ID NO: 7. In some embodiments, the scFv heavy chain variable domain comprises an amino acid sequence of at least 75 consecutive amino acid residues of SEQ ID NO: 7 In some embodiments, the scFv heavy chain variable domain comprises an amino acid sequence of at least 110 consecutive amino acid residues of SEQ ID NO: 7. In some embodiments, the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7. In some embodiments, the scFv heavy chain variable domain comprises an amino acid sequence according to SEQ ID NO: 7. In some embodiments, the scFv light chain variable domain comprises an amino acid sequence that has at least 80% sequence identity to the amino acid sequence according to SEQ ID NO: 8. In some embodiments, the scFv light chain variable domain comprises an amino acid sequence of at least 75 consecutive amino acid residues of SEQ ID NO: 8. In some embodiments, the scFv light chain variable domain comprises an amino acid sequence of at least 100 consecutive amino acid residues of SEQ ID NO: 8. In some embodiments, the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8. In some embodiments, the scFv light chain variable domain comprises an amino acid sequence according to SEQ ID NO: 8. In some embodiments, the scFv comprises an amino acid sequence that has at least 80% sequence identity to the amino acid sequence according to SEQ ID NO: 9. In some embodiments, the scFv comprises an amino acid sequence of at least 175 consecutive amino acid residues of SEQ ID NO: 9. In some embodiments, the scFv comprises an amino acid sequence of at least 210 consecutive amino acid residues of SEQ ID NO: 9. In some embodiments, the scFv comprises an amino acid sequence of at least 210 consecutive amino acid residues of SEQ ID NO: 9 and has at least 80% sequence identity to the at least 210 consecutive amino acid residues of SEQ ID NO: 9. In some embodiments, the scFv comprises an amino acid sequence according to SEQ ID NO: 9.

In some embodiments, the CD28 binding domain is linked to the peptide through a linking moiety (L₁). In some embodiments, L₁ is a substrate for a tumor specific protease. In some embodiments, L₁ is a peptide sequence having at least 5 to no more than 50 amino acids. In some embodiments, L₁ is a peptide sequence having at least 10 to no more than 30 amino acids. In some embodiments, L₁ is a peptide sequence having at least 10 amino acids. In some embodiments, L₁ is a peptide sequence having at least 18 amino acids. In some embodiments, L₁ is a peptide sequence having at least 26 amino acids. In some embodiments, L₁ comprises a formula comprising (G₂S)ₙ, wherein n is an integer from 1 to 3 (SEQ ID NO: 228). In some embodiments, L₁ comprises a formula comprising (G₂S)ₙ, wherein n is an integer of at least 1. In some embodiments, L₁ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments, L₁ comprises a urokinase cleavable amino acid sequence, a matriptase cleavable amino acid sequence, or a matrix metalloprotease cleavable amino acid sequence. In some embodiments, L₁ comprises a sequence according to SEQ ID NOs: 18-19, 62-88. In some embodiments, L₁ is bound to N-terminus of A₁. In some embodiments, L₁ is bound to C-terminus of A₁. In some embodiments, P₁ becomes unbound from A₁ when L₁ is cleaved by the tumor specific protease thereby exposing A₁ to CD28. In some embodiments, L₁ comprise a modified amino acid or non-natural amino acid, or a modified non-natural amino acid, or a combination thereof. In some embodiments, the modified amino acid or a modified non-natural amino acid comprises a post-translational modification.

In some embodiments, the isolated polypeptide or polypeptide complex further comprises a half-life extending molecule (H₁). In some embodiments, H₁ is connected to the peptide. In some embodiments, H₁ does not block the CD28 binding domain to CD28. In some embodiments, H₁ comprises a linking moiety (L₅) that connects H₁ to the peptide. In some embodiments, the half-life extending molecule (H₁) does not have binding affinity to CD28. In some embodiments, the half-life extending molecule (H₁) does not shield the isolated polypeptide or polypeptide complex from CD28. In some embodiments, H₁ comprises a sequence according to SEQ ID NOs: 54-57. In some embodiments, H₁ comprises an amino acid sequence that has repetitive sequence motifs. In some embodiments, H₁ comprises an amino acid sequence that has highly ordered secondary structure. In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, sIgA, IgM or IgD. In some embodiments, the serum protein is albumin. In some embodiments, the polypeptide is an antibody. In some embodiments, the antibody comprises a single domain antibody, a single chain variable fragment, a Fab, or a Fab′. In some embodiments, the single domain antibody comprises a single domain antibody that binds to albumin. In some embodiments, the single domain antibody is a human or humanized antibody. In some embodiments, the single domain antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10E and SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3. In some embodiments, H₁ comprises an amino acid sequence according to SEQ ID NO: 57. In some embodiments, H₁ comprises an amino acid sequence that has at least 80% sequence identity to SEQ ID NO: 57. In some embodiments, H₁ comprises an amino acid sequence that has at least 85% sequence identity to SEQ ID NO: 57. In some embodiments, H₁ comprises an amino acid sequence that has at least 90% sequence identity to SEQ ID NO: 57. In some embodiments, H₁ comprises an amino acid sequence that has at least 95% sequence identity to SEQ ID NO: 57. In some embodiments, H₁ comprises an amino acid sequence that has at least 99% sequence identity to SEQ ID NO: 57. In some embodiments, H₁ comprise a modified amino acid or non-natural amino acid, or a modified non-natural amino acid, or a combination thereof. In some embodiments, the modified amino acid or a modified non-natural amino acid comprises a post-translational modification. In some embodiments, H₁ comprises a linking moiety (L₅) that connects H₁ to P₁ or P₂. In some embodiments, L₅ is a peptide sequence having at least 5 to no more than 50 amino acids. In some embodiments, L₅ is a peptide sequence having at least 10 to no more than 30 amino acids. In some embodiments, L₅ is a peptide sequence having at least 10 amino acids. In some embodiments, L₅ is a peptide sequence having at least 18 amino acids. In some embodiments, L₅ is a peptide sequence having at least 26 amino acids. In some embodiments, L₅ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

### Methods of Treatment

Disclosed herein are methods of treating cancer in a subject in need thereof comprising administering to the subject the multispecific antibody of any of the embodiments described herein. In some embodiments, the multispecific antibody induces T cell mediated cytotoxicity of tumor cells. In some embodiments, the cancer is a hematological malignancy. In some embodiments, the cancer is leukemia or lymphoma. In some embodiments, the cancer is lymphoma, and wherein the lymphoma is B-cell lymphoma. In some embodiments, the cancer is a solid tumor. In some embodiments, the solid tumor expresses PD-L1. In some embodiments, the solid tumor is sarcoma, breast cancer, lung cancer, or carcinoma. In some embodiments, the solid tumor is lung cancer, and wherein the lung cancer is non-small cell lung cancer. In some embodiments, the multispecific antibody is administered in combination with an anti-cancer therapy. In some embodiments, the multispecific antibody and the anti-cancer therapy are administered in the same pharmaceutical composition. In some embodiments, the multispecific antibody and the anti-cancer therapy are administered as separate pharmaceutical compositions. In some embodiments, the subject is refractory to checkpoint inhibitor therapy. In some embodiments, the subject has relapsed from checkpoint inhibitor therapy. In some embodiments, the anti-cancer therapy comprises a small molecule, a cell-based therapy, or an antibody-based therapy.

In some embodiments, the administering to the subject of the multispecific antibody is sufficient to reduce or eliminate the cancer as compared to a baseline measurement of the cancer taken from the subject prior to the administering of the multispecific antibody. In some embodiments, the reduction is at least about 1-fold, 5-fold, 10-fold, 20-fold, 40-fold, 60-fold, 80-fold, or up to about 100 fold.

In some embodiments, the antibody-based therapy is a T cell engager. In some embodiments, the T cell engager comprises a formula according to: D₁-L₀-E₁ (Formula II), wherein D₁ comprises an effector cell binding domain that binds to an effector cell antigen, E₁ comprises a tumor antigen binding domain that binds to a tumor antigen, and L₀ comprises a linker that connects D₁ to E₁. In some embodiments, D₁ comprises a single chain variable fragment, a single domain antibody, a Fab fragment, or a Fab′. In some embodiments, D₁ comprises the single chain variable fragment. In some embodiments, E₁ comprises a single chain variable fragment, a single domain antibody, a Fab fragment, or a Fab′. In some embodiments, E₁ comprises the Fab fragment. In some embodiments, the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the effector cell binding domain comprises an amino acid sequence according to SEQ ID NOs: 89-101. In some embodiments, the tumor antigen comprises epidermal growth factor receptor (EGFR), prostate-specific membrane antigen (PSMA), or tumor-associated calcium signal transducer 2 (referred to herein after as TROP2).

In some embodiments, the tumor antigen comprises EGFR. In some embodiments, the tumor antigen binding domain comprises an amino acid sequence according to SEQ ID NOs: 102-111. In some embodiments, the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 105; HC-CDR2: SEQ ID NO: 106; HC-CDR3: SEQ ID NO: 107; and wherein the LC-CDR1, the LC-CDR2, and the LC-CDR3 comprise: LC-CDR1: SEQ ID NO: 102; LC-CDR2: SEQ ID NO: 103 (YAS); and LC-CDR3: SEQ ID NO: 104. In some embodiments, the tumor antigen comprises EGFR, and the T cell engager comprises amino acid sequences with at least 95% sequence identity according to SEQ ID NOs: 181 and 182. In some embodiments, the tumor antigen comprises EGFR, and the T cell engager comprises amino acid sequences according to SEQ ID NOs: 181 and 182. In some embodiments, the tumor antigen comprises EGFR, and the T cell engager comprises amino acid sequences with at least 95% sequence identity according to SEQ ID NOs: 214 and 215. In some embodiments, the tumor antigen comprises EGFR, and the T cell engager comprises amino acid sequences according to SEQ ID NOs: 214 and 215. In some embodiments, the cancer is colorectal cancer (CRC), squamous cell carcinoma of the head and Neck (SCCHN), non-small cell lung cancer (NSCLC), prostate cancer, breast cancer, colon/rectum cancer, head and neck cancer, esophagogastric cancer, liver cancer, glioblastoma, cervical cancer, ovarian cancer, bladder cancer, kidney cancer, or pancreatic cancer.

In some embodiments, the tumor antigen comprises TROP2. In some embodiments, the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 112; HC-CDR2: SEQ ID NO: 113; HC-CDR3: SEQ ID NO: 114; and wherein the LC-CDR1, the LC-CDR2, and the LC-CDR3 comprise: LC-CDR1: SEQ ID NO: 115; LC-CDR2: SEQ ID NO: 116 (SAS); and LC-CDR3: SEQ ID NO: 117. In some embodiments, the tumor antigen comprises TROP2, and the T cell engager comprises amino acid sequences with at least 95% sequence identity according to SEQ ID NOs: 187-192. In some embodiments, the tumor antigen comprises TROP2, and the T cell engager comprises amino acid sequences according to any one of SEQ ID NOs: 187-192. In some embodiments, the tumor antigen binding domain comprises an amino acid sequence according to SEQ ID NOs: 112-119. In some embodiments, the cancer is the cancer is lung, breast (e.g. HER2+; ER/PR+; TNBC), cervical, ovarian, colorectal, pancreatic, gastric, triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer. In some embodiments, the tumor antigen comprises PSMA. In some embodiments, the tumor antigen binding domain comprises an amino acid sequence according to SEQ ID NOs: 120-127.

In some embodiments, the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 120; HC-CDR2: SEQ ID NO: 121; HC-CDR3: SEQ ID NO: 122; and wherein the LC-CDR1, the LC-CDR2, and the LC-CDR3 comprise: LC-CDR1: SEQ ID NO: 123; LC-CDR2: SEQ ID NO: 124 (EA); and LC-CDR3: SEQ ID NO: 125. In some embodiments, the tumor antigen comprises PSMA, and the T cell engager comprises amino acid sequences with at least 95% sequence identity according to SEQ ID NOs: 173 and 174. In some embodiments, the tumor antigen comprises PSMA, and the T cell engager comprises amino acid sequences according to SEQ ID NOs: 173 and 174. In some embodiments, the cancer is cancer is lung, breast (e.g. HER2+; ER/PR+; TNBC), cervical, ovarian, colorectal, pancreatic or gastric.

In some embodiments, the T cell engager molecule is selectively activated in tumor microenvironments. In some embodiments, the T cell engager is according to the following subformula: P₃-L₃-D₁-L₀-E₁ (Formula IIa) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease. In some embodiments, the T cell engager is according to the following subformula: D₁-L₀-E₁-L₄-P₄ (Formula IIb) wherein D₁ comprises the CD3 binding domain; E₁ comprises the tumor antigen binding domain; L₀ comprises the linker that connects D₁ to E₁; P₄ comprises a peptide that binds to E₁ and L₄ comprises a linking moiety that connects E₁ to P₄ and is a substrate for a tumor specific protease. In some embodiments, the T cell engager is according to the following subformula: P₃-L₃-D₁-L₀-E₁-L₄-P₄ (Formula IIc) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease; P₄ comprises a peptide that binds to E₁ and L₄ comprises a linking moiety that connects E₁ to P₄ and is a substrate for a tumor specific protease.

In some embodiments, the T cell engager comprises H₁. In some embodiments, H₁ comprises a sequence according to SEQ ID NO: 54-57. In some embodiments, H₁ comprises a single domain antibody. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56. In some embodiments, L₃ or L₄ is a peptide sequence having at least 5 to no more than 50 amino acids. In some embodiments, L₃ or L₄ is a peptide sequence having at least 10 to no more than 30 amino acids. In some embodiments, L₃ or L₄ is a peptide sequence having at least 10 amino acids. In some embodiments, L₃ or L₄ is a peptide sequence having at least 18 amino acids. In some embodiments, L₃ or L₄ is a peptide sequence having at least 26 amino acids. In some embodiments, L₃ or L₄ comprises a formula comprising (G₂S)ₙ, wherein n is an integer from 1 to 3 (SEQ ID NO: 228). In some embodiments, L₃ or L₄ comprises a formula comprising (G₂S)ₙ, wherein n is an integer of at least 1. In some embodiments, L₃ or L₄ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments, L₃ or L₄ comprises a urokinase cleavable amino acid sequence, a matriptase cleavable amino acid sequence, or a matrix metalloprotease cleavable amino acid sequence. In some embodiments, L₃ or L₄ comprises a sequence according to SEQ ID NOs: 18-19, 62-88. In some embodiments, L₃ is bound to N-terminus of D₁. In some embodiments, L₃ is bound to C-terminus of D₁. In some embodiments, L₄ is bound to N-terminus of E₁. In some embodiments, L₄ is bound to C-terminus of E₁. In some embodiments, P₃ becomes unbound from D₁ when L₃ is cleaved by the tumor specific protease thereby exposing D₁ to CD3. In some embodiments, P₄ becomes unbound from E₁ when L₄ is cleaved by the tumor specific protease thereby exposing E₁ to the tumor antigen. In some embodiments, P₃ impairs binding of D₁ to CD3. In some embodiments, P₃ is bound to D₁ through ionic interactions, electrostatic interactions, hydrophobic interactions, Pi-stacking interactions, and H-bonding interactions, or a combination thereof. In some embodiments, P₃ is bound to D₁ at or near an antigen binding site. In some embodiments, P₃ becomes unbound from D₁ when L₃ is cleaved by the tumor specific protease thereby exposing D₁ to CD3.

In some embodiments, P₃ has less than 70% sequence identity to CD3. In some embodiments, P₃ has less than 85% sequence identity to CD3. In some embodiments, P₃ has less than 90% sequence identity to CD3. In some embodiments, P₃ has less than 95% sequence identity to CD3. In some embodiments, P₃ has less than 98% sequence identity to CD3. In some embodiments, P₃ has less than 99% sequence identity to CD3. In some embodiments, P₃ comprises the amino acid sequence according to SEQ ID NOs: 177-180. In some embodiments, P₃ comprises a de novo amino acid sequence that shares less than 10% sequence identity to CD3.

In some embodiments, P₄ impairs binding of E₁ to the tumor antigen. In some embodiments, P₄ is bound to E₁ through ionic interactions, electrostatic interactions, hydrophobic interactions, Pi-stacking interactions, and H-bonding interactions, or a combination thereof. In some embodiments, P₄ is bound to E₁ at or near an antigen binding site. In some embodiments, P₄ becomes unbound from E₁ when L₄ is cleaved by the tumor specific protease thereby exposing E₁ to the tumor antigen. In some embodiments, P₄ has less than 70% sequence identity to the tumor antigen. In some embodiments, P₄ has less than 80% sequence identity to the tumor antigen. In some embodiments, P₄ has less than 85% sequence identity to the tumor antigen. In some embodiments, P₄ has less than 90% sequence identity to the tumor antigen. In some embodiments, P₄ has less than 95% sequence identity to the tumor antigen. In some embodiments, P₄ comprises a de novo amino acid sequence that shares less than 10% sequence identity to the tumor antigen. In some embodiments, P₃ or P₄ comprises a peptide sequence of at least 5 amino acids in length. In some embodiments, P₃ or P₄ comprises a peptide sequence of at least 6 amino acids in length. In some embodiments, P₃ or P₄ comprises a peptide sequence of at least 10 amino acids in length. In some embodiments, P₃ or P₄ comprises a peptide sequence of at least 10 amino acids in length and no more than 20 amino acids in length. In some embodiments, P₃ or P₄ comprises a peptide sequence of at least 16 amino acids in length. In some embodiments, P₃ or P₄ comprises a peptide sequence of no more than 40 amino acids in length. In some embodiments, P₃ or P₄ comprises at least two cysteine amino acid residues. In some embodiments, P₃ or P₄ comprises a cyclic peptide or a linear peptide. In some embodiments, P₃ or P₄ comprises a cyclic peptide. In some embodiments, P₃ or P₄ comprises a linear peptide. In some embodiments, P₄ comprises the amino acid sequence according to SEQ ID NO: 185 or 186. In some embodiments, the tumor antigen comprises EGFR, and the T cell engager comprises the amino acid sequence of SEQ ID NOs: 183 and 184. In some embodiments, P₄ comprises the amino acid sequence according to SEQ ID NOs: 199-201. In some embodiments, the tumor antigen comprises TROP2, and the T cell engager comprises any one of amino acid sequences of SEQ ID NOs: 193-198. In some embodiments, the tumor antigen comprises PSMA, and the T cell engager comprises the amino acid sequence of SEQ ID NOs: 175 and 176.

### Production of Antibodies

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk−, hgprt− or aprt− cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Aim. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4 Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™ TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic.

The label or package insert indicates that the composition is used for treating the condition of choice. The article of manufacture in this embodiment of the disclosure may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Embodiments

Embodiment 1. An isolated multispecific antibody according to the following formula: P₁-L₁-A₁-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

Embodiment 2. The isolated multispecific antibody of embodiment 1, wherein the multispecific antibody is according to the following formula: P₁-L₁-A₁-L-B-L₂-P₂ (Formula Ia) wherein P₂ comprises a peptide that binds to B and L₂ comprises a linking moiety that connects B to P₂ and is a substrate for a tumor specific protease.

Embodiment 3. The isolated multispecific antibody embodiments 1 or 2, wherein P₁ comprises an amino acid sequence according to any one of SEQ ID NOs: 24-53, 128-148, and the amino acid sequences of Table 20.

Embodiment 4. The isolated multispecific antibody embodiments 1 or 2, wherein P₁ comprises an amino acid sequence according to any one of SEQ ID NOs: 42-53 or an amino acid sequence that has 1, 2, or 3 amino acid mutations, substitutions, or deletions relative to any one of SEQ ID NOs: 42-53.

Embodiment 5. The isolated multispecific antibody embodiments 1 or 2, wherein P₁ comprises an amino acid sequence according to any one of SEQ ID NOs: 42-53.

Embodiment 6. The isolated multispecific antibody embodiments 1 or 2, wherein P₁ comprises an amino acid sequence according to any one of the amino acid sequences of Table 20 or an amino acid sequence that has 1, 2, or 3 amino acid mutations, substitutions, or deletions relative to any one of the amino acid sequences of Table 20.

Embodiment 7. The isolated multispecific antibody embodiments 1 or 2, wherein P₁ comprises an amino acid sequence according to any one of the amino acid sequences of Table 20.

Embodiment 8. The isolated multispecific antibody embodiments 1 or 2, wherein P₁ comprises an amino acid sequence according to any one of SEQ ID NOs: 128-147 or an amino acid sequence that has 1, 2, or 3 amino acid mutations, substitutions, or deletions relative to any one of SEQ ID NOs: 128-147.

Embodiment 9. The isolated multispecific antibody embodiments 1 or 2, wherein P₁ comprises an amino acid sequence according to any one of SEQ ID NOs: 128-147.

Embodiment 10. The isolated multispecific antibody of embodiments 1 or 2, wherein P₁ comprises an amino acid sequence according to X₁-X₂-X₃-C-X₄-X₅-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₁, is selected from E, V, L, D, Y, R, Q, H, F, K, A, M, and N; X₁₁ is selected from F, Y, L, W, and V; and X₁₂ is selected from N, A, F, S, Y, H, D, T, and L.

Embodiment 11. The isolated multispecific antibody of embodiment 10, wherein X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₁, is selected from E, V, L, D, and H; X₁₁ is selected from F, Y, and L; and X₁₂ is selected from N, A, F, S, and Y.

Embodiment 12. The isolated multispecific antibody of embodiment 11, wherein X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F.

Embodiment 13. The isolated multispecific antibody of any one of embodiments 1-3, 10-12, wherein P₁ comprises an amino acid sequence according to SEQ ID NO: 32 or an amino acid sequence that has 1, 2, or 3 amino acid mutations, substitutions, or deletions relative to SEQ ID NO: 32.

Embodiment 14. The isolated multispecific antibody of any one of embodiments 1-3, 10-12, wherein P₁ comprises an amino acid sequence according to SEQ ID NO: 32.

Embodiment 15. The isolated multispecific antibody of any one of embodiments 1-3, 10-12, wherein P₁ comprises an amino acid sequence according to SEQ ID NO: 138 or an amino acid sequence that has 1, 2, or 3 amino acid mutations, substitutions, or deletions relative to SEQ ID NO: 138.

Embodiment 16. The isolated multispecific antibody of any one of embodiments 1-3, 10-12, wherein P₁ comprises an amino acid sequence according to SEQ ID NO: 138.

Embodiment 17. The isolated multispecific antibody of any one of embodiments 1-16, wherein P₁ impairs binding of A₁ to CD28.

Embodiment 18. The isolated multispecific antibody of any one of embodiments 1-17, wherein P₁ is bound to A₁ through ionic interactions, electrostatic interactions, hydrophobic interactions, Pi-stacking interactions, and H-bonding interactions, or a combination thereof

Embodiment 19. The isolated multispecific antibody of any one of embodiments 1-18, wherein P₁ is bound to A₁ at or near an antigen binding site.

Embodiment 20. The isolated multispecific antibody of any one of embodiments 1-18, wherein P₁ becomes unbound from A₁ when L1 is cleaved by the tumor specific protease thereby exposing A₁ to CD28.

Embodiment 21. The isolated multispecific antibody of any one of embodiments 1-20, wherein P₁ has less than 75% sequence identity to CD28.

Embodiment 22. The isolated multispecific antibody of any one of embodiments 1-21, wherein P₁ has less than 80% sequence identity to CD28.

Embodiment 23. The isolated multispecific antibody of any one of embodiments 1-22, wherein P₁ has less than 85% sequence identity to CD28.

Embodiment 24. The isolated multispecific antibody of any one of embodiments 1-23, wherein P₁ has less than 90% sequence identity to CD28.

Embodiment 25. The isolated multispecific antibody of any one of embodiments 1-24, wherein P₁ has less than 95% sequence identity to CD28.

Embodiment 26. The isolated multispecific antibody of any one of embodiments 1-25, wherein P₁ comprises a de novo amino acid sequence that shares less than 10% sequence identity to CD28.

Embodiment 27. The isolated multispecific antibody of any one of embodiments 2-26, wherein P₂ impairs binding of B to PD-L1.

Embodiment 28. The isolated multispecific antibody of any one of embodiments 2-27, wherein P₂ is bound to B through ionic interactions, electrostatic interactions, hydrophobic interactions, Pi-stacking interactions, and H-bonding interactions, or a combination thereof.

Embodiment 29. The isolated multispecific antibody of any one of embodiments 2-28, wherein P₂ is bound to B at or near an antigen binding site.

Embodiment 30. The isolated multispecific antibody of any one of embodiments 2-29, wherein P₂ becomes unbound from B when L2 is cleaved by the tumor specific protease thereby exposing B to the PD-L1.

Embodiment 31. The isolated multispecific antibody of any one of embodiments 2-30, wherein P₂ has less than 70% sequence identity to the PD-L1.

Embodiment 32. The isolated multispecific antibody of any one of embodiments 2-31, wherein P₂ has less than 75% sequence identity to the PD-L1.

Embodiment 33. The isolated multispecific antibody of any one of embodiments 2-32, wherein P₂ has less than 80% sequence identity to the PD-L1.

Embodiment 34. The isolated multispecific antibody of any one of embodiments 2-33, wherein P₂ has less than 85% sequence identity to the PD-L1.

Embodiment 35. The isolated multispecific antibody of any one of embodiments 2-34, wherein P₂ has less than 90% sequence identity to the PD-L1.

Embodiment 36. The isolated multispecific antibody of any one of embodiments 2-35, wherein P₂ has less than 95% sequence identity to the PD-L1.

Embodiment 37. The isolated multispecific antibody of any one of embodiments 2-36, wherein P₂ comprises a de novo amino acid sequence that shares less than 10% sequence identity to the PD-L1.

Embodiment 38. The isolated multispecific antibody of any one of embodiments 2-37, wherein P₂ comprises a peptide sequence of at least 5 amino acids in length.

Embodiment 39. The isolated multispecific antibody of any one of embodiments 2-38, wherein P₂ comprises a peptide sequence of at least 6 amino acids in length.

Embodiment 40. The isolated multispecific antibody of any one of embodiments 2-39, wherein P₂ comprises a peptide sequence of at least 10 amino acids in length.

Embodiment 41. The isolated multispecific antibody of any one of embodiments 2-40, wherein P₂ comprises a peptide sequence of at least 10 amino acids in length and no more than 20 amino acids in length.

Embodiment 42. The isolated multispecific antibody of any one of embodiments 2-41, wherein P₂ comprises a peptide sequence of at least 16 amino acids in length.

Embodiment 43. The isolated multispecific antibody of any one of embodiments 2-42, wherein P₂ comprises a peptide sequence of no more than 40 amino acids in length.

Embodiment 44. The isolated multispecific antibody of any one of embodiments 1-43, wherein P₁ or P₂ comprises at least two cysteine amino acid residues.

Embodiment 45. The isolated multispecific antibody of any one of embodiments 1-44, wherein P₁ or P₂ comprises a cyclic peptide or a linear peptide.

Embodiment 46. The isolated multispecific antibody of any one of embodiments 1-45, wherein P₁ or P₂ comprises a cyclic peptide.

Embodiment 47. The isolated multispecific antibody of any one of embodiments 1-46, wherein P₁ or P₂ comprises a linear peptide.

Embodiment 48. The isolated multispecific antibody of any one of embodiments 1-47, wherein P₁ or P₂ comprise a modified amino acid or non-natural amino acid, or a modified non-natural amino acid, or a combination thereof.

Embodiment 49. The isolated multispecific antibody of any one of embodiments 1-48, wherein P₁ or P₂ does not comprise albumin or an albumin fragment.

Embodiment 50. The isolated multispecific antibody of any one of embodiments 1-49, wherein P₁ or P₂ does not comprise an albumin binding domain.

Embodiment 51. The isolated multispecific antibody of any one of embodiments 1-50, wherein L₁ or L₂ is a peptide sequence having at least 5 to no more than 50 amino acids.

Embodiment 52. The isolated multispecific antibody of any one of embodiments 1-51, wherein L₁ or L₂ is a peptide sequence having at least 10 to no more than 30 amino acids.

Embodiment 53. The isolated multispecific antibody of any one of embodiments 1-52, wherein L₁ or L₂ is a peptide sequence having at least 10 amino acids.

Embodiment 54. The isolated multispecific antibody of any one of embodiments 1-53, wherein L₁ or L₂ is a peptide sequence having at least 18 amino acids.

Embodiment 55. The isolated multispecific antibody of any one of embodiments 1-54, wherein L₁ or L₂ is a peptide sequence having at least 26 amino acids.

Embodiment 56. The isolated multispecific antibody of any one of embodiments 1-55, wherein L₁ or L₂ comprises a formula comprising (G2S)n, wherein n is an integer from 1 to 3 (SEQ ID NO: 228).

Embodiment 57. The isolated multispecific antibody of any one of embodiments 1-56, wherein L₁ or L₂ comprises a formula comprising (G2S)n, wherein n is an integer of at least 1.

Embodiment 58. The isolated multispecific antibody of any one of embodiments 1-57, wherein L₁ or L₂ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 59. The isolated multispecific antibody of any one of embodiments 1-58, wherein the tumor specific protease is selected from the group consisting of metalloprotease, serine protease, cysteine protease, threonine protease, and aspartic protease.

Embodiment 60. The isolated multispecific antibody of any one of embodiments 1-58, wherein L₁ or L₂ comprises a urokinase cleavable amino acid sequence, a matriptase cleavable amino acid sequence, or a matrix metalloprotease cleavable amino acid sequence.

Embodiment 61. The isolated multispecific antibody of any one of embodiments 1-60, wherein L₁ or L₂ comprises a sequence according to SEQ ID NOs: 18-19, 62-88.

Embodiment 62. The isolated multispecific antibody of any one of embodiments 1-61, wherein L₁ is bound to N-terminus of A₁.

Embodiment 63. The isolated multispecific antibody of any one of embodiments 1-61, wherein L₁ is bound to C-terminus of A₁.

Embodiment 64. The isolated multispecific antibody of any one of embodiments 1-61, wherein L₂ is bound to N-terminus of B.

Embodiment 65. The isolated multispecific antibody of any one of embodiments 1-61, wherein L₂ is bound to C-terminus of B.

Embodiment 66. The isolated multispecific antibody of any one of embodiments 1-65, wherein the CD28 binding domain comprises a single chain variable fragment, a single domain antibody, a Fab, or a Fab′.

Embodiment 67. The isolated multispecific antibody of embodiment 66, wherein the CD28 binding domain comprises the single chain variable fragment.

Embodiment 68. The isolated multispecific antibody of embodiment 66, wherein the CD28 binding domain comprises the single domain antibody.

Embodiment 69. The isolated multispecific antibody of embodiment 66, wherein the CD28 binding domain comprises the Fab or the Fab′.

Embodiment 70. The isolated multispecific antibody of any one of embodiments 1-69, wherein the PD-L1 binding domain comprises a single chain variable fragment, a single domain antibody, a Fab, or a Fab′.

Embodiment 71. The isolated multispecific antibody of embodiment 70, wherein the PD-L1 binding domain comprises the Fab or the Fab′.

Embodiment 72. The isolated multispecific antibody of embodiment 70, wherein the PD-L1 binding domain comprises the Fab or the Fab′ and the CD28 binding domain comprises the single chain variable fragment.

Embodiment 73. The isolated multispecific antibody of embodiment 70, wherein the PD-L1 binding domain that comprises the Fab or the Fab′ comprises a Fab heavy chain polypeptide comprising a Fab heavy chain variable domain and a Fab light chain polypeptide comprising a Fab light chain variable domain.

Embodiment 74. The isolated multispecific antibody of embodiment 73, wherein the CD28 binding domain that comprises the single chain variable fragment comprises a scFv heavy chain variable domain and a scFv light chain variable domain.

Embodiment 75. The isolated multispecific antibody of any one of embodiments 1-74, wherein the linker connects the C-terminus of A₁ to an N-terminus of B.

Embodiment 76. The isolated multispecific antibody of any one of embodiments 1-74, wherein the linker connects the N-terminus of A₁ to a C-terminus of B.

Embodiment 77. The isolated multispecific antibody of embodiment 73, wherein the linker connects the C-terminus of A₁ to the N-terminus of the Fab heavy chain polypeptide.

Embodiment 78. The isolated multispecific antibody of embodiment 73, wherein the linker connects the N-terminus of A₁ to the C-terminus of the Fab heavy chain polypeptide.

Embodiment 79. The isolated multispecific antibody of embodiment 73, wherein the linker connects the C-terminus of A₁ to the N-terminus of the Fab light chain polypeptide.

Embodiment 80. The isolated multispecific antibody of embodiment 73, wherein the linker connects the N-terminus of A₁ to the C-terminus of the Fab light chain polypeptide.

Embodiment 81. The isolated multispecific antibody of embodiment 73, wherein the linker connects the Fab light chain polypeptide to the scFv light chain variable domain.

Embodiment 82. The isolated multispecific antibody of embodiment 73, wherein the linker connects the Fab light chain polypeptide to the scFv heavy chain variable domain.

Embodiment 83. The isolated multispecific antibody of embodiment 73, wherein the linker connects the Fab heavy chain polypeptide to the scFv light chain variable domain.

Embodiment 84. The isolated multispecific antibody of embodiment 73, wherein the linker connects the Fab heavy chain polypeptide to the scFv heavy chain variable domain.

Embodiment 85. The isolated multispecific antibody of embodiment 73, wherein the linker connects the Fab light chain polypeptide to the N-terminus of the scFv light chain variable domain.

Embodiment 86. The isolated multispecific antibody of embodiment 73, wherein the linker connects the Fab light chain polypeptide to the C-terminus of the scFv light chain variable domain.

Embodiment 87. The isolated multispecific antibody of embodiment 73, wherein the linker connects the Fab light chain polypeptide to the N-terminus of the scFv heavy chain variable domain.

Embodiment 88. The isolated multispecific antibody of embodiment 73, wherein the linker connects the Fab light chain polypeptide to the C-terminus of the scFv heavy chain variable domain.

Embodiment 89. The isolated multispecific antibody of embodiment 73, wherein the linker connects the Fab heavy chain polypeptide to the N-terminus of the scFv light chain variable domain.

Embodiment 90. The isolated multispecific antibody of embodiment 73, wherein the linker connects the Fab heavy chain polypeptide to the C-terminus of the scFv light chain variable domain.

Embodiment 91. The isolated multispecific antibody of embodiment 73, wherein the linker connects the Fab heavy chain polypeptide to the N-terminus of the scFv heavy chain variable domain.

Embodiment 92. The isolated multispecific antibody of embodiment 73, wherein the linker connects the Fab heavy chain polypeptide to the C-terminus of the scFv heavy chain variable domain.

Embodiment 93. The isolated multispecific antibody of any one of embodiments 1-92, wherein the linker is at least 5 amino acids in length.

Embodiment 94. The isolated multispecific antibody of any one of embodiments 1-93, wherein the linker is no more than 30 amino acids in length.

Embodiment 95. The isolated multispecific antibody of any one of embodiments 1-94, wherein the linker is at least 5 amino acids and no more than 30 amino acids in length.

Embodiment 96. The isolated multispecific antibody of any one of embodiments 1-95, wherein the linker is 5 amino acids in length.

Embodiment 97. The isolated multispecific antibody of any one of embodiments 1-96, wherein the linker is 15 amino acids in length.

Embodiment 98. The isolated multispecific antibody of any one of embodiments 1-97, wherein the linker comprises (G2S)n, (GS)n, (GSGGS)n (SEQ ID NO: 58), (GGGS)n (SEQ ID NO: 59), (GGGGS)n (SEQ ID NO: 60), and (GSSGGS)n (SEQ ID NO: 61), wherein n is an integer of at least 1.

Embodiment 99. The isolated multispecific antibody of any one of embodiments 1-98, wherein L comprises a formula comprising (G2S)n, wherein n is an integer from 1 to 3 (SEQ ID NO: 228).

Embodiment 100. The isolated multispecific antibody of any one of embodiments 1-97, wherein the L comprises an amino acid sequence of SEQ ID NO: 18 (GGGGSGGGGSGGGGS) or SEQ ID NO: 19 (GGGGS).

Embodiment 101. The isolated multispecific antibody of embodiment 73, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

Embodiment 102. The isolated multispecific antibody of embodiment 73, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5 (KA); and LC-CDR3: SEQ ID NO: 6, and wherein the CDRs comprise from 0-2 amino acid modifications in at least one of said LC-CDR1, LC-CDR2, or LC-CDR3.

Embodiment 103. The isolated multispecific antibody of any one of embodiments 1-100, wherein A₁ comprises complementarity determining regions (CDRs): LC-CDR1, LC-CDR2, and LC-CDR3, wherein the LC-CDR1, the LC-CDR2, and the LC-CDR3 of A₁ comprise: LC-CDR1: SEQ ID NO: 4; LC-CDR2: SEQ ID NO: 5 (KA); and LC-CDR3: SEQ ID NO: 6; wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 1; HC-CDR2: SEQ ID NO: 2; HC-CDR3: SEQ ID NO: 3.

Embodiment 104. The isolated multispecific antibody of embodiment 73, wherein the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12; and wherein said CDRs comprise from 0-2 amino acid modifications in at least one of said HC-CDR1, HC-CDR2, or HC-CDR3.

Embodiment 105. The isolated multispecific antibody of embodiment 73, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14 (DA); and LC-CDR3: SEQ ID NO: 15; and wherein the CDRs comprise from 0-2 amino acid modifications in at least one of said LC-CDR1, LC-CDR2, or LC-CDR3.

Embodiment 106. The isolated multispecific antibody of any one of embodiments 1-100, wherein B comprises complementarity determining region (CDRs): HC-CDR1, HC-CDR2, and HC-CDR3, wherein the HC-CDR1, the HC-CDR2, and the HC-CDR3 of B comprise: HC-CDR1: SEQ ID NO: 10; HC-CDR2: SEQ ID NO: 11; HC-CDR3: SEQ ID NO: 12; and wherein B comprises complementarity determining regions (CDRs): LC-CDR1, LC-CDR2, and LC-CDR3, wherein the LC-CDR1, the LC-CDR2, and the LC-CDR3 of B comprise: LC-CDR1: SEQ ID NO: 13; LC-CDR2: SEQ ID NO: 14 (DA); and LC-CDR3: SEQ ID NO: 15.

Embodiment 107. The isolated multispecific antibody of embodiment 73, wherein the scFv heavy chain variable domain comprises an amino acid sequence that has at least 80% sequence identity to the amino acid sequence according to SEQ ID NO: 7.

Embodiment 108. The isolated multispecific antibody of embodiment 73, wherein the scFv heavy chain variable domain comprises an amino acid sequence of at least 75 consecutive amino acid residues of SEQ ID NO: 7.

Embodiment 109. The isolated multispecific antibody of embodiment 73, wherein the scFv heavy chain variable domain comprises an amino acid sequence of at least 110 consecutive amino acid residues of SEQ ID NO: 7.

Embodiment 110. The isolated multispecific antibody of embodiment 73, wherein the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7.

Embodiment 111. The isolated multispecific antibody of embodiment 73, wherein the scFv heavy chain variable domain comprises an amino acid sequence according to SEQ ID NO: 7.

Embodiment 112. The isolated multispecific antibody of embodiment 73, wherein the scFv light chain variable domain comprises an amino acid sequence that has at least 80% sequence identity to the amino acid sequence according to SEQ ID NO: 8.

Embodiment 113. The isolated multispecific antibody of embodiment 73, wherein the scFv light chain variable domain comprises an amino acid sequence of at least 75 consecutive amino acid residues of SEQ ID NO: 8.

Embodiment 114. The isolated multispecific antibody of embodiment 73, wherein the scFv light chain variable domain comprises an amino acid sequence of at least 100 consecutive amino acid residues of SEQ ID NO: 8.

Embodiment 115. The isolated multispecific antibody of embodiment 73, wherein the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8.

Embodiment 116. The isolated multispecific antibody of embodiment 73, wherein the scFv light chain variable domain comprises an amino acid sequence according to SEQ ID NO: 8.

Embodiment 117. The isolated multispecific antibody of embodiment 73, wherein the scFv comprises an amino acid sequence that has at least 80% sequence identity to the amino acid sequence according to SEQ ID NO: 9.

Embodiment 118. The isolated multispecific antibody of embodiment 73, wherein the scFv comprises an amino acid sequence of at least 175 consecutive amino acid residues of SEQ ID NO: 9.

Embodiment 119. The isolated multispecific antibody of embodiment 73, wherein the scFv comprises an amino acid sequence of at least 210 consecutive amino acid residues of SEQ ID NO: 9.

Embodiment 120. The isolated multispecific antibody of embodiment 73, wherein the scFv comprises an amino acid sequence of at least 210 consecutive amino acid residues of SEQ ID NO: 9 and has at least 80% sequence identity to the at least 210 consecutive amino acid residues of SEQ ID NO: 9.

Embodiment 121. The isolated multispecific antibody of embodiment 73, wherein the scFv comprises an amino acid sequence according to SEQ ID NO: 9.

Embodiment 122. The isolated multispecific antibody of embodiment 73, wherein the Fab heavy chain polypeptide comprises an amino acid sequence that has at least 80% sequence identity to the amino acid sequence according to SEQ ID NO: 17.

Embodiment 123. The isolated multispecific antibody of embodiment 73, wherein the Fab heavy chain polypeptide comprises an amino acid sequence of at least 175 consecutive amino acid residues of SEQ ID NO: 17.

Embodiment 124. The isolated multispecific antibody of embodiment 73, wherein the Fab heavy chain polypeptide comprises an amino acid sequence of at least 215 consecutive amino acid residues of SEQ ID NO: 17.

Embodiment 125. The isolated multispecific antibody of embodiment 73, wherein the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17.

Embodiment 126. The isolated multispecific antibody of embodiment 73, wherein the Fab heavy chain polypeptide comprises an amino acid sequence according to SEQ ID NO: 17.

Embodiment 127. The isolated multispecific antibody of embodiment 73, wherein the Fab light chain polypeptide comprises an amino acid sequence that has at least 80% sequence identity to the amino acid sequence according to SEQ ID NO: 16.

Embodiment 128. The isolated multispecific antibody of embodiment 73, wherein the Fab light chain polypeptide comprises an amino acid sequence of at least 175 consecutive amino acid residues of SEQ ID NO: 16.

Embodiment 129. The isolated multispecific antibody of embodiment 73, wherein the Fab light chain polypeptide comprises an amino acid sequence of at least 200 consecutive amino acid residues of SEQ ID NO: 16.

Embodiment 130. The isolated multispecific antibody of embodiment 73, wherein the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16.

Embodiment 131. The isolated multispecific antibody of embodiment 73, wherein the Fab light chain polypeptide comprises an amino acid sequence according to SEQ ID NO: 16.

Embodiment 132. The isolated multispecific antibody of embodiment 73, wherein the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20 and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

Embodiment 133. The isolated multispecific antibody of embodiment 73, wherein the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO:21.

Embodiment 134. The isolated multispecific antibody of embodiment 73, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22.

Embodiment 135. The isolated multispecific antibody of embodiment 73, wherein the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO:22.

Embodiment 136. The isolated multispecific antibody of any one of embodiments 1-135, wherein the multispecific antibody further comprises a half-life extending molecule (H₁).

Embodiment 137. The isolated multispecific antibody of embodiment 136, wherein H₁ is connected to P₁.

Embodiment 138. The isolated multispecific antibody of embodiment 136, wherein H₁ is connected to P₂.

Embodiment 139. The isolated multispecific antibody of any one of embodiments 136-138, wherein H₁ does not block A₁ binding to CD28.

Embodiment 140. The isolated multispecific antibody of any one of embodiments 136-139, wherein H₁ does not block B binding to PD-L1.

Embodiment 141. The isolated multispecific antibody of any one of embodiments 136-140, H₁ comprises a linking moiety (L₅) that connects H₁ to P₁ or H₁ to P₂.

Embodiment 142. The isolated multispecific antibody of any one of embodiments 136-141, wherein the half-life extending molecule (H₁) does not have binding affinity to PD-L1.

Embodiment 143. The isolated multispecific antibody of any one of embodiments 136-142, wherein the half-life extending molecule (H₁) does not have binding affinity to CD28.

Embodiment 144. The isolated multispecific antibody of any one of embodiments 136-143, wherein the half-life extending molecule (H₁) does not shield the multispecific antibody from CD28.

Embodiment 145. The isolated multispecific antibody of any one of embodiments 136-144, wherein H₁ comprises a sequence according to SEQ ID NOs: 54-57.

Embodiment 146. The isolated multispecific antibody of any one of embodiments 136-144, wherein H₁ comprises an amino acid sequence that has repetitive sequence motifs.

Embodiment 147. The isolated multispecific antibody of any one of embodiments 136-144, wherein H₁ comprises an amino acid sequence that has highly ordered secondary structure.

Embodiment 148. The isolated multispecific antibody of any one of embodiments 136-144, wherein H₁ comprises a polymer.

Embodiment 149. The isolated multispecific antibody of embodiment 148, wherein the polymer is polyethylene glycol (PEG).

Embodiment 150. The isolated multispecific antibody of any one of embodiments 136-149, wherein H₁ comprises albumin.

Embodiment 151. The isolated multispecific antibody of any one of embodiments 136-150, wherein H₁ comprises an Fc domain.

Embodiment 152. The isolated multispecific antibody of embodiment 150, wherein the albumin is serum albumin.

Embodiment 153. The isolated multispecific antibody of embodiment 152, wherein the albumin is human serum albumin.

Embodiment 154. The isolated multispecific antibody of any one of embodiments 136-153, wherein H₁ comprises a polypeptide, a ligand, or a small molecule.

Embodiment 155. The isolated multispecific antibody of embodiment 153, wherein the polypeptide, the ligand or the small molecule binds serum protein or a fragment thereof, a circulating immunoglobulin or a fragment thereof, or CD35/CR1.

Embodiment 156. The isolated multispecific antibody of embodiment 155, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 157. The isolated multispecific antibody of embodiment 155, wherein the circulating immunoglobulin molecule comprises IgG1, IgG2, IgG3, IgG4, sIgA, IgM or IgD.

Embodiment 158. The isolated multispecific antibody of embodiment 155, wherein the serum protein is albumin.

Embodiment 159. The isolated multispecific antibody of embodiment 154, wherein the polypeptide is an antibody.

Embodiment 160. The isolated multispecific antibody of embodiment 159, wherein the antibody comprises a single domain antibody, a single chain variable fragment, a Fab, or a Fab′.

Embodiment 161. The isolated multispecific antibody of embodiment 160, wherein the single domain antibody comprises a single domain antibody that binds to albumin.

Embodiment 162. The isolated multispecific antibody of embodiment 160, wherein the single domain antibody is a human or humanized antibody.

Embodiment 163. The isolated multispecific antibody of embodiment 160, wherein the single domain antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10E and SA21.

Embodiment 164. The isolated multispecific antibody of embodiment 160, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3 or wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 204, HC-CDR2: SEQ ID NO: 205, and HC-CDR3: SEQ ID NO: 206; and wherein the CDRs comprise from 0-2 amino acid modifications in at least one of the HC-CDR1, HC-CDR2, or HC-CDR3.

Embodiment 165. The isolated multispecific antibody of embodiment 164, wherein H₁ comprises an amino acid sequence according to SEQ ID NO: 57 or SEQ ID NO: 207.

Embodiment 166. The isolated multispecific antibody of embodiment 165, wherein H₁ comprises an amino acid sequence that has at least 80% sequence identity to SEQ ID NO: 57 or SEQ ID NO: 207.

Embodiment 167. The isolated multispecific antibody of embodiment 165, wherein H₁ comprises an amino acid sequence that has at least 85% sequence identity to SEQ ID NO: 57 or SEQ ID NO: 207.

Embodiment 168. The isolated multispecific antibody of embodiment 165, wherein H₁ comprises an amino acid sequence that has at least 90% sequence identity to SEQ ID NO: 57 or SEQ ID NO: 207.

Embodiment 169. The isolated multispecific antibody of embodiment 165, wherein H₁ comprises an amino acid sequence that has at least 95% sequence identity to SEQ ID NO: 57 or SEQ ID NO: 207.

Embodiment 170. The isolated multispecific antibody of embodiment 165, wherein H₁ comprises an amino acid sequence that has at least 99% sequence identity to SEQ ID NO: 57 or SEQ ID NO: 207.

Embodiment 171. The isolated multispecific antibody of any one of embodiments 136-170, wherein H₁ comprise a modified amino acid or non-natural amino acid, or a modified non-natural amino acid, or a combination thereof.

Embodiment 172. The isolated multispecific antibody of embodiment 171, wherein the modified amino acid or a modified non-natural amino acid comprises a post-translational modification.

Embodiment 173. The isolated multispecific antibody of any one of embodiments 136-172, wherein H₁ comprises a linking moiety (L₅) that connects H₁ to P₁ or P₂.

Embodiment 174. The isolated multispecific antibody of embodiment 173, wherein L₅ is a peptide sequence having at least 5 to no more than 50 amino acids.

Embodiment 175. The isolated multispecific antibody of embodiment 173, wherein L₅ is a peptide sequence having at least 10 to no more than 30 amino acids.

Embodiment 176. The isolated multispecific antibody of embodiment 173, wherein L₅ is a peptide sequence having at least 10 amino acids.

Embodiment 177. The isolated multispecific antibody of embodiment 173, wherein L₅ is a peptide sequence having at least 18 amino acids.

Embodiment 178. The isolated multispecific antibody of embodiment 173, wherein L₅ is a peptide sequence having at least 26 amino acids.

Embodiment 179. The isolated multispecific antibody of embodiment 173, wherein L₅ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 180. The isolated multispecific antibody of any one of embodiments 1-179, wherein the isolated multispecific antibody comprises an amino acid sequence that has at least 80% sequence identity to SEQ ID NOs: 149-170.

Embodiment 181. The isolated multispecific antibody of any one of embodiments 1-180, wherein the isolated multispecific antibody comprises an amino acid sequence that has at least 85% sequence identity to SEQ ID NOs: 149-170.

Embodiment 182. The isolated multispecific antibody of any one of embodiments 1-181, wherein the isolated multispecific antibody comprises an amino acid sequence that has at least 90% sequence identity to SEQ ID NOs: 149-170.

Embodiment 183. The isolated multispecific antibody of any one of embodiments 1-182, wherein the isolated multispecific antibody comprises an amino acid sequence that has at least 95% sequence identity to SEQ ID NOs: 149-170.

Embodiment 184. The isolated multispecific antibody of any one of embodiments 1-183, wherein the isolated multispecific antibody comprises an amino acid sequence that has at least 99% sequence identity to SEQ ID NOs: 149-170.

Embodiment 185. The isolated multispecific antibody of any one of embodiments 1-183, wherein the isolated multispecific antibody comprises an amino acid sequence that has at least 95% sequence identity to SEQ ID NOs: 149 and 150.

Embodiment 186. The isolated multispecific antibody of any one of embodiments 1-183, wherein the isolated multispecific antibody comprises an amino acid sequence that has at least 99% sequence identity to SEQ ID NOs: 149 and 150.

Embodiment 187. The isolated multispecific antibody of any one of embodiments 1-183, wherein the isolated multispecific antibody comprises an amino acid sequence that has at least 95% sequence identity to SEQ ID NOs: 151 and 152.

Embodiment 188. The isolated multispecific antibody of any one of embodiments 1-183, wherein the isolated multispecific antibody comprises an amino acid sequence that has at least 99% sequence identity to SEQ ID NOs: 151 and 152.

Embodiment 189. The isolated multispecific antibody of any one of embodiments 1-183, wherein the isolated multispecific antibody comprises an amino acid sequence that has at least 95% sequence identity to SEQ ID NOs: 153 and 154.

Embodiment 190. The isolated multispecific antibody of any one of embodiments 1-183, wherein the isolated multispecific antibody comprises an amino acid sequence that has at least 99% sequence identity to SEQ ID NOs: 153 and 154.

Embodiment 191. The isolated multispecific antibody of any one of embodiments 1-183, wherein the isolated multispecific antibody comprises an amino acid sequence that has at least 95% sequence identity to SEQ ID NOs: 155 and 156.

Embodiment 192. The isolated multispecific antibody of any one of embodiments 1-183, wherein the isolated multispecific antibody comprises an amino acid sequence that has at least 99% sequence identity to SEQ ID NOs: 155 and 156.

Embodiment 193. The isolated multispecific antibody of any one of embodiments 1-183, wherein the isolated multispecific antibody comprises an amino acid sequence that has at least 95% sequence identity to SEQ ID NOs: 157 and 158.

Embodiment 194. The isolated multispecific antibody of any one of embodiments 1-183, wherein the isolated multispecific antibody comprises an amino acid sequence that has at least 99% sequence identity to SEQ ID NOs: 157 and 158.

Embodiment 195. The isolated multispecific antibody of any one of embodiments 1-183, wherein the isolated multispecific antibody comprises an amino acid sequence that has at least 95% sequence identity to SEQ ID NOs: 159 and 160.

Embodiment 196. The isolated multispecific antibody of any one of embodiments 1-183, wherein the isolated multispecific antibody comprises an amino acid sequence that has at least 99% sequence identity to SEQ ID NOs: 159 and 160.

Embodiment 197. The isolated multispecific antibody of any one of embodiments 1-183, wherein the isolated multispecific antibody comprises an amino acid sequence that has at least 95% sequence identity to SEQ ID NOs: 161 and 162.

Embodiment 198. The isolated multispecific antibody of any one of embodiments 1-183, wherein the isolated multispecific antibody comprises an amino acid sequence that has at least 99% sequence identity to SEQ ID NOs: 161 and 162.

Embodiment 199. The isolated multispecific antibody of any one of embodiments 1-183, wherein the isolated multispecific antibody comprises an amino acid sequence that has at least 95% sequence identity to SEQ ID NOs: 163 and 164.

Embodiment 200. The isolated multispecific antibody of any one of embodiments 1-183, wherein the isolated multispecific antibody comprises an amino acid sequence that has at least 99% sequence identity to SEQ ID NOs: 163 and 164.

Embodiment 201. The isolated multispecific antibody of any one of embodiments 1-183, wherein the isolated multispecific antibody comprises an amino acid sequence that has at least 95% sequence identity to SEQ ID NOs: 165 and 166.

Embodiment 202. The isolated multispecific antibody of any one of embodiments 1-183, wherein the isolated multispecific antibody comprises an amino acid sequence that has at least 99% sequence identity to SEQ ID NOs: 165 and 166.

Embodiment 203. The isolated multispecific antibody of any one of embodiments 1-183, wherein the isolated multispecific antibody comprises an amino acid sequence that has at least 95% sequence identity to SEQ ID NOs: 167 and 168.

Embodiment 204. The isolated multispecific antibody of any one of embodiments 1-183, wherein the isolated multispecific antibody comprises an amino acid sequence that has at least 99% sequence identity to SEQ ID NOs: 167 and 168.

Embodiment 205. The isolated multispecific antibody of any one of embodiments 1-183, wherein the isolated multispecific antibody comprises an amino acid sequence that has at least 95% sequence identity to SEQ ID NOs: 169 and 170 or at least 95% sequence identity to SEQ ID NOs: 208 and 209.

Embodiment 206. The isolated multispecific antibody of any one of embodiments 1-183, wherein the isolated multispecific antibody comprises an amino acid sequence that has at least 99% sequence identity to SEQ ID NOs: 169 and 170 or has at least 99% sequence identity to SEQ ID NOs: 208 and 209.

Embodiment 207. An isolated recombinant nucleic acid molecule encoding a polypeptide of the isolated multispecific antibody of any one of embodiments 1-206.

Embodiment 208. A pharmaceutical composition comprising:
(a) the isolated multispecific antibody of any one of embodiments 1-206; and
(b) a pharmaceutically acceptable excipient.

Embodiment 209. A pharmaceutical composition comprising: (a) the isolated multispecific antibody of any one of embodiments 1-206, (b) an anti-cancer therapy, and (c) a pharmaceutically acceptable excipient.

Embodiment 210. Embodiment 2. The pharmaceutical composition of embodiment 209, wherein the anti-cancer therapy comprises a small molecule, a cell-based therapy, or an antibody-based therapy.

Embodiment 211. The pharmaceutical composition of embodiment 210, wherein the antibody-based therapy is a T cell engager.

Embodiment 212. The pharmaceutical composition of embodiment 211, wherein the T cell engager comprises a formula according to: D₁-L₀-E₁ (Formula II), wherein D₁ comprises an effector cell binding domain that binds to an effector cell antigen, E₁ comprises a tumor antigen binding domain that binds to a tumor antigen, and L₀ comprises a linker that connects D₁ to E₁.

Embodiment 213. The pharmaceutical composition of embodiment 212, wherein D₁ comprises a single chain variable fragment, a single domain antibody, a Fab fragment, or a Fab′.

Embodiment 214. The pharmaceutical composition of embodiment 213, wherein D₁ comprises the single chain variable fragment.

Embodiment 215. The pharmaceutical composition of embodiment 212, wherein E₁ comprises a single chain variable fragment, a single domain antibody, a Fab fragment, or a Fab′.

Embodiment 216. The pharmaceutical composition of embodiment 215, wherein E₁ comprises the Fab fragment.

Embodiment 217. The pharmaceutical composition of embodiment 215, wherein the effector cell antigen comprises CD3.

Embodiment 218. The pharmaceutical composition of embodiment 217, wherein the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 219. The pharmaceutical composition of embodiment 217, wherein the effector cell binding domain comprises an amino acid sequence according to SEQ ID NOs: 89-101.

Embodiment 220. The pharmaceutical composition of any one of embodiments 212-219 wherein the tumor antigen comprises epidermal growth factor receptor (EGFR), prostate-specific membrane antigen (PSMA), or tumor-associated calcium signal transducer 2 (referred to herein after as TROP2).

Embodiment 221. The pharmaceutical composition of embodiment 220, wherein the tumor antigen comprises EGFR.

Embodiment 222. The pharmaceutical composition of embodiment 220, wherein the tumor antigen binding domain comprises an amino acid sequence according to SEQ ID NOs: 102-111.

Embodiment 223. The pharmaceutical composition of embodiment 220, wherein the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 105; HC-CDR2: SEQ ID NO: 106; HC-CDR3: SEQ ID NO: 107; and wherein the LC-CDR1, the LC-CDR2, and the LC-CDR3 comprise: LC-CDR1: SEQ ID NO: 102; LC-CDR2: SEQ ID NO: 103 (YAS); and LC-CDR3: SEQ ID NO: 104.

Embodiment 224. The pharmaceutical composition of embodiment 220, wherein the tumor antigen comprises EGFR, and the T cell engager comprises amino acid sequences with at least 95% sequence identity according to SEQ ID NOs: 181 and 182 or at least 95% sequence identity according to SEQ ID NOs: 214 and 215.

Embodiment 225. The pharmaceutical composition of embodiment 220, wherein the tumor antigen comprises EGFR, and the T cell engager comprises amino acid sequences according to SEQ ID NOs: 181 and 182 or according to SEQ ID NOs: 214 and 215.

Embodiment 226. The pharmaceutical composition of embodiment 220, wherein the tumor antigen comprises TROP2.

Embodiment 227. The pharmaceutical composition of embodiment 220, wherein the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 112; HC-CDR2: SEQ ID NO: 113; HC-CDR3: SEQ ID NO: 114; and wherein the LC-CDR1, the LC-CDR2, and the LC-CDR3 comprise: LC-CDR1: SEQ ID NO: 115; LC-CDR2: SEQ ID NO: 116 (SAS); and LC-CDR3: SEQ ID NO: 117.

Embodiment 228. The pharmaceutical composition of embodiment 220, wherein the tumor antigen comprises TROP2, and the T cell engager comprises amino acid sequences with at least 95% sequence identity according to SEQ ID NOs: 187-192.

Embodiment 229. The pharmaceutical composition of embodiment 220, wherein the tumor antigen comprises TROP2, and the T cell engager comprises amino acid sequences according to any one of SEQ ID NOs: 187-192.

Embodiment 230. The pharmaceutical composition of embodiment 220, wherein the tumor antigen binding domain comprises an amino acid sequence according to SEQ ID NOs: 112-119.

Embodiment 231. The pharmaceutical composition of embodiment 220, wherein the tumor antigen comprises PSMA.

Embodiment 232. The pharmaceutical composition of embodiment 220, wherein the tumor antigen binding domain comprises an amino acid sequence according to SEQ ID NOs: 120-127.

Embodiment 233. The pharmaceutical composition of embodiment 220, wherein the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 120; HC-CDR2: SEQ ID NO: 121; HC-CDR3: SEQ ID NO: 122; and wherein the LC-CDR1, the LC-CDR2, and the LC-CDR3 comprise: LC-CDR1: SEQ ID NO: 123; LC-CDR2: SEQ ID NO: 124 (EA); and LC-CDR3: SEQ ID NO: 125.

Embodiment 234. The pharmaceutical composition of embodiment 220, wherein the tumor antigen comprises PSMA, and the T cell engager comprises amino acid sequences with at least 95% sequence identity according to SEQ ID NOs: 173 and 174.

Embodiment 235. The pharmaceutical composition of embodiment 220, wherein the tumor antigen comprises PSMA, and the T cell engager comprises amino acid sequences according to SEQ ID NOs: 173 and 174.

Embodiment 236. The pharmaceutical composition of any one of embodiments 211-235, wherein the T cell engager molecule is selectively activated in tumor microenvironments.

Embodiment 237. The pharmaceutical composition of embodiment 236, wherein the T cell engager is according to the following subformula: P₃-L₃-D₁-L₀-E₁ (Formula IIa) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease.

Embodiment 238. The pharmaceutical composition of embodiment 236, wherein the T cell engager is according to the following subformula: D₁-L₀-E₁-L₄-P₄ (Formula IIb) wherein D₁ comprises the CD3 binding domain; E₁ comprises the tumor antigen binding domain; L₀ comprises the linker that connects D₁ to E₁; P₄ comprises a peptide that binds to E₁ and L₄ comprises a linking moiety that connects E₁ to P₄ and is a substrate for a tumor specific protease.

Embodiment 239. The pharmaceutical composition of embodiment 236, wherein the T cell engager is according to the following subformula: P₃-L₃-D₁-L₀-E₁-L₄-P₄ (Formula IIc) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease; P₄ comprises a peptide that binds to E₁ and L₄ comprises a linking moiety that connects E₁ to P₄ and is a substrate for a tumor specific protease.

Embodiment 240. The pharmaceutical composition of any one of embodiments 211-239, wherein the T cell engager comprises H₁.

Embodiment 241. The pharmaceutical composition of embodiment 240, wherein H₁ comprises a sequence according to SEQ ID NO: 54-57.

Embodiment 242. The pharmaceutical composition of embodiment 240, wherein H₁ comprises a single domain antibody.

Embodiment 243. The pharmaceutical composition of embodiment 240, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56.

Embodiment 244. The pharmaceutical composition of any one of embodiments 237-243, wherein L₃ or L₄ is a peptide sequence having at least 5 to no more than 50 amino acids.

Embodiment 245. The pharmaceutical composition of any one of embodiments 237-244, wherein L₃ or L₄ is a peptide sequence having at least 10 to no more than 30 amino acids.

Embodiment 246. The pharmaceutical composition of any one of embodiments 237-245, wherein L₃ or L₄ is a peptide sequence having at least 10 amino acids.

Embodiment 247. The pharmaceutical composition of any one of embodiments 237-246, wherein L₃ or L₄ is a peptide sequence having at least 18 amino acids.

Embodiment 248. The pharmaceutical composition of any one of embodiments 237-247, wherein L₃ or L₄ is a peptide sequence having at least 26 amino acids.

Embodiment 249. The pharmaceutical composition of any one of embodiments 237-243, wherein L₃ or L₄ comprises a formula comprising (G₂S)ₙ, wherein n is an integer from 1 to 3 (SEQ ID NO: 228).

Embodiment 250. The pharmaceutical composition of any one of embodiments 237-243, wherein L₃ or L₄ comprises a formula comprising (G₂S)ₙ, wherein n is an integer of at least 1.

Embodiment 251. The pharmaceutical composition of any one of embodiments 237-243, wherein L₃ or L₄ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 252. The pharmaceutical composition of any one of embodiments 237-243, wherein the tumor specific protease is selected from the group consisting of metalloprotease, serine protease, cysteine protease, threonine protease, and aspartic protease.

Embodiment 253. The pharmaceutical composition of any one of embodiments 237-243, wherein L₃ or L₄ comprises a urokinase cleavable amino acid sequence, a matriptase cleavable amino acid sequence, or a matrix metalloprotease cleavable amino acid sequence.

Embodiment 254. The pharmaceutical composition of any one of embodiments 237-243, wherein L₃ or L₄ comprises a sequence according to SEQ ID NOs: 18-19, 62-88.

Embodiment 255. The pharmaceutical composition of any one of embodiments 237-254, wherein L₃ is bound to N-terminus of D₁.

Embodiment 256. The pharmaceutical composition of any one of embodiments 237-254, wherein L₃ is bound to C-terminus of D₁.

Embodiment 257. The pharmaceutical composition of any one of embodiments 238-254, wherein L₄ is bound to N-terminus of E₁.

Embodiment 258. The pharmaceutical composition of any one of embodiments 238-254, wherein L₄ is bound to C-terminus of E₁.

Embodiment 259. The pharmaceutical composition of any one of embodiments 237-254, wherein P₃ becomes unbound from D₁ when L₃ is cleaved by the tumor specific protease thereby exposing D₁ to CD3.

Embodiment 260. The pharmaceutical composition of any one of embodiments 238-254, wherein P₄ becomes unbound from E₁ when L₄ is cleaved by the tumor specific protease thereby exposing E₁ to the tumor antigen.

Embodiment 261. The pharmaceutical composition of any one of embodiments 237-260, wherein P₃ impairs binding of D₁ to CD3.

Embodiment 262. The pharmaceutical composition of any one of embodiments 237-261, wherein P₃ is bound to D₁ through ionic interactions, electrostatic interactions, hydrophobic interactions, Pi-stacking interactions, and H-bonding interactions, or a combination thereof.

Embodiment 263. The pharmaceutical composition of any one of embodiments 237-262, wherein P₃ is bound to D₁ at or near an antigen binding site.

Embodiment 264. The pharmaceutical composition of any one of embodiments 237-263, wherein P₃ becomes unbound from D₁ when L₃ is cleaved by the tumor specific protease thereby exposing D₁ to CD3.

Embodiment 265. The pharmaceutical composition of any one of embodiments 237-264, wherein P₃ has less than 70% sequence identity to CD3.

Embodiment 266. The pharmaceutical composition of any one of embodiments 237-265, wherein P₃ has less than 85% sequence identity to CD3.

Embodiment 267. The pharmaceutical composition of any one of embodiments 237-266, wherein P₃ has less than 90% sequence identity to CD3.

Embodiment 268. The pharmaceutical composition of any one of embodiments 237-267, wherein P₃ has less than 95% sequence identity to CD3.

Embodiment 269. The pharmaceutical composition of any one of embodiments 237-268, wherein P₃ has less than 98% sequence identity to CD3.

Embodiment 270. The pharmaceutical composition of any one of embodiments 237-269, wherein P₃ has less than 99% sequence identity to CD3.

Embodiment 271. The pharmaceutical composition of any one of embodiments 237-270, wherein P₃ comprises the amino acid sequence according to SEQ ID NOs: 177-180.

Embodiment 272. The pharmaceutical composition of any one of embodiments 237-271, wherein P₃ comprises a de novo amino acid sequence that shares less than 10% sequence identity to CD3.

Embodiment 273. The pharmaceutical composition of any one of embodiments 238-272, wherein P₄ impairs binding of E₁ to the tumor antigen.

Embodiment 274. The pharmaceutical composition of any one of embodiments 238-273, wherein P₄ is bound to E₁ through ionic interactions, electrostatic interactions, hydrophobic interactions, Pi-stacking interactions, and H-bonding interactions, or a combination thereof.

Embodiment 275. The pharmaceutical composition of any one of embodiments 238-274, wherein P₄ is bound to E₁ at or near an antigen binding site.

Embodiment 276. The pharmaceutical composition of any one of embodiments 238-275, wherein P₄ becomes unbound from E₁ when L4 is cleaved by the tumor specific protease thereby exposing E₁ to the tumor antigen.

Embodiment 277. The pharmaceutical composition of any one of embodiments 238-276, wherein P₄ has less than 70% sequence identity to the tumor antigen.

Embodiment 278. The pharmaceutical composition of any one of embodiments 238-277, wherein P₄ has less than 80% sequence identity to the tumor antigen.

Embodiment 279. The pharmaceutical composition of any one of embodiments 238-278, wherein P₄ has less than 85% sequence identity to the tumor antigen.

Embodiment 280. The pharmaceutical composition of any one of embodiments 238-279, wherein P₄ has less than 90% sequence identity to the tumor antigen.

Embodiment 281. The pharmaceutical composition of any one of embodiments 238-280, wherein P₄ has less than 95% sequence identity to the tumor antigen.

Embodiment 282. The pharmaceutical composition of any one of embodiments 238-281, wherein P₄ comprises a de novo amino acid sequence that shares less than 10% sequence identity to the tumor antigen.

Embodiment 283. The pharmaceutical composition of any one of embodiments 237-282, wherein P₃ or P₄ comprises a peptide sequence of at least 5 amino acids in length.

Embodiment 284. The pharmaceutical composition of any one of embodiments 237-283, wherein P₃ or P₄ comprises a peptide sequence of at least 6 amino acids in length.

Embodiment 285. The pharmaceutical composition of any one of embodiments 237-284, wherein P₃ or P₄ comprises a peptide sequence of at least 10 amino acids in length.

Embodiment 286. The pharmaceutical composition of any one of embodiments 237-285, wherein P₃ or P₄ comprises a peptide sequence of at least 10 amino acids in length and no more than 20 amino acids in length.

Embodiment 287. The pharmaceutical composition of any one of embodiments 237-286, wherein P₃ or P₄ comprises a peptide sequence of at least 16 amino acids in length.

Embodiment 288. The pharmaceutical composition of any one of embodiments 237-287, wherein P₃ or P₄ comprises a peptide sequence of no more than 40 amino acids in length.

Embodiment 289. The pharmaceutical composition of any one of embodiments 237-288, wherein P₃ or P₄ comprises at least two cysteine amino acid residues.

Embodiment 290. The pharmaceutical composition of any one of embodiments 237-289, wherein P₃ or P₄ comprises a cyclic peptide or a linear peptide.

Embodiment 291. The pharmaceutical composition of any one of embodiments 237-290, wherein P₃ or P₄ comprises a cyclic peptide.

Embodiment 292. The pharmaceutical composition of any one of embodiments 237-291, wherein P₃ or P₄ comprises a linear peptide.

Embodiment 293. The pharmaceutical composition of any one of embodiments 238-292, wherein P₄ comprises the amino acid sequence according to SEQ ID NO: 185 or 186.

Embodiment 294. The pharmaceutical composition of any one of embodiments 237-293, wherein the tumor antigen comprises EGFR, and the T cell engager comprises the amino acid sequence of SEQ ID NOs: 183 and 184,

Embodiment 295. The pharmaceutical composition of any one of embodiments 238-292, wherein P₄ comprises the amino acid sequence according to SEQ ID NOs: 199-201.

Embodiment 296. The pharmaceutical composition of any one of embodiments 237-292, wherein the tumor antigen comprises TROP2, and the T cell engager comprises any one of amino acid sequences of SEQ ID NOs: 193-198.

Embodiment 297. The pharmaceutical composition of any one of embodiments 237-292, wherein the tumor antigen comprises PSMA, and the T cell engager comprises the amino acid sequence of SEQ ID NOs: 175 and 176.

Embodiment 298. An isolated polypeptide or polypeptide complex comprising a CD28 binding domain that is linked to a peptide that impairs binding of the CD28 binding domain to CD28 wherein the peptide comprises an amino acid sequence according to any one of SEQ ID NOs: 24-53, 128-148, and any one of the amino acid sequences of Table 20, or an amino acid sequence that has 1, 2, or 3 amino acid mutations, substitutions, or deletions relative to any one of SEQ ID NOs: 24-53, 128-148, and any one of the amino acid sequences of Table 20.

Embodiment 299. The isolated polypeptide or polypeptide complex of embodiment 298, wherein the peptide comprises an amino acid sequence according to any one of SEQ ID NOs: 24-53, 128-148, and the amino acid sequences of Table 20.

Embodiment 300. The isolated polypeptide or polypeptide complex of embodiment 298, wherein the peptide comprises an amino acid sequence according to any one of SEQ ID NOs: 42-53 or an amino acid sequence that has 1, 2, or 3 amino acid mutations, substitutions, or deletions relative to any one of SEQ ID NOs: 42-53.

Embodiment 301. The isolated polypeptide or polypeptide complex of embodiment 298, wherein the peptide comprises an amino acid sequence according to any one of SEQ ID NOs: 42-53.

Embodiment 302. The isolated polypeptide or polypeptide complex of embodiment 298, wherein the peptide comprises an amino acid sequence according to any one of the amino acid sequences of Table 20 or an amino acid sequence that has 1, 2, or 3 amino acid mutations, substitutions, or deletions relative to any one of the amino acid sequences of Table 20.

Embodiment 303. The isolated polypeptide or polypeptide complex of embodiment 298, wherein the peptide comprises an amino acid sequence according to any one of the amino acid sequences of Table 20.

Embodiment 304. The isolated polypeptide or polypeptide complex of embodiment 298, wherein the peptide comprises an amino acid sequence according to any one of SEQ ID NOs: 128-147 or an amino acid sequence that has 1, 2, or 3 amino acid mutations, substitutions, or deletions relative to any one of SEQ ID NOs: 128-147.

Embodiment 305. The isolated polypeptide or polypeptide complex of embodiment 298, wherein the peptide comprises an amino acid sequence according to any one of SEQ ID NOs: 128-147.

Embodiment 306. The isolated polypeptide or polypeptide complex of embodiment 298, wherein the peptide comprises an amino acid sequence according to X₁-X₂-X₃-C-X₄-X₅-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L.

Embodiment 307. The isolated polypeptide or polypeptide complex of embodiment 306, wherein X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y.

Embodiment 308. The isolated polypeptide or polypeptide complex of embodiment 307, wherein X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F.

Embodiment 309. The isolated polypeptide or polypeptide complex of any one of embodiments 298-302, or 306-308, wherein the peptide comprises an amino acid sequence according to SEQ ID NO: 32 or an amino acid sequence that has 1, 2, or 3 amino acid mutations, substitutions, or deletions relative to SEQ ID NO: 32.

Embodiment 310. The isolated polypeptide or polypeptide complex of any one of embodiments 298-302, or 306-308.

Embodiment 311. The isolated polypeptide or polypeptide complex of embodiment 298, wherein the peptide comprises an amino acid sequence according to SEQ ID NO: 32.

Embodiment 312. The isolated polypeptide or polypeptide complex of any one of embodiments 298-302, or 306-308, wherein the peptide comprises an amino acid sequence according to SEQ ID NO: 138 or an amino acid sequence that has 1, 2, or 3 amino acid mutations, substitutions, or deletions relative to SEQ ID NO: 138.

Embodiment 313. The isolated polypeptide or polypeptide complex of any one of embodiments 298-302, or 306-308, wherein the peptide comprises an amino acid sequence according to SEQ ID NO: 138.

Embodiment 314. The isolated polypeptide or polypeptide complex of any one of embodiments 298-312, wherein the CD28 binding domain comprises a single chain variable fragment, a single domain antibody, a Fab, or a Fab′.

Embodiment 315. The isolated polypeptide or polypeptide complex of embodiment 314, wherein the CD28 binding domain comprises the single chain variable fragment and the single chain variable fragment comprises a scFv heavy chain variable domain and a scFv light chain variable domain.

Embodiment 316. The isolated polypeptide or polypeptide complex of embodiment 314, wherein the CD28 binding domain comprises the single domain antibody.

Embodiment 317. The isolated polypeptide or polypeptide complex of embodiment 314, wherein the CD28 binding domain comprises the Fab or the Fab′.

Embodiment 318. The isolated polypeptide or polypeptide complex of embodiment 315, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

Embodiment 319. The isolated polypeptide or polypeptide complex of embodiment 315, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5 (KA); and LC-CDR3: SEQ ID NO: 6, and wherein the CDRs comprise from 0-2 amino acid modifications in at least one of said LC-CDR1, LC-CDR2, or LC-CDR3.

Embodiment 320. The isolated polypeptide or polypeptide complex of embodiment 315, wherein the scFv heavy chain variable domain comprises an amino acid sequence that has at least 80% sequence identity to the amino acid sequence according to SEQ ID NO: 7.

Embodiment 321. The isolated polypeptide or polypeptide complex of embodiment 315, wherein the scFv heavy chain variable domain comprises an amino acid sequence of at least 75 consecutive amino acid residues of SEQ ID NO: 7

Embodiment 322. The isolated polypeptide or polypeptide complex of embodiment 315, wherein the scFv heavy chain variable domain comprises an amino acid sequence of at least 110 consecutive amino acid residues of SEQ ID NO: 7.

Embodiment 323. The isolated polypeptide or polypeptide complex of embodiment 315, wherein the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7.

Embodiment 324. The isolated polypeptide or polypeptide complex of embodiment 315, wherein the scFv heavy chain variable domain comprises an amino acid sequence according to SEQ ID NO: 7.

Embodiment 325. The isolated polypeptide or polypeptide complex of embodiment 315, wherein the scFv light chain variable domain comprises an amino acid sequence that has at least 80% sequence identity to the amino acid sequence according to SEQ ID NO: 8.

Embodiment 326. The isolated polypeptide or polypeptide complex of embodiment 315, wherein the scFv light chain variable domain comprises an amino acid sequence of at least 75 consecutive amino acid residues of SEQ ID NO: 8.

Embodiment 327. The isolated polypeptide or polypeptide complex of embodiment 315, wherein the scFv light chain variable domain comprises an amino acid sequence of at least 100 consecutive amino acid residues of SEQ ID NO: 8.

Embodiment 328. The isolated polypeptide or polypeptide complex of embodiment 315, wherein the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8.

Embodiment 329. The isolated polypeptide or polypeptide complex of embodiment 315, wherein the scFv light chain variable domain comprises an amino acid sequence according to SEQ ID NO: 8.

Embodiment 330. The isolated polypeptide or polypeptide complex of embodiment 314, wherein the scFv comprises an amino acid sequence that has at least 80% sequence identity to the amino acid sequence according to SEQ ID NO: 9.

Embodiment 331. The isolated polypeptide or polypeptide complex of embodiment 315, wherein the scFv comprises an amino acid sequence of at least 175 consecutive amino acid residues of SEQ ID NO: 9.

Embodiment 332. The isolated polypeptide or polypeptide complex of embodiment 315, wherein the scFv comprises an amino acid sequence of at least 210 consecutive amino acid residues of SEQ ID NO: 9.

Embodiment 333. The isolated polypeptide or polypeptide complex of embodiment 315, wherein the scFv comprises an amino acid sequence of at least 210 consecutive amino acid residues of SEQ ID NO: 9 and has at least 80% sequence identity to the at least 210 consecutive amino acid residues of SEQ ID NO: 9.

Embodiment 334. The isolated polypeptide or polypeptide complex of embodiment 315, wherein the scFv comprises an amino acid sequence according to SEQ ID NO: 9.

Embodiment 335. The isolated polypeptide or polypeptide complex of any one of embodiments 298-334, wherein the CD28 binding domain is linked to the peptide through a linking moiety (L₁).

Embodiment 336. The isolated polypeptide or polypeptide complex of embodiment 335, wherein L₁ is a substrate for a tumor specific protease.

Embodiment 337. The isolated polypeptide or polypeptide complex of any one of embodiments 335-336, wherein L₁ is a peptide sequence having at least 5 to no more than 50 amino acids.

Embodiment 338. The isolated polypeptide or polypeptide complex of any one of embodiments 335-337, wherein L₁ is a peptide sequence having at least 10 to no more than 30 amino acids.

Embodiment 339. The isolated polypeptide or polypeptide complex of any one of embodiments 335-338, wherein L₁ is a peptide sequence having at least 10 amino acids.

Embodiment 340. The isolated polypeptide or polypeptide complex of any one of embodiments 335-339, wherein L₁ is a peptide sequence having at least 18 amino acids.

Embodiment 341. The isolated polypeptide or polypeptide complex of any one of embodiments 335-341, wherein L₁ is a peptide sequence having at least 26 amino acids.

Embodiment 342. The isolated polypeptide or polypeptide complex of any one of embodiments 335-341, wherein L₁ comprises a formula comprising (G₂S)ₙ, wherein n is an integer from 1 to 3 (SEQ ID NO: 228).

Embodiment 343. The isolated polypeptide or polypeptide complex of any one of embodiments 335-341, wherein L₁ comprises a formula comprising (G₂S)ₙ, wherein n is an integer of at least 1.

Embodiment 344. The isolated polypeptide or polypeptide complex of any one of embodiments 335-341, wherein L₁ comprises a formula selected from the group consisting of (G₂S)n, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 345. The isolated polypeptide or polypeptide complex of any one of embodiments 335-344, wherein the tumor specific protease is selected from the group consisting of metalloprotease, serine protease, cysteine protease, threonine protease, and aspartic protease.

Embodiment 346. The isolated polypeptide or polypeptide complex of any one of embodiments 335-344, wherein L₁ comprises a urokinase cleavable amino acid sequence, a matriptase cleavable amino acid sequence, or a matrix metalloprotease cleavable amino acid sequence.

Embodiment 347. The isolated polypeptide or polypeptide complex of any one of embodiments 335-346, wherein L₁ comprises a sequence according to SEQ ID NOs: 18-19, 62-88.

Embodiment 348. The isolated polypeptide or polypeptide complex of any one of embodiments 335-347, wherein L₁ is bound to N-terminus of A₁.

Embodiment 349. The isolated polypeptide or polypeptide complex of any one of embodiments 335-347, wherein L₁ is bound to C-terminus of A₁.

Embodiment 350. The isolated polypeptide or polypeptide complex of any one of embodiments 335-349, wherein P₁ becomes unbound from A₁ when L₁ is cleaved by the tumor specific protease thereby exposing A₁ to CD28.

Embodiment 351. The isolated polypeptide or polypeptide complex of any one of embodiments 335-350, wherein L₁ comprise a modified amino acid or non-natural amino acid, or a modified non-natural amino acid, or a combination thereof.

Embodiment 352. The isolated polypeptide or polypeptide complex of embodiment 351, wherein the modified amino acid or a modified non-natural amino acid comprises a post-translational modification.

Embodiment 353. The isolated polypeptide or polypeptide complex of any one of embodiments 298-352, wherein the isolated polypeptide or polypeptide complex further comprises a half-life extending molecule (H₁)

Embodiment 354. The isolated polypeptide or polypeptide complex of embodiment 353, wherein H₁ is connected to the peptide.

Embodiment 355. The isolated polypeptide or polypeptide complex of embodiment 353 or 354, wherein H₁ does not block the CD28 binding domain to CD28.

Embodiment 356. The isolated polypeptide or polypeptide complex of any one of embodiments 354-355, H₁ comprises a linking moiety (L₅) that connects H₁ to the peptide.

Embodiment 357. The isolated polypeptide or polypeptide complex of any one of embodiments 353-356, wherein the half-life extending molecule (H₁) does not have binding affinity to CD28.

Embodiment 358. The isolated polypeptide or polypeptide complex of any one of embodiments 353-357, wherein the half-life extending molecule (H₁) does not shield the isolated polypeptide or polypeptide complex from CD28.

Embodiment 359. The isolated polypeptide or polypeptide complex of any one of embodiments 353-358, wherein H₁ comprises a sequence according to SEQ ID NOs: 54-57.

Embodiment 360. The isolated polypeptide or polypeptide complex of any one of embodiments 353-359, wherein H₁ comprises an amino acid sequence that has repetitive sequence motifs.

Embodiment 361. The isolated polypeptide or polypeptide complex of any one of embodiments 353-360, wherein H₁ comprises an amino acid sequence that has highly ordered secondary structure.

Embodiment 362. The isolated polypeptide or polypeptide complex of any one of embodiments 353-361, wherein H₁ comprises a polymer.

Embodiment 363. The isolated polypeptide or polypeptide complex of embodiment 362, wherein the polymer is polyethylene glycol (PEG).

Embodiment 364. The isolated polypeptide or polypeptide complex of any one of embodiments 353-361, wherein H₁ comprises albumin.

Embodiment 365. The isolated polypeptide or polypeptide complex of any one of embodiments 353-361, wherein H₁ comprises an Fc domain.

Embodiment 366. The isolated polypeptide or polypeptide complex of embodiment 364, wherein the albumin is serum albumin.

Embodiment 367. The isolated polypeptide or polypeptide complex of embodiment 364, wherein the albumin is human serum albumin.

Embodiment 368. The isolated polypeptide or polypeptide complex of any one of embodiments 353-361, wherein H₁ comprises a polypeptide, a ligand, or a small molecule.

Embodiment 369. The isolated polypeptide or polypeptide complex of embodiment 368, wherein the polypeptide, the ligand or the small molecule binds serum protein or a fragment thereof, a circulating immunoglobulin or a fragment thereof, or CD35/CR1.

Embodiment 370. The isolated polypeptide or polypeptide complex of embodiment 369, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 371. The isolated polypeptide or polypeptide complex of embodiment 369, wherein the circulating immunoglobulin molecule comprises IgG1, IgG2, IgG3, IgG4, sIgA, IgM or IgD.

Embodiment 372. The isolated polypeptide or polypeptide complex of embodiment 369, wherein the serum protein is albumin.

Embodiment 373. The isolated polypeptide or polypeptide complex of embodiment 368, wherein the polypeptide is an antibody.

Embodiment 374. The isolated polypeptide or polypeptide complex of embodiment 373, wherein the antibody comprises a single domain antibody, a single chain variable fragment, a Fab, or a Fab′.

Embodiment 375. The isolated polypeptide or polypeptide complex of embodiment 374, wherein the single domain antibody comprises a single domain antibody that binds to albumin.

Embodiment 376. The isolated polypeptide or polypeptide complex of embodiment 374, wherein the single domain antibody is a human or humanized antibody.

Embodiment 377. The isolated polypeptide or polypeptide complex embodiment 374, wherein the single domain antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10E and SA21.

Embodiment 378. The isolated polypeptide or polypeptide complex embodiment 374, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3.

Embodiment 379. The isolated polypeptide or polypeptide complex of any one of embodiments 353-361, wherein H₁ comprises an amino acid sequence according to SEQ ID NO: 57.

Embodiment 380. The isolated polypeptide or polypeptide complex of any one of embodiments 353-361, wherein H₁ comprises an amino acid sequence that has at least 80% sequence identity to SEQ ID NO: 57.

Embodiment 381. The isolated polypeptide or polypeptide complex of any one of embodiments 353-361, wherein H₁ comprises an amino acid sequence that has at least 85% sequence identity to SEQ ID NO: 57.

Embodiment 382. The isolated polypeptide or polypeptide complex of any one of embodiments 353-361, wherein H₁ comprises an amino acid sequence that has at least 90% sequence identity to SEQ ID NO: 57.

Embodiment 383. The isolated polypeptide or polypeptide complex of any one of embodiments 353-361, wherein H₁ comprises an amino acid sequence that has at least 95% sequence identity to SEQ ID NO: 57.

Embodiment 384. The isolated polypeptide or polypeptide complex of any one of embodiments 353-361, wherein H₁ comprises an amino acid sequence that has at least 99% sequence identity to SEQ ID NO: 57.

Embodiment 385. The isolated polypeptide or polypeptide complex of any one of embodiments 353-361, wherein H₁ comprise a modified amino acid or non-natural amino acid, or a modified non-natural amino acid, or a combination thereof.

Embodiment 386. The isolated polypeptide or polypeptide complex of embodiment 385, wherein the modified amino acid or a modified non-natural amino acid comprises a post-translational modification.

Embodiment 387. The isolated polypeptide or polypeptide complex of any one of embodiments 353-387, wherein H₁ comprises a linking moiety (L₅) that connects H₁ to P₁ or P₂.

Embodiment 388. The isolated polypeptide or polypeptide complex of embodiment 387, wherein L₅ is a peptide sequence having at least 5 to no more than 50 amino acids.

Embodiment 389. The isolated polypeptide or polypeptide complex of any one of embodiments 387-388, wherein L₅ is a peptide sequence having at least 10 to no more than 30 amino acids.

Embodiment 390. The isolated polypeptide or polypeptide complex of any one of embodiments 387-389, wherein L₅ is a peptide sequence having at least 10 amino acids.

Embodiment 391. The isolated polypeptide or polypeptide complex of any one of embodiments 387-390, wherein L₅ is a peptide sequence having at least 18 amino acids.

Embodiment 392. The isolated polypeptide or polypeptide complex of any one of embodiments 387-391, wherein L₅ is a peptide sequence having at least 26 amino acids.

Embodiment 393. The isolated polypeptide or polypeptide complex of any one of embodiments 387-392, wherein L₅ comprises a formula selected from the group consisting of (G₂S)n, (GS)n, (GSGGS)n (SEQ ID NO: 58), (GGGS)n (SEQ ID NO: 59), (GGGGS)n (SEQ ID NO: 60), and (GSSGGS)n (SEQ ID NO: 61), wherein n is an integer of at least 1.

Embodiment 394. A method of treating cancer in a subject in need thereof comprising administering to the subject the multispecific antibody of any one of embodiments 1-180.

Embodiment 395. The method of embodiment 394, wherein the multispecific antibody induces T cell mediated cytotoxicity of tumor cells.

Embodiment 396. The method of embodiment 394 or 395, wherein the cancer is a hematological malignancy.

Embodiment 397. The method of embodiment 394 or 395, wherein the cancer is leukemia or lymphoma.

Embodiment 398. The method of embodiment 394 or 395, wherein the cancer is lymphoma, and wherein the lymphoma is B-cell lymphoma.

Embodiment 399. The method of embodiment 394 or 395, wherein the cancer is a solid tumor.

Embodiment 400. The method of embodiment 399, wherein the solid tumor expresses PD-L1.

Embodiment 401. The method of embodiment 399, wherein the solid tumor is sarcoma, breast cancer, lung cancer, or carcinoma.

Embodiment 402. The method of embodiment 399, wherein the solid tumor is lung cancer, and wherein the lung cancer is non-small cell lung cancer.

Embodiment 403. The method of any one of embodiments 394-402, wherein the multispecific antibody is administered in combination with an anti-cancer therapy.

Embodiment 404. The method of embodiment 403, wherein the multispecific antibody and the anti-cancer therapy are administered in the same pharmaceutical composition.

Embodiment 405. The method of embodiment 403, wherein the multispecific antibody and the anti-cancer therapy are administered as separate pharmaceutical compositions.

Embodiment 406. The method of any one of embodiments 403-405, wherein the subject is refractory to checkpoint inhibitor therapy.

Embodiment 407. The method of any one of embodiments 403-405, wherein the subject has relapsed from checkpoint inhibitor therapy.

Embodiment 408. The method of any one of embodiments 403-407, wherein the anti-cancer therapy comprises a small molecule, a cell-based therapy, or an antibody-based therapy.

Embodiment 409. The method of embodiment 408, wherein the antibody-based therapy is a T cell engager.

Embodiment 410. The method of embodiment 409, wherein the T cell engager comprises a formula according to: D₁-L₀-E₁ (Formula II), wherein D₁ comprises an effector cell binding domain that binds to an effector cell antigen, E₁ comprises a tumor antigen binding domain that binds to a tumor antigen, and L₀ comprises a linker that connects D₁ to E₁.

Embodiment 411. The method of embodiment 410, wherein D₁ comprises a single chain variable fragment, a single domain antibody, or a Fab fragment.

Embodiment 412. The method of embodiment 411, wherein D₁ comprises the single chain variable fragment.

Embodiment 413. The method of any one of embodiments 409-411, wherein E₁ comprises a single chain variable fragment, a single domain antibody, a Fab fragment, or a Fab′.

Embodiment 414. The method of embodiment 413, wherein E₁ comprises the Fab fragment.

Embodiment 415. The method of any one of embodiments 410-414, wherein the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 416. The method of any one of embodiments 410-415, wherein the effector cell binding domain comprises an amino acid sequence according to SEQ ID NOs: 89-101.

Embodiment 417. The method of any one of embodiments 410-416, wherein the tumor antigen comprises epidermal growth factor receptor (EGFR), prostate-specific membrane antigen (PSMA), or tumor-associated calcium signal transducer 2 (referred to herein after as TROP2).

Embodiment 418. The method of embodiment 417, wherein the tumor antigen comprises EGFR.

Embodiment 419. The method of embodiment 418, wherein the tumor antigen binding domain comprises an amino acid sequence according to SEQ ID NOs: 102-111.

Embodiment 420. The method of embodiment 417, wherein the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 105; HC-CDR2: SEQ ID NO: 106; HC-CDR3: SEQ ID NO: 107; and wherein the LC-CDR1, the LC-CDR2, and the LC-CDR3 comprise: LC-CDR1: SEQ ID NO: 102; LC-CDR2: SEQ ID NO: 103 (YAS); and LC-CDR3: SEQ ID NO: 104.

Embodiment 421. The method of embodiment 417, wherein the tumor antigen comprises EGFR, and the T cell engager comprises amino acid sequences with at least 95% sequence identity according to SEQ ID NOs: 181 and 182 or at least 95% sequence identity according to SEQ ID NOs: 214 and 215.

Embodiment 422. The method of embodiment 417, wherein the tumor antigen comprises EGFR, and the T cell engager comprises amino acid sequences according to SEQ ID NOs: 181 and 182 or according to SEQ ID NOs: 214 and 215.

Embodiment 423. The method of embodiment 417, wherein the cancer is colorectal cancer (CRC), squamous cell carcinoma of the head and Neck (SCCHN), non-small cell lung cancer (NSCLC), prostate cancer, breast cancer, colon/rectum cancer, head and neck cancer, esophagogastric cancer, liver cancer, glioblastoma, cervical cancer, ovarian cancer, bladder cancer, kidney cancer, or pancreatic cancer.

Embodiment 424. The method of embodiment 417, wherein the tumor antigen comprises TROP2.

Embodiment 425. The method of embodiment 416, wherein the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 112; HC-CDR2: SEQ ID NO: 113; HC-CDR3: SEQ ID NO: 114; and wherein the LC-CDR1, the LC-CDR2, and the LC-CDR3 comprise: LC-CDR1: SEQ ID NO: 115; LC-CDR2: SEQ ID NO: 116 (SAS); and LC-CDR3: SEQ ID NO: 117.

Embodiment 426. The method of embodiment 417, wherein the tumor antigen comprises TROP2, and the T cell engager comprises amino acid sequences with at least 95% sequence identity according to SEQ ID NOs: 187-192.

Embodiment 427. The method of embodiment 417, wherein the tumor antigen comprises TROP2, and the T cell engager comprises amino acid sequences according to any one of SEQ ID NOs: 187-192.

Embodiment 428. The method of embodiment 417, wherein the tumor antigen binding domain comprises an amino acid sequence according to SEQ ID NOs: 112-119.

Embodiment 429. The method of embodiment 417, wherein the cancer is the cancer is lung, breast (e.g. HER2+; ER/PR+; TNBC), cervical, ovarian, colorectal, pancreatic, gastric, triple-negative breast cancer (TNBC), urothelial cancer (UC), non-small cell lung cancer (NSCLC), small cell lung cancer (SCLC), gastric cancer, esophageal cancer, head and neck cancer, prostate cancer, or endometrial cancer.

Embodiment 430. The method of embodiment 417, wherein the tumor antigen comprises PSMA.

Embodiment 431. The method of embodiment 417, wherein the tumor antigen binding domain comprises an amino acid sequence according to SEQ ID NOs: 120-127.

Embodiment 432. The method of embodiment 417, wherein the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 120; HC-CDR2: SEQ ID NO: 121; HC-CDR3: SEQ ID NO: 122; and wherein the LC-CDR1, the LC-CDR2, and the LC-CDR3 comprise: LC-CDR1: SEQ ID NO: 123; LC-CDR2: SEQ ID NO: 124 (EA); and LC-CDR3: SEQ ID NO: 125.

Embodiment 433. The method of embodiment 417, wherein the tumor antigen comprises PSMA, and the T cell engager comprises amino acid sequences with at least 95% sequence identity according to SEQ ID NOs: 173 and 174.

Embodiment 434. The method of embodiment 417, wherein the tumor antigen comprises PSMA, and the T cell engager comprises amino acid sequences according to SEQ ID NOs: 173 and 174.

Embodiment 435. The method of embodiment 417, wherein the cancer is cancer is lung, breast (e.g. HER2+; ER/PR+; TNBC), cervical, ovarian, colorectal, pancreatic or gastric.

Embodiment 436. The method of any one of embodiments 408-435, wherein the T cell engager molecule is selectively activated in tumor microenvironments.

Embodiment 437. The method of embodiment 436, wherein the T cell engager is according to the following subformula: P₃-L₃-D₁-L₀-E₁ (Formula IIa) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease.

Embodiment 438. The method of embodiment 436, wherein the T cell engager is according to the following subformula: D₁-L₀-E₁-L₄-P₄ (Formula IIb) wherein D₁ comprises the CD3 binding domain; E₁ comprises the tumor antigen binding domain; L₀ comprises the linker that connects D₁ to E₁; P₄ comprises a peptide that binds to E₁ and L₄ comprises a linking moiety that connects E₁ to P₄ and is a substrate for a tumor specific protease.

Embodiment 439. The method of embodiment 436, wherein the T cell engager is according to the following subformula: P₃-L₃-D₁-L₀-E₁-L₄-P₄ (Formula IIc) wherein D₁ comprises the CD3 binding domain; E₁ comprises the tumor antigen binding domain; L₀ comprises the linker that connects D₁ to E₁; P₃ comprises a peptide that binds to D₁ and L₃ comprises a linking moiety that connects D₁ to P₃ and is a substrate for a tumor specific protease; P₄ comprises a peptide that binds to E₁ and L₄ comprises a linking moiety that connects E₁ to P₄ and is a substrate for a tumor specific protease.

Embodiment 440. The method of any one of embodiments 437-439, wherein the T cell engager comprises H₁.

Embodiment 441. The method of embodiment 440, wherein H₁ comprises a sequence according to SEQ ID NO: 54-57.

Embodiment 442. The method of embodiment 440, wherein H₁ comprises a single domain antibody.

Embodiment 443. The method of embodiment 440, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56.

Embodiment 444. The method of any one of embodiments 437-443, wherein L₃ or L₄ is a peptide sequence having at least 5 to no more than 50 amino acids.

Embodiment 445. The method of any one of embodiments 437-444, wherein L₃ or L₄ is a peptide sequence having at least 10 to no more than 30 amino acids.

Embodiment 446. The method of any one of embodiments 437-445, wherein L₃ or L₄ is a peptide sequence having at least 10 amino acids.

Embodiment 447. The method of any one of embodiments 437-446, wherein L₃ or L₄ is a peptide sequence having at least 18 amino acids.

Embodiment 448. The method of any one of embodiments 437-447, wherein L₃ or L₄ is a peptide sequence having at least 26 amino acids.

Embodiment 449. The method of any one of embodiments 437-448, wherein L₃ or L₄ comprises a formula comprising (G2S)n, wherein n is an integer from 1 to 3 (SEQ ID NO: 228).

Embodiment 450. The method of any one of embodiments 437-449, wherein L₃ or L₄ comprises a formula comprising (G2S)n, wherein n is an integer of at least 1.

Embodiment 451. The method of any one of embodiments 437-443, wherein L₃ or L₄ comprises a formula selected from the group consisting of (G₂S)n, (GS)n, (GSGGS)n (SEQ ID NO: 58), (GGGS)n (SEQ ID NO: 59), (GGGGS)n (SEQ ID NO: 60), and (GSSGGS)n (SEQ ID NO: 61), wherein n is an integer of at least 1.

Embodiment 452. The method of any one of embodiments 437-451, wherein the tumor specific protease is selected from the group consisting of metalloprotease, serine protease, cysteine protease, threonine protease, and aspartic protease.

Embodiment 453. The method of any one of embodiments 437-452, wherein L₃ or L₄ comprises a urokinase cleavable amino acid sequence, a matriptase cleavable amino acid sequence, or a matrix metalloprotease cleavable amino acid sequence.

Embodiment 454. The method of any one of embodiments 437-453, wherein L₃ or L₄ comprises a sequence according to SEQ ID NOs: 18-19, 62-88.

Embodiment 455. The method of any one of embodiments 437-454, wherein L₃ is bound to N-terminus of D₁.

Embodiment 456. The method of any one of embodiments 437-454, wherein L₃ is bound to C-terminus of D₁.

Embodiment 457. The method of any one of embodiments 438-454, wherein L₄ is bound to N-terminus of E₁.

Embodiment 458. The method of any one of embodiments 438-454, wherein L₄ is bound to C-terminus of E₁.

Embodiment 459. The method of any one of embodiments 437-458, wherein P₃ becomes unbound from D₁ when L₃ is cleaved by the tumor specific protease thereby exposing D₁ to CD3.

Embodiment 460. The method of any one of embodiments 438-459, wherein P₄ becomes unbound from E₁ when L₄ is cleaved by the tumor specific protease thereby exposing E₁ to the tumor antigen.

Embodiment 461. The method of any one of embodiments 437-460, wherein P₃ impairs binding of D₁ to CD3.

Embodiment 462. The method of any one of embodiments 437-461, wherein P₃ is bound to D₁ through ionic interactions, electrostatic interactions, hydrophobic interactions, Pi-stacking interactions, and H-bonding interactions, or a combination thereof.

Embodiment 463. The method of any one of embodiments 437-462, wherein P₃ is bound to D₁ at or near an antigen binding site.

Embodiment 464. The method of any one of embodiments 437-463, wherein P₃ becomes unbound from D₁ when L₃ is cleaved by the tumor specific protease thereby exposing D₁ to CD3.

Embodiment 465. The method of any one of embodiments 437-464, wherein P₃ has less than 70% sequence identity to CD3.

Embodiment 466. The method of any one of embodiments 437-465, wherein P₃ has less than 85% sequence identity to CD3.

Embodiment 467. The method of any one of embodiments 437-465, wherein P₃ has less than 90% sequence identity to CD3.

Embodiment 468. The method of any one of embodiments 437-467, wherein P₃ has less than 95% sequence identity to CD3.

Embodiment 469. The method of any one of embodiments 437-468, wherein P₃ has less than 98% sequence identity to CD3.

Embodiment 470. The method of any one of embodiments 437-469, wherein P₃ has less than 99% sequence identity to CD3.

Embodiment 471. The method of any one of embodiments 437-470 wherein P₃ comprises the amino acid sequence according to SEQ ID NOs: 177-180.

Embodiment 472. The method of any one of embodiments 437-470, wherein P₃ comprises a de novo amino acid sequence that shares less than 10% sequence identity to CD3.

Embodiment 473. The method of any one of embodiments 437-471, wherein P₄ impairs binding of E₁ to the tumor antigen.

Embodiment 474. The method of any one of embodiments 437-473, wherein P₄ is bound to E₁ through ionic interactions, electrostatic interactions, hydrophobic interactions, Pi-stacking interactions, and H-bonding interactions, or a combination thereof.

Embodiment 475. The method of any one of embodiments 437-474, wherein P₄ is bound to E₁ at or near an antigen binding site.

Embodiment 476. The method of any one of embodiments 437-475, wherein P₄ becomes unbound from E₁ when L₄ is cleaved by the tumor specific protease thereby exposing E₁ to the tumor antigen.

Embodiment 477. The method of any one of embodiments 437-476, wherein P₄ has less than 70% sequence identity to the tumor antigen.

Embodiment 478. The method of any one of embodiments 437-477, wherein P₄ has less than 80% sequence identity to the tumor antigen.

Embodiment 479. The method of any one of embodiments 437-478, wherein P₄ has less than 85% sequence identity to the tumor antigen.

Embodiment 480. The method of any one of embodiments 437-479, wherein P₄ has less than 90% sequence identity to the tumor antigen.

Embodiment 481. The method of any one of embodiments 437-480, wherein P₄ has less than 95% sequence identity to the tumor antigen.

Embodiment 482. The method of any one of embodiments 437-481, wherein P₄ comprises a de novo amino acid sequence that shares less than 10% sequence identity to the tumor antigen.

Embodiment 483. The method of any one of embodiments 436-482, wherein P₃ or P₄ comprises a peptide sequence of at least 5 amino acids in length.

Embodiment 484. The method of any one of embodiments 436-483, wherein P₃ or P₄ comprises a peptide sequence of at least 6 amino acids in length.

Embodiment 485. The method of any one of embodiments 436-484, wherein P₃ or P₄ comprises a peptide sequence of at least 10 amino acids in length.

Embodiment 486. The method of any one of embodiments 436-485, wherein P₃ or P₄ comprises a peptide sequence of at least 10 amino acids in length and no more than 20 amino acids in length.

Embodiment 487. The method of any one of embodiments 436-486, wherein P₃ or P₄ comprises a peptide sequence of at least 16 amino acids in length.

Embodiment 488. The method of any one of embodiments 436-487, wherein P₃ or P₄ comprises a peptide sequence of no more than 40 amino acids in length.

Embodiment 489. The method of any one of embodiments 436-488, wherein P₃ or P₄ comprises at least two cysteine amino acid residues.

Embodiment 490. The method of any one of embodiments 436-489, wherein P₃ or P₄ comprises a cyclic peptide or a linear peptide.

Embodiment 491. The method of any one of embodiments 436-490, wherein P₃ or P₄ comprises a cyclic peptide.

Embodiment 492. The method of any one of embodiments 436-490, wherein P₃ or P₄ comprises a linear peptide.

Embodiment 493. The method of any one of embodiments 437-492, wherein P₄ comprises the amino acid sequence according to SEQ ID NO: 185 or 186.

Embodiment 494. The method of any one of embodiments 437-492 wherein the tumor antigen comprises EGFR, and the T cell engager comprises the amino acid sequence of SEQ ID NOs: 183 and 184.

Embodiment 495. The method of any one of embodiments 437-492, wherein P₄ comprises the amino acid sequence according to SEQ ID NOs: 199-201.

Embodiment 496. The method of any one of embodiments 437-492, wherein the tumor antigen comprises TROP2, and the T cell engager comprises any one of amino acid sequences of SEQ ID NOs: 193-198.

Embodiment 497. The method of any one of embodiments 437-492, wherein the tumor antigen comprises PSMA, and the T cell engager comprises the amino acid sequence of SEQ ID NOs: 175 and 176.

Embodiment 498. The pharmaceutical composition of embodiment 210, wherein the antibody-based therapy comprises an anti-PD-1 antibody therapy.

Embodiment 499. The pharmaceutical composition of embodiment 498, wherein the anti-PD-1 antibody therapy comprises the complementary determining regions (CDRs) of Pembrolizumab or Nivolumab.

Embodiment 500. The pharmaceutical composition of embodiment 498, wherein the anti-PD-1 antibody therapy comprises the amino acid sequence of SEQ ID NOs: 222 and 223.

Embodiment 501. The pharmaceutical composition of embodiment 498, wherein the anti-PD-1 antibody therapy comprises the amino acid sequence of SEQ ID NOs: 226 and 227.

Embodiment 502. The method of embodiment 403, wherein the antibody-based therapy comprises an anti-PD-1 antibody therapy.

Embodiment 503. The method of embodiment 502, wherein the anti-PD-1 antibody therapy comprises the complementary determining regions (CDRs) of Pembrolizumab or Nivolumab.

Embodiment 504. The method of embodiment 502, wherein the anti-PD-1 antibody therapy comprises the amino acid sequence of SEQ ID NOs: 222 and 223.

Embodiment 505. The method of embodiment 502, wherein the anti-PD-1 antibody therapy comprises the amino acid sequence of SEQ ID NOs: 226 and 227.

### EXAMPLES

### Example 1. Discovery of Peptides that Bind to Anti-CD28 scFv

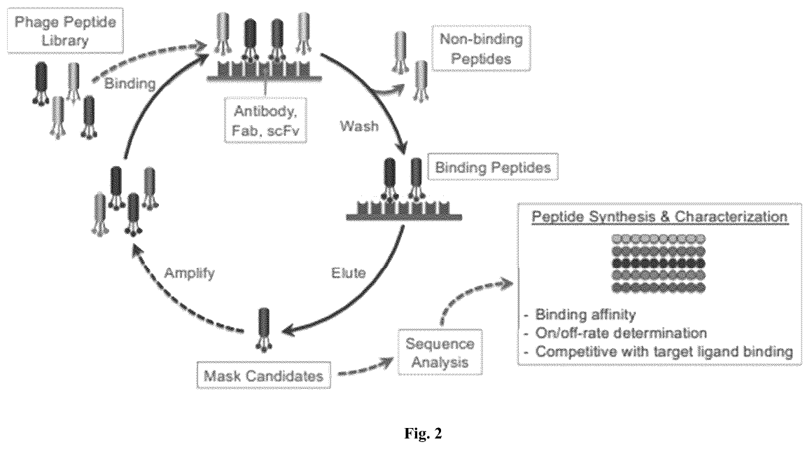

Lead peptides that mask the anti-CD28 scFv according to SEQ ID NO: 9 were identified by phage display according to the method of FIG. 2. Lead hits were then synthesized as peptides and evaluated as described below. Synthetic peptides were evaluated for their ability to bind human anti-CD28 scFv in a standard enzyme linked immunosorbent assay (ELISA) format. Briefly, biotinylated peptides were captured on neutravidin coated plates. Anti-CD28 scFv or Ab-12 diluted in buffer was then added to the peptide captured plates. Bound anti-CD28 scFv was detected using a standard horse radish peroxidase conjugate secondary antibody. The concentration of anti-CD28 scFv or Ab-12 required to achieve 50% maximal signal (EC50) was calculated using Graphpad Prism software. Peptides were also evaluated for their ability to inhibit anti-CD28 scFv or Ab-12 from binding its cognate antigen, CD28. Briefly, biotinylated CD28 antigen was captured on neutravidin coated plates. Anti-CD28 scFv at 2 nM or Ab-12 at 5 nM were pre-incubated with 0-100 uM titrated peptides. After a short pre-incubation period the mixture of titrated peptides with fixed anti-CD28 scFv (2 nM) or Ab-12 (5 nM) were added to the CD28 antigen captured plates. After a short incubation on the plates, bound anti-CD28 scFv or Ab-12 were detected with a standard horse radish peroxidase conjugated secondary antibody. The concentration of peptide required to reduce the max signal by 50% (IC50) was calculated in Graphpad Prism software.

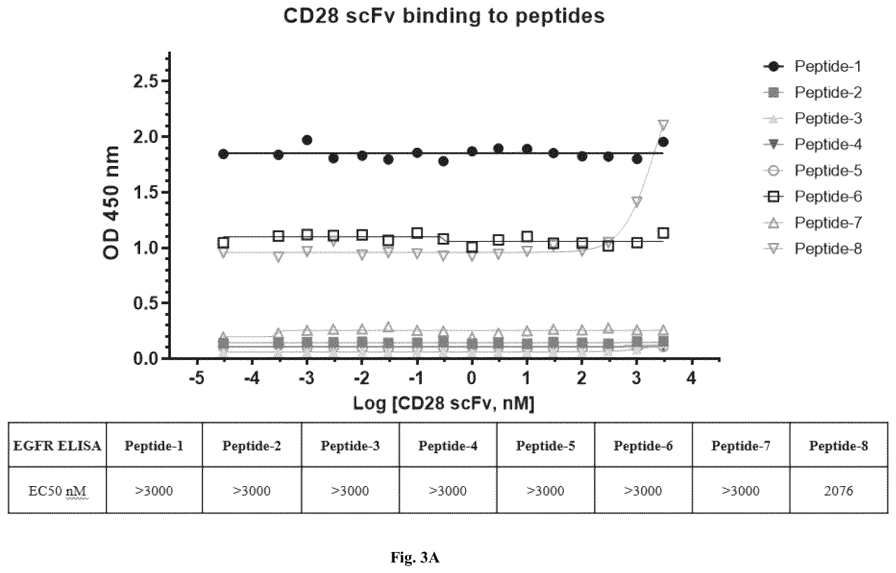

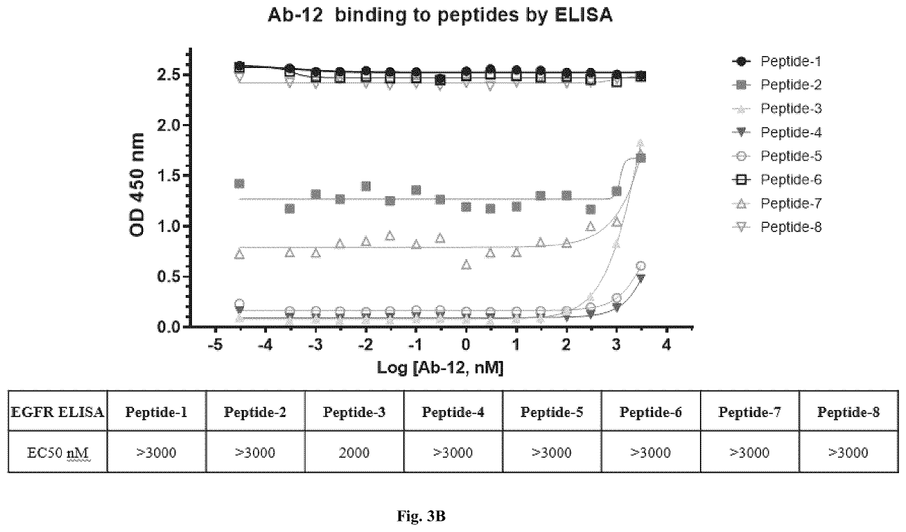

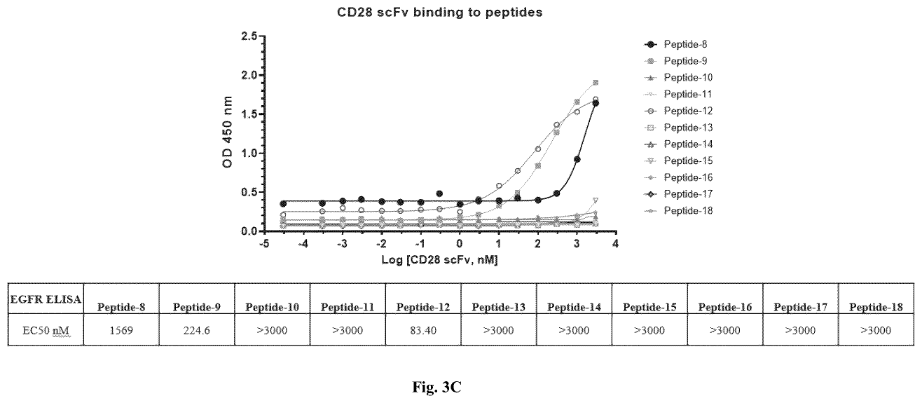

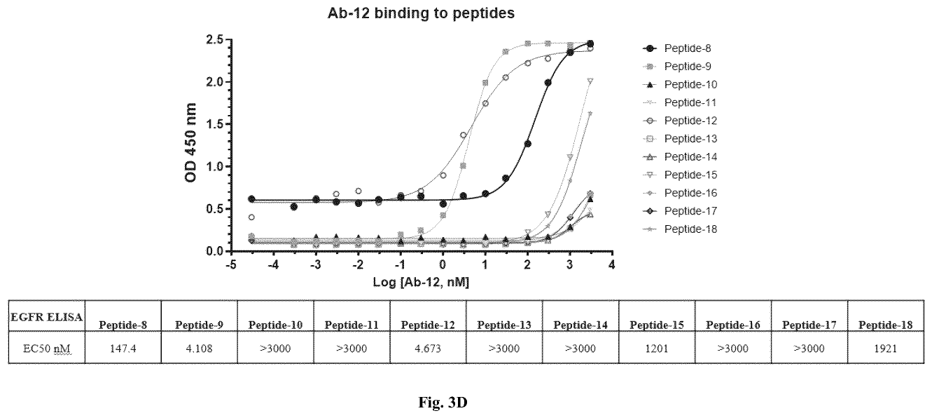

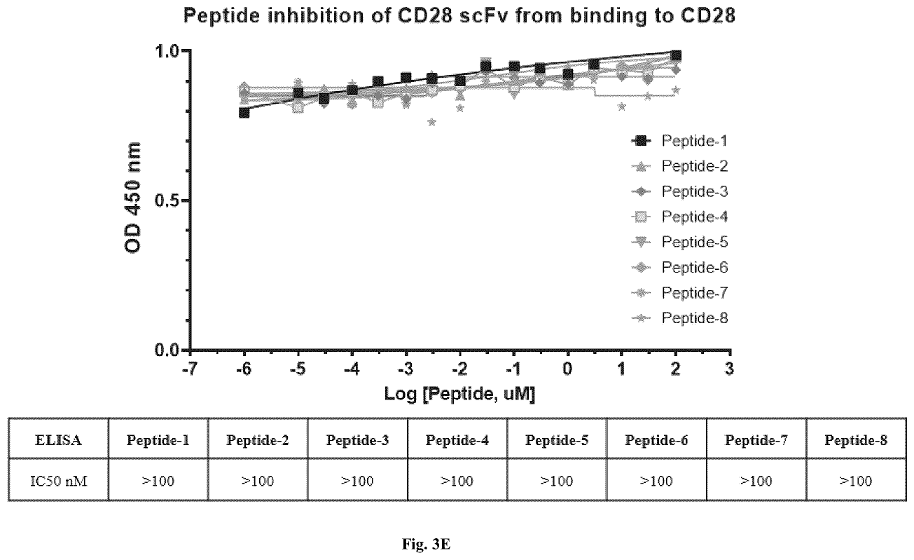

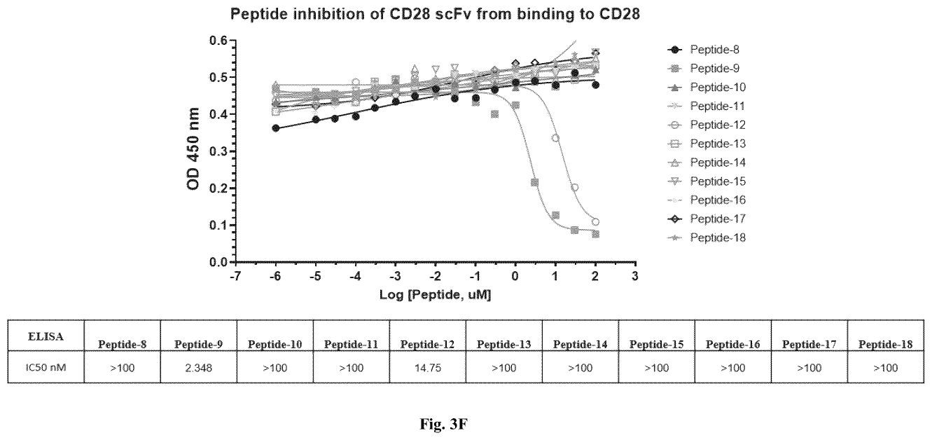

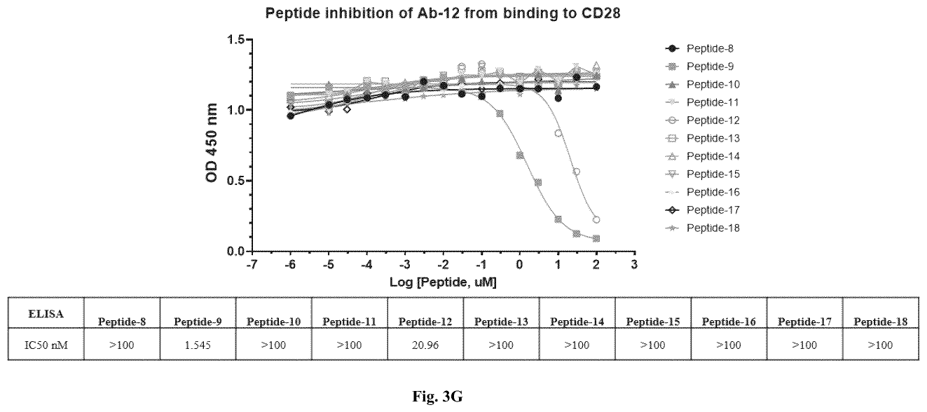

FIG. 3A illustrates anti-CD28 scFv (SEQ ID NO: 9) binding to peptides measured by ELISA. FIG. 3B illustrates Ab-12 binding to peptides measured by ELISA. Ab-12 is an anti-PD-L1×CD28 (unmasked) antibody in Vh format. FIG. 3C illustrates anti-CD28 scFv binding to peptides measured by ELISA. FIG. 3D illustrates Ab-12 binding to peptides measured by ELISA. FIGS. 3E-3F illustrate that peptides inhibit anti-CD28 scFv from binding to CD28 antigen as measured by ELISA. FIG. 3G illustrates that peptides inhibit Ab-12 from binding CD28 as measured by ELISA.

### Example 2. Kinetic Binding Assays of Anti-CD28 scFv (SEQ ID NO: 9) or Ab-12, an Anti-PD-L1×CD28 Non-Masked Antibody in Vh Format, to Peptides-9 and -12

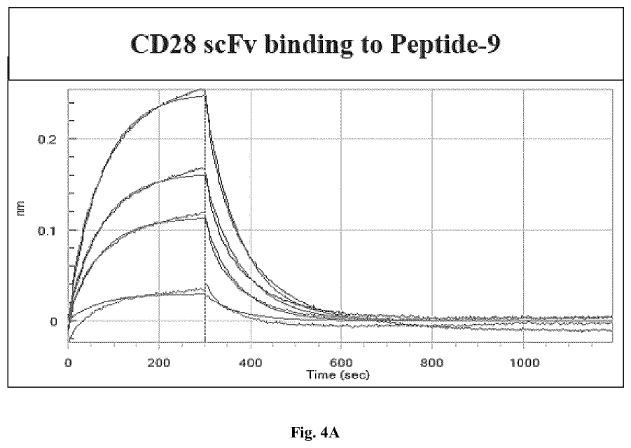

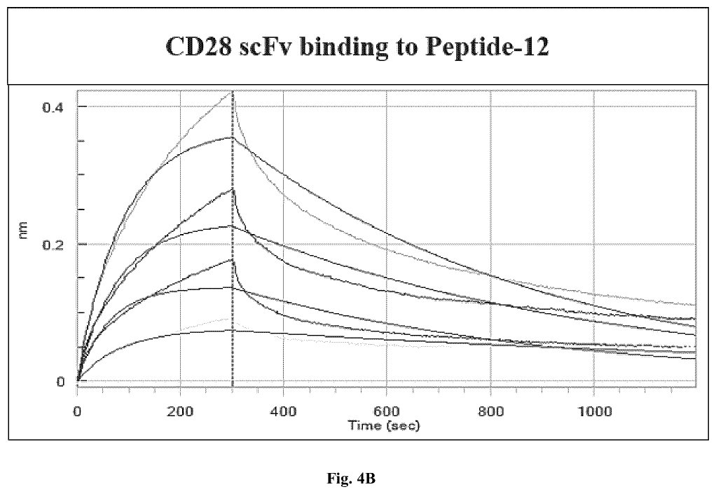

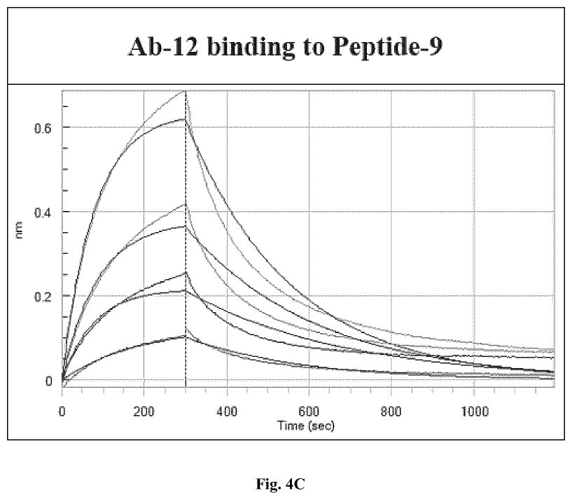

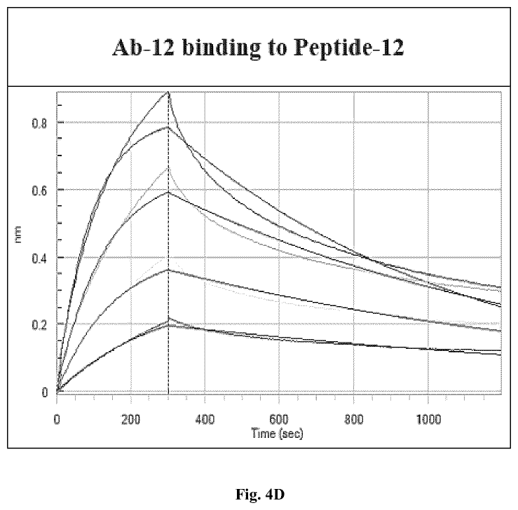

This Example assesses binding of anti-CD28 scFv or Ab-12 to Peptide-9 and Peptide-12 in an in vitro kinetic binding assay. Kinetic binding of anti-CD28 scFv or Ab-12 to Peptide-9 and Peptide-12 were evaluated by bio-layer interferometry using an Octet RED96 instrument. Briefly, streptavidin biosensors were loaded with biotinylated peptides and baselined in buffer. Anti-CD28 scFv or Ab-12 were titrated in solution at 100 nM, 50 nM, 25 nM, and 12.5 nM, then associated onto the peptide loaded sensors. After a short association period, sensors were transferred into buffer and the dissociation of bound anti-CD28 scFv or Ab-12 was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, KD. Peptide-9 and peptide-12 kinetic binding sensorgrams are shown in FIGS. 4A-4D and are summarized in Tables 17-19.

TABLE 17
Timing and Steps of Assay

| Step | Time |
|---|---|
| Baseline: Buffer | 60 sec |
| Load: | 300 sec |
| 200 nM Peptide-9 | |
| 200 nM Peptide-12 | |
| Baseline: Buffer | 300 sec |
| Association in octet buffer | 300 sec |
| 100 nM Ab-12 or CD28 scFv | |
| 50 nM Ab-12 or CD28 scFv | |
| 25 nM Ab-12 or CD28 scFv | |
| 12.5 nM Ab-12 or CD28 scFv | |
| Dissociation: Buffer | 900 sec |

TABLE 18
Binding Kinetics Summary of anti-CD28
scFv to Peptide-9 and Peptide-12

| Binding domain | Peptide | KD (M) | kon(1/Ms) | kdis(1/s) |
|---|---|---|---|---|
| CD28 scFv | Peptide-9 | 3.77E−07 | 3.25E+04 | 1.23E−02 |
| CD28 scFv | Peptide-12 | 1.70E−08 | 9.87E+04 | 1.67E−03 |

TABLE 19
Binding Kinetics Summary of Ab-12 to Peptide-9 and Peptide-12

| Binding | | | | |
|---|---|---|---|---|
| domain | Peptide | KD (M) | kon(1/Ms) | kdis(1/s) |
| Ab-12 | Peptide-9 | 1.88E−08 | 1.68E+05 | 3.17E−03 |
| Ab-12 | Peptide-12_200 nM | 1.31E−08 | 9.66E+04 | 1.27E−03 |

### Example 3: Optimized Phage Library Construction-Anti-CD28 scFv (SEQ ID NO: 9) Peptide-9

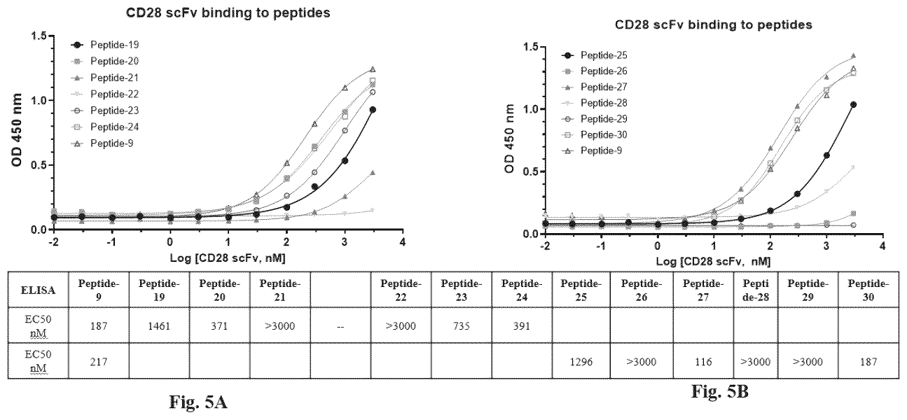

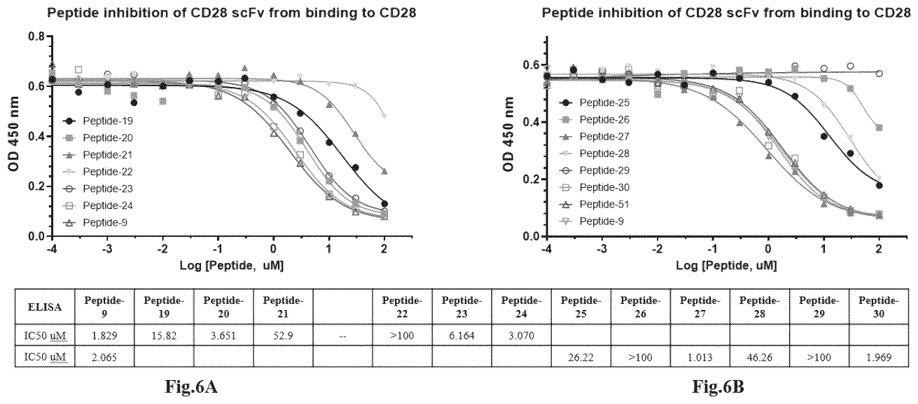

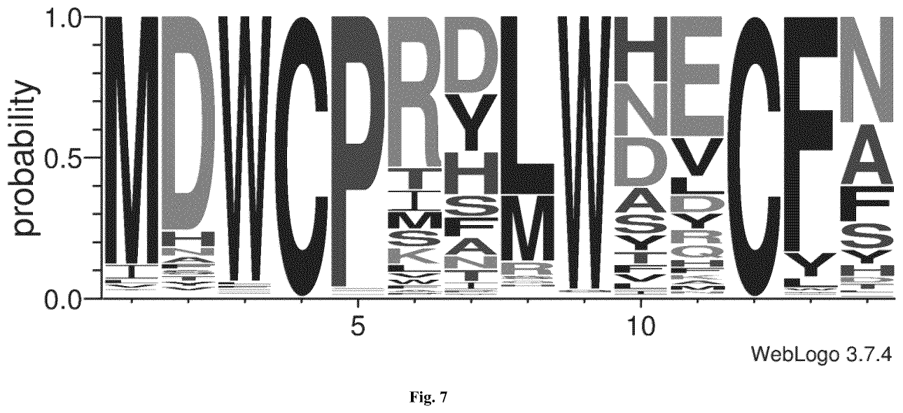

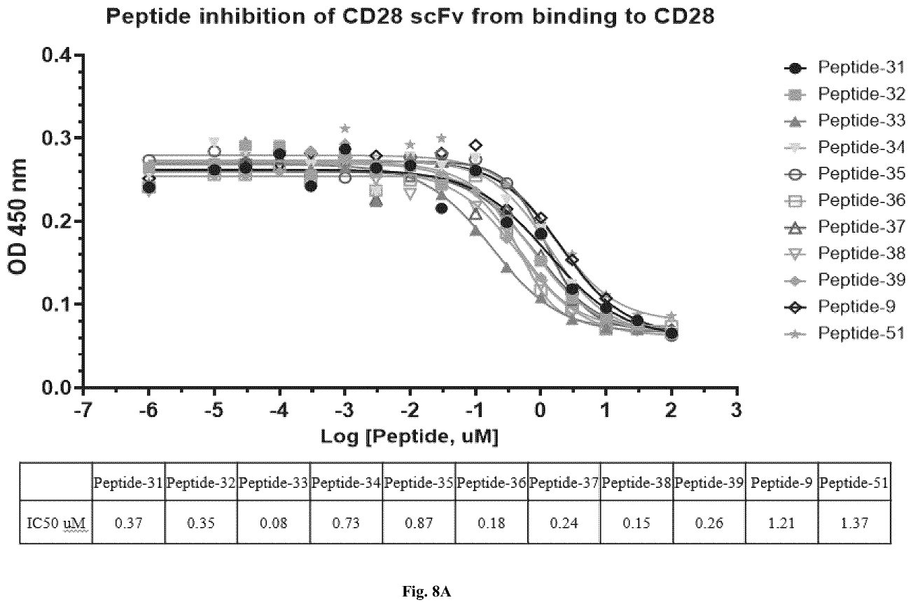

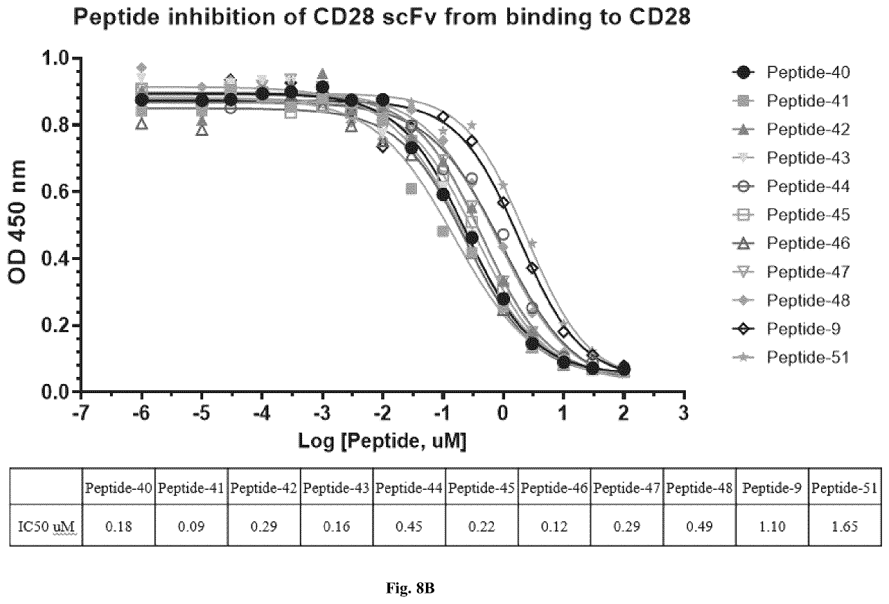

Sequence activity relationships were established for Peptide-9 by mutating each individual residue within the peptide to alanine and measuring binding and inhibition against anti-CD28 scFv. Peptide residues whose alanine mutations significantly weakened binding and inhibition were considered key residues where mutations were not tolerated. Peptide residues whose alanine mutations performed similarly to the non-mutated sequence were considered non-critical sites where mutations were indeed tolerated. Using the peptide sequence activity relationships (SAR), DNA oligo libraries were constructed where codons encoding critical residues within each peptide sequence were minimally mutated and codons encoding non-critical residues were heavily mutated. The resulting oligos were cloned into bacteriophage vectors used to display the SAR guided peptides via fusion to the pill filament of the bacteriophage. The relevant vectors were then used to produce the phage optimization libraries via amplification in bacteria using standard techniques in the field. FIG. 5A and FIG. 5B demonstrate anti-CD28 scFv binding of alanine scanning peptides of Peptide-9 according to the ELISA protocol of Example 1. FIG. 6A and FIG. 6B demonstrate anti-CD28 scFv inhibition of alanine scanning peptides of Peptide-9 according to the ELISA protocol of Example 1.

### Example 4: Panning ELISAs—Anti-CD28 scFv Peptides

Clonal phage were harvested as crude supernatants and screened via standard enzyme linked immunosorbent assays (ELISAs). Briefly, biotinylated anti-CD28 scFv was captured on neutravidin coated plates. Prior to the addition of clonal phage, wells were incubated with blocking buffer and CD28 soluble protein or blocking buffer alone. Without washing or aspirating, clonal phage supernatants were then added to the wells and incubated for a short time. Wells were then washed followed by detection of bound phage using a horse radish peroxidase conjugated anti-M13 antibody. Clonal phage of interest were then sent for sequence analysis.

Phage panning results of anti-CD28 scFv Peptide-9 library sequences are shown in Table 20. 453 clonal phage sequences were identified. The consensus sequence calculated from all the sequences of Table 20 is shown in FIG. 7 and was generated using WebLogo 3.7.4.

TABLE 20
Phage panning results of Anti-CD28 scFv Peptide-9 library sequences. (-) indicates same
amino acid as in anti-CD28 scFv Peptide-9 corresponding position (e.g. Phage-1 position).

| | | | | | | | | | | | | | | | | | Phage binding ELISA | | | |
|---|---|---|---|---|---|---|---|---|---|---|---|---|---|---|---|---|---|---|---|---|
| | | | | | | | | | | | | | | | | | | | CD28 | |
| | Cloncal | | | | | | | | | | | | | | | | | | scFv | |
| | phage | SEQ | | | | | | | | | | | | | | | | CD28 | signal in | |
| | peptide | ID | Amino acid position sequence | | | | | | | | | | | | | | Backgroud | scFv | presence | SEQ ID |
| ID | sequence | NO: | 1 | 2 | 3 | 4 | 5 | 6 | 7 | 8 | 9 | # | # | # | # | # | signal | signal | of CD28 | NO: |
| Phage- | MDWCPRER | 32 | M | D | W | C | P | R | E | R | W | V | D | C | F | F | 0.084 | 2.042 | 0.489 | 2 |
| 9 | WVDCFF | | | | | | | | | | | | | | | | | | | |
| Phage- | MDWCPIDL | 128 | — | — | — | — | — | I | D | L | — | N | E | — | — | — | 0.066 | 2.515 | 0.187 | 128 |
| 19 | WNECFF | | | | | | | | | | | | | | | | | | | |
| Phage- | MDWCPIHL | 129 | — | — | — | — | — | I | H | L | — | H | V | — | — | N | 0.082 | 2.526 | 0.167 | 129 |
| 20 | WHVCFN | | | | | | | | | | | | | | | | | | | |
| Phage- | MDWCPIYL | 130 | — | — | — | — | — | I | Y | L | — | S | E | — | — | N | 0.075 | 2.635 | 0.513 | 130 |
| 21 | WSECFN | | | | | | | | | | | | | | | | | | | |
| Phage- | MNWCPKDI | 131 | — | N | — | — | — | K | D | I | — | Y | L | — | — | N | 0.073 | 2.625 | 0.168 | 131 |
| 22 | WYLCFN | | | | | | | | | | | | | | | | | | | |
| Phage- | MDWCPLHM | 132 | — | — | — | — | — | L | H | M | — | H | E | — | — | S | 0.086 | 2.511 | 0.151 | 132 |
| 23 | WHECFS | | | | | | | | | | | | | | | | | | | |
| Phage- | MDWCPLYL | 133 | — | — | — | — | — | L | Y | L | — | N | E | — | — | N | 0.065 | 2.612 | 0.247 | 133 |
| 24 | WNECFN | | | | | | | | | | | | | | | | | | | |
| Phage- | MDWCPRDL | 134 | — | — | — | — | — | — | D | L | — | D | L | — | — | A | 0.078 | 2.696 | 0.219 | 134 |
| 25 | WDLCFA | | | | | | | | | | | | | | | | | | | |
| Phage- | MDWCPRDL | 135 | — | — | — | — | — | — | D | L | — | H | E | — | — | A | 0.063 | 2.710 | 0.277 | 135 |
| 26 | WHECFA | | | | | | | | | | | | | | | | | | | |
| Phage- | MDWCPRDL | 136 | — | — | — | — | — | — | D | L | — | H | L | — | — | S | 0.059 | 2.592 | 0.290 | 136 |
| 27 | WHLCFS | | | | | | | | | | | | | | | | | | | |
| Phage- | MDWCPRDL | 137 | — | — | — | — | — | — | D | L | — | S | E | — | — | — | 0.068 | 2.574 | 0.218 | 137 |
| 28 | WSECFF | | | | | | | | | | | | | | | | | | | |
| Phage- | MDWCPRDL | 138 | — | — | — | — | — | — | D | L | — | — | H | — | — | A | 0.062 | 2.554 | 0.179 | 138 |
| 29 | WVHCFA | | | | | | | | | | | | | | | | | | | |
| Phage- | MDWCPRDM | 139 | — | — | — | — | — | — | D | M | — | D | E | — | — | A | 0.116 | 2.593 | 0.250 | 139 |
| 30 | WDECFA | | | | | | | | | | | | | | | | | | | |
| Phage- | MDWCPRDM | 140 | — | — | — | — | — | — | D | M | — | S | E | — | — | A | 0.069 | 2.701 | 0.293 | 140 |
| 31 | WSECFA | | | | | | | | | | | | | | | | | | | |
| Phage- | MDWCPRDM | 141 | — | — | — | — | — | — | D | M | — | S | V | — | — | S | 0.062 | 2.619 | 0.207 | 141 |
| 32 | WSVCFS | | | | | | | | | | | | | | | | | | | |
| Phage- | MDWCPRFM | 142 | — | — | — | — | — | — | F | M | — | D | E | — | — | N | 0.079 | 2.680 | 0.256 | 142 |
| 33 | WDECFN | | | | | | | | | | | | | | | | | | | |
| Phage- | MDWCPRHM | 143 | — | — | — | — | — | — | H | M | — | N | Y | — | — | A | 0.089 | 2.712 | 0.242 | 143 |
| 34 | WNYCFA | | | | | | | | | | | | | | | | | | | |
| Phage- | MDWCPRSL | 144 | — | — | — | — | — | — | S | L | — | H | E | — | — | A | 0.064 | 2.544 | 0.255 | 144 |
| 35 | WHECFA | | | | | | | | | | | | | | | | | | | |
| Phage- | MDWCPRYL | 145 | — | — | — | — | — | — | Y | L | — | H | V | — | — | A | 0.072 | 2.475 | 0.226 | 145 |
| 36 | WHVCFA | | | | | | | | | | | | | | | | | | | |
| Phage- | MHWCPVDL | 146 | — | H | — | — | — | V | D | L | — | Y | L | — | Y | N | 0.079 | 2.659 | 0.417 | 146 |
| 37 | WYLCYN | | | | | | | | | | | | | | | | | | | |
| Phage- | MDWCPVHL | 147 | — | — | — | — | — | V | H | L | — | S | V | — | — | A | 0.072 | 2.650 | 0.302 | 147 |
| 38 | WSVCFA | | | | | | | | | | | | | | | | | | | |
| Phage- | MDWCPMHL | 229 | — | — | — | — | — | M | H | L | — | H | Q | — | — | N | 0.073 | 2.555 | 0.299 | 229 |
| 39 | WHQCFN | | | | | | | | | | | | | | | | | | | |
| Phage- | MDWCPIDM | 230 | — | — | — | — | — | I | D | M | — | D | Q | — | — | N | 0.061 | 2.661 | 0.297 | 230 |
| 40 | WDQCFN | | | | | | | | | | | | | | | | | | | |
| Phage- | MAWCPRDK | 231 | — | A | — | — | — | — | D | K | — | S | E | — | — | S | 0.062 | 0.853 | 0.147 | 231 |
| 41 | WSECFS | | | | | | | | | | | | | | | | | | | |
| Phage- | MDWCPRHL | 232 | — | — | — | — | — | — | H | L | — | | H | — | — | N | 0.071 | 2.505 | 0.254 | 232 |
| 42 | WVHCFN | | | | | | | | | | | | | | | | | | | |
| Phage- | MDWCPRAL | 233 | — | — | — | — | — | — | A | L | — | H | E | — | — | Y | 0.115 | 2.499 | 0.252 | 233 |
| 43 | WHECFY | | | | | | | | | | | | | | | | | | | |
| Phage- | MDWCPIAL | 234 | — | — | — | — | — | I | A | L | — | A | E | — | — | N | 0.111 | 2.458 | 0.224 | 234 |
| 44 | WAECFN | | | | | | | | | | | | | | | | | | | |
| Phage- | MDWCPRPL | 235 | — | — | — | — | — | — | P | L | — | H | E | — | — | S | 0.078 | 2.346 | 0.121 | 235 |
| 45 | WHECFS | | | | | | | | | | | | | | | | | | | |
| Phage- | MHWCPIDL | 236 | — | H | — | — | — | I | D | L | — | A | E | — | Y | A | 0.082 | 2.278 | 0.225 | 236 |
| 46 | WAECYA | | | | | | | | | | | | | | | | | | | |
| Phage- | MAWCPVYL | 237 | — | A | — | — | — | V | Y | L | — | H | E | — | — | N | 0.094 | 2.273 | 0.351 | 237 |
| 47 | WHECFN | | | | | | | | | | | | | | | | | | | |
| Phage- | IDWCPRYL | 238 | I | — | — | — | — | — | Y | L | — | D | E | — | Y | N | 0.061 | 2.243 | 0.153 | 238 |
| 48 | WDECYN | | | | | | | | | | | | | | | | | | | |
| Phage- | MSWCPIHL | 239 | — | S | — | — | — | I | H | L | — | N | E | — | — | N | 0.083 | 2.057 | 0.284 | 239 |
| 49 | WNECFN | | | | | | | | | | | | | | | | | | | |
| Phage- | MDWCPPYL | 240 | — | — | — | — | — | P | Y | L | — | N | V | — | — | S | 0.063 | 1.639 | 0.100 | 240 |
| 50 | WNVCFS | | | | | | | | | | | | | | | | | | | |
| Phage- | MDWCPMDL | 241 | — | — | — | — | — | M | D | L | — | D | Y | — | — | N | 0.071 | 1.635 | 0.107 | 241 |
| 51 | WDYCFN | | | | | | | | | | | | | | | | | | | |
| Phage- | MDWCPINL | 242 | — | — | — | — | — | I | N | L | — | D | E | — | — | S | 0.062 | 1.498 | 0.096 | 242 |
| 52 | WDECFS | | | | | | | | | | | | | | | | | | | |
| Phage- | MDWCPMHL | 243 | — | — | — | — | — | M | H | L | — | N | K | — | — | N | 0.071 | 1.043 | 0.122 | 243 |
| 53 | WNKCFN | | | | | | | | | | | | | | | | | | | |
| Phage- | MNWCPRDM | 244 | — | N | — | — | — | — | D | M | — | Y | Q | — | — | N | 0.068 | 1.948 | 0.177 | 244 |
| 54 | WYQCFN | | | | | | | | | | | | | | | | | | | |
| Phage- | QDWCPRHM | 245 | Q | — | — | — | — | — | H | M | — | S | F | — | — | H | 0.138 | 0.074 | 0.081 | 245 |
| 55 | WSFCFH | | | | | | | | | | | | | | | | | | | |
| Phage- | MHWC PMDQ | 246 | — | H | — | — | — | M | D | Q | — | S | N | — | — | N | 0.062 | 0.515 | 0.084 | 246 |
| 56 | WSNCFN | | | | | | | | | | | | | | | | | | | |
| Phage- | MDWCPRHR | 247 | — | — | — | — | — | — | H | — | — | — | — | — | — | Y | 0.073 | 0.478 | 0.110 | 247 |
| 57 | WVDCFY | | | | | | | | | | | | | | | | | | | |
| Phage- | MHWCPIDR | 248 | — | H | — | — | — | I | D | — | — | A | — | — | — | N | 0.074 | 0.287 | 0.077 | 248 |
| 58 | WADCFN | | | | | | | | | | | | | | | | | | | |
| Phage- | MDWCPRDS | 249 | — | — | — | — | — | — | D | S | — | H | L | — | — | A | 0.073 | 2.446 | 0.147 | 249 |
| 59 | WHLCFA | | | | | | | | | | | | | | | | | | | |
| Phage- | MDWCPRTL | 250 | — | — | — | — | — | — | T | L | — | Y | L | — | — | N | 0.508 | 2.593 | 1.198 | 250 |
| 60 | WYLCFN | | | | | | | | | | | | | | | | | | | |
| Phage- | MVSCPTTM | 251 | — | V | S | — | — | T | T | M | — | N | R | — | — | N | 0.079 | 1.329 | 0.072 | 251 |
| 61 | WNRCFN | | | | | | | | | | | | | | | | | | | |
| Phage- | MDWCPSYL | 252 | — | — | — | — | — | S | Y | L | — | N | E | — | — | — | 0.065 | 1.995 | 0.133 | 252 |
| 62 | WNECFF | | | | | | | | | | | | | | | | | | | |
| Phage- | MDWCPRAL | 253 | — | — | — | — | — | — | A | L | — | A | E | — | — | N | 0.068 | 2.676 | 0.339 | 253 |
| 63 | WAECFN | | | | | | | | | | | | | | | | | | | |
| Phage- | MDWCPMYL | 254 | — | — | — | — | — | M | Y | L | — | N | E | — | — | N | 0.064 | 2.609 | 0.471 | 254 |
| 65 | WNECFN | | | | | | | | | | | | | | | | | | | |
| Phage- | MDWCPRYL | 255 | — | — | — | — | — | — | Y | L | — | N | E | — | — | Y | 0.079 | 2.537 | 0.358 | 255 |
| 56 | WNECFY | | | | | | | | | | | | | | | | | | | |
| Phage- | MNWCPTAL | 256 | — | N | — | — | — | T | A | L | — | H | V | — | — | N | 0.067 | 1.644 | 0.169 | 256 |
| 67 | WHVCFN | | | | | | | | | | | | | | | | | | | |
| Phage- | MDWCPMYM | 257 | — | — | — | — | — | M | Y | M | — | Y | E | — | — | A | 0.080 | 2.479 | 0.358 | 257 |
| 68 | WYECFA | | | | | | | | | | | | | | | | | | | |
| Phage- | MDWCPIHM | 258 | — | — | — | — | — | I | H | M | — | A | — | — | — | A | 0.065 | 2.457 | 0.143 | 258 |
| 69 | WADCFA | | | | | | | | | | | | | | | | | | | |
| Phage- | ITWCPSSM | 259 | I | T | — | — | — | S | S | M | — | N | R | — | — | I | 0.065 | 0.379 | 0.110 | 259 |
| 70 | WNRCFI | | | | | | | | | | | | | | | | | | | |
| Phage- | MDWCPRYL | 260 | — | — | — | — | — | — | Y | L | — | H | E | — | — | A | 0.071 | 2.628 | 0.429 | 260 |
| 71 | WHECFA | | | | | | | | | | | | | | | | | | | |
| Phage- | MFWCPTTM | 261 | — | F | — | — | — | T | T | M | — | N | R | — | — | T | 0.111 | 0.662 | 0.338 | 261 |
| 72 | WNRCFT | | | | | | | | | | | | | | | | | | | |
| Phage- | MDWCPRAL | 262 | — | — | — | — | — | — | A | L | — | H | E | — | — | N | 0.071 | 2.686 | 0.401 | 262 |
| 73 | WHECFN | | | | | | | | | | | | | | | | | | | |
| Phage- | MDWCPRDM | 263 | — | — | — | — | — | — | D | M | — | L | F | — | Y | N | 0.097 | 2.707 | 1.023 | 263 |
| 74 | WLFCYN | | | | | | | | | | | | | | | | | | | |
| Phage- | MDWCPRSH | 264 | — | — | — | — | — | — | S | H | — | H | V | — | Y | N | 0.072 | 1.951 | 0.281 | 264 |
| 75 | WHVCYN | | | | | | | | | | | | | | | | | | | |
| Phage- | MDWCPRYL | 265 | — | — | — | — | — | — | Y | L | — | S | — | — | — | A | 0.084 | 2.727 | 1.530 | 265 |
| 76 | WSDCFA | | | | | | | | | | | | | | | | | | | |
| Phage- | MDWCPFYL | 266 | — | — | — | — | — | F | Y | L | — | D | E | — | — | N | 0.092 | 2.672 | 0.291 | 266 |
| 77 | WDECFN | | | | | | | | | | | | | | | | | | | |
| Phage- | MDWCPRHL | 267 | — | — | — | — | — | — | H | L | — | H | E | — | — | N | 0.070 | 2.654 | 0.704 | 267 |
| 78 | WHECFN | | | | | | | | | | | | | | | | | | | |
| Phage- | MDWCPRDL | 268 | — | — | — | — | — | — | D | L | — | H | E | — | — | S | 0.066 | 2.649 | 0.382 | 268 |
| 79 | WHECFS | | | | | | | | | | | | | | | | | | | |
| Phage- | MDWCPMYL | 269 | — | — | — | — | — | M | Y | L | — | N | E | — | — | S | 0.071 | 2.641 | 0.411 | 269 |
| 80 | WNECFS | | | | | | | | | | | | | | | | | | | |
| Phage- | MDWCPRFL | 270 | — | — | — | — | — | — | F | L | — | S | V | — | — | N | 0.515 | 2.637 | 1.151 | 270 |
| 81 | WSVCFN | | | | | | | | | | | | | | | | | | | |
| Phage- | MDWCPRDL | 271 | — | — | — | — | — | — | D | L | — | T | E | — | — | A | 0.060 | 2.634 | 0.151 | 271 |
| 82 | WTECFA | | | | | | | | | | | | | | | | | | | |
| Phage- | MDWCPRFL | 272 | — | — | — | — | — | — | F | L | — | D | E | — | — | N | 0.065 | 2.629 | 0.483 | 272 |
| 83 | WDECEN | | | | | | | | | | | | | | | | | | | |
| Phage- | MDWCPKYL | 273 | — | — | — | — | — | K | Y | L | — | S | V | — | — | — | 0.184 | 2.622 | 0.826 | 273 |
| 84 | WSVCFF | | | | | | | | | | | | | | | | | | | |
| Phage- | MDWCPMDL | 274 | — | — | — | — | — | M | D | L | — | Y | Q | — | — | N | 0.063 | 2.617 | 0.187 | 274 |
| 85 | WYQCFN | | | | | | | | | | | | | | | | | | | |
| Phage- | MDWCPRHL | 275 | — | — | — | — | — | — | H | L | — | A | E | — | — | — | 0.066 | 2.611 | 0.292 | 275 |
| 86 | WAECFF | | | | | | | | | | | | | | | | | | | |
| Phage- | MDWCPKDL | 276 | — | — | — | — | — | K | D | L | — | Y | L | — | — | A | 0.200 | 2.608 | 0.213 | 276 |
| 87 | WYLCFA | | | | | | | | | | | | | | | | | | | |
| Phage- | MDWCPIHL | 277 | — | — | — | — | — | I | H | L | — | H | Y | — | — | N | 0.078 | 2.602 | 0.453 | 277 |
| 88 | WHYCFN | | | | | | | | | | | | | | | | | | | |
| Phage- | MDWCPRAL | 278 | — | — | — | — | — | — | A | L | — | N | V | — | — | N | 0.065 | 2.598 | 0.472 | 278 |
| 89 | WNVCFN | | | | | | | | | | | | | | | | | | | |
| Phage- | MDWCPIDL | 279 | — | — | — | — | — | I | D | L | — | H | L | — | — | Y | 0.062 | 2.590 | 0.488 | 279 |
| 90 | WHLCFY | | | | | | | | | | | | | | | | | | | |
| Phage- | MDWCPRDL | 280 | — | — | — | — | — | — | D | L | — | F | L | — | Y | N | 0.060 | 2.587 | 0.424 | 280 |
| 91 | WFLCYN | | | | | | | | | | | | | | | | | | | |
| Phage- | MDWCPRHL | 281 | — | — | — | — | — | — | H | L | — | H | E | — | — | — | 0.364 | 2.583 | 0.373 | 281 |
| 92 | WHECFF | | | | | | | | | | | | | | | | | | | |
| Phage- | MDWCPRYL | 282 | — | — | — | — | — | — | Y | L | — | T | V | — | — | S | 0.115 | 2.582 | 0.372 | 282 |
| 93 | WTVCFS | | | | | | | | | | | | | | | | | | | |
| Phage- | MDWCPRDL | 283 | — | — | — | — | — | — | D | L | — | S | L | — | — | Y | 0.065 | 2.573 | 0.246 | 283 |
| 94 | WSLCFY | | | | | | | | | | | | | | | | | | | |
| Phage- | MDWCPRFL | 284 | — | — | — | — | — | — | F | L | — | S | E | — | — | N | 0.062 | 2.571 | 0.227 | 284 |
| 95 | WSECFN | | | | | | | | | | | | | | | | | | | |
| Phage- | MDWCPRTL | 285 | — | — | — | — | — | — | T | L | — | A | Y | — | — | N | 0.070 | 2.571 | 0.584 | 285 |
| 96 | WAYCFN | | | | | | | | | | | | | | | | | | | |
| Phage- | MDWCPKDL | 286 | — | — | — | — | — | K | D | L | — | D | Y | — | — | A | 0.078 | 2.568 | 0.165 | 286 |
| 97 | WDYCFA | | | | | | | | | | | | | | | | | | | |
| Phage- | MDWCPKYL | 287 | — | — | — | — | — | K | Y | L | — | D | V | — | — | N | 0.096 | 2.566 | 0.422 | 287 |
| 98 | WDVCFN | | | | | | | | | | | | | | | | | | | |
| Phage- | MDWCPRYL | 288 | — | — | — | — | — | — | Y | L | — | N | M | — | — | H | 0.114 | 2.564 | 0.404 | 288 |
| 99 | WNMCFH | | | | | | | | | | | | | | | | | | | |
| Phage- | MDWCPRYL | 289 | — | — | — | — | — | — | Y | L | — | T | E | — | — | N | 0.062 | 2.560 | 0.549 | 289 |
| 100 | WTECFN | | | | | | | | | | | | | | | | | | | |
| Phage- | MDWCPRSL | 290 | — | — | — | — | — | — | S | L | — | H | Y | — | — | A | 0.140 | 2.552 | 0.338 | 290 |
| 101 | WHYCFA | | | | | | | | | | | | | | | | | | | |
| Phage- | MDWCPRYL | 291 | — | — | — | — | — | — | Y | L | — | A | E | — | — | Y | 0.063 | 2.552 | 0.354 | 291 |
| 102 | WAECFY | | | | | | | | | | | | | | | | | | | |
| Phage- | MDWCPRDL | 292 | — | — | — | — | — | — | D | L | — | H | E | — | — | N | 0.074 | 2.552 | 0.218 | 292 |
| 103 | WHECFN | | | | | | | | | | | | | | | | | | | |
| Phage- | MDWCPRDL | 293 | — | — | — | — | — | — | D | L | — | D | L | — | — | — | 0.076 | 2.549 | 0.156 | 293 |
| 104 | WDLCFF | | | | | | | | | | | | | | | | | | | |
| Phage- | MDWCPRYL | 294 | — | — | — | — | — | — | Y | L | — | N | V | — | — | N | 0.087 | 2.546 | 0.570 | 294 |
| 105 | WNVCFN | | | | | | | | | | | | | | | | | | | |
| Phage- | MDWCPIYL | 295 | — | — | — | — | — | I | Y | L | — | D | E | — | — | N | 0.061 | 2.546 | 0.215 | 295 |
| 106 | WDECFN | | | | | | | | | | | | | | | | | | | |
| Phage- | MDWCPRDL | 296 | — | — | — | — | — | — | D | L | — | A | E | — | — | N | 0.061 | 2.537 | 0.207 | 296 |
| 107 | WAECFN | | | | | | | | | | | | | | | | | | | |
| Phage- | MDWCPRAL | 297 | — | — | — | — | — | — | A | L | — | H | E | — | — | T | 0.075 | 2.536 | 0.191 | 297 |
| 108 | WHECFT | | | | | | | | | | | | | | | | | | | |
| Phage- | MDWCPKNL | 298 | — | — | — | — | — | K | N | L | — | H | V | — | — | N | 0.081 | 2.530 | 0.248 | 298 |
| 109 | WHVCFN | | | | | | | | | | | | | | | | | | | |
| Phage- | MDWCPRYL | 299 | — | — | — | — | — | — | Y | L | — | D | E | — | — | N | 0.069 | 2.529 | 0.283 | 299 |
| 110 | WDECFN | | | | | | | | | | | | | | | | | | | |
| Phage- | MDWCPFYL | 300 | — | — | — | — | — | F | Y | L | — | N | E | — | — | Y | 0.099 | 2.528 | 0.594 | 300 |
| 111 | WNECFY | | | | | | | | | | | | | | | | | | | |
| Phage- | MHWCPRAL | 301 | — | H | — | — | — | — | A | L | — | D | V | — | Y | N | 0.077 | 2.528 | 0.240 | 301 |
| 112 | WDVCYN | | | | | | | | | | | | | | | | | | | |
| Phage- | MDWCPRDL | 302 | — | — | — | — | — | — | D | L | — | N | V | — | — | — | 0.164 | 2.520 | 0.137 | 302 |
| 113 | WNVCFF | | | | | | | | | | | | | | | | | | | |
| Phage- | MDWCPRYL | 303 | — | — | — | — | — | — | Y | L | — | F | E | — | — | A | 0.066 | 2.504 | 0.210 | 303 |
| 114 | WFECFA | | | | | | | | | | | | | | | | | | | |
| Phage- | MDWCPRYL | 304 | — | — | — | — | — | — | Y | L | — | H | E | — | — | N | 0.105 | 2.490 | 0.421 | 304 |
| 115 | WHECFN | | | | | | | | | | | | | | | | | | | |
| Phage- | MDWCPRDL | 305 | — | — | — | — | — | — | D | L | — | Y | A | — | — | A | 0.072 | 2.474 | 0.242 | 305 |
| 116 | WYACFA | | | | | | | | | | | | | | | | | | | |
| Phage- | MDWCPRYL | 306 | — | — | — | — | — | — | Y | L | — | F | E | — | — | S | 0.079 | 2.462 | 0.235 | 306 |
| 117 | WFECFS | | | | | | | | | | | | | | | | | | | |
| Phage- | MDWCPRHL | 307 | — | — | — | — | — | — | H | L | — | D | E | — | — | — | 0.074 | 2.459 | 0.663 | 307 |
| 118 | WDECFF | | | | | | | | | | | | | | | | | | | |
| Phage- | MDWCPRYL | 308 | — | — | — | — | — | — | Y | L | — | H | M | — | Y | S | 0.129 | 2.419 | 1.166 | 308 |
| 119 | WHMCYS | | | | | | | | | | | | | | | | | | | |
| Phage- | MDWCPRDL | 309 | — | — | — | — | — | — | D | L | — | H | A | — | — | S | 0.074 | 2.414 | 0.173 | 309 |
| 120 | WHACFS | | | | | | | | | | | | | | | | | | | |
| Phage- | MDWCPRDL | 310 | — | — | — | — | — | — | D | L | — | H | V | — | — | — | 0.068 | 2.412 | 0.268 | 310 |
| 121 | WHVCFF | | | | | | | | | | | | | | | | | | | |
| Phage- | MDWCPRDL | 311 | — | — | — | — | — | — | D | L | — | D | Q | — | Y | A | 0.059 | 2.406 | 0.201 | 311 |
| 122 | WDQCYA | | | | | | | | | | | | | | | | | | | |
| Phage- | MDWCPIHL | 312 | — | — | — | — | I | — | H | L | — | N | E | — | — | A | 0.104 | 2.405 | 0.203 | 312 |
| 123 | WNECFA | | | | | | | | | | | | | | | | | | | |
| Phage- | MDWCPRPL | 313 | — | — | — | — | — | — | P | L | — | D | M | — | — | — | 0.063 | 2.403 | 0.138 | 313 |
| 124 | WDMCFF | | | | | | | | | | | | | | | | | | | |
| Phage- | MDWCPVSL | 314 | — | — | — | — | — | V | S | L | — | H | V | — | — | Y | 0.107 | 2.401 | 0.409 | 314 |
| 125 | WHVCFY | | | | | | | | | | | | | | | | | | | |
| Phage- | MDWCPRFL | 315 | — | — | — | — | — | — | F | L | — | N | E | — | — | N | 0.072 | 2.400 | 0.503 | 315 |
| 126 | WNECFN | | | | | | | | | | | | | | | | | | | |
| Phage- | MDWCPRAL | 316 | — | — | — | — | — | — | A | L | V | N | E | — | — | A | 0.080 | 2.385 | 0.375 | 316 |
| 127 | WNECFA | | | | | | | | | | | | | | | | | | | |
| Phage- | MDWCPRDL | 317 | — | — | — | — | — | — | D | L | — | I | E | — | — | — | 0.084 | 2.372 | 0.137 | 317 |
| 128 | WIECFF | | | | | | | | | | | | | | | | | | | |
| Phage- | MDWCPSYL | 318 | — | — | — | — | — | S | Y | L | — | T | V | — | — | A | 0.062 | 2.340 | 0.096 | 318 |
| 129 | WTVCFA | | | | | | | | | | | | | | | | | | | |
| Phage- | MDWCPRYL | 319 | — | — | — | — | — | — | Y | L | — | D | A | — | — | — | 0.066 | 2.337 | 0.168 | 319 |
| 130 | WDACFF | | | | | | | | | | | | | | | | | | | |
| Phage- | MDWCPRSL | 320 | — | — | — | — | — | — | S | L | — | I | Y | — | — | N | 0.113 | 2.336 | 0.401 | 320 |
| 131 | WIYCFN | | | | | | | | | | | | | | | | | | | |
| Phage- | MDWCPTYL | 321 | — | — | — | — | — | T | Y | L | — | F | E | — | — | N | 0.205 | 2.329 | 0.224 | 321 |
| 132 | WFECFN | | | | | | | | | | | | | | | | | | | |
| Phage- | MDWCPRFL | 322 | — | — | — | — | — | — | F | L | — | D | E | — | — | — | 0.064 | 2.284 | 0.246 | 322 |
| 133 | WDECFF | | | | | | | | | | | | | | | | | | | |
| Phage- | MDWCPSYL | 323 | — | — | — | — | — | S | Y | L | — | H | E | — | — | A | 0.096 | 2.284 | 0.198 | 323 |
| 134 | WHECFA | | | | | | | | | | | | | | | | | | | |
| Phage- | MDWCPKFL | 324 | — | — | — | — | — | K | F | L | — | H | E | — | — | S | 0.065 | 2.279 | 0.142 | 324 |
| 135 | WHECFS | | | | | | | | | | | | | | | | | | | |
| Phage- | MHWCPIYL | 325 | — | H | — | — | — | I | Y | L | — | D | E | — | — | N | 0.071 | 2.234 | 0.193 | 325 |
| 136 | WDECFN | | | | | | | | | | | | | | | | | | | |
| Phage- | MDWCPRYL | 326 | — | — | — | — | — | — | Y | L | — | H | E | — | — | H | 0.101 | 2.193 | 0.308 | 326 |
| 137 | WHECFH | | | | | | | | | | | | | | | | | | | |
| Phage- | MDWCPTNL | 327 | — | — | — | — | — | T | N | L | — | H | E | — | — | A | 0.061 | 2.163 | 0.096 | 327 |
| 138 | WHECFA | | | | | | | | | | | | | | | | | | | |
| Phage- | MDWCPRDL | 328 | — | — | — | — | — | — | D | L | — | D | V | — | — | A | 0.096 | 2.158 | 0.280 | 328 |
| 139 | WDVCFA | | | | | | | | | | | | | | | | | | | |
| Phage- | MDWCPMDL | 329 | — | — | — | — | — | M | D | L | — | D | V | — | — | N | 0.090 | 2.149 | 0.263 | 329 |
| 140 | WDVCFN | | | | | | | | | | | | | | | | | | | |
| Phage- | MDWCPRSL | 330 | — | — | — | — | — | — | S | L | — | N | V | — | — | — | 0.106 | 2.131 | 0.356 | 330 |
| 141 | WNVCFF | | | | | | | | | | | | | | | | | | | |
| Phage- | MDWCPVIL | 331 | — | — | — | — | — | V | I | L | — | D | F | — | — | N | 0.066 | 2.030 | 0.173 | 331 |
| 142 | WDFCFN | | | | | | | | | | | | | | | | | | | |
| Phage- | LDWCPLNL | 332 | L | — | — | — | — | L | N | L | — | D | L | — | Y | — | 0.062 | 2.028 | 0.095 | 332 |
| 143 | WDLCYF | | | | | | | | | | | | | | | | | | | |
| Phage- | MDWCPRHL | 333 | — | — | — | — | — | — | H | L | — | Y | A | — | — | N | 0.061 | 2.012 | 0.159 | 333 |
| 144 | WYACFN | | | | | | | | | | | | | | | | | | | |
| Phage- | MDWCPKHL | 334 | — | — | — | — | — | K | H | L | — | — | E | — | — | A | 0.065 | 1.977 | 0.132 | 334 |
| 145 | WIECFA | | | | | | | | | | | | | | | | | | | |
| Phage- | MDWCPRHL | 335 | — | — | — | — | — | — | H | L | — | S | E | — | — | Y | 0.136 | 1.918 | 0.199 | 335 |
| 146 | WSECFY | | | | | | | | | | | | | | | | | | | |
| Phage- | MHWCPRDL | 336 | — | H | — | — | — | — | D | L | — | — | V | — | — | N | 0.070 | 1.834 | 0.112 | 336 |
| 147 | WVVCFN | | | | | | | | | | | | | | | | | | | |
| Phage- | MHWCPEYL | 337 | — | H | — | — | — | E | Y | L | — | N | E | — | — | A | 0.074 | 1.687 | 0.092 | 337 |
| 148 | WNECFA | | | | | | | | | | | | | | | | | | | |
| Phage- | MDWCPRDL | 338 | — | — | — | — | — | — | D | L | — | A | V | — | — | A | 0.070 | 1.669 | 0.089 | 338 |
| 149 | WAVCFA | | | | | | | | | | | | | | | | | | | |
| Phage- | MDWCPRHL | 339 | — | — | — | — | — | — | H | L | — | N | V | — | — | S | 0.058 | 1.613 | 0.176 | 339 |
| 150 | WNVCFS | | | | | | | | | | | | | | | | | | | |
| Phage- | MDFCPISL | 340 | — | — | F | — | — | I | S | L | — | H | E | — | — | — | 0.081 | 1.585 | 0.129 | 340 |
| 151 | WHECFF | | | | | | | | | | | | | | | | | | | |
| Phage- | MDWCPKYL | 341 | — | — | — | — | — | K | Y | L | — | D | K | — | — | H | 0.073 | 1.540 | 0.109 | 341 |
| 152 | WDKCFH | | | | | | | | | | | | | | | | | | | |
| Phage- | MDWCPRHL | 342 | — | — | — | — | — | — | H | L | — | D | L | — | — | — | 0.093 | 1.395 | 0.182 | 342 |
| 153 | WDLCFF | | | | | | | | | | | | | | | | | | | |
| Phage- | MDWCPRDL | 343 | — | — | — | — | — | — | D | L | — | N | V | — | — | A | 0.114 | 1.382 | 0.145 | 343 |
| 154 | WNVCFA | | | | | | | | | | | | | | | | | | | |
| Phage- | MHWCPLHL | 344 | — | H | — | — | — | L | H | L | — | N | E | — | Y | H | 0.063 | 1.370 | 0.113 | 344 |
| 155 | WNECYH | | | | | | | | | | | | | | | | | | | |
| Phage- | MDWCPKHL | 345 | — | — | — | — | — | K | H | L | — | H | Q | — | — | H | 0.058 | 1.351 | 0.108 | 345 |
| 156 | WHQCFH | | | | | | | | | | | | | | | | | | | |
| Phage- | MDWCPRSI | 346 | — | — | — | — | — | — | S | L | — | S | Y | — | — | H | 0.066 | 1.335 | 0.152 | 346 |
| 157 | WSYCFH | | | | | | | | | | | | | | | | | | | |
| Phage- | MDWCPRYL | 347 | — | — | — | — | — | — | Y | L | — | T | E | — | — | — | 0.110 | 1.265 | 0.244 | 347 |
| 158 | WTECFF | | | | | | | | | | | | | | | | | | | |
| Phage- | MAWCPMNL | 348 | — | A | — | — | — | M | N | L | — | D | Q | — | — | — | 0.070 | 1.202 | 0.145 | 348 |
| 159 | WDQCFF | | | | | | | | | | | | | | | | | | | |
| Phage- | MHWCPRAL | 349 | — | H | — | — | — | — | A | L | — | H | E | — | — | N | 0.070 | 1.178 | 0.144 | 349 |
| 160 | WHECFN | | | | | | | | | | | | | | | | | | | |
| Phage- | MDWCPRHL | 350 | — | — | — | — | — | — | H | L | — | D | Q | — | — | A | 0.150 | 1.122 | 0.144 | 350 |
| 161 | WDQCFA | | | | | | | | | | | | | | | | | | | |
| Phage- | MNWCPTDL | 351 | — | N | — | — | — | T | D | L | — | H | E | — | — | N | 0.087 | 1.093 | 0.095 | 351 |
| 162 | WHECFN | | | | | | | | | | | | | | | | | | | |
| Phage- | MFWCPRYL | 352 | — | F | — | — | — | — | Y | L | — | H | E | — | — | N | 0.086 | 1.078 | 0.175 | 352 |
| 163 | WHECFN | | | | | | | | | | | | | | | | | | | |
| Phage- | MDWCPKFL | 353 | — | — | — | — | — | K | F | L | — | D | L | — | — | A | 0.100 | 1.075 | 0.139 | 353 |
| 164 | WDLCFA | | | | | | | | | | | | | | | | | | | |
| Phage- | MDWCPFYL | 354 | — | — | — | — | — | F | Y | L | — | D | E | — | — | L | 0.070 | 1.024 | 0.141 | 354 |
| 165 | WDECFL | | | | | | | | | | | | | | | | | | | |
| Phage- | MDWCPRHL | 355 | — | — | — | — | — | — | H | L | — | D | L | — | — | A | 0.061 | 0.925 | 0.096 | 355 |
| 166 | WDLCFA | | | | | | | | | | | | | | | | | | | |
| Phage- | MSWCPQDL | 356 | — | S | — | — | — | Q | D | L | — | H | V | — | — | N | 0.095 | 0.860 | 0.112 | 356 |
| 167 | WHVCFN | | | | | | | | | | | | | | | | | | | |
| Phage- | MDWCPKDL | 357 | — | — | — | — | — | K | D | L | — | H | E | — | — | N | 0.073 | 0.762 | 0.100 | 357 |
| 168 | WHECFN | | | | | | | | | | | | | | | | | | | |
| Phage- | MDWCPRDL | 358 | — | — | — | — | — | — | D | L | — | N | V | — | — | N | 0.100 | 0.740 | 0.108 | 358 |
| 169 | WNVCFN | | | | | | | | | | | | | | | | | | | |
| Phage- | MNWCPSDL | 359 | — | N | — | — | — | S | D | L | — | H | L | — | — | N | 0.071 | 0.739 | 0.131 | 359 |
| 170 | WHLCFN | | | | | | | | | | | | | | | | | | | |
| Phage- | MNWCPSHL | 360 | — | N | — | — | — | S | H | L | — | H | M | — | Y | — | 0.195 | 0.702 | 0.192 | 360 |
| 171 | WHMCYF | | | | | | | | | | | | | | | | | | | |
| Phage- | MDWCPPYL | 361 | — | — | — | — | — | P | Y | L | — | Y | E | — | — | A | 0.068 | 0.692 | 0.094 | 361 |
| 172 | WYECFA | | | | | | | | | | | | | | | | | | | |
| Phage- | MDWCPMNL | 362 | — | — | — | — | — | M | N | L | — | S | E | — | — | N | 0.168 | 0.670 | 0.124 | 362 |
| 173 | WSECFN | | | | | | | | | | | | | | | | | | | |
| Phage- | MDWCPKHL | 363 | — | — | — | — | — | K | H | L | — | N | E | — | — | N | 0.063 | 0.663 | 0.105 | 363 |
| 174 | WNECFN | | | | | | | | | | | | | | | | | | | |
| Phage- | MDWCPAYL | 364 | — | — | — | — | — | A | Y | L | V | A | E | — | — | S | 0.082 | 0.640 | 0.120 | 364 |
| 175 | WAECFS | | | | | | | | | | | | | | | | | | | |
| Phage- | MDWCPSDL | 365 | — | — | — | — | — | S | D | L | — | H | E | — | — | H | 0.075 | 0.629 | 0.164 | 365 |
| 176 | WHECFH | | | | | | | | | | | | | | | | | | | |
| Phage- | MDWCPVSL | 366 | — | — | — | — | — | V | S | L | — | D | H | — | — | N | 0.075 | 0.616 | 0.100 | 366 |
| 177 | WDHCFN | | | | | | | | | | | | | | | | | | | |
| Phage- | LDWCPRDL | 367 | L | — | — | — | — | — | D | L | — | H | V | — | — | — | 0.176 | 0.610 | 0.105 | 367 |
| 178 | WHVCFF | | | | | | | | | | | | | | | | | | | |
| Phage- | MDWCPWIL | 368 | — | — | — | — | — | W | I | L | — | N | E | — | — | N | 0.141 | 0.581 | 0.137 | 368 |
| 179 | WNECFN | | | | | | | | | | | | | | | | | | | |
| Phage- | MHWCPRYL | 369 | — | H | — | — | — | — | Y | L | — | D | E | | — | N | 0.132 | 0.550 | 0.127 | 369 |
| 180 | WDECFN | | | | | | | | | | | | | | | | | | | |
| Phage- | MYWCPRDL | 370 | — | Y | — | — | — | — | D | L | — | D | V | — | — | N | 0.145 | 0.512 | 0.102 | 370 |
| 181 | WDVCFN | | | | | | | | | | | | | | | | | | | |
| Phage- | MHWCPRSL | 371 | — | H | — | — | — | — | S | L | — | N | E | — | Y | — | 0.168 | 0.456 | 0.115 | 371 |
| 182 | WNECYF | | | | | | | | | | | | | | | | | | | |
| Phage- | IDWCPRDL | 372 | — | — | — | — | — | — | D | L | — | A | L | — | — | N | 0.127 | 0.370 | 0.129 | 372 |
| 183 | WALCFN | | | | | | | | | | | | | | | | | | | |
| Phage- | MHWCPINL | 373 | — | H | — | — | — | I | N | L | — | N | E | — | — | S | 0.081 | 0.316 | 0.098 | 373 |
| 184 | WNECFS | | | | | | | | | | | | | | | | | | | |
| Phage- | MERCPRFL | 374 | — | E | R | — | — | — | F | L | — | N | E | — | — | N | 0.132 | 0.216 | 0.093 | 374 |
| 185 | WNECFN | | | | | | | | | | | | | | | | | | | |
| Phage- | QDWCPTYL | 375 | Q | — | — | — | — | T | Y | L | — | H | H | — | — | N | 0.101 | 0.123 | 0.122 | 375 |
| 186 | WHHCFN | | | | | | | | | | | | | | | | | | | |
| Phage- | IGKLTLCL | 376 | I | G | K | L | T | L | C | L | N | A | — | L | V | I | 0.274 | 0.116 | 0.100 | 376 |
| 187 | NADLVI | | | | | | | | | | | | | | | | | | | |
| Phage- | MDWCPSYL | 377 | — | — | — | — | — | S | Y | L | — | D | Q | — | — | — | 0.100 | 0.083 | 0.079 | 377 |
| 188 | WDQCFF | | | | | | | | | | | | | | | | | | | |
| Phage- | VDWCPRYL | 378 | V | — | — | — | — | — | Y | L | — | H | V | — | Y | N | 0.064 | 0.065 | 0.061 | 378 |
| 189 | WHVCYN | | | | | | | | | | | | | | | | | | | |
| Phage- | MDWCPRDM | 379 | — | — | — | — | — | — | D | M | — | A | E | — | — | — | 0.099 | 2.687 | 2.259 | 379 |
| 190 | WAECFF | | | | | | | | | | | | | | | | | | | |
| Phage- | MDWCPRDM | 380 | — | — | — | — | — | — | D | M | — | Y | E | — | — | N | 0.099 | 2.564 | 0.235 | 380 |
| 191 | WYECFN | | | | | | | | | | | | | | | | | | | |
| Phage- | DVWCPKYM | 381 | D | V | — | — | — | K | Y | M | — | S | L | — | — | N | 0.101 | 2.529 | 0.418 | 381 |
| 192 | WSLCFN | | | | | | | | | | | | | | | | | | | |
| Phage- | MDWCPMDM | 382 | — | — | — | — | — | M | D | M | — | | N | — | — | N | 0.059 | 2.516 | 0.277 | 382 |
| 193 | WVNCFN | | | | | | | | | | | | | | | | | | | |
| Phage- | MDWCPSDM | 383 | — | — | — | — | — | S | D | M | — | H | E | — | Y | A | 0.062 | 2.474 | 0.200 | 383 |
| 194 | WHECYA | | | | | | | | | | | | | | | | | | | |
| Phage- | MDWCPKHM | 384 | — | — | — | — | — | K | H | M | — | F | M | — | — | N | 0.101 | 2.395 | 0.372 | 384 |
| 195 | WFMCFN | | | | | | | | | | | | | | | | | | | |
| Phage- | MDWCPRYM | 385 | — | — | — | — | — | — | Y | M | — | Y | Q | — | — | S | 0.079 | 2.364 | 0.357 | 385 |
| 196 | WYQCFS | | | | | | | | | | | | | | | | | | | |
| Phage- | MDWCPRHM | 386 | — | — | — | — | — | — | H | M | — | Y | E | — | — | — | 0.129 | 2.342 | 0.298 | 386 |
| 197 | WYECFF | | | | | | | | | | | | | | | | | | | |
| Phage- | MDWCPRAM | 387 | — | — | — | — | — | — | A | M | — | N | H | — | — | N | 0.321 | 2.326 | 0.426 | 387 |
| 198 | WNHCFN | | | | | | | | | | | | | | | | | | | |
| Phage- | MDWCPRNM | 388 | — | — | — | — | — | — | N | M | — | A | Q | — | — | A | 0.102 | 2.315 | 0.209 | 388 |
| 199 | WAQCFA | | | | | | | | | | | | | | | | | | | |
| Phage- | MFWCPFDM | 389 | — | F | — | — | — | F | D | M | — | H | F | — | — | N | 0.150 | 2.292 | 0.469 | 389 |
| 200 | WHFCFN | | | | | | | | | | | | | | | | | | | |
| Phage- | MDWCPRDM | 390 | — | — | — | — | — | — | D | M | — | D | Q | — | — | D | 0.072 | 2.292 | 0.119 | 390 |
| 201 | WDQCFD | | | | | | | | | | | | | | | | | | | |
| Phage- | MFWCPMDM | 391 | — | F | — | — | — | M | D | M | — | D | Q | — | — | N | 0.091 | 2.260 | 0.207 | 391 |
| 202 | WDQCFN | | | | | | | | | | | | | | | | | | | |
| Phage- | MSWCPRDM | 392 | — | S | — | — | — | — | D | M | — | F | Y | — | Y | A | 0.065 | 2.248 | 0.174 | 392 |
| 203 | WFYCYA | | | | | | | | | | | | | | | | | | | |
| Phage- | MDWCPRHM | 393 | — | — | — | — | — | — | H | M | — | N | V | — | — | S | 0.067 | 2.245 | 0.130 | 393 |
| 204 | WNVCFS | | | | | | | | | | | | | | | | | | | |
| Phage- | MDWCPTDM | 394 | — | — | — | — | — | T | D | M | — | H | H | — | — | L | 0.061 | 2.201 | 0.126 | 394 |
| 205 | WHHCFL | | | | | | | | | | | | | | | | | | | |
| Phage- | IHWCPINM | 395 | — | H | — | — | — | I | N | M | — | D | K | — | Y | N | 0.060 | 2.080 | 0.203 | 395 |
| 206 | WDKCYN | | | | | | | | | | | | | | | | | | | |
| Phage- | MDWCPRAM | 396 | — | — | — | — | — | — | A | M | — | H | E | — | — | Y | 0.069 | 1.915 | 0.129 | 396 |
| 207 | WHECFY | | | | | | | | | | | | | | | | | | | |
| Phage- | MDWCPTDM | 397 | — | — | — | — | — | T | D | M | — | I | V | — | — | A | 0.363 | 1.495 | 0.172 | 397 |
| 208 | WIVCFA | | | | | | | | | | | | | | | | | | | |
| Phage- | IDWCPQDM | 398 | I | — | — | — | — | Q | D | M | — | F | Y | — | — | N | 0.069 | 1.416 | 0.160 | 398 |
| 209 | WFYCFN | | | | | | | | | | | | | | | | | | | |
| Phage- | MDWCPRDM | 399 | — | — | — | — | — | — | D | M | — | F | E | — | — | A | 0.076 | 1.310 | 0.143 | 399 |
| 210 | WFECFA | | | | | | | | | | | | | | | | | | | |
| Phage- | MDWCPRNM | 400 | — | — | — | — | — | — | N | M | — | T | V | — | — | L | 0.060 | 1.116 | 0.093 | 400 |
| 211 | WTVCFL | | | | | | | | | | | | | | | | | | | |
| Phage- | MDWCPRAM | 401 | — | — | — | — | — | — | A | M | — | D | K | — | — | — | 0.073 | 0.985 | 0.094 | 401 |
| 212 | WDKCFF | | | | | | | | | | | | | | | | | | | |
| Phage- | MNWCPSYM | 402 | — | N | — | — | — | S | Y | M | — | D | Q | — | — | A | 0.125 | 0.854 | 0.105 | 402 |
| 213 | WDQCFA | | | | | | | | | | | | | | | | | | | |
| Phage- | MDWCPTYM | 403 | — | — | — | — | — | T | Y | M | — | S | E | — | — | N | 0.086 | 0.754 | 0.107 | 403 |
| 214 | WSECFN | | | | | | | | | | | | | | | | | | | |
| Phage- | MDWCPRYM | 404 | — | — | — | — | — | — | Y | M | — | N | E | — | — | N | 0.064 | 0.687 | 0.128 | 404 |
| 215 | WNECFN | | | | | | | | | | | | | | | | | | | |
| Phage- | MDWCPMNM | 405 | — | — | — | — | — | M | N | M | — | Y | Q | — | — | N | 0.110 | 0.639 | 0.137 | 405 |
| 216 | WYQCFN | | | | | | | | | | | | | | | | | | | |
| Phage- | MDWCPWDM | 406 | — | — | — | — | — | W | D | M | — | D | K | — | — | N | 0.101 | 0.586 | 0.108 | 406 |
| 217 | WDKCFN | | | | | | | | | | | | | | | | | | | |
| Phage- | TFGCPTTM | 407 | T | F | G | — | — | T | T | M | — | N | R | — | — | A | 0.117 | 0.181 | 0.068 | 407 |
| 218 | WNRCFA | | | | | | | | | | | | | | | | | | | |
| Phage- | NYWCPSSM | 408 | N | Y | — | — | — | S | S | M | — | N | R | — | L | H | 0.295 | 0.158 | 0.088 | 408 |
| 219 | WNRCLH | | | | | | | | | | | | | | | | | | | |
| Phage- | FDFCPTTM | 409 | F | — | F | — | — | T | T | M | — | T | Y | — | Q | H | 0.084 | 0.086 | 0.081 | 409 |
| 220 | WTYCQH | | | | | | | | | | | | | | | | | | | |
| Phage- | TTWCPTSM | 410 | T | T | — | — | — | T | S | M | — | L | H | — | — | D | 0.096 | 0.074 | 0.072 | 410 |
| 221 | WLHCFD | | | | | | | | | | | | | | | | | | | |
| Phage- | MDWCPRDQ | 411 | — | — | — | — | — | — | D | Q | — | H | N | — | — | N | 0.075 | 0.200 | 0.078 | 411 |
| 222 | WHNCFN | | | | | | | | | | | | | | | | | | | |
| Phage- | MDWCPRDR | 412 | — | — | — | — | — | — | D | — | — | — | — | — | — | — | 0.066 | 1.577 | 0.140 | 412 |
| 223 | WVDCFF | | | | | | | | | | | | | | | | | | | |
| Phage- | MDWCPKDR | 413 | — | — | — | — | — | K | D | — | — | N | — | — | Y | — | 0.078 | 1.091 | 0.093 | 413 |
| 224 | WNDCYF | | | | | | | | | | | | | | | | | | | |
| Phage- | MDWCPRDR | 414 | — | — | — | — | — | — | D | — | — | A | — | — | — | — | 0.069 | 0.392 | 0.089 | 414 |
| 225 | WADCFF | | | | | | | | | | | | | | | | | | | |
| Phage- | MDWCPRDR | 415 | — | — | — | — | — | — | D | — | — | I | — | — | — | N | 0.218 | 0.339 | 0.131 | 415 |
| 226 | WIDCFN | | | | | | | | | | | | | | | | | | | |
| Phage- | MDWCPRDR | 416 | — | — | — | — | — | — | D | — | — | S | — | — | — | N | 0.074 | 0.102 | 0.074 | 416 |
| 227 | WSDCFN | | | | | | | | | | | | | | | | | | | |
| Phage- | ITWCHVIS | 417 | I | T | — | — | H | V | I | S | G | L | E | — | W | N | 0.076 | 0.193 | 0.100 | 417 |
| 228 | GLECWN | | | | | | | | | | | | | | | | | | | |
| Phage- | VPWCQIIS | 418 | V | P | — | — | Q | I | I | S | G | L | E | — | L | T | 0.076 | 0.174 | 0.077 | 418 |
| 229 | GLECLT | | | | | | | | | | | | | | | | | | | |
| Phage- | APWCQIIS | 419 | A | P | — | — | Q | — | — | S | G | L | E | — | L | T | 0.080 | 0.169 | 0.078 | 419 |
| 230 | GLECLT | | | | | | | | | | | | | | | | | | | |
| Phage- | VPWCLIIS | 420 | V | P | — | — | L | — | — | S | G | L | — | — | L | N | 0.455 | 0.107 | 0.090 | 420 |
| 231 | GLDCLN | | | | | | | | | | | | | | | | | | | |
| Phage- | MDWCARFV | 421 | — | — | — | — | A | — | F | V | G | Y | G | — | L | D | 0.948 | 0.065 | 0.074 | 421 |
| 232 | GYGCLD | | | | | | | | | | | | | | | | | | | |
| Phage- | MTWCPTSF | 422 | — | T | — | — | — | T | S | F | — | N | R | — | L | D | 0.142 | 0.392 | 0.405 | 422 |
| 233 | WNRCLD | | | | | | | | | | | | | | | | | | | |
| Phage- | MDWCPRAL | 423 | — | — | — | — | — | — | A | L | — | F | E | — | — | — | 0.111 | 2.668 | 0.331 | 423 |
| 234 | WFECFF | | | | | | | | | | | | | | | | | | | |
| Phage- | MDWCPRYL | 424 | — | — | — | — | — | — | Y | L | — | H | E | — | — | S | 0.063 | 2.654 | 0.303 | 424 |
| 235 | WHECFS | | | | | | | | | | | | | | | | | | | |
| Phage- | MDWCPRDL | 425 | — | — | — | — | — | — | D | L | — | N | L | — | — | — | 0.084 | 2.639 | 0.436 | 425 |
| 236 | WNLCFF | | | | | | | | | | | | | | | | | | | |
| Phage- | MDWCPSYL | 426 | — | — | — | — | — | S | Y | L | V | H | E | — | — | — | 0.067 | 2.606 | 0.447 | 426 |
| 237 | WHECFF | | | | | | | | | | | | | | | | | | | |
| Phage- | MDWCPPYL | 427 | — | — | — | — | — | P | Y | L | — | S | E | — | — | A | 0.539 | 2.604 | 0.234 | 427 |
| 238 | WSECFA | | | | | | | | | | | | | | | | | | | |
| Phage- | MDWCPRYL | 428 | — | — | — | — | — | — | Y | L | — | H | V | — | — | N | 0.187 | 2.603 | 0.409 | 428 |
| 239 | WHVCFN | | | | | | | | | | | | | | | | | | | |
| Phage- | MDWCPRTL | 429 | — | — | — | — | — | — | T | L | I | H | V | — | — | N | 0.317 | 2.601 | 0.551 | 429 |
| 240 | WHVCFN | | | | | | | | | | | | | | | | | | | |
| Phage- | MDWCPRHL | 430 | — | — | — | — | — | — | H | L | — | H | E | — | Y | S | 0.066 | 2.584 | 0.374 | 430 |
| 241 | WHECYS | | | | | | | | | | | | | | | | | | | |
| Phage- | MDWCPKHL | 431 | — | — | — | — | — | K | H | L | — | T | E | — | — | A | 0.098 | 2.491 | 0.145 | 431 |
| 242 | WTECFA | | | | | | | | | | | | | | | | | | | |
| Phage- | MDWCPRHL | 432 | — | — | — | — | — | — | H | L | — | Y | E | — | — | N | 0.063 | 2.414 | 0.167 | 432 |
| 243 | WYECFN | | | | | | | | | | | | | | | | | | | |
| Phage- | MDWCPRYL | 433 | — | — | — | — | — | — | Y | L | — | H | E | — | — | D | 0.071 | 2.066 | 0.177 | 433 |
| 244 | WHECFD | | | | | | | | | | | | | | | | | | | |
| Phage- | MDWCPRNL | 434 | — | — | — | — | — | — | N | L | — | H | L | — | — | A | 0.082 | 1.397 | 0.164 | 434 |
| 245 | WHLCFA | | | | | | | | | | | | | | | | | | | |
| Phage- | MDWCPKHL | 435 | — | — | — | — | — | K | H | L | — | N | K | — | — | N | 0.075 | 1.161 | 0.092 | 435 |
| 246 | WNKCFN | | | | | | | | | | | | | | | | | | | |
| Phage- | MDWCPRFM | 436 | — | — | — | — | — | — | F | M | — | A | E | — | — | N | 0.073 | 2.603 | 0.341 | 436 |
| 247 | WAECFN | | | | | | | | | | | | | | | | | | | |
| Phage- | MDWCPRNM | 437 | — | — | — | — | — | — | N | M | — | H | H | — | — | D | 0.205 | 1.597 | 0.524 | 437 |
| 248 | WHHCFD | | | | | | | | | | | | | | | | | | | |
| Phage- | MDWCPSDM | 438 | — | — | — | — | — | S | D | M | — | A | H | — | — | N | 0.191 | 1.209 | 0.109 | 438 |
| 249 | WAHCYN | | | | | | | | | | | | | | | | | | | |
| Phage- | MDWCPKVM | 439 | — | — | — | — | — | K | V | M | — | H | Y | — | — | A | 0.136 | 1.138 | 0.252 | 439 |
| 250 | WHYCFA | | | | | | | | | | | | | | | | | | | |
| Phage- | MDWCPRFM | 440 | — | — | — | — | — | — | F | M | — | S | E | — | — | S | 0.092 | 1.008 | 0.137 | 440 |
| 251 | WSECFS | | | | | | | | | | | | | | | | | | | |
| Phage- | MDWCPRHM | 441 | — | — | — | — | — | — | H | M | — | — | N | — | — | A | 0.091 | 0.920 | 0.165 | 441 |
| 252 | WINCFA | | | | | | | | | | | | | | | | | | | |
| Phage- | MVGCATSM | 442 | — | V | G | — | A | T | S | M | — | N | R | — | L | T | 0.256 | 0.770 | 0.746 | 442 |
| 253 | WNRCLT | | | | | | | | | | | | | | | | | | | |
| Phage- | MDWCPRYL | 443 | — | — | — | — | — | — | Y | L | — | S | E | — | — | A | 0.078 | 2.705 | 0.513 | 443 |
| 254 | WSECFA | | | | | | | | | | | | | | | | | | | |
| Phage- | MDWCPRHL | 444 | — | — | — | — | — | — | H | L | — | S | V | — | — | N | 0.064 | 2.649 | 0.825 | 444 |
| 255 | WSVCFN | | | | | | | | | | | | | | | | | | | |
| Phage- | MDWCPFDL | 445 | — | — | — | — | — | F | D | L | — | H | V | — | Y | N | 0.089 | 2.602 | 0.651 | 445 |
| 256 | WHVCYN | | | | | | | | | | | | | | | | | | | |
| Phage- | MDWCPRFL | 446 | — | — | — | — | — | — | F | L | — | H | E | — | — | N | 0.079 | 2.586 | 0.351 | 446 |
| 257 | WHECFN | | | | | | | | | | | | | | | | | | | |
| Phage- | MDWCPRSL | 447 | — | — | — | — | — | — | S | L | — | H | V | — | — | N | 0.098 | 2.412 | 0.238 | 447 |
| 258 | WHVCFN | | | | | | | | | | | | | | | | | | | |
| Phage- | MDWCPRNL | 448 | — | — | — | — | — | — | N | L | — | H | A | — | — | N | 0.071 | 1.571 | 0.112 | 448 |
| 259 | WHACFN | | | | | | | | | | | | | | | | | | | |
| Phage- | MDWCPVFM | 449 | — | — | — | — | — | V | F | M | — | N | E | — | — | N | 0.079 | 2.661 | 0.356 | 449 |
| 260 | WNECFN | | | | | | | | | | | | | | | | | | | |
| Phage- | MDWCPMFM | 450 | — | — | — | — | — | M | F | M | — | H | E | — | — | N | 0.102 | 2.235 | 1.045 | 450 |
| 261 | WHECFN | | | | | | | | | | | | | | | | | | | |
| Phage- | MYWCATSM | 451 | — | Y | — | — | A | T | S | M | — | N | R | — | — | V | 0.248 | 0.398 | 0.415 | 451 |
| 262 | WNRCFV | | | | | | | | | | | | | | | | | | | |
| Phage- | MDWCPRHM | 452 | — | — | — | — | — | — | H | M | — | H | E | — | — | A | 0.074 | 2.518 | 0.298 | 452 |
| 263 | WHECFA | | | | | | | | | | | | | | | | | | | |
| Phage- | MDWCPRHL | 453 | — | — | — | — | — | — | H | L | — | H | V | — | — | N | 0.070 | 2.667 | 0.636 | 453 |
| 264 | WHVCFN | | | | | | | | | | | | | | | | | | | |
| Phage- | MDWCPRFM | 454 | — | — | — | — | — | — | F | M | — | D | L | — | — | A | 0.270 | 2.638 | 0.725 | 454 |
| 265 | WDLCFA | | | | | | | | | | | | | | | | | | | |
| Phage- | VTSCPTTM | 455 | V | T | S | — | — | T | T | M | — | N | R | — | — | S | 0.396 | 1.220 | 0.546 | 455 |
| 266 | WNRCFS | | | | | | | | | | | | | | | | | | | |
| Phage- | MTLCLSVD | 456 | — | T | L | — | L | S | V | D | L | — | H | — | W | Y | 0.114 | 0.632 | 0.093 | 456 |
| 267 | LVHCWY | | | | | | | | | | | | | | | | | | | |
| Phage- | DSFCTWSA | 457 | D | S | F | — | T | W | S | A | — | Q | E | — | G | R | 0.072 | 0.130 | 0.101 | 457 |
| 269 | WQECGR | | | | | | | | | | | | | | | | | | | |
| Phage- | MDWCPRDF | 458 | — | — | — | — | — | — | D | F | — | A | F | — | — | — | 0.118 | 2.365 | 0.655 | 458 |
| 270 | WAFCFF | | | | | | | | | | | | | | | | | | | |
| Phage- | MDWCPTSF | 459 | — | — | — | — | — | T | S | F | — | N | R | — | — | H | 0.102 | 0.087 | 0.092 | 459 |
| 271 | WNRCFH | | | | | | | | | | | | | | | | | | | |
| Phage- | MVWCMSVG | 460 | — | V | — | — | M | S | V | G | — | A | V | — | L | N | 0.289 | 0.271 | 0.284 | 460 |
| 272 | WAVCLN | | | | | | | | | | | | | | | | | | | |
| Phage- | IDWCPTAG | 461 | I | — | — | — | — | T | A | G | — | T | Y | — | W | — | 0.071 | 0.136 | 0.075 | 461 |
| 273 | WTYCWF | | | | | | | | | | | | | | | | | | | |
| Phage- | WVSCLRHH | 462 | W | V | S | — | L | — | H | H | — | L | E | — | — | H | 0.781 | 0.723 | 0.858 | 462 |
| 274 | WLECFH | | | | | | | | | | | | | | | | | | | |
| Phage- | MDWCPRDH | 463 | — | — | — | — | — | — | D | H | — | — | — | — | Y | S | 0.172 | 0.671 | 0.092 | 463 |
| 275 | WVDCYS | | | | | | | | | | | | | | | | | | | |
| Phage- | IDWCPKIH | 464 | — | — | — | — | — | K | — | H | — | D | L | — | Y | — | 0.072 | 0.295 | 0.082 | 464 |
| 276 | WDLCYF | | | | | | | | | | | | | | | | | | | |
| Phage- | MDWCPRSH | 465 | — | — | — | — | — | — | S | H | — | S | E | — | — | S | 0.080 | 0.253 | 0.087 | 465 |
| 277 | WSECFS | | | | | | | | | | | | | | | | | | | |
| Phage- | MDWCPRSI | 466 | — | — | — | — | — | — | S | — | — | Y | L | — | — | N | 0.383 | 2.548 | 1.337 | 466 |
| 278 | WYLCFN | | | | | | | | | | | | | | | | | | | |
| Phage- | MDWCPRYL | 467 | — | — | — | — | — | — | Y | L | — | A | E | — | — | N | 0.068 | 2.742 | 1.141 | 467 |
| 279 | WAECFN | | | | | | | | | | | | | | | | | | | |
| Phage- | MDWCPRYL | 468 | — | — | — | — | — | — | Y | L | — | D | Y | — | — | A | 0.073 | 2.709 | 0.382 | 468 |
| 280 | WDYCFA | | | | | | | | | | | | | | | | | | | |
| Phage- | MDWCPRSL | 469 | — | — | — | — | — | — | S | L | — | F | L | — | — | N | 1.110 | 2.709 | 1.774 | 469 |
| 281 | WFLCFN | | | | | | | | | | | | | | | | | | | |
| Phage- | MDWCPRDL | 470 | — | — | — | — | — | — | D | L | — | F | A | — | — | A | 0.060 | 2.658 | 0.794 | 470 |
| 282 | WFACFA | | | | | | | | | | | | | | | | | | | |
| Phage- | MDWCPRHL | 471 | — | — | — | — | — | — | H | L | — | N | E | — | — | N | 0.079 | 2.650 | 0.393 | 471 |
| 283 | WNECFN | | | | | | | | | | | | | | | | | | | |
| Phage- | MDWCPRFL | 472 | — | — | — | — | — | — | F | L | — | H | V | — | — | S | 2.091 | 2.643 | 2.480 | 472 |
| 284 | WHVCFS | | | | | | | | | | | | | | | | | | | |
| Phage- | MDWCPRDL | 473 | — | — | — | — | — | — | D | L | — | A | M | — | — | A | 0.071 | 2.627 | 0.889 | 473 |
| 285 | WAMCFA | | | | | | | | | | | | | | | | | | | |
| Phage- | MERCPRYL | 474 | — | E | R | — | — | — | Y | L | — | H | M | — | Y | S | 0.164 | 2.589 | 0.840 | 474 |
| 286 | WHMCYS | | | | | | | | | | | | | | | | | | | |
| Phage- | MDWCPIAL | 475 | — | — | — | — | — | I | A | L | — | D | F | — | — | A | 0.073 | 2.522 | 0.492 | 475 |
| 288 | WDFCFA | | | | | | | | | | | | | | | | | | | |
| Phage- | MDWCPRYL | 476 | — | — | — | — | — | — | Y | L | — | N | V | — | — | A | 0.070 | 2.513 | 0.280 | 476 |
| 289 | WNVCFA | | | | | | | | | | | | | | | | | | | |
| Phage- | MDWCPRDL | 477 | — | — | — | — | — | — | D | L | — | S | V | — | — | S | 0.090 | 2.505 | 0.116 | 477 |
| 290 | WSVCFS | | | | | | | | | | | | | | | | | | | |
| Phage- | MDWCPRYL | 478 | — | — | — | — | — | — | Y | L | — | H | V | — | — | Y | 0.227 | 2.498 | 1.137 | 478 |
| 291 | WHVCFY | | | | | | | | | | | | | | | | | | | |
| Phage- | MDWCPRAL | 479 | — | — | — | — | — | — | A | L | — | D | H | — | — | — | 0.081 | 2.492 | 0.394 | 479 |
| 292 | WDHCFF | | | | | | | | | | | | | | | | | | | |
| Phage- | MDWCPRHL | 480 | — | — | — | — | — | — | H | L | — | F | E | — | — | N | 0.064 | 2.489 | 0.171 | 480 |
| 293 | WFECFN | | | | | | | | | | | | | | | | | | | |
| Phage- | MDWCPRSL | 481 | — | — | — | — | — | — | S | L | — | D | Y | — | — | A | 0.115 | 2.488 | 0.235 | 481 |
| 294 | WDYCFA | | | | | | | | | | | | | | | | | | | |
| Phage- | MDWCPRHL | 482 | — | — | — | — | — | — | H | L | — | T | E | — | — | S | 0.074 | 2.482 | 0.243 | 482 |
| 297 | WTECFS | | | | | | | | | | | | | | | | | | | |
| Phage- | MDWCPLYL | 483 | — | — | — | — | — | L | Y | L | — | A | E | — | — | N | 0.094 | 2.476 | 0.193 | 483 |
| 298 | WAECFN | | | | | | | | | | | | | | | | | | | |
| Phage- | MDWCPIYL | 484 | — | — | — | — | — | I | Y | L | — | A | E | — | — | N | 0.078 | 2.453 | 0.307 | 484 |
| 299 | WAECFN | | | | | | | | | | | | | | | | | | | |
| Phage- | MDWCPRHL | 485 | — | — | — | — | — | — | H | L | — | F | E | — | — | — | 0.178 | 2.451 | 0.479 | 485 |
| 300 | WFECFF | | | | | | | | | | | | | | | | | | | |
| Phage- | MDWCPRYL | 486 | — | — | — | — | — | — | Y | L | — | H | E | — | — | — | 0.082 | 2.450 | 0.682 | 486 |
| 301 | WHECFF | | | | | | | | | | | | | | | | | | | |
| Phage- | MDWCPRYL | 487 | — | — | — | — | — | — | Y | L | — | H | V | — | — | D | 0.068 | 2.445 | 0.310 | 487 |
| 302 | WHVCFD | | | | | | | | | | | | | | | | | | | |
| Phage- | MDWCPRYL | 488 | — | — | — | — | — | — | Y | L | — | T | E | — | — | S | 0.074 | 2.421 | 0.329 | 488 |
| 303 | WTECFS | | | | | | | | | | | | | | | | | | | |
| Phage- | MDWCPKFL | 489 | — | — | — | — | — | K | F | L | V | D | E | — | — | A | 0.311 | 2.406 | 0.376 | 489 |
| 304 | WDECFA | | | | | | | | | | | | | | | | | | | |
| Phage- | MDWCPRDL | 490 | — | — | — | — | — | — | D | L | — | T | E | — | — | S | 0.080 | 2.401 | 0.174 | 490 |
| 305 | WTECFS | | | | | | | | | | | | | | | | | | | |
| Phage- | MDWCPRHL | 491 | — | — | — | — | — | — | H | L | — | N | E | — | — | A | 0.068 | 2.394 | 0.170 | 491 |
| 306 | WNECFA | | | | | | | | | | | | | | | | | | | |
| Phage- | MDWCPRYL | 492 | — | — | — | — | — | — | Y | L | — | P | V | — | — | H | 0.179 | 2.392 | 0.602 | 492 |
| 307 | WPVCFH | | | | | | | | | | | | | | | | | | | |
| Phage- | MDWCPKSL | 493 | — | — | — | — | — | K | S | L | — | A | E | — | — | N | 0.395 | 2.377 | 0.162 | 493 |
| 308 | WAECFN | | | | | | | | | | | | | | | | | | | |
| Phage- | MDWCPMFL | 494 | — | — | — | — | — | M | F | L | — | H | E | — | — | N | 0.170 | 2.375 | 0.591 | 494 |
| 309 | WHECFN | | | | | | | | | | | | | | | | | | | |
| Phage- | MDWCPRDL | 495 | — | — | — | — | — | — | D | L | — | D | E | — | — | N | 0.066 | 2.374 | 0.248 | 495 |
| 310 | WDECFN | | | | | | | | | | | | | | | | | | | |
| Phage- | MDWCPRDL | 496 | — | — | — | — | — | — | D | L | — | Y | Q | — | — | N | 0.069 | 2.353 | 0.609 | 496 |
| 311 | WYQCFN | | | | | | | | | | | | | | | | | | | |
| Phage- | MDWCPRSL | 497 | — | — | — | — | — | — | S | L | — | N | Y | — | — | N | 0.076 | 2.350 | 0.209 | 497 |
| 313 | WNYCFN | | | | | | | | | | | | | | | | | | | |
| Phage- | MDWCPIHL | 498 | — | — | — | — | — | I | H | L | — | N | E | — | — | N | 0.074 | 2.347 | 0.157 | 498 |
| 314 | WNECFN | | | | | | | | | | | | | | | | | | | |
| Phage- | MDWCPTYL | 499 | — | — | — | — | — | T | Y | L | — | H | V | — | — | S | 0.089 | 2.324 | 0.160 | 499 |
| 315 | WHVCFS | | | | | | | | | | | | | | | | | | | |
| Phage- | MDWCPRSL | 500 | — | — | — | — | — | — | S | L | — | N | Y | — | — | A | 0.067 | 2.316 | 0.198 | 500 |
| 316 | WNYCFA | | | | | | | | | | | | | | | | | | | |
| Phage- | MDWCPRHL | 501 | — | — | — | — | — | — | H | L | — | H | E | — | — | T | 0.103 | 2.297 | 0.320 | 501 |
| 317 | WHECFT | | | | | | | | | | | | | | | | | | | |
| Phage- | MDWCPMFL | 502 | — | — | — | — | — | M | F | L | — | D | E | — | — | N | 0.132 | 2.287 | 0.203 | 502 |
| 318 | WDECFN | | | | | | | | | | | | | | | | | | | |
| Phage- | MSWCPRDL | 503 | — | S | — | — | — | — | D | L | — | H | L | — | — | — | 0.077 | 2.265 | 0.319 | 503 |
| 319 | WHLCFF | | | | | | | | | | | | | | | | | | | |
| Phage- | MDWCPTYL | 504 | — | — | — | — | — | T | Y | L | — | N | E | — | — | N | 0.120 | 2.244 | 0.606 | 504 |
| 320 | WNECFN | | | | | | | | | | | | | | | | | | | |
| Phage- | MDWCPRDL | 505 | — | — | — | — | — | — | D | L | — | Y | V | — | — | A | 0.095 | 2.221 | 0.138 | 505 |
| 321 | WYVCFA | | | | | | | | | | | | | | | | | | | |
| Phage- | MDWCPRHL | 506 | — | — | — | — | — | — | H | L | — | — | E | — | — | N | 0.339 | 2.214 | 0.376 | 506 |
| 322 | WIECFN | | | | | | | | | | | | | | | | | | | |
| Phage- | MDWCPTYL | 507 | — | — | — | — | — | T | Y | L | — | N | V | — | — | A | 0.085 | 2.211 | 0.132 | 507 |
| 323 | WNVCFA | | | | | | | | | | | | | | | | | | | |
| Phage- | MDWCPRHL | 508 | — | — | — | — | — | — | H | L | — | Y | E | — | — | S | 0.216 | 2.167 | 0.163 | 508 |
| 324 | WYECFS | | | | | | | | | | | | | | | | | | | |
| Phage- | MDWCPAYL | 509 | — | — | — | — | — | A | Y | L | — | D | E | — | — | A | 0.096 | 2.166 | 0.163 | 509 |
| 325 | WDECFA | | | | | | | | | | | | | | | | | | | |
| Phage- | MFWCPITL | 510 | — | F | — | — | — | — | T | L | — | N | E | — | — | N | 0.075 | 2.153 | 0.143 | 510 |
| 326 | WNECFN | | | | | | | | | | | | | | | | | | | |
| Phage- | MHWCPRDL | 511 | — | H | — | — | — | — | D | L | — | H | V | — | — | N | 0.071 | 2.149 | 0.150 | 511 |
| 327 | WHVCFN | | | | | | | | | | | | | | | | | | | |
| Phage- | MDWCPRAL | 512 | — | — | — | — | — | — | A | L | — | D | H | — | — | N | 0.323 | 2.145 | 0.293 | 512 |
| 328 | WDHCFN | | | | | | | | | | | | | | | | | | | |
| Phage- | MDWCPRAL | 513 | — | — | — | — | — | — | A | L | — | N | V | — | — | A | 0.071 | 2.140 | 0.249 | 513 |
| 329 | WNVCFA | | | | | | | | | | | | | | | | | | | |
| Phage- | MDWCPRFL | 514 | — | — | — | — | — | — | F | L | — | D | V | — | — | N | 0.100 | 2.139 | 0.309 | 514 |
| 330 | WDVCFN | | | | | | | | | | | | | | | | | | | |
| Phage- | MDWCPRHL | 515 | — | — | — | — | — | — | H | L | — | D | E | — | — | Y | 0.098 | 2.127 | 0.146 | 515 |
| 331 | WDECFY | | | | | | | | | | | | | | | | | | | |
| Phage- | IDWCPRAL | 516 | I | — | — | — | — | — | A | L | — | D | A | — | L | A | 0.269 | 2.103 | 0.164 | 516 |
| 332 | WDACLA | | | | | | | | | | | | | | | | | | | |
| Phage- | MDWCPTDL | 517 | — | — | — | — | — | T | D | L | — | H | E | — | — | A | 0.078 | 2.071 | 0.154 | 517 |
| 333 | WHECFA | | | | | | | | | | | | | | | | | | | |
| Phage- | MDWCPRYL | 518 | — | — | — | — | — | — | Y | L | — | D | E | — | — | S | 0.069 | 2.064 | 0.164 | 518 |
| 334 | WDECFS | | | | | | | | | | | | | | | | | | | |
| Phage- | MDWCPRFL | 519 | — | — | — | — | — | — | F | L | — | D | Y | — | — | A | 0.146 | 2.034 | 0.207 | 519 |
| 335 | WDYCFA | | | | | | | | | | | | | | | | | | | |
| Phage- | MDWCPRDL | 520 | — | — | — | — | — | — | D | L | — | N | W | — | — | N | 0.139 | 2.026 | 0.390 | 520 |
| 336 | WNWCFN | | | | | | | | | | | | | | | | | | | |
| Phage- | MDWCPKPL | 521 | — | — | — | — | — | K | P | L | — | H | V | — | — | A | 0.069 | 2.022 | 0.152 | 521 |
| 337 | WHVCFA | | | | | | | | | | | | | | | | | | | |
| Phage- | MDWCPRFL | 522 | — | — | — | — | — | — | F | L | — | N | E | — | — | Y | 0.143 | 1.996 | 0.287 | 522 |
| 338 | WNECFY | | | | | | | | | | | | | | | | | | | |
| Phage- | MDWCPRTL | 523 | — | — | — | — | — | — | T | L | — | D | Q | — | — | — | 0.088 | 1.992 | 0.314 | 523 |
| 339 | WDQCFF | | | | | | | | | | | | | | | | | | | |
| Phage- | MSWCPIDL | 524 | — | S | — | — | — | I | D | L | — | S | E | — | — | A | 0.072 | 1.986 | 0.148 | 524 |
| 340 | WSECFA | | | | | | | | | | | | | | | | | | | |
| Phage- | MDWCPRHL | 525 | — | — | — | — | — | — | H | L | — | D | E | — | — | N | 0.084 | 1.978 | 0.268 | 525 |
| 341 | WDECFN | | | | | | | | | | | | | | | | | | | |
| Phage- | MDWCPRNL | 526 | — | — | — | — | — | — | N | L | — | H | E | — | — | A | 0.067 | 1.965 | 0.175 | 526 |
| 342 | WHECFA | | | | | | | | | | | | | | | | | | | |
| Phage- | MDWCPRDL | 527 | — | — | — | — | — | — | D | L | — | T | Q | — | — | — | 0.065 | 1.932 | 0.119 | 527 |
| 343 | WTQCFF | | | | | | | | | | | | | | | | | | | |
| Phage- | MDWCPRHL | 528 | — | — | — | — | — | — | H | L | — | — | H | — | — | S | 0.096 | 1.914 | 0.173 | 528 |
| 344 | WVHCFS | | | | | | | | | | | | | | | | | | | |
| Phage- | MDFCPRFL | 529 | — | — | F | — | — | — | F | L | — | H | E | — | — | N | 0.063 | 1.908 | 0.225 | 529 |
| 345 | WHECFN | | | | | | | | | | | | | | | | | | | |
| Phage- | MDWCPRHL | 530 | — | — | — | — | — | — | H | L | — | H | A | — | — | S | 0.073 | 1.826 | 0.167 | 530 |
| 346 | WHACFS | | | | | | | | | | | | | | | | | | | |
| Phage- | MDWCPLFL | 531 | — | — | — | — | — | L | F | L | — | D | Q | — | — | N | 0.116 | 1.819 | 0.264 | 531 |
| 347 | WDQCFN | | | | | | | | | | | | | | | | | | | |
| Phage- | MAWCPWYL | 532 | — | A | — | — | — | W | Y | L | — | D | E | — | — | N | 0.200 | 1.810 | 0.267 | 532 |
| 348 | WDECFN | | | | | | | | | | | | | | | | | | | |
| Phage- | LDWCPRHL | 533 | L | — | — | — | — | — | H | L | — | A | L | — | — | N | 0.073 | 1.780 | 0.240 | 533 |
| 349 | WALCFN | | | | | | | | | | | | | | | | | | | |
| Phage- | MDWCPWFL | 534 | — | — | — | — | — | W | F | L | — | N | E | — | — | N | 0.158 | 1.774 | 0.279 | 534 |
| 350 | WNECFN | | | | | | | | | | | | | | | | | | | |
| Phage- | MDWCPMNL | 535 | — | — | — | — | — | M | N | L | — | H | E | — | — | A | 0.080 | 1.742 | 0.160 | 535 |
| 351 | WHECFA | | | | | | | | | | | | | | | | | | | |
| Phage- | MDWCPIHL | 536 | — | — | — | — | — | I | H | L | — | Y | E | — | — | N | 0.080 | 1.734 | 0.203 | 536 |
| 352 | WYECFN | | | | | | | | | | | | | | | | | | | |
| Phage- | IDWCPLHL | 537 | I | — | — | — | — | L | H | L | — | H | E | — | Y | H | 0.067 | 1.693 | 0.182 | 537 |
| 353 | WHECYH | | | | | | | | | | | | | | | | | | | |
| Phage- | MDWCPRYL | 538 | — | — | — | — | — | — | Y | L | — | L | E | — | — | N | 0.083 | 1.685 | 0.368 | 538 |
| 354 | WLECFN | | | | | | | | | | | | | | | | | | | |
| Phage- | MDWCPMYL | 539 | — | — | — | — | — | M | Y | L | — | D | E | — | — | — | 0.082 | 1.652 | 0.178 | 539 |
| 356 | WDECFF | | | | | | | | | | | | | | | | | | | |
| Phage- | MDWCPRLL | 540 | — | — | — | — | — | — | L | L | — | H | E | — | — | N | 0.071 | 1.601 | 0.109 | 540 |
| 357 | WHECFN | | | | | | | | | | | | | | | | | | | |
| Phage- | MDWCPPHL | 541 | — | — | — | — | — | P | H | L | — | H | E | — | — | — | 0.069 | 1.449 | 0.136 | 541 |
| 358 | WHECFF | | | | | | | | | | | | | | | | | | | |
| Phage- | MDWCPRPL | 542 | — | — | — | — | — | — | P | L | — | H | E | — | — | A | 0.063 | 1.439 | 0.138 | 542 |
| 359 | WHECFA | | | | | | | | | | | | | | | | | | | |
| Phage- | MDWCPMFL | 543 | — | — | — | — | — | M | F | L | — | H | E | — | — | S | 0.092 | 1.435 | 0.193 | 543 |
| 360 | WHECFS | | | | | | | | | | | | | | | | | | | |
| Phage- | MHWCPRHL | 544 | — | H | — | — | — | — | H | L | — | S | E | — | — | N | 0.165 | 1.430 | 0.478 | 544 |
| 361 | WSECFN | | | | | | | | | | | | | | | | | | | |
| Phage- | MDWCPRIL | 545 | — | — | — | — | — | — | I | L | — | H | E | — | — | S | 0.064 | 1.391 | 0.174 | 545 |
| 362 | WHECFS | | | | | | | | | | | | | | | | | | | |
| Phage- | MNWCPMHL | 546 | — | N | — | — | — | M | H | L | — | A | E | — | — | N | 0.087 | 1.390 | 0.140 | 546 |
| 363 | WAECFN | | | | | | | | | | | | | | | | | | | |
| Phage- | MDWCPRSL | 547 | — | — | — | — | — | — | S | L | — | A | Q | — | — | Y | 0.067 | 1.387 | 0.140 | 547 |
| 364 | WAQCFY | | | | | | | | | | | | | | | | | | | |
| Phage- | MDWCPSYL | 548 | — | — | — | — | — | S | Y | L | — | P | V | — | — | N | 0.079 | 1.178 | 0.131 | 548 |
| 365 | WPVCFN | | | | | | | | | | | | | | | | | | | |
| Phage- | MHWCPLYL | 549 | — | H | — | — | — | L | Y | L | — | D | E | — | — | Y | 0.153 | 1.159 | 0.376 | 549 |
| 366 | WDECFY | | | | | | | | | | | | | | | | | | | |
| Phage- | MDWCPSFL | 550 | — | — | — | — | — | S | F | L | — | Y | E | — | — | N | 0.063 | 1.130 | 0.104 | 550 |
| 367 | WYECFN | | | | | | | | | | | | | | | | | | | |
| Phage- | MSWCPPYL | 551 | — | S | — | — | — | P | Y | L | — | T | V | — | Y | N | 0.176 | 1.104 | 0.326 | 551 |
| 368 | WTVCYN | | | | | | | | | | | | | | | | | | | |
| Phage- | MHWCPRDL | 552 | — | H | — | — | — | — | D | L | — | Y | E | — | — | A | 0.072 | 0.995 | 0.108 | 552 |
| 369 | WYECFA | | | | | | | | | | | | | | | | | | | |
| Phage- | MTWCPAYL | 553 | — | T | — | — | — | A | Y | L | — | H | E | — | — | N | 0.086 | 0.985 | 0.139 | 553 |
| 370 | WHECFN | | | | | | | | | | | | | | | | | | | |
| Phage- | MAWCPRYL | 554 | — | A | — | — | — | — | Y | L | — | A | E | — | — | — | 0.510 | 0.918 | 0.216 | 554 |
| 371 | WAECFF | | | | | | | | | | | | | | | | | | | |
| Phage- | MDWCPRYL | 555 | — | — | — | — | — | — | Y | L | — | — | — | — | — | A | 0.095 | 0.917 | 0.218 | 555 |
| 372 | WVDCFA | | | | | | | | | | | | | | | | | | | |
| Phage- | MDWCPRIL | 556 | — | — | — | — | — | — | I | L | — | S | — | — | — | N | 0.082 | 0.847 | 0.113 | 556 |
| 373 | WSDCFN | | | | | | | | | | | | | | | | | | | |
| Phage- | MAWCPLDL | 557 | — | A | — | — | — | L | D | L | — | D | K | — | — | Y | 0.079 | 0.770 | 0.141 | 557 |
| 374 | WDKCFY | | | | | | | | | | | | | | | | | | | |
| Phage- | MNWCPRAL | 558 | — | N | — | — | — | — | A | L | — | H | E | — | — | L | 0.085 | 0.759 | 0.107 | 558 |
| 375 | WHECFL | | | | | | | | | | | | | | | | | | | |
| Phage- | MDWCPRHL | 559 | — | — | — | — | — | — | H | L | — | T | Y | — | — | H | 0.109 | 0.757 | 0.207 | 559 |
| 376 | WTYCFH | | | | | | | | | | | | | | | | | | | |
| Phage- | MDWCPFDL | 560 | — | — | — | — | — | F | D | L | — | L | E | — | — | N | 0.087 | 0.650 | 0.120 | 560 |
| 377 | WLECFN | | | | | | | | | | | | | | | | | | | |
| Phage- | MHWCPLHL | 561 | — | H | — | — | — | L | H | L | — | N | E | — | — | A | 0.065 | 0.575 | 0.083 | 561 |
| 378 | WNECFA | | | | | | | | | | | | | | | | | | | |
| Phage- | MDWCPRNL | 562 | — | — | — | — | — | — | N | L | — | A | E | — | — | S | 0.061 | 0.538 | 0.080 | 562 |
| 379 | WAECFS | | | | | | | | | | | | | | | | | | | |
| Phage- | MDYCPSYL | 563 | — | — | Y | — | — | S | Y | L | — | H | E | — | — | A | 0.065 | 0.470 | 0.090 | 563 |
| 380 | WHECFA | | | | | | | | | | | | | | | | | | | |
| Phage- | MAWCPRIL | 564 | — | A | — | — | — | — | I | L | — | H | Q | — | — | N | 0.113 | 0.322 | 0.129 | 564 |
| 381 | WHQCFN | | | | | | | | | | | | | | | | | | | |
| Phage- | ITWCPTSL | 565 | — | T | — | — | — | T | S | L | — | N | R | — | — | V | 0.077 | 0.242 | 0.089 | 565 |
| 382 | WNRCLV | | | | | | | | | | | | | | | | | | | |
| Phage- | LAGCQRDL | 566 | L | A | G | — | Q | — | D | L | A | T | V | — | V | I | 0.068 | 0.229 | 0.116 | 566 |
| 383 | ATVCVI | | | | | | | | | | | | | | | | | | | |
| Phage- | IMWCPTSL | 567 | I | M | — | — | — | T | S | L | L | N | R | — | V | T | 0.104 | 0.161 | 0.129 | 567 |
| 384 | WNRCVT | | | | | | | | | | | | | | | | | | | |
| Phage- | ILRCQMNL | 568 | I | L | R | — | Q | M | N | L | Q | D | E | — | L | N | 0.083 | 0.083 | 0.077 | 568 |
| 385 | QDECLN | | | | | | | | | | | | | | | | | | | |
| Phage- | NHWCPTTL | 569 | N | H | — | — | — | T | T | L | — | N | R | — | V | A | 0.293 | 0.078 | 0.093 | 569 |
| 386 | WNRCVA | | | | | | | | | | | | | | | | | | | |
| Phage- | MDWCPRHL | 570 | — | — | — | — | — | — | H | L | — | L | E | — | — | N | 0.064 | 0.066 | 0.074 | 570 |
| 387 | WLECFN | | | | | | | | | | | | | | | | | | | |
| Phage- | MDWCPRFM | 571 | — | — | — | — | — | — | F | M | — | N | F | — | — | — | 2.200 | 2.621 | 2.539 | 571 |
| 388 | WNFCFF | | | | | | | | | | | | | | | | | | | |
| Phage- | MDWCPSDM | 572 | — | — | — | — | — | S | D | M | — | A | N | — | — | — | 0.110 | 2.621 | 0.316 | 572 |
| 389 | WANCFF | | | | | | | | | | | | | | | | | | | |
| Phage- | IDWCPMHM | 573 | I | — | — | — | — | M | H | M | — | D | F | — | Y | N | 0.078 | 2.544 | 0.232 | 573 |
| 390 | WDFCYN | | | | | | | | | | | | | | | | | | | |
| Phage- | MDWCPFDM | 574 | — | — | — | — | — | F | D | M | — | A | — | — | — | — | 0.063 | 2.532 | 0.215 | 574 |
| 391 | WADCFF | | | | | | | | | | | | | | | | | | | |
| Phage- | LHWCPTSM | 575 | L | H | — | — | — | T | S | M | — | T | Y | — | Y | Y | 0.688 | 2.475 | 2.456 | 575 |
| 392 | WTYCYY | | | | | | | | | | | | | | | | | | | |
| Phage- | MDWCPRDM | 576 | — | — | — | — | — | — | D | M | — | F | E | — | — | N | 0.069 | 2.422 | 0.778 | 576 |
| 393 | WFECFN | | | | | | | | | | | | | | | | | | | |
| Phage- | MDWCPRYM | 577 | — | — | — | — | — | — | Y | M | — | S | — | — | — | — | 0.082 | 2.280 | 0.377 | 577 |
| 394 | WSDCFF | | | | | | | | | | | | | | | | | | | |
| Phage- | MDWCPKDM | 578 | — | — | — | — | — | K | D | M | — | A | E | — | — | N | 0.083 | 2.106 | 0.457 | 578 |
| 395 | WAECFN | | | | | | | | | | | | | | | | | | | |
| Phage- | TDWCPRDM | 579 | T | — | — | — | — | — | D | M | — | Y | L | — | — | N | 0.082 | 2.106 | 0.301 | 579 |
| 396 | WYLCFN | | | | | | | | | | | | | | | | | | | |
| Phage- | MDWC PRAM | 580 | — | — | — | — | — | — | A | M | — | D | Y | — | — | Y | 0.123 | 2.106 | 0.339 | 580 |
| 397 | WDYCFY | | | | | | | | | | | | | | | | | | | |
| Phage- | MDWCPRNM | 581 | — | — | — | — | — | — | N | M | — | N | E | — | — | — | 0.095 | 2.099 | 0.430 | 581 |
| 398 | WNECFF | | | | | | | | | | | | | | | | | | | |
| Phage- | MDWCPRSM | 582 | — | — | — | — | — | — | S | M | — | D | S | — | — | N | 0.332 | 2.019 | 0.142 | 582 |
| 399 | WDSCFN | | | | | | | | | | | | | | | | | | | |
| Phage- | MHWCPTYM | 583 | — | H | — | — | — | T | Y | M | — | S | E | — | — | A | 0.070 | 1.839 | 0.161 | 583 |
| 400 | WSECFA | | | | | | | | | | | | | | | | | | | |
| Phage- | MDWCPLDM | 584 | — | — | — | — | — | L | D | M | — | — | L | — | — | A | 0.085 | 1.837 | 0.169 | 584 |
| 401 | WVLCFA | | | | | | | | | | | | | | | | | | | |
| Phage- | MDWCPRHM | 585 | — | — | — | — | — | — | H | M | — | H | E | — | — | H | 0.067 | 1.815 | 0.171 | 585 |
| 402 | WHECFH | | | | | | | | | | | | | | | | | | | |
| Phage- | MSWCPWDM | 586 | — | S | — | — | — | W | D | M | — | N | E | — | — | A | 0.069 | 1.521 | 0.108 | 586 |
| 403 | WNECFA | | | | | | | | | | | | | | | | | | | |
| Phage- | MDWCPRDM | 587 | — | — | — | — | — | — | D | M | — | T | S | — | — | N | 0.100 | 1.414 | 0.179 | 587 |
| 404 | WTSCFN | | | | | | | | | | | | | | | | | | | |
| Phage- | MDWCPMSM | 588 | — | — | — | — | — | M | S | M | — | A | F | — | — | D | 0.102 | 1.412 | 0.160 | 588 |
| 405 | WAFCFD | | | | | | | | | | | | | | | | | | | |
| Phage- | MNWCPIDM | 589 | — | N | — | — | — | I | D | M | — | T | E | — | — | N | 0.245 | 1.403 | 0.175 | 589 |
| 406 | WTECFN | | | | | | | | | | | | | | | | | | | |
| Phage- | MNWCPIHM | 590 | — | N | — | — | — | I | H | M | — | N | Q | — | — | A | 0.085 | 1.373 | 0.133 | 590 |
| 407 | WNQCFA | | | | | | | | | | | | | | | | | | | |
| Phage- | MFWCPKDM | 591 | — | F | — | — | — | K | D | M | — | A | E | — | — | A | 0.099 | 1.258 | 0.113 | 591 |
| 408 | WAECFA | | | | | | | | | | | | | | | | | | | |
| Phage- | LHWCPITM | 592 | L | H | — | — | — | I | T | M | — | T | Y | — | Y | Y | 0.229 | 1.212 | 0.516 | 592 |
| 409 | WTYCYY | | | | | | | | | | | | | | | | | | | |
| Phage- | IDWCPRYM | 593 | — | — | — | — | — | — | Y | M | — | H | E | — | — | — | 0.098 | 1.178 | 0.164 | 593 |
| 410 | WHECFF | | | | | | | | | | | | | | | | | | | |
| Phage- | MSWCPRFM | 594 | — | S | — | — | — | — | F | M | — | H | E | — | — | N | 0.081 | 1.111 | 0.246 | 594 |
| 411 | WHECFN | | | | | | | | | | | | | | | | | | | |
| Phage- | QHWCPRDM | 595 | Q | H | — | — | — | — | D | M | — | N | — | — | Y | A | 0.066 | 1.076 | 0.149 | 595 |
| 412 | WNDCYA | | | | | | | | | | | | | | | | | | | |
| Phage- | MDWCPSDM | 596 | — | — | — | — | — | S | D | M | — | S | N | — | — | N | 0.070 | 1.075 | 0.121 | 596 |
| 413 | WSNCFN | | | | | | | | | | | | | | | | | | | |
| Phage- | LYWCPRDM | 597 | L | Y | — | — | — | — | D | M | — | A | Y | — | Y | S | 0.068 | 1.019 | 0.157 | 597 |
| 414 | WAYCYS | | | | | | | | | | | | | | | | | | | |
| Phage- | MDWCPMYM | 598 | — | — | — | — | — | M | Y | M | — | H | K | — | — | H | 0.099 | 0.987 | 0.168 | 598 |
| 415 | WHKCFH | | | | | | | | | | | | | | | | | | | |
| Phage- | MDWCPSNM | 599 | — | — | — | — | — | S | N | M | — | N | E | — | — | — | 0.072 | 0.983 | 0.136 | 599 |
| 416 | WNECFF | | | | | | | | | | | | | | | | | | | |
| Phage- | IYWCPTAM | 600 | I | Y | — | — | — | T | A | M | — | N | R | — | S | A | 0.086 | 0.817 | 0.073 | 600 |
| 417 | WNRCSA | | | | | | | | | | | | | | | | | | | |
| Phage- | MYWCPKYM | 601 | — | Y | — | — | — | K | Y | M | — | S | E | — | — | A | 0.072 | 0.741 | 0.099 | 601 |
| 418 | WSECFA | | | | | | | | | | | | | | | | | | | |
| Phage- | VDWCPAHM | 602 | V | — | — | — | — | A | H | M | — | N | E | — | Y | N | 0.208 | 0.664 | 0.085 | 602 |
| 419 | WNECYN | | | | | | | | | | | | | | | | | | | |
| Phage- | MFWCPSTM | 603 | — | F | — | — | — | S | T | M | — | N | R | — | — | D | 0.063 | 0.464 | 0.138 | 603 |
| 420 | WNRCFD | | | | | | | | | | | | | | | | | | | |
| Phage- | TIFCPSTM | 604 | T | I | F | — | — | S | T | M | — | N | R | — | W | T | 0.138 | 0.262 | 0.085 | 604 |
| 421 | WNRCWT | | | | | | | | | | | | | | | | | | | |
| Phage- | MYWCPINM | 605 | — | Y | — | — | — | I | N | M | — | D | Y | — | Y | A | 0.122 | 0.109 | 0.088 | 605 |
| 422 | WDYCYA | | | | | | | | | | | | | | | | | | | |
| Phage- | INLCPTPM | 606 | I | N | L | — | — | T | P | M | — | N | R | — | W | L | 0.119 | 0.103 | 0.101 | 606 |
| 423 | WNRCWL | | | | | | | | | | | | | | | | | | | |
| Phage- | ITQCPSTM | 607 | — | T | Q | — | — | S | T | M | — | N | R | — | S | V | 0.067 | 0.098 | 0.108 | 607 |
| 424 | WNRCSV | | | | | | | | | | | | | | | | | | | |
| Phage- | MDLCPTAM | 608 | — | — | L | — | — | T | A | M | — | N | R | — | — | Y | 0.063 | 0.097 | 0.084 | 608 |
| 425 | WNRCFY | | | | | | | | | | | | | | | | | | | |
| Phage- | MFLCPSAM | 609 | — | F | L | — | — | S | A | M | — | N | R | — | — | Y | 0.069 | 0.094 | 0.114 | 609 |
| 426 | WNRCFY | | | | | | | | | | | | | | | | | | | |
| Phage- | VILCPTTM | 610 | V | I | L | — | — | T | T | M | — | N | R | — | — | H | 0.100 | 0.072 | 0.084 | 610 |
| 427 | WNRCFH | | | | | | | | | | | | | | | | | | | |
| Phage- | MNWCPSTM | 611 | — | N | — | — | — | S | T | M | — | N | R | — | L | T | 0.067 | 0.065 | 0.070 | 611 |
| 428 | WNRCLT | | | | | | | | | | | | | | | | | | | |
| Phage- | IHLCHWVP | 612 | I | H | L | — | H | W | V | P | — | — | K | — | S | H | 2.516 | 2.550 | 2.508 | 612 |
| 430 | WIKCSH | | | | | | | | | | | | | | | | | | | |
| Phage- | MAWCPADQ | 613 | — | A | — | — | — | A | D | Q | | H | E | — | — | N | 0.076 | 1.845 | 0.456 | 613 |
| 431 | WHECFN | | | | | | | | | | | | | | | | | | | |
| Phage- | IDWCPIIQ | 614 | I | — | — | — | — | I | I | Q | G | L | P | — | — | A | 0.058 | 0.368 | 0.065 | 614 |
| 432 | GLPCFA | | | | | | | | | | | | | | | | | | | |
| Phage- | MAWCPWAQ | 615 | — | A | — | — | — | W | A | Q | F | D | E | — | L | A | 0.073 | 0.071 | 0.078 | 615 |
| 433 | FDECLA | | | | | | | | | | | | | | | | | | | |
| Phage- | IVWCPTTQ | 616 | I | V | — | — | — | T | T | Q | | N | R | — | A | T | 0.113 | 0.067 | 0.083 | 616 |
| 434 | WNRCAT | | | | | | | | | | | | | | | | | | | |
| Phage- | MDWCPRDR | 617 | — | — | — | — | — | — | D | — | — | — | — | — | Y | A | 0.157 | 1.484 | 0.403 | 617 |
| 435 | WVDCYA | | | | | | | | | | | | | | | | | | | |
| Phage- | MDWCPRDR | 618 | — | — | — | — | — | — | D | — | — | A | — | — | — | N | 0.063 | 1.351 | 0.080 | 618 |
| 436 | WADCFN | | | | | | | | | | | | | | | | | | | |
| Phage- | MDWCPVDR | 619 | — | — | — | — | — | V | D | — | — | A | — | — | — | N | 0.071 | 1.156 | 0.315 | 619 |
| 437 | WADCFN | | | | | | | | | | | | | | | | | | | |
| Phage- | MDWCPMSR | 620 | — | — | — | — | — | M | S | — | — | A | F | — | — | D | 0.071 | 1.000 | 0.115 | 620 |
| 438 | WAFCFD | | | | | | | | | | | | | | | | | | | |
| Phage- | MDWCPRDR | 621 | — | — | — | — | — | — | D | — | — | D | V | — | — | A | 0.073 | 0.924 | 0.096 | 621 |
| 439 | WDVCFA | | | | | | | | | | | | | | | | | | | |
| Phage- | MDWCPRDR | 622 | — | — | — | — | — | — | D | — | — | — | — | — | — | N | 0.106 | 0.911 | 0.132 | 622 |
| 440 | WVDCFN | | | | | | | | | | | | | | | | | | | |
| Phage- | MDWCPRDR | 623 | — | — | — | — | — | — | D | — | — | D | — | — | — | — | 0.114 | 0.899 | 0.183 | 623 |
| 441 | WDDCFF | | | | | | | | | | | | | | | | | | | |
| Phage- | IYWCPIDR | 624 | I | Y | — | — | — | I | D | — | — | N | — | — | Y | N | 0.177 | 0.867 | 0.164 | 624 |
| 442 | WNDCYN | | | | | | | | | | | | | | | | | | | |
| Phage- | MDWCPMTR | 625 | — | — | — | — | — | M | T | — | — | N | — | — | Y | — | 0.081 | 0.671 | 0.078 | 625 |
| 443 | WNDCYF | | | | | | | | | | | | | | | | | | | |
| Phage- | VNQCTRYR | 626 | V | N | Q | — | T | — | Y | — | — | A | E | — | L | N | 0.465 | 0.566 | 0.135 | 626 |
| 444 | WAECLN | | | | | | | | | | | | | | | | | | | |
| Phage- | MDWCPRAR | 627 | — | — | — | — | — | — | A | — | — | H | — | — | — | — | 0.097 | 0.497 | 0.138 | 627 |
| 445 | WHDCFF | | | | | | | | | | | | | | | | | | | |
| Phage- | MDWCPRDR | 628 | — | — | — | — | — | — | D | — | — | D | — | — | — | N | 0.069 | 0.450 | 0.077 | 628 |
| 446 | WDDCFN | | | | | | | | | | | | | | | | | | | |
| Phage- | MDWCPRDR | 629 | — | — | — | — | — | — | D | — | — | D | V | — | Y | Y | 0.073 | 0.074 | 0.073 | 629 |
| 447 | WDVCYY | | | | | | | | | | | | | | | | | | | |
| Phage- | MAWCPWAS | 630 | — | A | — | — | — | W | A | S | F | D | E | — | L | A | 0.104 | 0.089 | 0.109 | 630 |
| 448 | FDECLA | | | | | | | | | | | | | | | | | | | |
| Phage- | MVDCPLIS | 631 | — | V | D | — | — | L | I | S | F | D | E | — | L | A | 0.246 | 0.070 | 0.076 | 631 |
| 449 | FDECLA | | | | | | | | | | | | | | | | | | | |
| Phage- | EDWCPTDV | 632 | E | — | — | — | — | T | D | V | — | P | Y | — | — | S | 0.100 | 0.711 | 0.157 | 632 |
| 450 | WPYCFS | | | | | | | | | | | | | | | | | | | |
| Phage- | MDWCPRFW | 633 | — | — | — | — | — | — | F | W | — | H | E | — | Y | A | 0.246 | 2.397 | 0.503 | 633 |
| 451 | WHECYA | | | | | | | | | | | | | | | | | | | |
| Phage- | MDWCPRDW | 634 | — | — | — | — | — | — | D | W | — | H | M | V | — | N | 0.100 | 2.115 | 0.243 | 634 |
| 452 | WHMCFN | | | | | | | | | | | | | | | | | | | |
| Phage- | MDWCPSDY | 635 | — | — | — | — | — | S | D | Y | — | Y | V | — | — | A | 0.065 | 0.707 | 0.080 | 635 |
| 453 | WYVCFA | | | | | | | | | | | | | | | | | | | |

### Example 5. Peptides Inhibit Anti-CD28 scFv and Ab-12 from Binding CD28 Antigen by ELISA

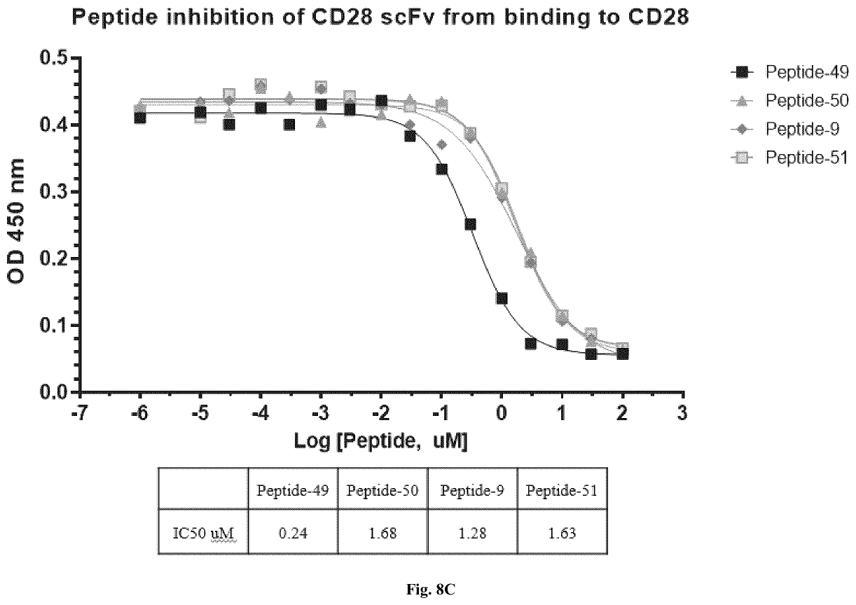

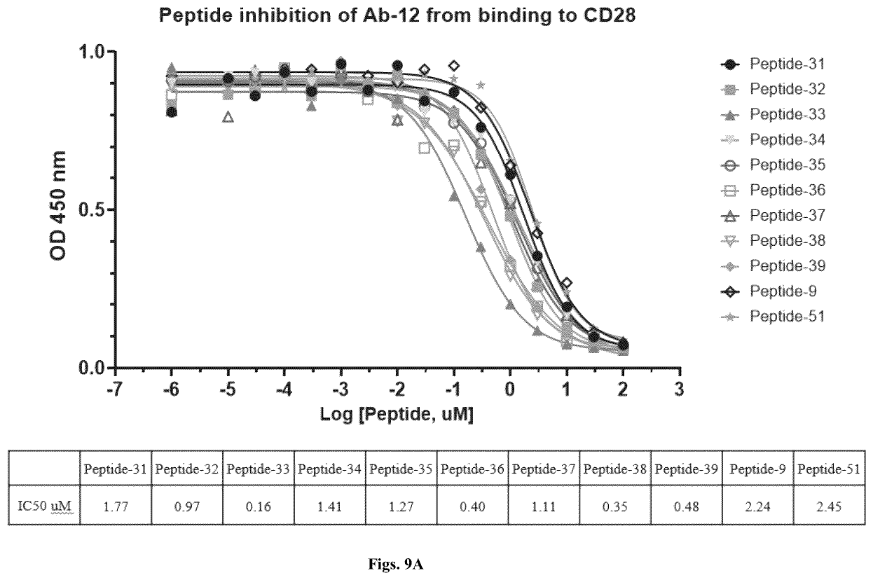

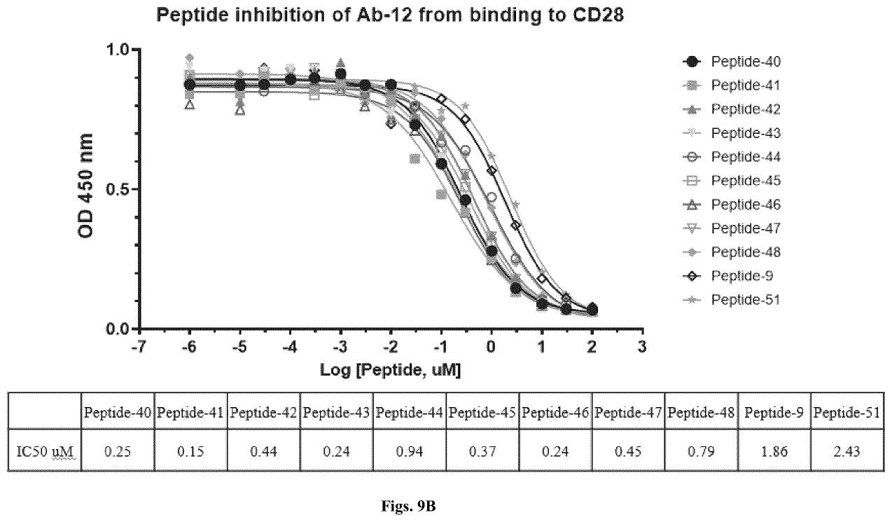

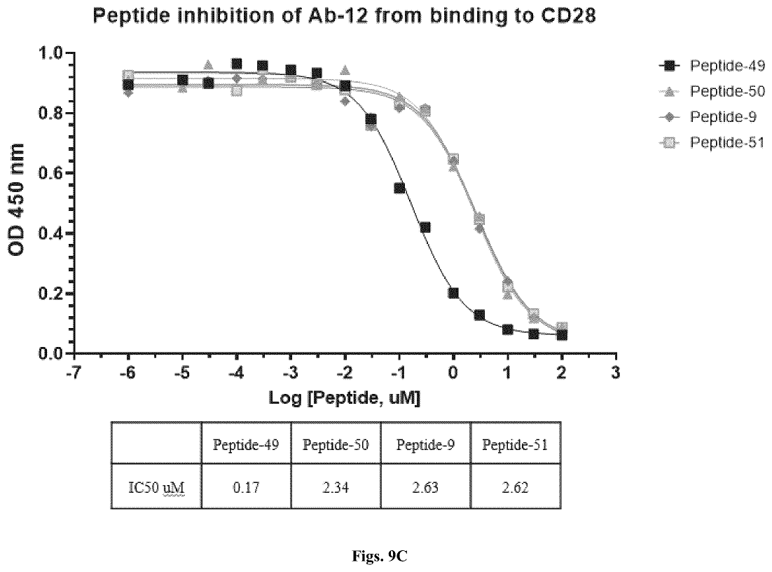

Peptides were evaluated for their ability to inhibit the anti-CD28 scFv or Ab-12 from binding to the CD28 antigen in a standard enzyme linked immunosorbent assay (ELISA) format. Briefly, biotinylated CD28 antigen was captured on neutravidin coated plates. Anti-CD28 scFv at 2 nM or Ab-12 at 5 nM were pre-incubated with 0-100 uM titrated peptides. After a short pre-incubation period the mixture of titrated peptides with fixed anti-CD28 scFv (2 nM) or Ab-12 (5 nM) were added to the CD28 antigen captured plates. After a short incubation on the plates, bound anti-CD28 scFv or Ab-12 were detected with a standard horse radish peroxidase conjugated secondary antibody. The concentration of peptide required to reduce the max signal by 50% (IC50) was calculated in Graphpad Prism software. FIGS. 8A-8C illustrate peptides that inhibit the anti-CD28 scFv from binding the CD28 antigen measured by ELISA. FIGS. 9A-9C illustrate peptides that inhibit Ab-12 from binding the CD28 antigen by ELISA.

### Example 6. Anti-CD28 scFv Kinetic Binding to Peptides by Octet

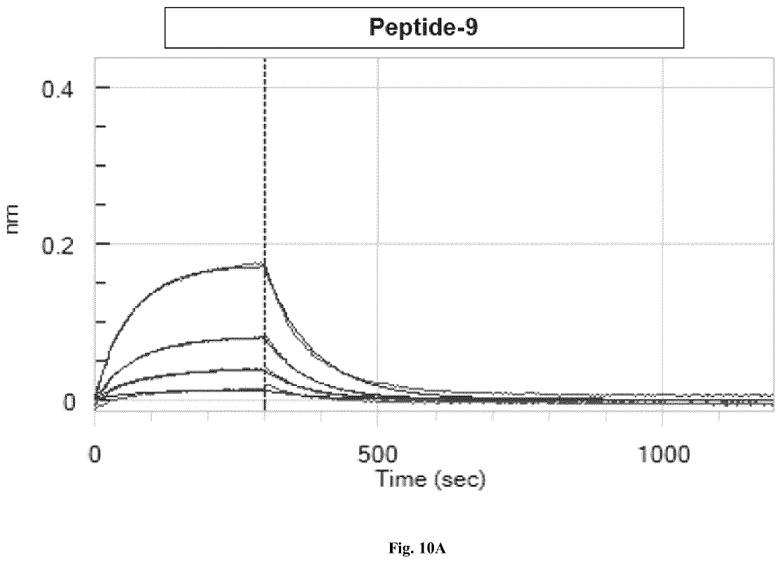

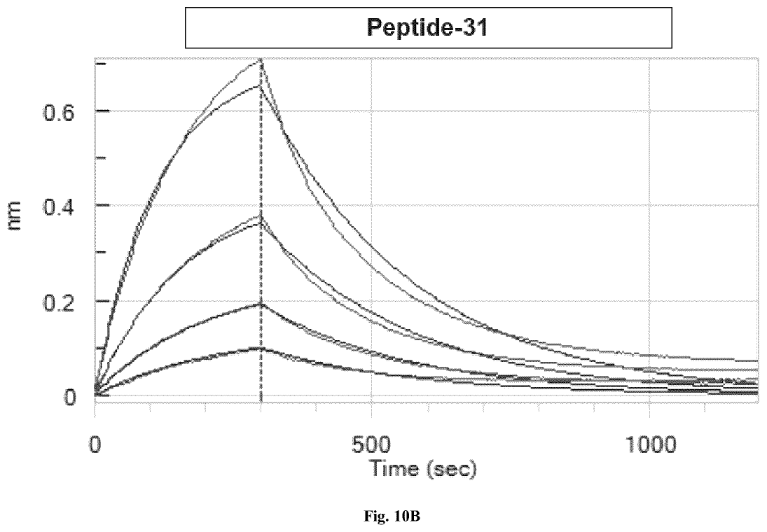

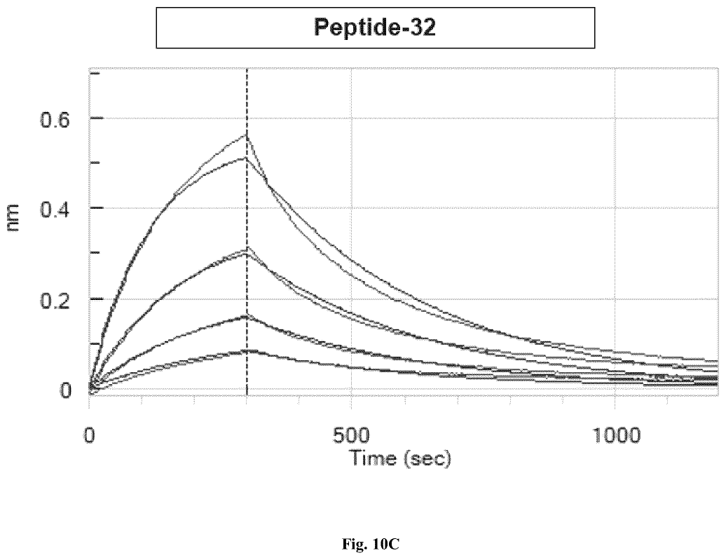

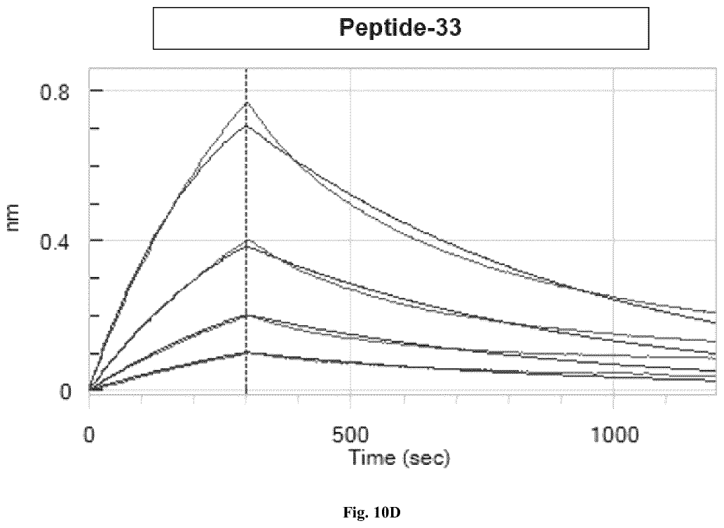

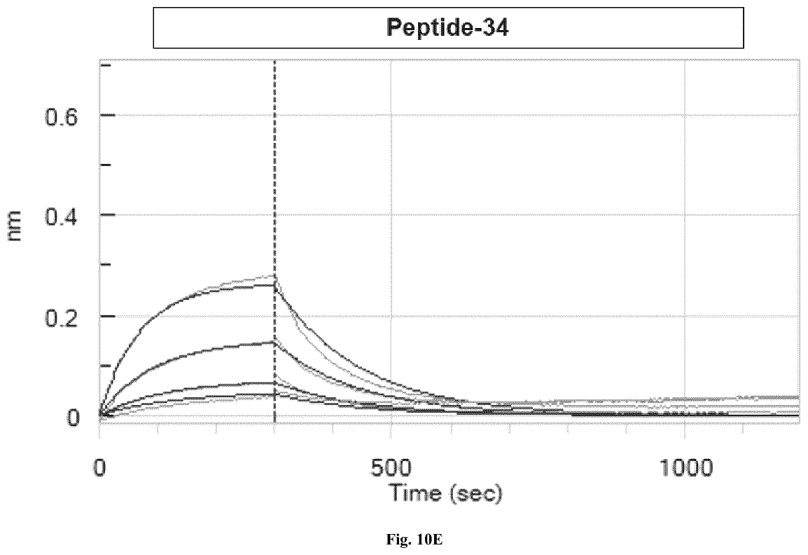

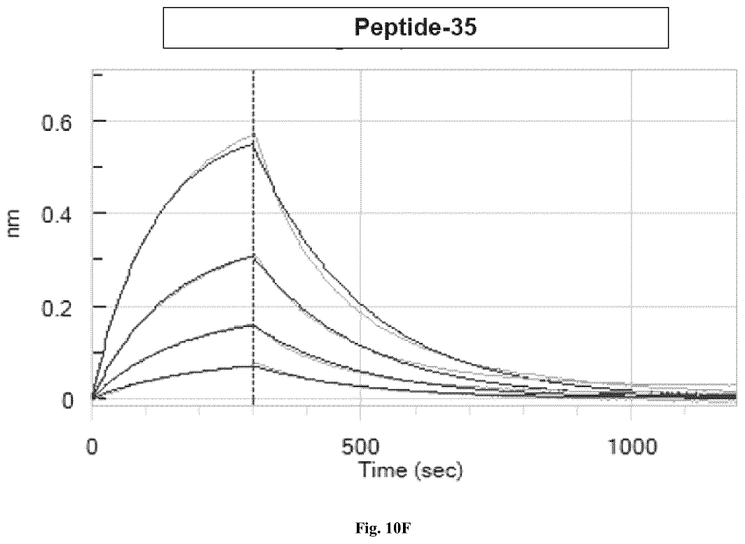

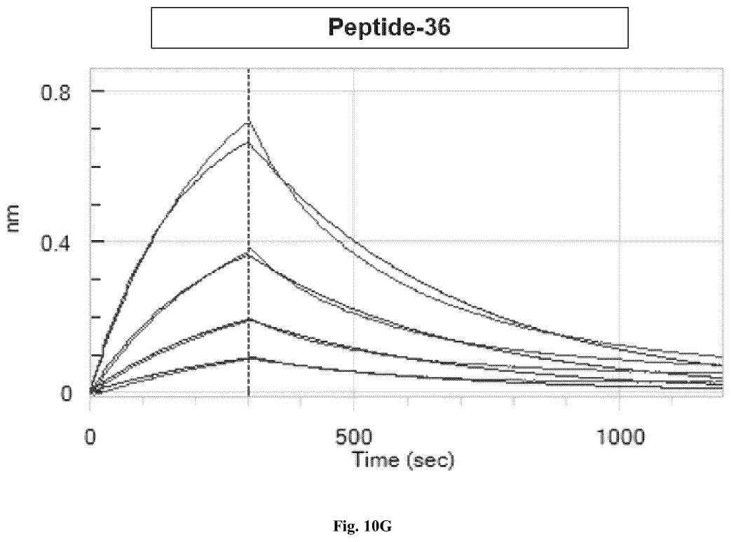

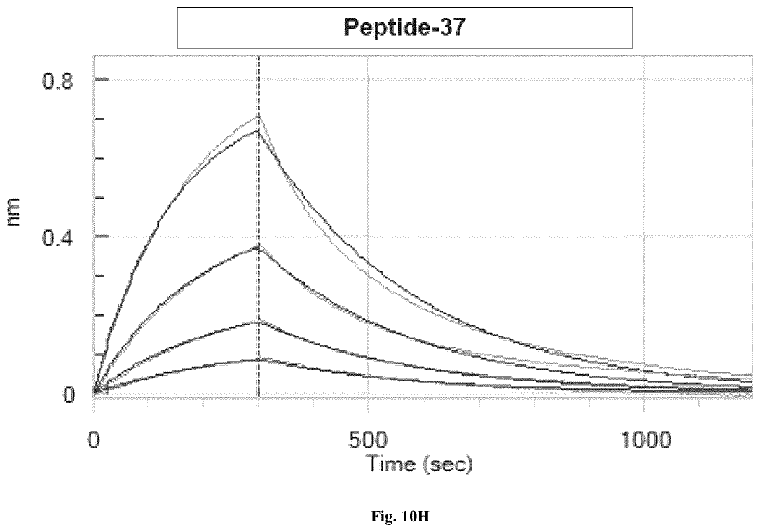

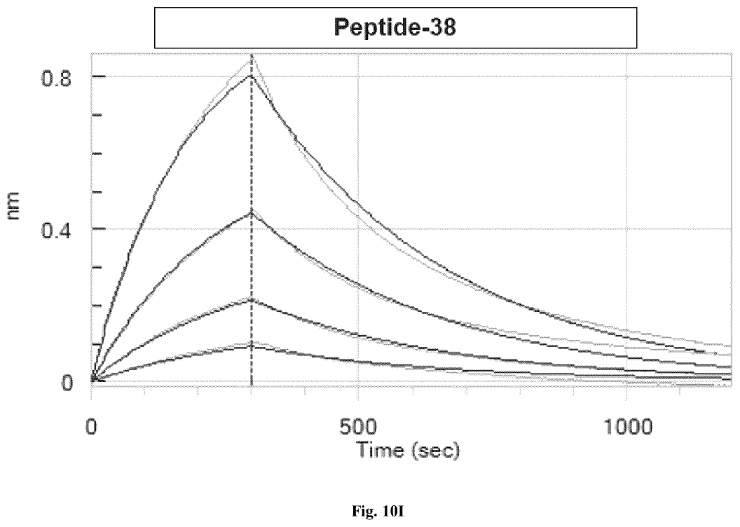

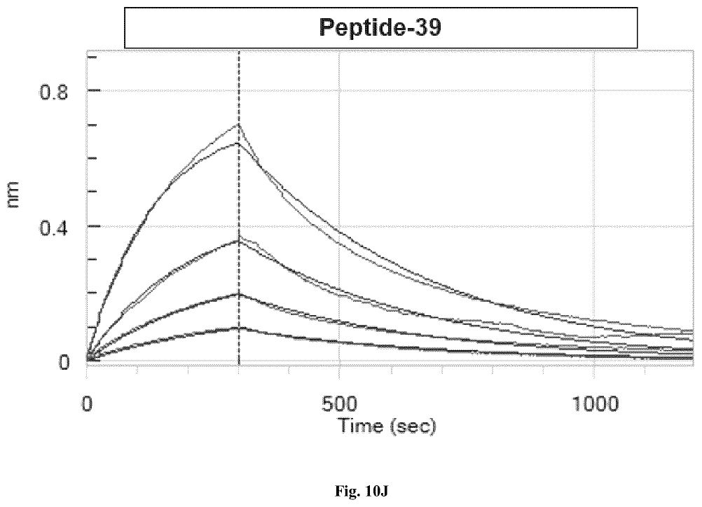

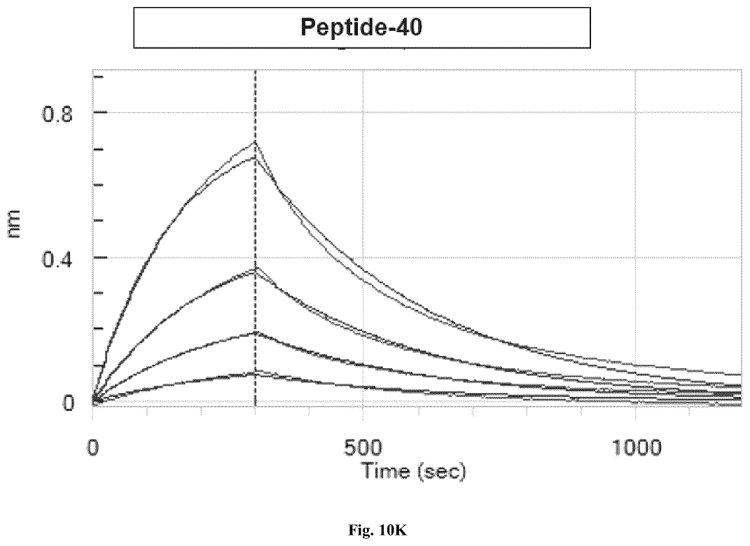

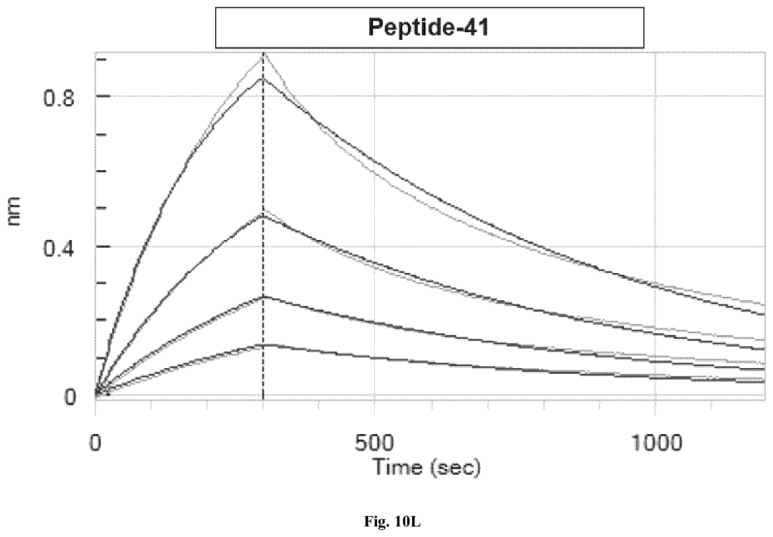

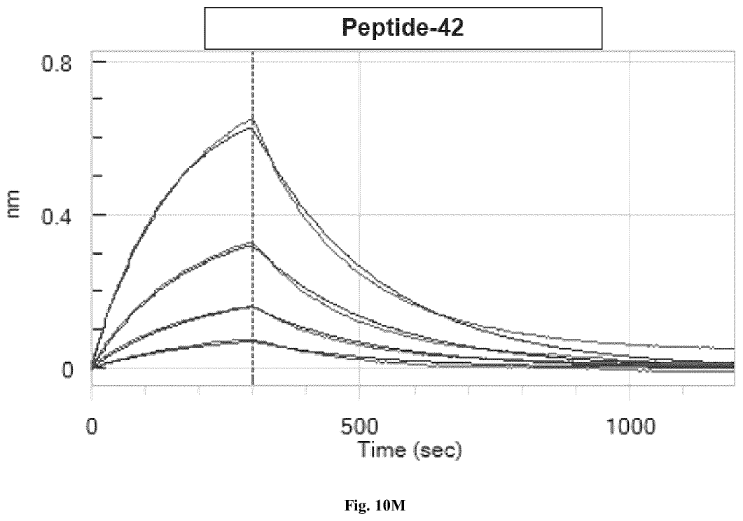

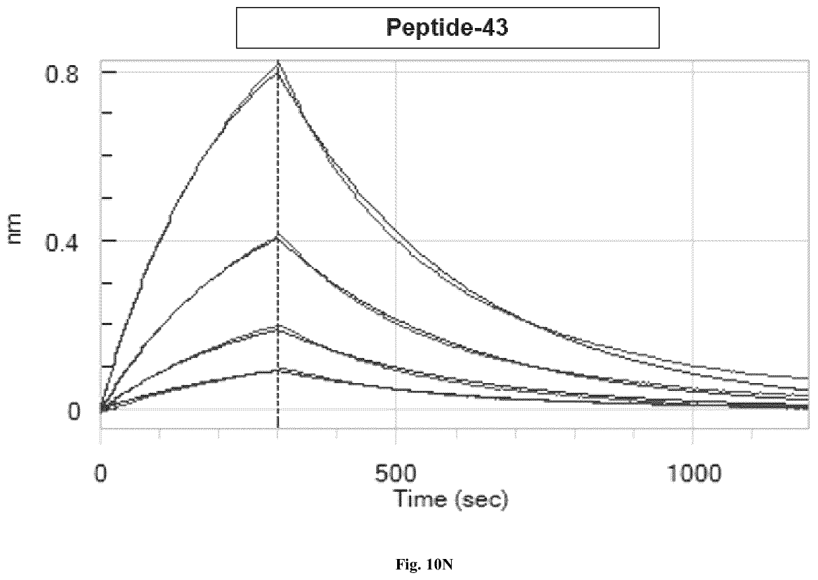

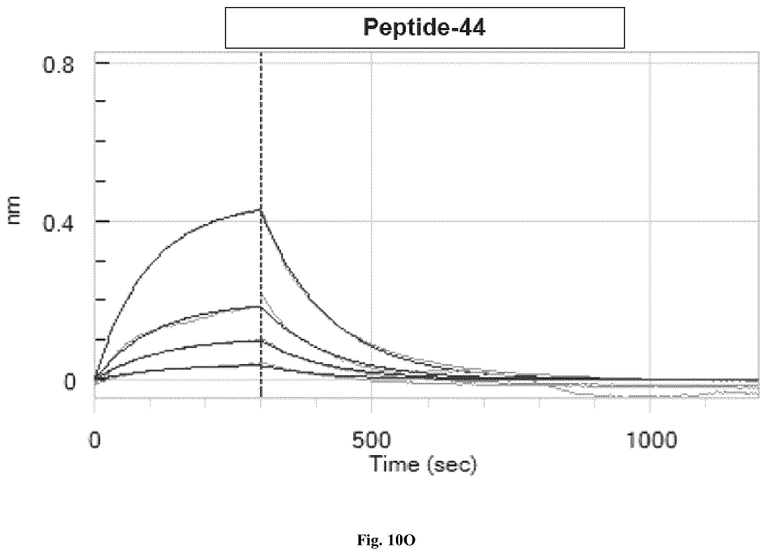

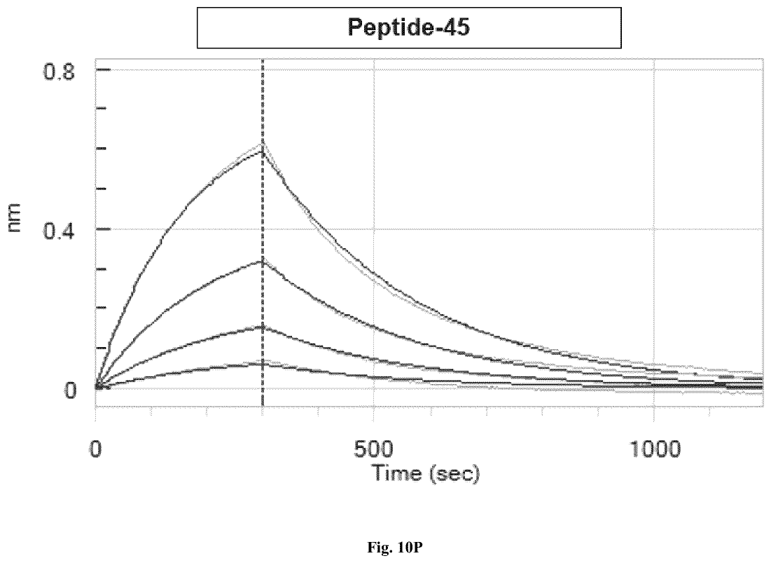

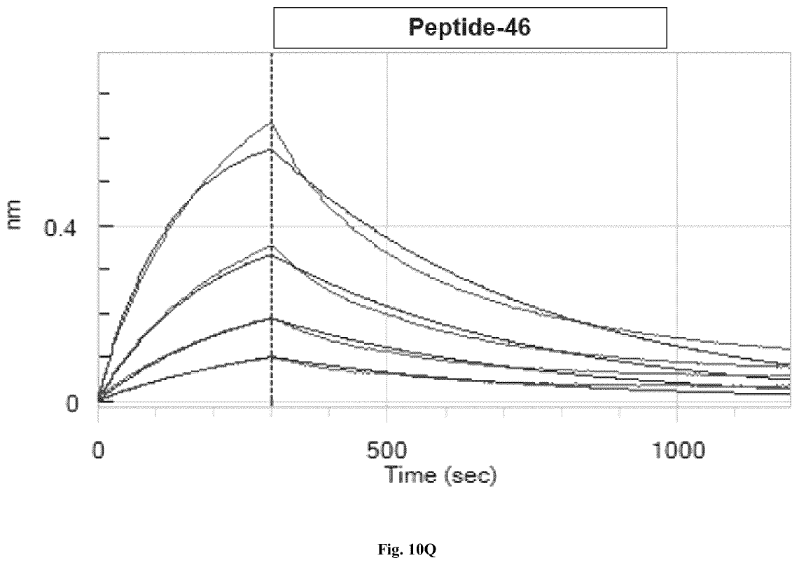

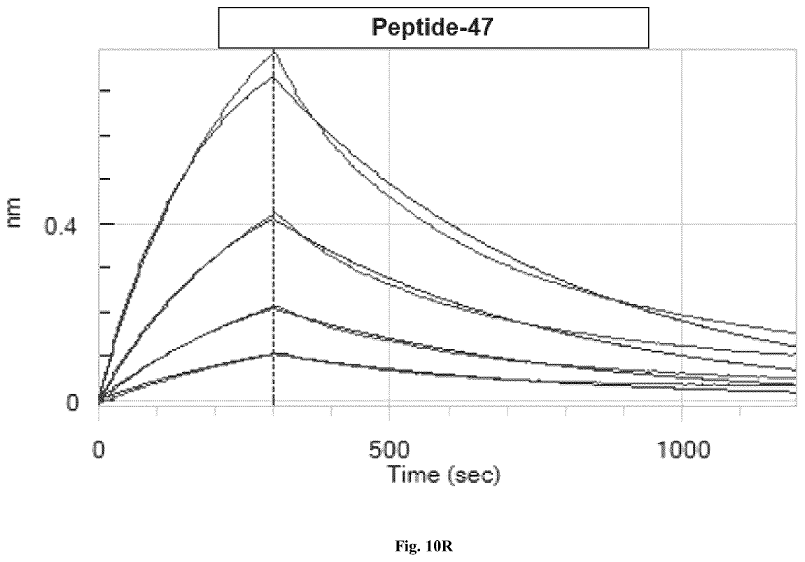

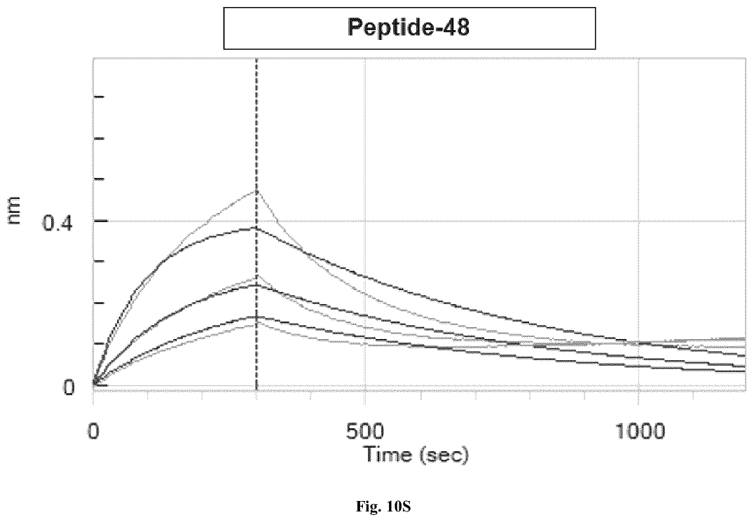

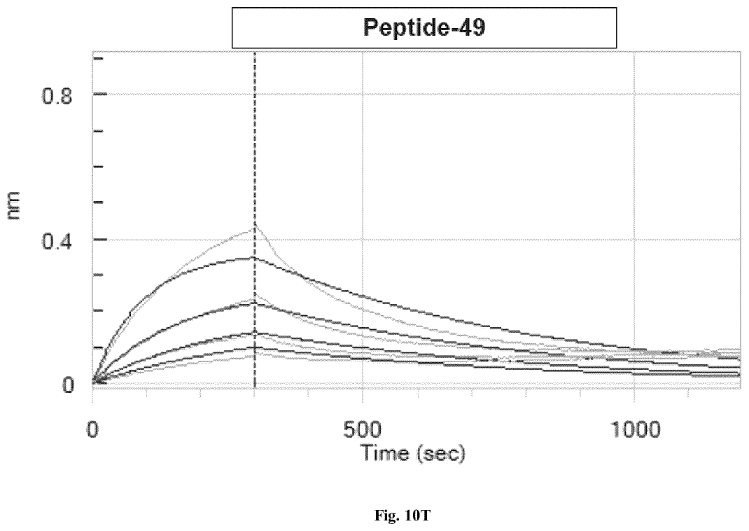

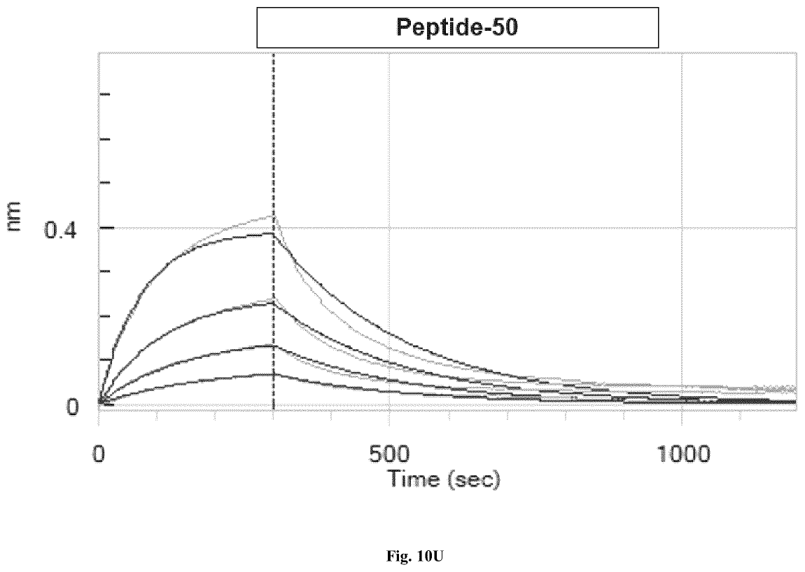

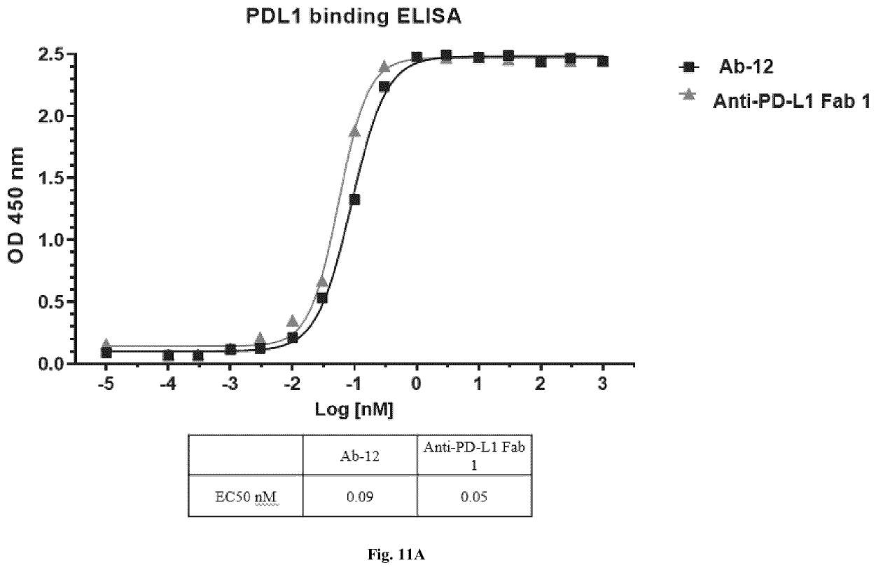

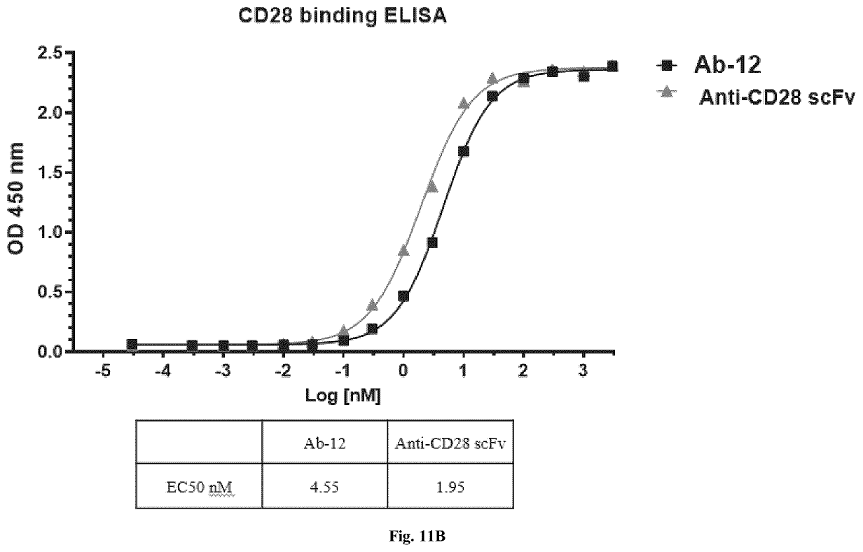

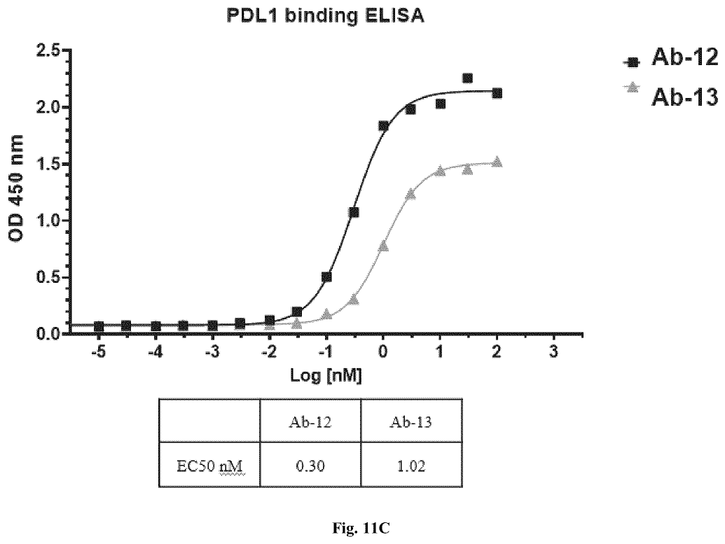

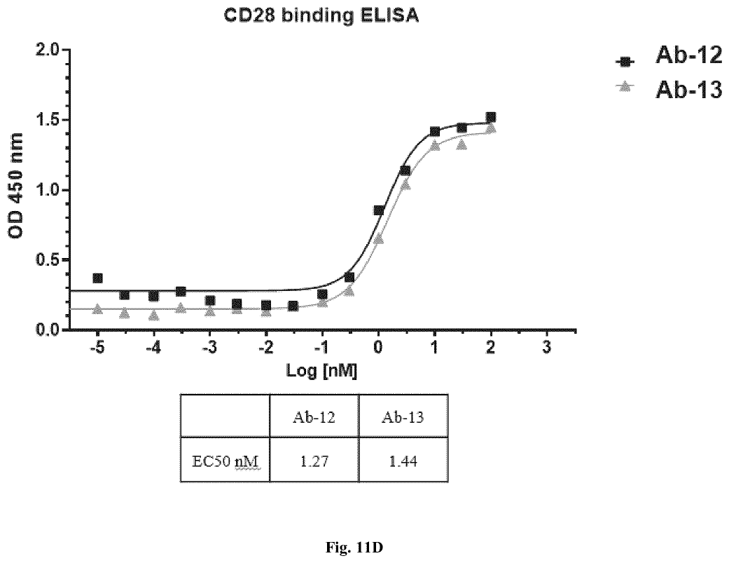

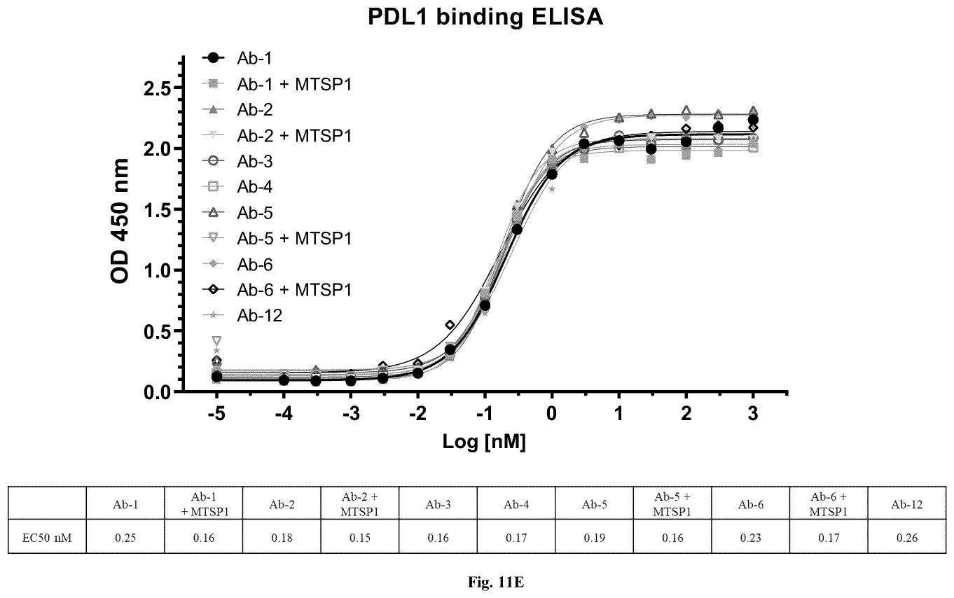

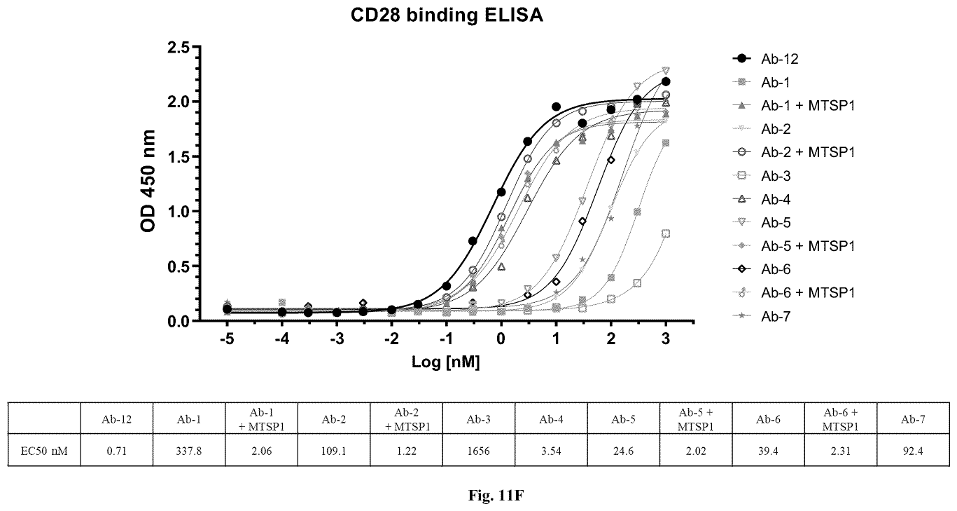

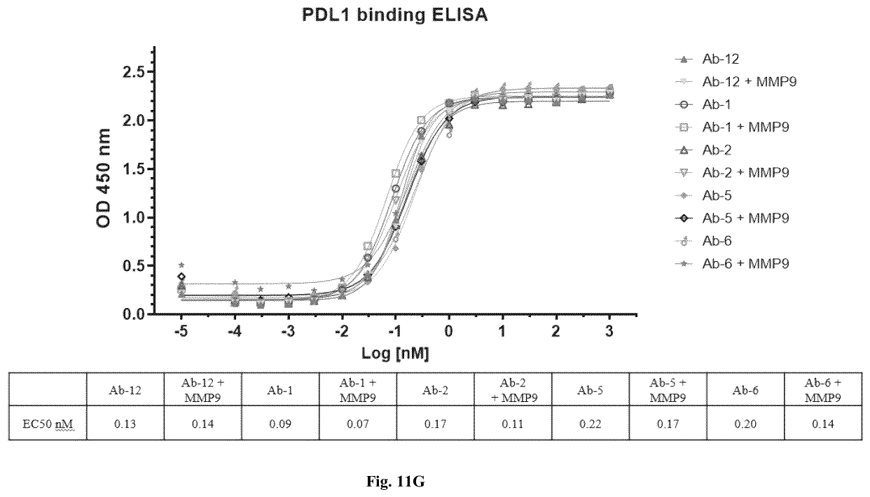

Kinetic binding of anti-CD28 scFv to peptides were evaluated by bio-layer interferometry using an Octet RED96 instrument. Briefly, streptavidin biosensors were loaded with biotinylated peptides and baselined in buffer. Anti-CD28 scFv or Ab-12 were titrated in solution at 100 nM, 50 nM, 25 nM, and 12.5 nM, then associated onto the peptide loaded sensors. After a short association period, sensors were transferred into buffer and the dissociation of bound anti-CD28 scFv was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, KD. FIGS. 10A-10F illustrate kinetic binding of anti-CD28 scFv binding to peptides as measured by Octet. FIGS. 10G-10U illustrate kinetic binding of peptides to the anti-CD28 scFv as measured by Octet.

TABLE 21
Timing and Steps of Assay

| Step | Time |
|---|---|
| Baseline: Buffer | 60 sec |
| Load: | 300 sec |
| 200 nM Peptide | |
| Baseline: Buffer | 300 sec |
| Association in octet buffer | 300 sec |
| 100 nM CD28 scFv | |
| 50 nM CD28 scFv | |
| 25 nM CD28 scFv | |
| 12.5 nM CD28 scFv | |
| Dissociation: Buffer | 900 sec |

TABLE 22
Summary of Kinetic Data

| | Loading Sample | | | |
|---|---|---|---|---|
| Sample ID | ID | KD (M) | kon(1/Ms) | kdis(1/s) |
| Anti-CD28 | Peptide-31 | 7.18E−08 | 5.14E+04 | 3.69E−03 |
| scFv | | | | |
| Anti-CD28 | Peptide-32 | 4.99E−08 | 5.91E+04 | 2.95E−03 |
| scFv | | | | |
| Anti-CD28 | Peptide-33 | 5.28E−08 | 2.90E+04 | 1.53E−03 |
| scFv | | | | |
| Anti-CD28 | Peptide-34 | 8.88E−08 | 7.70E+04 | 6.84E−03 |
| scFv | | | | |
| Peptide-35 | Peptide-35 | 1.27E−07 | 3.90E+04 | 4.97E−03 |
| Anti-CD28 | Peptide-36 | 6.87E−08 | 3.67E+04 | 2.52E−03 |
| scFv | | | | |
| Anti-CD28 | Peptide-37 | 9.78E−08 | 3.59E+04 | 3.51E−03 |
| scFv | | | | |
| Anti-CD28 | Peptide-38 | 9.23E−08 | 3.00E+04 | 2.77E−03 |
| scFv | | | | |
| Anti-CD28 | Peptide-39 | 5.19E−08 | 5.11E+04 | 2.65E−03 |
| scFv | | | | |
| Anti-CD28 | Peptide-40 | 7.99E−08 | 3.88E+04 | 3.10E−03 |
| scFv | | | | |
| Anti-CD28 | Peptide-41 | 4.23E−08 | 3.64E+04 | 1.54E−03 |
| scFv | | | | |
| Anti-CD28 | Peptide-42 | 1.52E−07 | 2.84E+04 | 4.33E−03 |
| scFv | | | | |
| Anti-CD28 | Peptide-43 | 1.92E−07 | 1.67E+04 | 3.20E−03 |
| scFv | | | | |
| Anti-CD28 | Peptide-44 | 3.74E−07 | 2.17E+04 | 8.12E−03 |
| scFv | | | | |
| Anti-CD28 | Peptide-45 | 1.34E−07 | 2.71E+04 | 3.64E−03 |
| scFv | | | | |
| Anti-CD28 | Peptide-46 | 3.52E−08 | 6.19E+04 | 2.18E−03 |
| scFv | | | | |
| Anti-CD28 | Peptide-47 | 5.18E−08 | 3.89E+04 | 2.01E−03 |
| scFv | | | | |
| Anti-CD28 | Peptide-48 | 2.11E−08 | 8.89E+04 | 1.87E−03 |
| scFv | | | | |
| Anti-CD28 | Peptide-49 | 2.11E−08 | 8.82E+04 | 1.86E−03 |
| scFv | | | | |
| Anti-CD28 | Peptide-50 | 4.80E−08 | 9.23E+04 | 4.43E−03 |
| scFv | | | | |
| Anti-CD28 | Peptide-9 | 2.78E−07 | 4.15E+04 | 1.15E−02 |
| scFv | | | | |

### Example 7. Binding of PD-L1 and/or CD28 in a Standard ELISA Assay

Antibodies were evaluated for their ability to bind human PD-L1 or CD28 in a standard enzyme linked immunosorbent assay (ELISA) format. Briefly, biotinylated antigen was captured on neutravidin coated plates. Antibodies diluted in buffer were then added to the antigen coated plates. Bound antibodies detected using a standard horse radish peroxidase conjugate secondary antibody. The concentration of antibody required to achieve 50% maximal signal (EC50) was calculated using Graphpad Prism software.

FIG. 11A illustrates binding of Ab-12 and an anti-PD-L1 Fab 1 (SEQ ID NOs: 16 and 17) to PD-L1. FIG. 11B illustrates binding of Ab-12 and an anti-CD28 scFv (SEQ ID NO: 9) to CD28. FIG. 11C illustrates binding of Ab-12 and Ab-13 to PD-L1. FIG. 11D illustrates binding of Ab-12 and Ab-13 to CD28. FIG. 11E illustrates binding of Ab-1, Ab-2, Ab-3, Ab-4, Ab-5, Ab-6, and Ab-12 to PD-L1. In some circumstances, the antibodies are incubated with the protease, MTSP1. FIG. 11F illustrates binding of Ab-12, Ab-1, anti-PD-L1 Fab 1, anti-CD28 scFv, Ab-5, Ab-6, and Ab-7 to CD28. In some circumstances, the antibodies are incubated with MTSP1. FIG. 11G illustrates binding of Ab-12, Ab-2, Ab-1, Ab-5, and Ab-6 to PD-L1. In some circumstances, the antibodies are incubated with MMP9. FIG. 1113 illustrates binding of Ab-12, Ab-1, Ab-2, Ab-5, and Ab-6 to CD28. In some circumstances, the antibodies are incubated with MMP9. FIG. 11I illustrates binding of Ab-12, Ab-8, Ab-9, Ab-10, and Ab-11 to CD28. In some circumstances, the antibodies are incubated with MTSP1. FIG. 11J illustrates binding of Ab-12, Ab-5, Ab-1, and Ab-9 to CD28. FIG. 11K illustrates binding of Ab-12, Ab-5, Ab-1, and Ab-9 to PD-L1. FIG. 11L illustrates binding of Ab-12, Ab-9, and Ab-9+MTSP1 to PD-L1. FIG. 11M illustrates binding of Ab-12, Ab-9, and Ab-9+MTSP1 to CD28.

### Example 8. Immune Cell Activation Assays

This example demonstrates activation of human PBMCs using target coated beads and titrated test compounds. An exemplary schema of the assay is seen in FIG. 12E.

Briefly, immune cell activation was measured via IL-2 release after co-culture of target coated beads and PBMCs. M280 magnetic streptavidin beads were treated with soluble biotinylated PD-L1 and soluble biotinylated TROP2. M280 beads were washed and seeded in a 96 well plate at 200,000 beads per well. Compounds were then titrated as single agents and in combination and were then added to the wells followed by 100,000 PBMCs. Human T cell activator CD3/CD28 beads (Invitrogen) were used as a positive control in the absence of compound. After 48 hours of co-culture, cytokines were measured in the supernatant using Cytometric Bead Array (CBA) Kit from BD Biosciences. The concentration of cytokines was calculated using a standard curve per manufacturer's instructions.

FIGS. 12A-12C show data for compounds, Ab-12, an anti-PD-L1×CD28 non-masked antibody in Vh format (sequences provided below); Ab-5, an anti-PD-L1×CD28 antibody that is masked with Peptide-9; Ab-5 incubated with protease MTSP1, Ab-12 in combination with Ab-14, an anti-TROP2 T cell engager (sequence provided below); Ab-5 in combination with Ab-14, Ab-5 in combination with Ab-14 and incubated with protease MTSP1, and Ab-14 alone. FIG. 12A shows data for IL-2. FIG. 12B shows data for IFNγ. FIG. 12C shows data for TNFα. FIG. 12D shows data for compounds Ab-12, Ab-13 an anti-PD-L1×CD28 non-masked antibody in VI format (sequence provided below), and masked anti-PD-L1×CD28 antibodies Ab-8, Ab-10, Ab-9, and Ab-11 in combination with Ab-14, with or without incubation of the protease MTSP1.

TABLE 23
Amino acid sequences of Ab-12, Ab-13, and Ab-14

| | Amino Acid Sequence | SEQ ID |
|---|---|---|
| Construct Description | (N to C) | NO: |
| Ab-12 LC | EIVLTQSPATLSLSPGERATLSC | 20 |
| PDL1xCD28 non- | RASQSVSSYLAWYQQKPGQA | |
| masked (Vh) | PRLLIYDASNRATGIPARFSGS | |
| | GSGTDFTLTISSLEPEDFAVYY | |
| | CQQRSNWPTFGQGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| Ab-12 HC | QVQLVQSGAEVKKPGASVKV | 21 |
| PDL1xCD28 non- | SCKASGYTFTSYYIHWVRQAP | |
| masked (Vh) | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGGG | |
| | SGGGGSGGGGSDIQMTQSPSS | |
| | LSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTYP | |
| | YTFGGGTKVEIKGGGGSQVQL | |
| | VQSGAEVKKPGSSVKVSCKTS | |
| | GDTFSTYAISWVRQAPGQGLE | |
| | WMGGIIPIFGKAHYAQKFQGR | |
| | VTITADESTSTAYMELSSLRSE | |
| | DTAVYFCARKFHFVSGSPFGM | |
| | DVWGQGTTVTVSSASTKGPSV | |
| | FPLAPSSKSTSGGTAALGCLVK | |
| | DYFPEPVTVSWNSGALTSGVH | |
| | TFPAVLQSSGLYSLSSVVTVPS | |
| | SSLGTQTYICNVNHKPSNTKV | |
| | DKKVEPKSC | |
| Ab-13 LC | QVQLVQSGAEVKKPGASVKV | 22 |
| PDL1xCD28 non- | SCKASGYTFTSYYIHWVRQAP | |
| masked (VL) | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGGG | |
| | SGGGGSGGGGSDIQMTQSPSS | |
| | LSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTYP | |
| | YTFGGGTKVEIKGGGGSEIVLT | |
| | QSPATLSLSPGERATLSCRASQ | |
| | SVSSYLAWYQQKPGQAPRLLI | |
| | YDASNRATGIPARFSGSGSGT | |
| | DFTLTISSLEPEDFAVYYCQQR | |
| | SNWPTFGQGTKVEIKRTVAAP | |
| | SVFIFPPSDEQLKSGTASVVCL | |
| | LNNFYPREAKVQWKVDNALQ | |
| | SGNSQESVTEQDSKDSTYSLSS | |
| | TLTLSKADYEKHKVYACEVT | |
| | HQGLSSPVTKSFNRGEC | |
| Ab-13 HC | QVQLVQSGAEVKKPGSSVKVS | 23 |
| PDL1xCD28 non- | CKTSGDTFSTYAISWVRQAPG | |
| masked (VL) | QGLEWMGGIIPIFGKAHYAQK | |
| | FQGRVTITADESTSTAYMELSS | |
| | LRSEDTAVYFCARKFHFVSGS | |
| | PFGMDVWGQGTTVTVSSAST | |
| | KGPSVFPLAPSSKSTSGGTAAL | |
| | GCLVKDYFPEPVTVSWNSGAL | |
| | TSGVHTFPAVLQSSGLYSLSSV | |
| | VTVPSSSLGTQTYICNVNHKPS | |
| | NTKVDKKVEPKSC | |
| Ab-14 LC | QTVVTQEPSLTVSPGGTVTLT | 202 |
| TROP2 T cell engager | CRSSTGAVTTSNYANWVQQK | |
| | PGQAPRGLIGGTNKRAPGTPA | |
| | RFSGSLLGGKAALTLSGVQPE | |
| | DEAEYYCALWYSNLWVFGGG | |
| | TKLTVLGGGGSGGGGSGGGG | |
| | SEVQLVESGGGLVQPGGSLKL | |
| | SCAASGFTFNTYAMNWVRQA | |
| | PGKGLEWVARIRSKYNNYAT | |
| | YYADSVKDRFTISRDDSKNTA | |
| | YLQMNNLKTEDTAVYYCVRH | |
| | GNFGNSYVSWFAYWGQGTLV | |
| | TVSSGGGGSDIQLTQSPSSLSA | |
| | SVGDRVSITCKASQDVSIAVA | |
| | WYQQKPGKAPKLLIYSASYRY | |
| | TGVPDRFSGSGSGTDFTLTISS | |
| | LQPEDFAVYYCQQHYITPLTF | |
| | GAGTKVEIKRTVAAPSVFIFPP | |
| | SDEQLKSGTASVVCLLNNFYP | |
| | REAKVQWKVDNALQSGNSQE | |
| | SVTEQDSKDSTYSLSSTLTLSK | |
| | ADYEKHKVYACEVTHQGLSS | |
| | PVTKSFNRGEC | |
| Ab-14 HC | QVQLQQSGSELKKPGASVKVS | 203 |
| TROP2 T cell engager | CKASGYTFTNYGMNWVKQAP | |
| | GQGLKWMGWINTYTGEPTYT | |
| | DDFKGRFAFSLDTSVSTAYLQI | |
| | SSLKADDTAVYFCARGGFGSS | |
| | YWYFDVWGQGSLVTVSSAST | |
| | KGPSVFPLAPSSKSTSGGTAAL | |
| | GCLVKDYFPEPVTVSWNSGAL | |
| | TSGVHTFPAVLQSSGLYSLSSV | |
| | VTVPSSSLGTQTYICNVNHKPS | |
| | NTKVDKKVEPKSC | |

### Example 9. Immune Cell Activation Measured by IL-2 Release

Immune cell activation was measured via IL-2 release after co-culture of target coated beads and PBMCs. Briefly, M280 magnetic streptavidin beads were treated with soluble biotinylated PD-L1 and soluble biotinylated TROP2. M280 beads were washed and seeded in a 96 well plate at 200,000 beads per well. Compounds were then titrated as single agents and in combination then added to the wells followed by 100,000 human or cynomolgus monkey PBMCs. After 48 hours of co-culture, cytokines were measured in the supernatant using Cytometric Bead Array (CBA) Kit from BD Biosciences. The concentration of cytokines was calculated using a standard curve per manufacturer's instructions. FIG. 13A shows data for test compounds Ab-14 in combination with Ab-9 and Ab-14 in combination with Ab-12. FIG. 13B shows data for Ab-14 in combination with Ab-12 and Ab-14 in combination with Ab-9, and Ab-14 alone.

### Example 10. Activation of hPBMCs in Co-Culture Assays with LNCaP Cells

Compounds were evaluated in a functional in vitro tumor cell killing and cytokine release assays using the PD-L1 positive tumor cell line, LNCaP. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. Tumor cells were added and allowed to adhere overnight on a 96 well E-Plate. The following day compounds as single agents or in combination with a T cell engager, a T cell engager masked with a peptide, or pre-cleaved T cell engager masked with a peptide were titrated in human serum supplemented medium along with PBMCs and added to the wells. Cell index measurements were taken every 10 minutes for an additional 120 hours. The cell index times number of hours (tumor cell growth kinetics) was then plotted versus concentration of polypeptide complex where the concentration required to reduce the tumor growth 50% (IC50) was calculated using Graphpad Prism software. Cytokines were measured at study endpoint using the Th1/Th2/Th17 cytometric bead array from BD Biosciences.

FIG. 14A shows data for Ab-12 at various concentrations plotted against Ab-15, an anti-PSMA T cell engager, the sequence of which is provided below. FIG. 14B shows data for Ab-5 at various concentrations plotted against Ab-15. FIG. 14C shows data for Ab-5 at various concentrations with MTSP1 plotted against Ab-15. FIG. 14D shows data for Ab-5 at various concentrations plotted against Ab-16, an anti-PSMA T cell engager masked with a peptide, the sequence of which is provided below. FIG. 14E shows data for MTSP1 treated Ab-5 at various concentrations plotted against MTSP1 treated Ab-16. FIG. 14F shows data for Ab-12 at various concentrations plotted against Ab-15. The data demonstrate that the test compounds synergize with a T cell engager to enhance tumor cell killing in the presence of human PBMCs.

TABLE 24
Amino acid sequences of Ab-15 and Ab-16

| | Amino Acid Sequence | SEQ ID |
|---|---|---|
| Construct Description | (N to C) | NO: |
| Ab-15 LC | EVQLVESGGGLVQPGGSLKLS | 173 |
| Anti-PSMA T cell | CAASGFTFNKYAMNWVRQAP | |
| engager | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSDIQMTQSPSSLSAS | |
| | VGDRVTITCRASQGISNYLAW | |
| | YQQKTGKVPKFLIYEASTLQS | |
| | GVPSRFSGGGSGTDFTLTISSL | |
| | QPEDVATYYCQNYNSAPFTFG | |
| | PGTKVDIKRTVAAPSVFIFPPS | |
| | DEQLKSGTASVVCLLNNFYPR | |
| | EAKVQWKVDNALQSGNSQES | |
| | VTEQDSKDSTYSLSSTLTLSKA | |
| | DYEKHKVYACEVTHQGLSSP | |
| | VTKSFNRGEC | |
| Ab-15 HC | QVQLVESGGGVVQPGRSLRLS | 174 |
| Anti-PSMA T cell | CAASGFAFSRYGMHWVRQAP | |
| engager | GKGLEWVAVIWYDGSNKYYA | |
| | DSVKGRFTISRDNSKNTQYLQ | |
| | MNSLRAEDTAVYYCARGGDF | |
| | LYYYYYGMDVWGQGTTVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |
| Ab-16 LC | EVQLVESGGGLVQPGGSLRLS | 175 |
| Anti-PSMA masked T | CAASGSTFYTAVMGWVRQAP | |
| cell engager | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSDIQMTQSPSSLSASVGDRVT | |
| | ITCRASQGISNYLAWYQQKTG | |
| | KVPKFLIYEASTLQSGVPSRFS | |
| | GGGSGTDFTLTISSLQPEDVAT | |
| | YYCQNYNSAPFTFGPGTKVDI | |
| | KRTVAAPSVFIFPPSDEQLKSG | |
| | TASVVCLLNNFYPREAKVQW | |
| | KVDNALQSGNSQESVTEQDSK | |
| | DSTYSLSSTLTLSKADYEKHK | |
| | VYACEVTHQGLSSPVTKSFNR | |
| | GEC | |
| Ab-16 HC | QVQLVESGGGVVQPGRSLRLS | 176 |
| Anti-PSMA masked T | CAASGFAFSRYGMHWVRQAP | |
| cell engager | GKGLEWVAVIWYDGSNKYYA | |
| | DSVKGRFTISRDNSKNTQYLQ | |
| | MNSLRAEDTAVYYCARGGDF | |
| | LYYYYYGMDVWGQGTTVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |

### Example 11. Proof of Concept Study that Anti-PD-L1×CD28 Antibodies Enhance T Cell Activation in Combination with a T Cell Engager

Immune cell activation was measured via IL-2 induction after co-culture PBMCs with MDAMB231 tumor cells. Briefly, 30,000 MDAMB231 cells and 90,000 PBMCs were co-cultured in a 96 well plate. Compounds were then titrated as single agents and in combination then added to the wells. After 72 hours of co-culture, cytokines were measured in the supernatant using Cytometric Bead Array (CBA) Cytokine Kit from BD Biosciences. FIG. 15A illustrates a cartoon configuration of a multispecific antibody that targets CD28 and PD-L1 that is administered in combination with a T cell engager (TCE) that targets a tumor associated antigen (TAA) such as TROP2 and CD3 of T cell. FIG. 15B illustrates immune cell activation measured via IL-2 induction after co-culture PBMCs with MDAMB231 tumor cells. Shown are plots for various combinations of Ab-14, anti-PD-L1 Fab 1, Ab-9, and Ab-12.

### Example 12. Activation of hPBMCs in Co-Culture Assays with MDAMB231 Tumor Cells

Immune cell activation was measured via IL-2 induction after co-culture PBMCs with MDAMB231 tumor cells. Briefly, 30,000 MDAMB231 cells and 90,000 PBMCs were co-cultured in a 96 well plate. Compounds were then titrated as single agents and in combination then added to the wells. After 72 hours of co-culture, cytokines were measured in the supernatant using Cytometric Bead Array (CBA) Cytokine Kit from BD Biosciences. FIG. 16A shows Ab-9 in combination with Ab-14 and Ab-12 in combination with Ab-14 and Ab-14 alone. FIG. 16B shows a schematic of the assay. FIG. 16C shows Ab-11 in combination with Ab-14 and Ab-13 in combination with Ab-14, and Ab-14 alone.

### Example 13. Pharmacokinetics of Ab-12 and Ab-9 in Cynomolgus Monkey

Pharmacokinetics and exploratory safety of Ab-12 and Ab-9 were evaluated in cynomolgus monkeys. Briefly, cynomolgus monkeys of approximately 3 kg bodyweight were administered test agents as an IV bolus and observed daily for signs of adverse events. No in-life adverse events were observed. After dosing, blood was collected in K2 EDTA tubes at specific timepoints and processed to plasma. Plasma was stored frozen until analysis. Concentration of test agents in plasma was measured via standard ELISA techniques relative to a reference standard diluted in control cyno plasma. Plasma concentration curves were fit to a standard two phase exponential equation representing distribution and elimination phases. Fitting of pharmacokinetics enabled the calculation of Cmax, half-life, volume of distribution, clearance, and 7-day area under the curve (AUC) shown in Table 20. FIG. 17 illustrates pharmacokinetics of Ab-12 and Ab-9 in cynomolgus monkey after a single IV bolus injection.

TABLE 25
FIG. 17 pharmacokinetic summary of Ab-12 and Ab-9

| | Ab-12 | Ab-9 |
|---|---|---|
| Dose (ug/kg) | 30 | 100 |
| C_{MAX} (nM) | 10.7 | 60 |
| t_{1/2} (hr) | 2.15 | 40 |
| Vd (L) | 0.11 | 0.05 |
| CL | 12.1 | 0.3 |
| (ml/hr/kg) | | |

### Example 14. Cytokine Release in Cynomolgus Monkey after Single IV Bolus Injection of Ab-12 and Ab-9

Cytokine release after Ab-12 or Ab-9 administration by IV bolus was evaluated in cynomolgus monkeys. Briefly, cynomolgus monkeys of approximately 3 kg bodyweight were administered test agents as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for cytokines using a non-human primate cytometric Th1/Th2 bead array kit from BD biosciences following the manufacturer's instructions. Interferon gamma, tumor necrosis factor alpha, interleukin 6, and interleukin 2 levels in plasma were calculated relative to reference standards provided with the bead array kit. FIG. 18A-18C illustrates cytokine release in cynomolgus monkey after a single IV bolus injection of Ab-12 and Ab-9.

### Example 15. Analysis of Liver Enzymes in Cynomolgus Monkey after Single IV Bolus Injection of Ab-12 and Ab-9

Systemic liver enzymes after test agent administration by IV bolus was evaluated in cynomolgus monkeys. Briefly, cynomolgus monkeys of approximately 3 kg bodyweight were administered test agents as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for the presence of liver enzymes aspartate transaminase (AST) and alanine aminotransferase (ALT) as signs of potential liver toxicity. AST and ALT were quantified following the instructions provided in a commercially available kit from Millipore. AST and ALT levels were calculated according to manufacturer's instructions relative to a positive control reference standard. FIG. 19A-19D illustrate serum liver enzymes in cynomolgus monkey after a single IV bolus injection of Ab-12 or Ab-9.

### Example 16. Binding of Ab-12, Ab-9, and Ab-19 to Human and Monkey T Cells

PBMCs derived from blood of cynomolgus monkey or healthy human donors were seeded in a flat-bottom 96-wells plate at a concentration of 1×10\hat{ }6 per ml in total of 100 uL. Cells were pelleted and stained with a live/dead exclusion dye for 15 min, at room temperature in the dark. After two rounds of washing with PBS, cells were stained with antibodies against the following surface markers: CD3, CD4, CD8 and PD-1. Surface marker staining was performed at +4° C. for 20 min, after which cells were washed with PBS. Prepared dilutions of test compounds in PBS were added to cells in a final volume of 100 uL and incubated for 1h in a CO2, 37° C. incubator. To detect cell surface-bound test compounds, cells were washed 3 times with PBS and incubated with Alexa fluor 647-conjugated secondary goat anti-human IgG (H+L) antibody for 30 min at +4° C. After washing with PBS cells were fixed and analyzed using BD FACSymphony flow cytometer. Data was processed and analyzed using FlowJo software. Geometric mean fluorescent intensities (GMFI) of Alexa fluor-647 were used to calculate percent of CD28 binding and plot the binding curve. FIGS. 20A-20D illustrate binding results of Ab-12 (a non-masked antibody that binds to PD-L1 and CD28 in Vh format), Ab-9, and Ab-19 (an antibody that binds to PD-L1 and CD28 in a non-cleavable masked Vh format).

The results demonstrate that masking of the CD28 binding domain reduces the concentration dependent binding to T cells.

### Example 17. PD-1 Reporter Assay with Ab-12, Ab-9, Pembrolizumab, Atezolizumab, and Nivolumab

To evaluate the potency of compounds to antagonize the PD-1/PD-L1 pathway a commercially available bioluminescent cell reporter-based systems were used (Promega J1250).

The PD-1 reporter system relies on a recombinant Jurkat T cell line expressing T cell receptor (TCR), human PD-1, and a luciferase reporter driven by NFAT response element (NFAT-RE). This cell line is combined with artificial antigen presenting cells (aAPCs) (PD-L1 aAPC/CHO-Ki cells), expressing human PD-L1 and engineered cell surface protein designed to activate cognate TCR expressed on the Jurkat reporter cells. When the two types of cells are cocultured, the PD-1/PD-L1 interaction inhibits TCR signaling and NFAT-RE-mediated luminescence. Incubation with anti-PD-(L)1 blocking antibody releases the inhibitory signal and results in TCR activation and NFAT-RE-mediated luminescence.

All the assays were performed according to the manufacturer's instructions. In brief, 40,000 PD-L1 aAPC cells was seeded per well of a 96-well plate and incubated overnight in a 37° C., 5% CO2 humidified incubator. The following day, appropriate serial dilutions of the test articles were prepared in cell culture medium and added to wells containing PD-L1 aAPCs. 90,000 Jurkat reporter cells were resuspended in cell culture medium and added in appropriate wells. Each test condition was setup in quadruplicate. Jurkat PD-1 reporter cells were incubated with PD-L1 aAPCs and test articles for 72h in a 37° C., 5% CO2 humidified incubator. After the 5-hour incubation, Bio-Glo reagent was added to the wells and incubated for 10 min at room temperature. Luminescence was measured using Tecan Spark microplate reader. Logarithmic concentrations of test compounds were plotted against the normalized luminescent signal.

FIG. 21 illustrates results of the PD-1 reporter assay for Ab-12, Ab-9, Pembrolizumab, Atezolizumab, and Nivolumab. The sequences of Pembrolizumab, Atezolizumab, and Nivolumab are provided in Table 26. The results demonstrate that the activity observed in the PD-1 reporter assay was similar across CD28 masked or non-masked molecules.

TABLE 26
Additional Sequences

| | Amino Acid Sequence | SEQ ID |
|---|---|---|
| Construct Description | (N to C) | NO: |
| TGN1412 LC | DIQMTQSPSSLSASVGDRVTIT | 220 |
| | CHASQNIYVWLNWYQQKPGK | |
| | APKLLIYKASNLHTGVPSRFSG | |
| | SGSGTDFTLTISSLQPEDFATY | |
| | YCQQGQTYPYTFGGGTKVEIK | |
| | RTVAAPSVFIFPPSDEQLKSGT | |
| | ASVVCLLNNFYPREAKVQWK | |
| | VDNALQSGNSQESVTEQDSKD | |
| | STYSLSSTLTLSKADYEKHKV | |
| | YACEVTHQGLSSPVTKSFNRG | |
| | EC | |
| TGN1412 HC | QVQLVQSGAEVKKPGASVKV | 221 |
| | SCKASGYTFTSYYIHWVRQAP | |
| | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSASTK | |
| | GPSVFPLAPSSKSTSGGTAALG | |
| | CLVKDYFPEPVTVSWNSGALT | |
| | SGVHTFPAVLQSSGLYSLSSVV | |
| | TVPSSSLGTQTYICNVNHKPSN | |
| | TKVDKKVEPKSCDKTHTCPPC | |
| | PAPELLGGPSVFLFPPKPKDTL | |
| | MISRTPEVTCVVVDVSHEDPE | |
| | VKFNWYVDGVEVHNAKTKPR | |
| | EEQYNSTYRVVSVLTVLHQD | |
| | WLNGKEYKCKVSNKALPAPIE | |
| | KTISKAKGQPREPQVYTLPPSR | |
| | DELTKNQVSLTCLVKGFYPSDI | |
| | AVEWESNGQPENNYKTTPPVL | |
| | DSDGSFFLYSKLTVDKSRWQQ | |
| | GNVFSCSVMHEALHNHYTQK | |
| | SLSLSPGK | |
| Pembrolizumab LC | EIVLTQSPATLSLSPGERATLSC | 222 |
| | RASKGVSTSGYSYLHWYQQK | |
| | PGQAPRLLIYLASYLESGVPAR | |
| | FSGSGSGTDFTLTISSLEPEDFA | |
| | VYYCQHSRDLPLTFGGGTKVE | |
| | IKRTVAAPSVFIFPPSDEQLKSG | |
| | TASVVCLLNNFYPREAKVQW | |
| | KVDNALQSGNSQESVTEQDSK | |
| | DSTYSLSSTLTLSKADYEKHK | |
| | VYACEVTHQGLSSPVTKSFNR | |
| | GEC | |
| Pembrolizumab HC | QVQLVQSGVEVKKPGASVKV | 223 |
| | SCKASGYTFTNYYMYWVRQA | |
| | PGQGLEWMGGINPSNGGTNF | |
| | NEKFKNRVTLTTDSSTTTAYM | |
| | ELKSLQFDDTAVYYCARRDY | |
| | RFDMGFDYWGQGTTVTVSSA | |
| | STKGPSVFPLAPCSRSTSESTA | |
| | ALGCLVKDYFPEPVTVSWNSG | |
| | ALTSGVHTFPAVLQSSGLYSLS | |
| | SVVTVPSSSLGTKTYTCNVDH | |
| | KPSNTKVDKRVESKYGPPCPP | |
| | CPAPEFLGGPSVFLFPPKPKDT | |
| | LMISRTPEVTCVVVDVSQEDP | |
| | EVQFNWYVDGVEVHNAKTKP | |
| | REEQFNSTYRVVSVLTVLHQD | |
| | WLNGKEYKCKVSNKGLPSSIE | |
| | KTISKAKGQPREPQVYTLPPSQ | |
| | EEMTKNQVSLTCLVKGFYPSD | |
| | IAVEWESNGQPENNYKTTPPV | |
| | LDSDGSFFLYSRLTVDKSRWQ | |
| | EGNVFSCSVMHEALHNHYTQ | |
| | KSLSLSLGK | |
| Atezolizumab LC | DIQMTQSPSSLSASVGDRVTIT | 224 |
| | CRASQDVSTAVAWYQQKPGK | |
| | APKLLIYSASFLYSGVPSRFSG | |
| | SGSGTDFTLTISSLQPEDFATY | |
| | YCQQYLYHPATFGQGTKVEIK | |
| | RTVAAPSVFIFPPSDEQLKSGT | |
| | ASVVCLLNNFYPREAKVQWK | |
| | VDNALQSGNSQESVTEQDSKD | |
| | STYSLSSTLTLSKADYEKHKV | |
| | YACEVTHQGLSSPVTKSFNRG | |
| | EC | |
| Atezolizumab HC | EVQLVESGGGLVQPGGSLRLS | 225 |
| | CAASGFTFSDSWIHWVRQAPG | |
| | KGLEWVAWISPYGGSTYYAD | |
| | SVKGRFTISADTSKNTAYLQM | |
| | NSLRAEDTAVYYCARRHWPG | |
| | GFDYWGQGTLVTVSSASTKGP | |
| | SVFPLAPSSKSTSGGTAALGCL | |
| | VKDYFPEPVTVSWNSGALTSG | |
| | VHTFPAVLQSSGLYSLSSVVT | |
| | VPSSSLGTQTYICNVNHKPSNT | |
| | KVDKKVEPKSCDKTHTCPPCP | |
| | APELLGGPSVFLFPPKPKDTLM | |
| | ISRTPEVTCVVVDVSHEDPEV | |
| | KFNWYVDGVEVHNAKTKPRE | |
| | EQYASTYRVVSVLTVLHQDW | |
| | LNGKEYKCKVSNKALPAPIEK | |
| | TISKAKGQPREPQVYTLPPSRE | |
| | EMTKNQVSLTCLVKGFYPSDI | |
| | AVEWESNGQPENNYKTTPPVL | |
| | DSDGSFFLYSKLTVDKSRWQQ | |
| | GNVFSCSVMHEALHNHYTQK | |
| | SLSLSPGK | |
| Nivolumab LC | EIVLTQSPATLSLSPGERATLSC | 226 |
| | RASQSVSSYLAWYQQKPGQA | |
| | PRLLIYDASNRATGIPARFSGS | |
| | GSGTDFTLTISSLEPEDFAVYY | |
| | CQQSSNWPRTFGQGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| Nivolumab HC | QVQLVESGGGVVQPGRSLRLD | 227 |
| | CKASGITFSNSGMHWVRQAPG | |
| | KGLEWVAVIWYDGSKRYYAD | |
| | SVKGRFTISRDNSKNTLFLQM | |
| | NSLRAEDTAVYYCATNDDYW | |
| | GQGTLVTVSSASTKGPSVFPL | |
| | APCSRSTSESTAALGCLVKDY | |
| | FPEPVTVSWNSGALTSGVHTF | |
| | PAVLQSSGLYSLSSVVTVPSSS | |
| | LGTKTYTCNVDHKPSNTKVD | |
| | KRVESKYGPPCPPCPAPEFLGG | |
| | PSVFLFPPKPKDTLMISRTPEV | |
| | TCVVVDVSQEDPEVQFNWYV | |
| | DGVEVHNAKTKPREEQFNSTY | |
| | RVVSVLTVLHQDWLNGKEYK | |
| | CKVSNKGLPSSIEKTISKAKGQ | |
| | PREPQVYTLPPSQEEMTKNQV | |
| | SLTCLVKGFYPSDIAVEWESN | |
| | GQPENNYKTTPPVLDSDGSFF | |
| | LYSRLTVDKSRWQEGNVFSCS | |
| | VMHEALHNHYTQKSLSLSLG | |
| | K | |

### Example 18. CD28 Reporter Assay with Ab-12, Ab-9, Ab-19 and TGN1412

To evaluate the potency of compounds to agonize CD28 co-stimulatory pathway a commercially available bioluminescent cell reporter-based systems were used (Promega JA6701).

The CD28 reporter system relies on a recombinant Jurkat T cell line expressing T cell receptor (TCR), CD3, and CD28 receptors as well as a luciferase reporter driven by a CD28 pathway-dependent promoter. CD28 reporter Jurkat cells were co-cultured with artificial APCs (PD-L1 aAPC/CHO-Ki cells) from PD-1 reporter kit (Promega J1250) that express human PD-L1 and engineered cell surface protein designed to activate cognate TCR expressed on Jurkat reporter cells. In the absence of CD28 agonist antibody, CD28 is not activated and luminescence is low. Incubation with CD28 agonist antibodies induces CD28 pathway and increases luminescence.

All the assays were performed according to the manufacturer's instructions. In brief, 40,000 PD-L1 aAPC cells was seeded per well of a 96-wells plate and incubated overnight in a 37° C., 5% CO2 humidified incubator. The following day, appropriate serial dilutions of the test articles were prepared in cell culture medium and added to wells containing PD-L1 aAPCs. 90,000 Jurkat CD28 reporter cells were resuspended in cell culture medium and added in appropriate wells. Each test condition was setup in quadruplicate. Jurkat CD28 reporter cells were incubated with PD-L1 aAPCs and test articles for 72h in a 37° C., 5% CO2 humidified incubator. After the 5-hour incubation, Bio-Glo reagent was added to the wells and incubated for 10 min at room temperature. Luminescence was measured using Tecan Spark microplate reader. Logarithmic concentrations of test compounds were plotted against the normalized luminescent signal.

FIG. 22 illustrates results of the CD28 reporter assay of Ab-12, Ab-9, Ab-19, and TGN1412. The sequences of TGN1412 are provided in Table 26. The results demonstrate that masked molecules exhibit reduced activity in the CD28 reporter assay compared to the non-masked version.

### Example 19. Functional Activity Assay as Single Agents or in Combination with Pembrolizumab as Measured by IL-2 Induction in Tumor Cells in a Mixed Lymphocyte Reaction (MLR) System

To evaluate the functional activity of test compounds as single agents or in combination with Pembrolizumab a mixed lymphocyte reaction (MLR) system was established based on coculture of healthy human donor PBMCs and triple negative breast tumor cell line, MDA-MB231. Where indicated test compounds were pre-treated with protease MMP9 or MTSP1. To ensure presence of active antigen presentation, MDA-MB231 cells were loaded with HLA-A*0201-restricted CMV peptide (NLVPMVATV). To ensure T cell recognition of this peptide, PBMCs derived from HLA-A*0201+CMV+donors were used in the MLR reaction. In brief, 50,000 MDA-MB231 cells were seeded per well of 96-well plates in presence of 1 ug/mL of CMV peptide, and incubated overnight in a 37° C., 5% CO2 humidified incubator. The following day, appropriate serial dilutions of the test articles were prepared in a complete cell culture medium and added to wells containing peptide-coated MDA-MB231 tumor cells. Healthy donor PBMCs (150,000 cells) were resuspended in cell culture medium and added in appropriate wells. Each test condition was setup in quadruplicate. PBMCs were incubated with test articles and MDA-MB231 cells for 72h in a 37° C., 5% CO2 humidified incubator. Cell culture supernatants were harvested and stored at −20° C. until cytokine analysis. Soluble IL-2 was measured in cell culture supernatants using MSD platform. IL-2 induction was plotted against logarithmic concentration of test compounds.

FIG. 23A illustrates results of IL-2 induction of Ab-12, Ab-9, and Ab-19. Ab-9 is also shown in combination with MMP9 or MTSP1. FIG. 23B illustrates results of Ab-12 in combination with Pembrolizumab, Ab-9 in combination with Pembrolizumab, Ab-9 in combination with MMP9 and Pembrolizumab, and Ab-9 in combination with MTSP1 and Pembrolizumab.

### Example 20. Binding of Ab-12, Ab-9, and Ab-19 to PD-L1 on Tumor Cells

Binding of test compounds to PD-L1 expressed on tumor cells was evaluated using PD-L1-expressing MDA-MB231 tumor cell line. In brief, 100,000 MDA-MB231 cells were seeded per well of a 96-wells plate and incubated for 1h in a 37° C., 5% CO2 humidified incubator with appropriate serial dilutions of test compounds. To detect cell surface-bound test compounds, cells were washed 3 times with PBS and incubated with Alexa fluor 647-conjugated secondary anti-human IgG antibody for 30 min at +4° C. After washing with PBS cells were fixed and analyzed using BD FACSymphony. Data was processed and analyzed using FlowJo software. Geometric mean fluorescent intensities (GMFI) or Alexa fluor-647 were used to calculate percent of PD-L1 binding and plot the binding curve.

FIG. 24 illustrates results of Ab-12, Ab-9, and Ab-19 binding to PD-L1 on PD-L1-expressing MDA MB231 tumor cell line. The results demonstrate that binding to PD-L1 on MDA-MB231 tumor cells is similar across the CD28 masked and non-masked molecules.

### Example 21. Tumor Activated Multispecific Antibodies that Bind to CD28 and PD-L1 in Combination with T Cell Engagers Enhance T Cell Functional Activation

Polypeptide complexes were evaluated in a functional in vitro tumor cell killing and cytokine release assays using the PDL1 positive tumor cell line, CAL27. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. Tumor cells were added and allowed to adhere overnight on a 96 well E-Plate. The following day cleavable or non-cleavable polypeptide complexes as single agents or in combination with a TCE were titrated in human serum supplemented medium along with human PBMCs and added to the wells. Cell index measurements were taken every 10 minutes for an additional 120 hours. The cell index times number of hours (tumor cell growth kinetics) was then plotted versus concentration of polypeptide complex where the concentration required to reduce the tumor growth 50% (IC50) was calculated using Graphpad Prism software. Cytokines were measured at study endpoint using the Th1/Th2/Th17 cytometric bead array from BD Biosciences.

FIG. 25A illustrates a cartoon configuration of a multispecific antibody that targets CD28 and PD-L1 that is administered in combination with a T cell engager that targets a tumor associated antigen (TAA) such as EGFR and CD3 of T cell.

FIG. 25B-25C illustrate tumor cell killing of CAL27 tumor cells by Ab-12, Ab-9, Ab-18 alone or in combination with 1 pM of Ab-20, an EGFR T cell engager. Results of the plots are also summarized in Table 28. Table 27 summarizes the PD-L1 and EGFR densities on CAL27 cells.

FIG. 25D-25F illustrate cytokine induction (IFNγ, TNF, and IL-2) by Ab-12 or Ab-12 in combination with 1 pM of Ab-20 when titrated in human serum supplemented medium along with human PBMCs.

FIG. 25G-25I illustrate cytokine induction (IFNγ, TNF, and IL-2) by Ab-9 or Ab-9 in combination with 1 pM of Ab-20 and also Ab-18 or Ab-18 in combination with 1 pM of Ab-20 when titrated in human serum supplemented medium along with human PBMCs.

The results demonstrate that the in vitro functional activity of PD-L1 and CD28 targeted molecules in combination with a T cell engager targeting EGFR and CD3 is mask and cleavage dependent. The non-masked PD-L1×CD28 bispecific exhibits stronger potency than the masked versions and the masked non-cleavable version exhibits weaker activity compared to the masked cleavable version. The difference in activity between masked cleavable and masked non-cleavable molecules implies proteolytic cleavage in the assay coming from either the tumor cells or PBMCs or both.

TABLE 27
CAL27 Densities

| | PD-L1 Density | EGFR Density |
|---|---|---|
| Cell Line | (copies per cell) | (copies per cell) |
| CAL27 | 22,000 | 170,000 |

TABLE 28

| | | |
|---|---|---|
| | With Ab-20 | Absent Ab-20 |
| Compound | (EGFR TCE) | (EGFR TCE) |

| | | | |
|---|---|---|---|
| Ab-12 (non-masked) | 70 | pM | >100,000 pM |
| Ab-9 (cleavable) | 2,226 | pM | >100,000 pM |
| Ab-18 (non-cleavable) | 64,463 | pM | >100,000 pM |

### Example 22. Anti-Tumor Efficacy of Tumor Activated Multispecific Antibodies that Bind to CD28 and PD-L1 in Combination with an Antibody that Binds TROP2 and CD3

Test compounds were tested for anti-tumor activity in a mouse model of triple negative breast cancer. Female NCG mice were subcutaneously inoculated with 5×10⁶ MDAMB231 tumor cells in the rear hind flank. When tumors became palpable (50-80 mm³) mice were randomized into groups (N=10 per group) and administered 15×10⁶ human PBMCs by intraperitoneal injection. When tumors reached 200-300 mm³ test articles were administered to animals every day for 10 days via intravenous injection through the tail vein. Tumor volumes were measured using calipers overtime. Animals were euthanized when tumor volumes reached 2000 mm³ or signs of graft versus host disease were evident. Tumor growth kinetics was evaluated by plotting mean tumor volumes versus time.

FIG. 26 illustrates mean tumor volume after treatment with Ab-22 in combination with Ab-18, or treatment with Ab-21 and Ab-17 in combination, or treatment with Ab-17 alone, or treatment with Ab-21 alone.

The results demonstrate that the in vivo anti-tumor activity of PD-L1 and CD28 targeted molecules in combination with a TROP2 and CD3 targeted molecule is cleavage dependent. While the masked cleavable PD-L1 and CD28 targeted molecule in combination with a TROP2 and CD3 targeted molecule inhibits tumor growth, the masked non-cleavable versions do not inhibit tumor growth in the same combination. Single-agent PD-L1 and CD28 targeted molecule did not inhibit tumor growth due to a lack of immune recognition of the tumor from the engrafted PBMCs. The PBMCs utilized were specifically chosen for their lack of endogenous activity against MDAMB231 tumors in vivo.

### Example 23. Non-Human Primate Studies after IV Dosing of Ab-9 at 1 mg/kg, 5 mg/kg, and 15 mg/kg

Pharmacokinetics and safety of Ab-9 were assessed according to the procedures of Example 13, except with treatment doses at 1 mg/kg, 5 mg/kg, and 15 mg/kg. Cytokine release after administration was measured using a standard Luminex cytokine panel, that included IL-2, IL-10, TNFa, IL-6, and IFNg. Clinical chemistry parameters were measured in NHP serum using a standard panel and method. The clinical chemistry panel included liver enzymes, AST and ALT, as well as total bilirubin (TBIL), creatinine (CRE), and blood urea (UREA) as indirect measures of liver and kidney function.

FIG. 27 illustrates non-human primate pharmacokinetics for dosing at 15 mg/kg, 5 mg/kg, and 1 mg/kg of Ab-9.

FIG. 28A-28E illustrate cytokine release (IFNγ, TNF, IL-2, IL-6, and IL-10) in non-human primates after administration of 15 mg/kg, 5 mg/kg, and 1 mg/kg of Ab-9.

FIG. 29A-29E illustrate non-human primate clinical chemistry results (AST, ALT, TBIL, CRE, urea) for dosing at 15 mg/kg, 5 mg/kg, and 1 mg/kg of Ab-9.

The results demonstrate that no meaningful changes in the measured cytokines or clinical chemistries were induced after dosing up to 15 mg/kg. The maximum tolerated dose (MTD) was not reached in this study.

What is claimed is:

1. An isolated multispecific antibody according to the following formula: P₁-L₁-A₁-L-B (Formula I) wherein A₁ comprises a CD28 binding domain; B comprises a PD-L1 binding domain; L comprises a linker that connects A₁ to B; P₁ comprises a peptide that binds to A₁ and L₁ comprises a linking moiety that connects A₁ to P₁ and is a substrate for a tumor specific protease wherein P₁ comprises an amino acid sequence according to X₁-X₂-X₃-C-X₄-X₅-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q; X₉ is selected from H, N, D, A, S, Y, T, F, V, L, and I; X₁₁, is selected from E, V, L, D, Y, R, Q, H, F, K, A, M, and N; X₁₁ is selected from F, Y, L, W, and V; and X₁₂ is selected from N, A, F, S, Y, H, D, T, and L.

2. The isolated multispecific antibody of claim 1, wherein X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₁, is selected from E, V, L, D, and H; X₁₁ is selected from F, Y, and L; and X₁₂ is selected from N, A, F, S, and Y.

3. The isolated multispecific antibody of claim 2, wherein X₁ is M; X₂ is selected from D and H; X₃ is W; X₄ is P; X₅ is selected from R, T, and I; X₆ is selected from E, D, and Y; X₇ is selected from L, M, and R; X₈ is W; X₉ is selected from H, N, D, and V; X₁₀ is selected from E, V, L, D, and H; X₁₁ is F; and X₁₂ is selected from N, A, and F.

4. The isolated multispecific antibody of claim 1, wherein P₁ comprises an amino acid sequence according to SEQ ID NO: 32 or an amino acid sequence that has 1, 2, or 3 amino acid mutations, substitutions, or deletions relative to SEQ ID NO: 32.

5. The isolated multispecific antibody of claim 1, wherein P₁ comprises an amino acid sequence according to SEQ ID NO: 32.

6. The isolated multispecific antibody of claim 1, wherein P₁ comprises an amino acid sequence according to SEQ ID NO: 138 or an amino acid sequence that has 1, 2, or 3 amino acid mutations, substitutions, or deletions relative to SEQ ID NO: 138.

7. The isolated multispecific antibody of claim 1, wherein P₁ comprises an amino acid sequence according to SEQ ID NO: 138.

8. The isolated multispecific antibody of claim 1, wherein P₁ impairs binding of A₁ to CD28.

9. The isolated multispecific antibody of claim 1, wherein P₁ is bound to A₁ through ionic interactions, electrostatic interactions, hydrophobic interactions, Pi-stacking interactions, and H-bonding interactions, or a combination thereof.

10. The isolated multispecific antibody of claim 1, wherein P₁ is bound to A₁ at or near an antigen binding site.

11. The isolated multispecific antibody of claim 1, wherein P₁ becomes unbound from A₁ when L₁ is cleaved by the tumor specific protease thereby exposing A₁ to CD28.

12. The isolated multispecific antibody of claim 1, wherein P₁ has less than 75% sequence identity to CD28.

13. The isolated multispecific antibody of claim 1, wherein P₁ comprises a de novo amino acid sequence that shares less than 10% sequence identity to CD28.

14. The isolated multispecific antibody of claim 1, wherein P₁ comprise a modified amino acid or non-natural amino acid, or a modified non-natural amino acid, or a combination thereof.

15. The isolated multispecific antibody of claim 1, wherein P₁ does not comprise albumin or an albumin fragment.

16. The isolated multispecific antibody of claim 1, wherein P₁ does not comprise an albumin binding domain.

17. The isolated multispecific antibody of claim 1, wherein L₁ is a peptide sequence having at least 5 to no more than 50 amino acids.

18. The isolated multispecific antibody of claim 1, wherein L₁ is a peptide sequence having at least 10 to no more than 30 amino acids.

19. The isolated multispecific antibody of claim 1, wherein L₁ is a peptide sequence having at least 10 amino acids.

20. The isolated multispecific antibody of claim 1, wherein L₁ is a peptide sequence having at least 18 amino acids.

21. The isolated multispecific antibody of claim 1, wherein L₁ is a peptide sequence having at least 26 amino acids.

22. The isolated multispecific antibody of claim 1, wherein L₁ comprises a formula comprising (G₂S)ₙ, wherein n is an integer from 1 to 3 (SEQ ID NO: 228).

23. The isolated multispecific antibody of claim 1, wherein L₁ comprises a formula comprising (G₂S)ₙ, wherein n is an integer of at least 1.

24. The isolated multispecific antibody of claim 1, wherein L₁ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

25. The isolated multispecific antibody of claim 1, wherein the tumor specific protease is selected from the group consisting of metalloprotease, serine protease, cysteine protease, threonine protease, and aspartic protease.

26. The isolated multispecific antibody of claim 1, wherein L₁ comprises a urokinase cleavable amino acid sequence, a matriptase cleavable amino acid sequence, or a matrix metalloprotease cleavable amino acid sequence.

27. The isolated multispecific antibody of claim 1, wherein L₁ comprises a sequence according to SEQ ID NOs: 18-19, 62-88.

28. The isolated multispecific antibody of claim 1, wherein L₁ is bound to N-terminus of A₁.

29. The isolated multispecific antibody of claim 1, wherein L₁ is bound to C-terminus of A₁.

30. The isolated multispecific antibody of claim 1, wherein the CD28 binding domain comprises a single chain variable fragment, a single domain antibody, a Fab, or a Fab′.

31. The isolated multispecific antibody of claim 30, wherein the CD28 binding domain comprises the single chain variable fragment.

32. The isolated multispecific antibody of claim 1, wherein the PD-L1 binding domain comprises a single chain variable fragment, a single domain antibody, a Fab, or a Fab′.

33. The isolated multispecific antibody of claim 32, wherein the PD-L1 binding domain comprises the Fab or the Fab′.

34. The isolated multispecific antibody of claim 32, wherein the PD-L1 binding domain comprises the Fab or the Fab′ and the CD28 binding domain comprises the single chain variable fragment.

35. The isolated multispecific antibody of claim 32, wherein the PD-L1 binding domain that comprises the Fab or the Fab′ comprises a Fab heavy chain polypeptide comprising a Fab heavy chain variable domain and a Fab light chain polypeptide comprising a Fab light chain variable domain.

36. The isolated multispecific antibody of claim 35, wherein the CD28 binding domain that comprises the single chain variable fragment comprises a scFv heavy chain variable domain and a scFv light chain variable domain.

37. The isolated multispecific antibody of claim 1, wherein the linker connects the C-terminus of A₁ to an N-terminus of B.

38. The isolated multispecific antibody of claim 1, wherein the linker connects the N-terminus of A₁ to a C-terminus of B.

39. The isolated multispecific antibody of claim 36, wherein the linker connects the C-terminus of A₁ to the N-terminus of the Fab heavy chain polypeptide.

40. The isolated multispecific antibody of claim 36, wherein the linker connects the N-terminus of A₁ to the C-terminus of the Fab heavy chain polypeptide.

41. The isolated multispecific antibody of claim 36, wherein the linker connects the C-terminus of A₁ to the N-terminus of the Fab light chain polypeptide.

42. The isolated multispecific antibody of claim 36, wherein the linker connects the N-terminus of A₁ to the C-terminus of the Fab light chain polypeptide.

43. The isolated multispecific antibody of claim 36, wherein the linker connects the Fab light chain polypeptide to the scFv light chain variable domain.

44. The isolated multispecific antibody of claim 36, wherein the linker connects the Fab light chain polypeptide to the scFv heavy chain variable domain.

45. The isolated multispecific antibody of claim 36, wherein the linker connects the Fab heavy chain polypeptide to the scFv light chain variable domain.

46. The isolated multispecific antibody of claim 36, wherein the linker connects the Fab heavy chain polypeptide to the scFv heavy chain variable domain.

47. The isolated multispecific antibody of claim 36, wherein the linker connects the Fab light chain polypeptide to the N-terminus of the scFv light chain variable domain.

48. The isolated multispecific antibody of claim 36, wherein the linker connects the Fab light chain polypeptide to the C-terminus of the scFv light chain variable domain.

49. The isolated multispecific antibody of claim 36, wherein the linker connects the Fab light chain polypeptide to the N-terminus of the scFv heavy chain variable domain.

50. The isolated multispecific antibody of claim 36, wherein the linker connects the Fab light chain polypeptide to the C-terminus of the scFv heavy chain variable domain.

51. The isolated multispecific antibody of claim 36, wherein the linker connects the Fab heavy chain polypeptide to the N-terminus of the scFv light chain variable domain.

52. The isolated multispecific antibody of claim 36, wherein the linker connects the Fab heavy chain polypeptide to the C-terminus of the scFv light chain variable domain.

53. The isolated multispecific antibody of claim 36, wherein the linker connects the Fab heavy chain polypeptide to the N-terminus of the scFv heavy chain variable domain.

54. The isolated multispecific antibody of claim 36, wherein the linker connects the Fab heavy chain polypeptide to the C-terminus of the scFv heavy chain variable domain.

55. The isolated multispecific antibody of claim 1, wherein the linker is at least 5 amino acids in length.

56. The isolated multispecific antibody of claim 1, wherein the linker comprises (G₂S)n, (GS)n, (GSGGS)n (SEQ ID NO: 58), (GGGS)n (SEQ ID NO: 59), (GGGGS)n (SEQ ID NO: 60), and (GSSGGS)n (SEQ ID NO: 61), wherein n is an integer of at least 1.

57. The isolated multispecific antibody of claim 1, wherein L comprises a formula comprising (G2S)n, wherein n is an integer from 1 to 3 (SEQ ID NO: 228).

58. The isolated multispecific antibody of claim 1, wherein the L comprises an amino acid sequence of SEQ ID NO: 18 (GGGGSGGGGSGGGGS) or SEQ ID NO: 19 (GGGGS).

59. The isolated multispecific antibody of claim 36, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

60. The isolated multispecific antibody of claim 36, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5 (KA); and LC-CDR3: SEQ ID NO: 6, and wherein the CDRs comprise from 0-2 amino acid modifications in at least one of said LC-CDR1, LC-CDR2, or LC-CDR3.

61. The isolated multispecific antibody of claim 1, wherein A₁ comprises complementarity determining regions (CDRs): LC-CDR1, LC-CDR2, and LC-CDR3, wherein the LC-CDR1, the LC-CDR2, and the LC-CDR3 of A₁ comprise: LC-CDR1: SEQ ID NO: 4; LC-CDR2: SEQ ID NO: 5 (KA); and LC-CDR3: SEQ ID NO: 6; wherein A₁ comprises complementarity determining regions (CDRs): HC-CDR1, HC-CDR2, and HC-CDR3, wherein the HC-CDR1, the HC-CDR2, and the HC-CDR3 of A₁ comprise: HC-CDR1: SEQ ID NO: 1; HC-CDR2: SEQ ID NO: 2; HC-CDR3: SEQ ID NO: 3.

62. The isolated multispecific antibody of claim 36, wherein the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12; and wherein said CDRs comprise from 0-2 amino acid modifications in at least one of said HC-CDR1, HC-CDR2, or HC-CDR3.

63. The isolated multispecific antibody of claim 36, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise: LC-CDR1: SEQ ID NO: 13; LC-CDR2: SEQ ID NO: 14 (DA); and LC-CDR3: SEQ ID NO: 15; and wherein the CDRs comprise from 0-2 amino acid modifications in at least one of said LC-CDR1, LC-CDR2, or LC-CDR3.

64. The isolated multispecific antibody of claim 1, wherein B comprises complementarity determining region (CDRs): HC-CDR1, HC-CDR2, and HC-CDR3, wherein the HC-CDR1, the HC-CDR2, and the HC-CDR3 of B comprise: HC-CDR1: SEQ ID NO: 10; HC-CDR2: SEQ ID NO: 11; HC-CDR3: SEQ ID NO: 12; and wherein B comprises complementarity determining regions (CDRs): LC-CDR1, LC-CDR2, and LC-CDR3, wherein the LC-CDR1, the LC-CDR2, and the LC-CDR3 of B comprise: LC-CDR1: SEQ ID NO: 13; LC-CDR2: SEQ ID NO: 14 (DA); and LC-CDR3: SEQ ID NO: 15.

65. The isolated multispecific antibody of claim 36, wherein the scFv heavy chain variable domain comprises an amino acid sequence that has at least 80% sequence identity to the amino acid sequence according to SEQ ID NO: 7.

66. The isolated multispecific antibody of claim 36, wherein the scFv heavy chain variable domain comprises an amino acid sequence of at least 75 consecutive amino acid residues of SEQ ID NO: 7.

67. The isolated multispecific antibody of claim 36, wherein the scFv heavy chain variable domain comprises an amino acid sequence of at least 110 consecutive amino acid residues of SEQ ID NO: 7.

68. The isolated multispecific antibody of claim 36, wherein the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7.

69. The isolated multispecific antibody of claim 36, wherein the scFv heavy chain variable domain comprises an amino acid sequence according to SEQ ID NO: 7.

70. The isolated multispecific antibody of claim 36, wherein the scFv light chain variable domain comprises an amino acid sequence that has at least 80% sequence identity to the amino acid sequence according to SEQ ID NO: 8.

71. The isolated multispecific antibody of claim 36, wherein the scFv light chain variable domain comprises an amino acid sequence of at least 75 consecutive amino acid residues of SEQ ID NO: 8.

72. The isolated multispecific antibody of claim 36, wherein the scFv light chain variable domain comprises an amino acid sequence of at least 100 consecutive amino acid residues of SEQ ID NO: 8.

73. The isolated multispecific antibody of claim 36, wherein the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8.

74. The isolated multispecific antibody of claim 36, wherein the scFv light chain variable domain comprises an amino acid sequence according to SEQ ID NO: 8.

75. The isolated multispecific antibody of claim 36, wherein the scFv comprises an amino acid sequence that has at least 80% sequence identity to the amino acid sequence according to SEQ ID NO: 9.

76. The isolated multispecific antibody of claim 36, wherein the scFv comprises an amino acid sequence of at least 175 consecutive amino acid residues of SEQ ID NO: 9.

77. The isolated multispecific antibody of claim 36, wherein the scFv comprises an amino acid sequence of at least 210 consecutive amino acid residues of SEQ ID NO: 9.

78. The isolated multispecific antibody of claim 36, wherein the scFv comprises an amino acid sequence of at least 210 consecutive amino acid residues of SEQ ID NO: 9 and has at least 80% sequence identity to the at least 210 consecutive amino acid residues of SEQ ID NO: 9.

79. The isolated multispecific antibody of claim 36, wherein the scFv comprises an amino acid sequence according to SEQ ID NO: 9.

80. The isolated multispecific antibody of claim 36, wherein the Fab heavy chain polypeptide comprises an amino acid sequence that has at least 80% sequence identity to the amino acid sequence according to SEQ ID NO: 17.

81. The isolated multispecific antibody of claim 36, wherein the Fab heavy chain polypeptide comprises an amino acid sequence of at least 175 consecutive amino acid residues of SEQ ID NO: 17.

82. The isolated multispecific antibody of claim 36, wherein the Fab heavy chain polypeptide comprises an amino acid sequence of at least 215 consecutive amino acid residues of SEQ ID NO: 17.

83. The isolated multispecific antibody of claim 36, wherein the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17.

84. The isolated multispecific antibody of claim 36, wherein the Fab heavy chain polypeptide comprises an amino acid sequence according to SEQ ID NO: 17.

85. The isolated multispecific antibody of claim 36, wherein the Fab light chain polypeptide comprises an amino acid sequence that has at least 80% sequence identity to the amino acid sequence according to SEQ ID NO: 16.

86. The isolated multispecific antibody of claim 36, wherein the Fab light chain polypeptide comprises an amino acid sequence of at least 175 consecutive amino acid residues of SEQ ID NO: 16.

87. The isolated multispecific antibody of claim 36, wherein the Fab light chain polypeptide comprises an amino acid sequence of at least 200 consecutive amino acid residues of SEQ ID NO: 16.

88. The isolated multispecific antibody of claim 36, wherein the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16.

89. The isolated multispecific antibody of claim 36, wherein the Fab light chain polypeptide comprises an amino acid sequence according to SEQ ID NO: 16.

90. The isolated multispecific antibody of claim 36, wherein the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20 and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

91. The isolated multispecific antibody of claim 36, wherein the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO:21.

92. The isolated multispecific antibody of claim 36, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22.

93. The isolated multispecific antibody of claim 36, wherein the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO:22.

94. The isolated multispecific antibody of claim 1, wherein the multispecific antibody further comprises a half-life extending molecule (H₁).

95. The isolated multispecific antibody of claim 94, wherein H₁ is connected to P₁.

96. The isolated multispecific antibody of claim 94, wherein H₁ does not block A₁ binding to CD28.

97. The isolated multispecific antibody of claim 94, wherein H₁ does not block B binding to PD-L1.

98. The isolated multispecific antibody of claim 94, H₁ comprises a linking moiety (L₅) that connects H₁ to P₁.

99. The isolated multispecific antibody of claim 94, wherein the half-life extending molecule (H₁) does not have binding affinity to PD-L1.

100. The isolated multispecific antibody of claim 94, wherein the half-life extending molecule (H₁) does not have binding affinity to CD28.

101. The isolated multispecific antibody of claim 94, wherein the half-life extending molecule (H₁) does not shield the multispecific antibody from CD28.

102. The isolated multispecific antibody of claim 94, wherein H₁ comprises a sequence according to SEQ ID NOs: 54-57.

103. The isolated multispecific antibody of claim 94, wherein H₁ comprises an amino acid sequence that has repetitive sequence motifs.

104. The isolated multispecific antibody of claim 94, wherein H₁ comprises an amino acid sequence that has highly ordered secondary structure.

105. The isolated multispecific antibody of claim 94, wherein H₁ comprises a polymer.

106. The isolated multispecific antibody of claim 105, wherein the polymer is polyethylene glycol (PEG).

107. The isolated multispecific antibody of claim 94, wherein H₁ comprises albumin.

108. The isolated multispecific antibody of claim 94, wherein H₁ comprises an Fc domain.

109. The isolated multispecific antibody of claim 107, wherein the albumin is serum albumin.

110. The isolated multispecific antibody of claim 107, wherein the albumin is human serum albumin.

111. The isolated multispecific antibody of claim 94, wherein H₁ comprises a polypeptide, a ligand, or a small molecule.

112. The isolated multispecific antibody of claim 111, wherein the polypeptide, the ligand or the small molecule binds serum protein or a fragment thereof, a circulating immunoglobulin or a fragment thereof, or CD35/CR1.

113. The isolated multispecific antibody of claim 112, wherein the serum protein comprises a thyroxine-binding protein, a transthyretin, a 1-acid glycoprotein, a transferrin, transferrin receptor or a transferrin-binding portion thereof, a fibrinogen, or an albumin.

114. The isolated multispecific antibody of claim 112, wherein the circulating immunoglobulin molecule comprises IgG1, IgG2, IgG3, IgG4, sIgA, IgM or IgD.

115. The isolated multispecific antibody of claim 112, wherein the serum protein is albumin.

116. The isolated multispecific antibody of claim 112, wherein the polypeptide is an antibody.

117. The isolated multispecific antibody of claim 112, wherein the antibody comprises a single domain antibody, a single chain variable fragment, a Fab, or a Fab′.

118. The isolated multispecific antibody of claim 117, wherein the single domain antibody comprises a single domain antibody that binds to albumin.

119. The isolated multispecific antibody of claim 118, wherein the single domain antibody is a human or humanized antibody.

120. The isolated multispecific antibody of claim 117, wherein the single domain antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10E and SA21.

121. The isolated multispecific antibody of claim 117, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3.

122. The isolated multispecific antibody of claim 94, wherein H₁ comprises an amino acid sequence according to SEQ ID NO: 57.

123. The isolated multispecific antibody of claim 94, wherein H₁ comprises an amino acid sequence that has at least 90% sequence identity to SEQ ID NO: 57.

124. The isolated multispecific antibody of claim 94, wherein H₁ comprises an amino acid sequence that has at least 95% sequence identity to SEQ ID NO: 57.

125. The isolated multispecific antibody of claim 94, wherein H₁ comprises an amino acid sequence that has at least 99% sequence identity to SEQ ID NO: 57.

126. The isolated multispecific antibody of claim 94, wherein H₁ comprise a modified amino acid or non-natural amino acid, or a modified non-natural amino acid, or a combination thereof.

127. The isolated multispecific antibody of claim 126, wherein the modified amino acid or a modified non-natural amino acid comprises a post-translational modification.

128. The isolated multispecific antibody of claim 94, wherein H₁ comprises a linking moiety (L₅) that connects H₁ to P₁.

129. The isolated multispecific antibody of claim 128, wherein L₅ is a peptide sequence having at least 5 to no more than 50 amino acids.

130. The isolated multispecific antibody of claim 128, wherein L₅ is a peptide sequence having at least 26 amino acids.

131. The isolated multispecific antibody of claim 128, wherein L₅ comprises a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

132. The isolated multispecific antibody of claim 1, wherein the isolated multispecific antibody comprises an amino acid sequence that has at least 80% sequence identity to SEQ ID NOs: 149-170.

133. The isolated multispecific antibody of claim 1, wherein the isolated multispecific antibody comprises an amino acid sequence that has at least 95% sequence identity to SEQ ID NOs: 149-170.

134. The isolated multispecific antibody of claim 1, wherein the isolated multispecific antibody comprises an amino acid sequence that has at least 99% sequence identity to SEQ ID NOs: 149-170.

135. The isolated multispecific antibody of claim 1, wherein the isolated multispecific antibody comprises an amino acid sequence that has at least 95% sequence identity to SEQ ID NOs: 149 and 150.

136. The isolated multispecific antibody of claim 1, wherein the isolated multispecific antibody comprises an amino acid sequence that has at least 99% sequence identity to SEQ ID NOs: 149 and 150.

137. The isolated multispecific antibody of claim 1, wherein the isolated multispecific antibody comprises an amino acid sequence that has at least 95% sequence identity to SEQ ID NOs: 151 and 152.

138. The isolated multispecific antibody of claim 1, wherein the isolated multispecific antibody comprises an amino acid sequence that has at least 99% sequence identity to SEQ ID NOs: 151 and 152.

139. The isolated multispecific antibody of claim 1, wherein the isolated multispecific antibody comprises an amino acid sequence that has at least 95% sequence identity to SEQ ID NOs: 153 and 154.

140. The isolated multispecific antibody of claim 1, wherein the isolated multispecific antibody comprises an amino acid sequence that has at least 99% sequence identity to SEQ ID NOs: 153 and 154.

141. The isolated multispecific antibody of claim 1, wherein the isolated multispecific antibody comprises an amino acid sequence that has at least 95% sequence identity to SEQ ID NOs: 155 and 156.

142. The isolated multispecific antibody of claim 1, wherein the isolated multispecific antibody comprises an amino acid sequence that has at least 99% sequence identity to SEQ ID NOs: 155 and 156.

143. The isolated multispecific antibody of claim 1, wherein the isolated multispecific antibody comprises an amino acid sequence that has at least 95% sequence identity to SEQ ID NOs: 157 and 158.

144. The isolated multispecific antibody of claim 1, wherein the isolated multispecific antibody comprises an amino acid sequence that has at least 99% sequence identity to SEQ ID NOs: 157 and 158.

145. The isolated multispecific antibody of claim 1, wherein the isolated multispecific antibody comprises an amino acid sequence that has at least 95% sequence identity to SEQ ID NOs: 159 and 160.

146. The isolated multispecific antibody of claim 1, wherein the isolated multispecific antibody comprises an amino acid sequence that has at least 99% sequence identity to SEQ ID NOs: 159 and 160.

147. The isolated multispecific antibody of claim 1, wherein the isolated multispecific antibody comprises an amino acid sequence that has at least 95% sequence identity to SEQ ID NOs: 161 and 162.

148. The isolated multispecific antibody of claim 1, wherein the isolated multispecific antibody comprises an amino acid sequence that has at least 99% sequence identity to SEQ ID NOs: 161 and 162.

149. The isolated multispecific antibody of claim 1, wherein the isolated multispecific antibody comprises an amino acid sequence that has at least 95% sequence identity to SEQ ID NOs: 163 and 164.

150. The isolated multispecific antibody of claim 1, wherein the isolated multispecific antibody comprises an amino acid sequence that has at least 99% sequence identity to SEQ ID NOs: 163 and 164.

151. The isolated multispecific antibody of claim 1, wherein the isolated multispecific antibody comprises an amino acid sequence that has at least 95% sequence identity to SEQ ID NOs: 165 and 166.

152. The isolated multispecific antibody of claim 1, wherein the isolated multispecific antibody comprises an amino acid sequence that has at least 99% sequence identity to SEQ ID NOs: 165 and 166.

153. The isolated multispecific antibody of claim 1, wherein the isolated multispecific antibody comprises an amino acid sequence that has at least 95% sequence identity to SEQ ID NOs: 167 and 168.

154. The isolated multispecific antibody of claim 1, wherein the isolated multispecific antibody comprises an amino acid sequence that has at least 99% sequence identity to SEQ ID NOs: 167 and 168.

155. The isolated multispecific antibody of claim 1, wherein the isolated multispecific antibody comprises an amino acid sequence that has at least 95% sequence identity to SEQ ID NOs: 169 and 170.

156. The isolated multispecific antibody of claim 1, wherein the isolated multispecific antibody comprises an amino acid sequence that has at least 99% sequence identity to SEQ ID NOs: 169 and 170.

157. The isolated multispecific antibody of claim 1, wherein the isolated multispecific antibody comprises an amino acid sequence that has at least 95% sequence identity to SEQ ID NOs: 208 and 209.

158. The isolated multispecific antibody of claim 1, wherein the isolated multispecific antibody comprises an amino acid sequence that has at least 99% sequence identity to SEQ ID NOs: 208 and 209.

159. An isolated recombinant nucleic acid molecule encoding a polypeptide of the isolated multispecific antibody of claim 1.

160. A pharmaceutical composition comprising:
(a) the isolated multispecific antibody of claim 1; and
(b) a pharmaceutically acceptable excipient.


SEQUENCE LISTING (635 sequences)

SEQ ID NO: 1 - protein, 8 aa, synthetic construct
GYTFTSYY

SEQ ID NO: 2 - protein, 8 aa, synthetic construct
IYPGNVNT

SEQ ID NO: 3 - protein, 13 aa, synthetic construct
TRSHYGLDWNFDV

SEQ ID NO: 4 - protein, 6 aa, synthetic construct
QNIYVW

SEQ ID NO: 5 - skipped

SEQ ID NO: 6 - protein, 9 aa, synthetic construct
QQGQTYPYT

SEQ ID NO: 7 - protein, 120 aa, synthetic construct
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSS

SEQ ID NO: 8 - protein, 107 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 9 - protein, 242 aa, synthetic construct
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 10 - protein, 8 aa, synthetic construct
GDTFSTYA

SEQ ID NO: 11 - protein, 8 aa, synthetic construct
IIPIFGKA

SEQ ID NO: 12 - protein, 16 aa, synthetic construct
ARKFHFVSGSPFGMDV

SEQ ID NO: 13 - protein, 6 aa, synthetic construct
QSVSSY

SEQ ID NO: 14 - skipped

SEQ ID NO: 15 - protein, 8 aa, synthetic construct
QQRSNWPT

SEQ ID NO: 16 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 226 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 15 aa, synthetic construct
GGGGSGGGGSGGGGS

SEQ ID NO: 19 - protein, 5 aa, synthetic construct
GGGGS

SEQ ID NO: 20 - protein, 213 aa, synthetic construct; identical to SEQ ID NO: 16

SEQ ID NO: 21 - protein, 473 aa, synthetic construct
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 22 - protein, 460 aa, synthetic construct
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 226 aa, synthetic construct; identical to SEQ ID NO: 17

SEQ ID NO: 24 - protein, 14 aa, synthetic construct
YWYCSPSIVRCVLV

SEQ ID NO: 25 - protein, 14 aa, synthetic construct
LICKSGSILILCAQ

SEQ ID NO: 26 - protein, 14 aa, synthetic construct
SPCGLFQWMEICEF

SEQ ID NO: 27 - protein, 14 aa, synthetic construct
SFCGLFLDLWICEF

SEQ ID NO: 28 - protein, 14 aa, synthetic construct
DNCYVIWGFEWQCR

SEQ ID NO: 29 - protein, 14 aa, synthetic construct
VNCMRVHRTLTWCV

SEQ ID NO: 30 - protein, 14 aa, synthetic construct
FCTPREWSFLNFVC

SEQ ID NO: 31 - protein, 14 aa, synthetic construct
CFAYLWIDSWIRVC

SEQ ID NO: 32 - protein, 14 aa, synthetic construct
MDWCPRERWVDCFF

SEQ ID NO: 33 - protein, 14 aa, synthetic construct
GQHCATSMWRYCMF

SEQ ID NO: 34 - protein, 14 aa, synthetic construct
WICDKSGSIMLCRA

SEQ ID NO: 35 - protein, 14 aa, synthetic construct
GYCHYWGDMVMWCG

SEQ ID NO: 36 - protein, 14 aa, synthetic construct
DNCHYIWGFEWQCG

SEQ ID NO: 37 - protein, 14 aa, synthetic construct
IDCIMVHMVKPWCF

SEQ ID NO: 38 - protein, 14 aa, synthetic construct
NCQPWYWNMFAFGC

SEQ ID NO: 39 - protein, 14 aa, synthetic construct
NCQPWYWNMIAFGC

SEQ ID NO: 40 - protein, 14 aa, synthetic construct
GCFTWSQRTFPFTC

SEQ ID NO: 41 - protein, 14 aa, synthetic construct
CFYAEYYDQVYSFC

SEQ ID NO: 42 - protein, 14 aa, synthetic construct
ADWCPRERWVDCFF

SEQ ID NO: 43 - protein, 14 aa, synthetic construct
MAWCPRERWVDCFF

SEQ ID NO: 44 - protein, 14 aa, synthetic construct
MDACPRERWVDCFF

SEQ ID NO: 45 - protein, 14 aa, synthetic construct
MDWCARERWVDCFF

SEQ ID NO: 46 - protein, 14 aa, synthetic construct
MDWCPAERWVDCFF

SEQ ID NO: 47 - protein, 14 aa, synthetic construct
MDWCPRARWVDCFF

SEQ ID NO: 48 - protein, 14 aa, synthetic construct
MDWCPREAWVDCFF

SEQ ID NO: 49 - protein, 14 aa, synthetic construct
MDWCPRERAVDCFF

SEQ ID NO: 50 - protein, 14 aa, synthetic construct
MDWCPRERWADCFF

SEQ ID NO: 51 - protein, 14 aa, synthetic construct
MDWCPRERWVACFF

SEQ ID NO: 52 - protein, 14 aa, synthetic construct
MDWCPRERWVDCAF

SEQ ID NO: 53 - protein, 14 aa, synthetic construct
MDWCPRERWVDCFA

SEQ ID NO: 54 - protein, 8 aa, synthetic construct
GSTFYTAV

SEQ ID NO: 55 - protein, 8 aa, synthetic construct
IRWTALTT

SEQ ID NO: 56 - protein, 17 aa, synthetic construct
AARGTLGLFTTADSYDY

SEQ ID NO: 57 - protein, 124 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSS

SEQ ID NO: 58 - protein, 5 aa, synthetic construct
GSGGS

SEQ ID NO: 59 - protein, 4 aa, synthetic construct
GGGS

SEQ ID NO: 60 - protein, 5 aa, synthetic construct
GGGGS

SEQ ID NO: 61 - protein, 6 aa, synthetic construct
GSSGGS

SEQ ID NO: 62 - protein, 9 aa, synthetic construct
GGGGSGGGS

SEQ ID NO: 63 - protein, 28 aa, synthetic construct
GGGGSGGGLSGRSDAGSPLGLAGSGGGS

SEQ ID NO: 64 - protein, 18 aa, synthetic construct
GGGGSLSGRSDNHGSSGT

SEQ ID NO: 65 - protein, 26 aa, synthetic construct
GGGGSSGGSGGSGLSGRSDNHGSSGT

SEQ ID NO: 66 - protein, 8 aa, synthetic construct
ASGRSDNH

SEQ ID NO: 67 - protein, 8 aa, synthetic construct
LAGRSDNH

SEQ ID NO: 68 - protein, 13 aa, synthetic construct
ISSGLASGRSDNH

SEQ ID NO: 69 - protein, 13 aa, synthetic construct
ISSGLLAGRSDNH

SEQ ID NO: 70 - protein, 8 aa, synthetic construct
LSGRSDNH

SEQ ID NO: 71 - protein, 13 aa, synthetic construct
ISSGLLSGRSDNP

SEQ ID NO: 72 - protein, 13 aa, synthetic construct
ISSGLLSGRSDNH

SEQ ID NO: 73 - protein, 16 aa, synthetic construct
LSGRSDNHSPLGLAGS

SEQ ID NO: 74 - protein, 16 aa, synthetic construct
SPLGLAGSLSGRSDNH

SEQ ID NO: 75 - protein, 12 aa, synthetic construct
SPLGLSGRSDNH

SEQ ID NO: 76 - protein, 16 aa, synthetic construct
LAGRSDNHSPLGLAGS

SEQ ID NO: 77 - protein, 16 aa, synthetic construct
LSGRSDNHVPLSLKMG

SEQ ID NO: 78 - protein, 16 aa, synthetic construct
LSGRSDNHVPLSLSMG

SEQ ID NO: 79 - protein, 31 aa, synthetic construct
GSSGGSGGSGGSGISSGLLSGRSDNHGSSGT

SEQ ID NO: 80 - protein, 28 aa, synthetic construct
GSSGGSGGSGGISSGLLSGRSDNHGGGS

SEQ ID NO: 81 - protein, 8 aa, synthetic construct
ASGRSDNH

SEQ ID NO: 82 - protein, 8 aa, synthetic construct
LAGRSDNH

SEQ ID NO: 83 - protein, 13 aa, synthetic construct
ISSGLASGRSDNH

SEQ ID NO: 84 - protein, 8 aa, synthetic construct
LSGRSDAG

SEQ ID NO: 85 - protein, 13 aa, synthetic construct
ISSGLLSGRSDAG

SEQ ID NO: 86 - protein, 18 aa, synthetic construct
AAGLLAPPGGLSGRSDAG

SEQ ID NO: 87 - protein, 12 aa, synthetic construct
SPLGLSGRSDAG

SEQ ID NO: 88 - protein, 15 aa, synthetic construct
LSGRSDAGSPLGLAG

SEQ ID NO: 89 - protein, 8 aa, synthetic construct
GFTFNKYA

SEQ ID NO: 90 - protein, 10 aa, synthetic construct
IRSKYNNYAT

SEQ ID NO: 91 - protein, 16 aa, synthetic construct
VRHGNFGNSYISYWAY

SEQ ID NO: 92 - protein, 9 aa, synthetic construct
TGAVTSGNY

SEQ ID NO: 93 - skipped

SEQ ID NO: 94 - protein, 9 aa, synthetic construct
VLWYSNRWV

SEQ ID NO: 95 - protein, 8 aa, synthetic construct
GFTFNTYA

SEQ ID NO: 96 - protein, 16 aa, synthetic construct
VRHGNFGNSYVSWFAY

SEQ ID NO: 97 - protein, 9 aa, synthetic construct
TGAVTTSNY

SEQ ID NO: 98 - skipped

SEQ ID NO: 99 - protein, 9 aa, synthetic construct
ALWYSNLWV

SEQ ID NO: 100 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 101 - protein, 249 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 102 - protein, 6 aa, synthetic construct
QSIGTN

SEQ ID NO: 103 - skipped

SEQ ID NO: 104 - protein, 9 aa, synthetic construct
QQNNNWPTT

SEQ ID NO: 105 - protein, 8 aa, synthetic construct
GFSLTNYG

SEQ ID NO: 106 - protein, 7 aa, synthetic construct
IWSGGNT

SEQ ID NO: 107 - protein, 13 aa, synthetic construct
ARALTYYDYEFAY

SEQ ID NO: 108 - protein, 214 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 222 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 111 - protein, 222 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 112 - protein, 8 aa, synthetic construct
GYTFTNYG

SEQ ID NO: 113 - protein, 8 aa, synthetic construct
INTYTGEP

SEQ ID NO: 114 - protein, 14 aa, synthetic construct
ARGGFGSSYWYFDV

SEQ ID NO: 115 - protein, 6 aa, synthetic construct
QDVSIA

SEQ ID NO: 116 - skipped

SEQ ID NO: 117 - protein, 9 aa, synthetic construct
QQHYITPLT

SEQ ID NO: 118 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 120 - protein, 8 aa, synthetic construct
GFAFSRYG

SEQ ID NO: 121 - protein, 8 aa, synthetic construct
IWYDGSNK

SEQ ID NO: 122 - protein, 16 aa, synthetic construct
ARGGDFLYYYYYGMDV

SEQ ID NO: 123 - protein, 6 aa, synthetic construct
QGISNY

SEQ ID NO: 124 - skipped

SEQ ID NO: 125 - protein, 9 aa, synthetic construct
QNYNSAPFT

SEQ ID NO: 126 - protein, 214 aa, synthetic construct
DIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 226 aa, synthetic construct
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 128 - protein, 14 aa, synthetic construct
MDWCPIDLWNECFF

SEQ ID NO: 129 - protein, 14 aa, synthetic construct
MDWCPIHLWHVCFN

SEQ ID NO: 130 - protein, 14 aa, synthetic construct
MDWCPIYLWSECFN

SEQ ID NO: 131 - protein, 14 aa, synthetic construct
MNWCPKDIWYLCFN

SEQ ID NO: 132 - protein, 14 aa, synthetic construct
MDWCPLHMWHECFS

SEQ ID NO: 133 - protein, 14 aa, synthetic construct
MDWCPLYLWNECFN

SEQ ID NO: 134 - protein, 14 aa, synthetic construct
MDWCPRDLWDLCFA

SEQ ID NO: 135 - protein, 14 aa, synthetic construct
MDWCPRDLWHECFA

SEQ ID NO: 136 - protein, 14 aa, synthetic construct
MDWCPRDLWHLCFS

SEQ ID NO: 137 - protein, 14 aa, synthetic construct
MDWCPRDLWSECFF

SEQ ID NO: 138 - protein, 14 aa, synthetic construct
MDWCPRDLWVHCFA

SEQ ID NO: 139 - protein, 14 aa, synthetic construct
MDWCPRDMWDECFA

SEQ ID NO: 140 - protein, 14 aa, synthetic construct
MDWCPRDMWSECFA

SEQ ID NO: 141 - protein, 14 aa, synthetic construct
MDWCPRDMWSVCFS

SEQ ID NO: 142 - protein, 14 aa, synthetic construct
MDWCPRFMWDECFN

SEQ ID NO: 143 - protein, 14 aa, synthetic construct
MDWCPRHMWNYCFA

SEQ ID NO: 144 - protein, 14 aa, synthetic construct
MDWCPRSLWHECFA

SEQ ID NO: 145 - protein, 14 aa, synthetic construct
MDWCPRYLWHVCFA

SEQ ID NO: 146 - protein, 14 aa, synthetic construct
MHWCPVDLWYLCYN

SEQ ID NO: 147 - protein, 14 aa, synthetic construct
MDWCPVHLWSVCFA

SEQ ID NO: 148 - protein, 14 aa, synthetic construct
MDWCPRERWVDCFF

SEQ ID NO: 149 - protein, 213 aa, synthetic construct; identical to SEQ ID NO: 16

SEQ ID NO: 150 - protein, 641 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGMDWCPRERWVDCFFGGGGSGGGLSGRSDAGSPLGLAGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 151 - protein, 213 aa, synthetic construct; identical to SEQ ID NO: 16

SEQ ID NO: 152 - protein, 641 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGMDWCPRERWVDCFFGSSGGSAAGLLAPPGGLSGRSDAGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 153 - protein, 213 aa, synthetic construct; identical to SEQ ID NO: 16

SEQ ID NO: 154 - protein, 641 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGMDWCPRERWVDCFFGGGGSGGGSGGGGSGGASSGAGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 155 - protein, 213 aa, synthetic construct; identical to SEQ ID NO: 16

SEQ ID NO: 156 - protein, 641 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGGGSGSSGGASSGGSGGGGSGGGSGGGGSGGASSGAGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 157 - protein, 213 aa, synthetic construct; identical to SEQ ID NO: 16

SEQ ID NO: 158 - protein, 650 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGMDWCPRERWVDCFFGGGGSGGGLSGRSDAGSPLGLAGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 159 - protein, 213 aa, synthetic construct; identical to SEQ ID NO: 16

SEQ ID NO: 160 - protein, 650 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGMDWCPRERWVDCFFGSSGGSAAGLLAPPGGLSGRSDAGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 161 - protein, 213 aa, synthetic construct; identical to SEQ ID NO: 16

SEQ ID NO: 162 - protein, 650 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGMDWCPRERWVDCFFGGGGSGGGSGGGGSGGASSGAGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 163 - protein, 213 aa, synthetic construct; identical to SEQ ID NO: 16

SEQ ID NO: 164 - protein, 650 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGMDWCPIYLWSECFNGSSGGSGGLSGRSDAGSPLGLAGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 165 - protein, 213 aa, synthetic construct; identical to SEQ ID NO: 16

SEQ ID NO: 166 - protein, 650 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGMDWCPRDLWVHCFAGSSGGSGGLSGRSDAGSPLGLAGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 167 - protein, 637 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGMDWCPIYLWSECFNGSSGGSGGLSGRSDAGSPLGLAGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 226 aa, synthetic construct; identical to SEQ ID NO: 17

SEQ ID NO: 169 - protein, 637 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGMDWCPRDLWVHCFAGSSGGSGGLSGRSDAGSPLGLAGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 226 aa, synthetic construct; identical to SEQ ID NO: 17

SEQ ID NO: 171-172 - skipped (2 entries)

SEQ ID NO: 173 - protein, 468 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 226 aa, synthetic construct; identical to SEQ ID NO: 127

SEQ ID NO: 175 - protein, 645 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 226 aa, synthetic construct; identical to SEQ ID NO: 127

SEQ ID NO: 177 - protein, 14 aa, synthetic construct
GSQCLGPEWEVCPY

SEQ ID NO: 178 - protein, 14 aa, synthetic construct
VYCGPEFDESVGCM

SEQ ID NO: 179 - protein, 14 aa, synthetic construct
VYCGPEFDESVGCA

SEQ ID NO: 180 - protein, 14 aa, synthetic construct
YLCGPDGDETLACY

SEQ ID NO: 181 - protein, 214 aa, synthetic construct; identical to SEQ ID NO: 108

SEQ ID NO: 182 - protein, 476 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 183 - protein, 256 aa, synthetic construct
GGPCRSHIDVAKPICVGGGGSGGLSGRSDAGSPLGLAGSGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 653 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 185 - protein, 14 aa, synthetic construct
PCRSHIDVAKPICV

SEQ ID NO: 186 - protein, 14 aa, synthetic construct
PCLFHFDPAKPICS

SEQ ID NO: 187 - protein, 214 aa, synthetic construct; identical to SEQ ID NO: 118

SEQ ID NO: 188 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 189 - protein, 214 aa, synthetic construct; identical to SEQ ID NO: 118

SEQ ID NO: 190 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 191 - protein, 214 aa, synthetic construct; identical to SEQ ID NO: 118

SEQ ID NO: 192 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 193 - protein, 256 aa, synthetic construct
GGIDFCMLYNWPICAGGGGGSGGLSGRSDAGSPLGLAGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 655 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 195 - protein, 256 aa, synthetic construct
GGVDFCGLYHWPICYQGGGGSGGLSGRSDAGSPLGLAGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 655 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 197 - protein, 256 aa, synthetic construct
GGVDFCALYHWPICYQGGGGSGGLSGRSDAGSPLGLAGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 655 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 199 - protein, 13 aa, synthetic construct
IDFCMLYNWPICA

SEQ ID NO: 200 - protein, 14 aa, synthetic construct
VDFCGLYHWPICYQ

SEQ ID NO: 201 - protein, 14 aa, synthetic construct
VDFCALYHWPICYQ

SEQ ID NO: 202 - protein, 468 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 224 aa, synthetic construct; identical to SEQ ID NO: 119

SEQ ID NO: 204 - protein, 8 aa, synthetic construct
GFTFSKFG

SEQ ID NO: 205 - protein, 8 aa, synthetic construct
ISGSGRDT

SEQ ID NO: 206 - protein, 8 aa, synthetic construct
TIGGSLSV

SEQ ID NO: 207 - protein, 115 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 208 - protein, 213 aa, synthetic construct; identical to SEQ ID NO: 16

SEQ ID NO: 209 - protein, 641 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGMDWCPRDLWVHCFAGSSGGSGGLSGRSDAGSPLGLAGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 210 - protein, 213 aa, synthetic construct; identical to SEQ ID NO: 16

SEQ ID NO: 211 - protein, 641 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGMDWCPRDLWVHCFAGGGGSGGGSGGGGSGGASSGAGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 212 - protein, 213 aa, synthetic construct; identical to SEQ ID NO: 16

SEQ ID NO: 213 - protein, 650 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGMDWCPRDLWVHCFAGGGGSGGGSGGGGSGGASSGAGGSGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 214 - protein, 214 aa, synthetic construct; identical to SEQ ID NO: 108

SEQ ID NO: 215 - protein, 476 aa, synthetic construct; identical to SEQ ID NO: 182

SEQ ID NO: 216 - protein, 256 aa, synthetic construct; identical to SEQ ID NO: 193

SEQ ID NO: 217 - protein, 646 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 218 - protein, 259 aa, synthetic construct
GGIDFCMLYNWPICAGGGGGSGGGGGSGGGGSGGASSGAGGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 646 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGSGGGGSGGASSGAGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 220 - protein, 214 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 450 aa, synthetic construct
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 224 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 228 - protein, 9 aa, synthetic construct
  VARIANT 1..9: note = This sequence may encompass 1-3 GGS repeating units
GGSGGSGGS

SEQ ID NO: 229 - protein, 14 aa, synthetic construct
MDWCPMHLWHQCFN

SEQ ID NO: 230 - protein, 14 aa, synthetic construct
MDWCPIDMWDQCFN

SEQ ID NO: 231 - protein, 14 aa, synthetic construct
MAWCPRDKWSECFS

SEQ ID NO: 232 - protein, 14 aa, synthetic construct
MDWCPRHLWVHCFN

SEQ ID NO: 233 - protein, 14 aa, synthetic construct
MDWCPRALWHECFY

SEQ ID NO: 234 - protein, 14 aa, synthetic construct
MDWCPIALWAECFN

SEQ ID NO: 235 - protein, 14 aa, synthetic construct
MDWCPRPLWHECFS

SEQ ID NO: 236 - protein, 14 aa, synthetic construct
MHWCPIDLWAECYA

SEQ ID NO: 237 - protein, 14 aa, synthetic construct
MAWCPVYLWHECFN

SEQ ID NO: 238 - protein, 14 aa, synthetic construct
IDWCPRYLWDECYN

SEQ ID NO: 239 - protein, 14 aa, synthetic construct
MSWCPIHLWNECFN

SEQ ID NO: 240 - protein, 14 aa, synthetic construct
MDWCPPYLWNVCFS

SEQ ID NO: 241 - protein, 14 aa, synthetic construct
MDWCPMDLWDYCFN

SEQ ID NO: 242 - protein, 14 aa, synthetic construct
MDWCPINLWDECFS

SEQ ID NO: 243 - protein, 14 aa, synthetic construct
MDWCPMHLWNKCFN

SEQ ID NO: 244 - protein, 14 aa, synthetic construct
MNWCPRDMWYQCFN

SEQ ID NO: 245 - protein, 14 aa, synthetic construct
QDWCPRHMWSFCFH

SEQ ID NO: 246 - protein, 14 aa, synthetic construct
MHWCPMDQWSNCFN

SEQ ID NO: 247 - protein, 14 aa, synthetic construct
MDWCPRHRWVDCFY

SEQ ID NO: 248 - protein, 14 aa, synthetic construct
MHWCPIDRWADCFN

SEQ ID NO: 249 - protein, 14 aa, synthetic construct
MDWCPRDSWHLCFA

SEQ ID NO: 250 - protein, 14 aa, synthetic construct
MDWCPRTLWYLCFN

SEQ ID NO: 251 - protein, 14 aa, synthetic construct
MVSCPTTMWNRCFN

SEQ ID NO: 252 - protein, 14 aa, synthetic construct
MDWCPSYLWNECFF

SEQ ID NO: 253 - protein, 14 aa, synthetic construct
MDWCPRALWAECFN

SEQ ID NO: 254 - protein, 14 aa, synthetic construct
MDWCPMYLWNECFN

SEQ ID NO: 255 - protein, 14 aa, synthetic construct
MDWCPRYLWNECFY

SEQ ID NO: 256 - protein, 14 aa, synthetic construct
MNWCPTALWHVCFN

SEQ ID NO: 257 - protein, 14 aa, synthetic construct
MDWCPMYMWYECFA

SEQ ID NO: 258 - protein, 14 aa, synthetic construct
MDWCPIHMWADCFA

SEQ ID NO: 259 - protein, 14 aa, synthetic construct
ITWCPSSMWNRCFI

SEQ ID NO: 260 - protein, 14 aa, synthetic construct
MDWCPRYLWHECFA

SEQ ID NO: 261 - protein, 14 aa, synthetic construct
MFWCPTTMWNRCFT

SEQ ID NO: 262 - protein, 14 aa, synthetic construct
MDWCPRALWHECFN

SEQ ID NO: 263 - protein, 14 aa, synthetic construct
MDWCPRDMWLFCYN

SEQ ID NO: 264 - protein, 14 aa, synthetic construct
MDWCPRSHWHVCYN

SEQ ID NO: 265 - protein, 14 aa, synthetic construct
MDWCPRYLWSDCFA

SEQ ID NO: 266 - protein, 14 aa, synthetic construct
MDWCPFYLWDECFN

SEQ ID NO: 267 - protein, 14 aa, synthetic construct
MDWCPRHLWHECFN

SEQ ID NO: 268 - protein, 14 aa, synthetic construct
MDWCPRDLWHECFS

SEQ ID NO: 269 - protein, 14 aa, synthetic construct
MDWCPMYLWNECFS

SEQ ID NO: 270 - protein, 14 aa, synthetic construct
MDWCPRFLWSVCFN

SEQ ID NO: 271 - protein, 14 aa, synthetic construct
MDWCPRDLWTECFA

SEQ ID NO: 272 - protein, 14 aa, synthetic construct
MDWCPRFLWDECFN

SEQ ID NO: 273 - protein, 14 aa, synthetic construct
MDWCPKYLWSVCFF

SEQ ID NO: 274 - protein, 14 aa, synthetic construct
MDWCPMDLWYQCFN

SEQ ID NO: 275 - protein, 14 aa, synthetic construct
MDWCPRHLWAECFF

SEQ ID NO: 276 - protein, 14 aa, synthetic construct
MDWCPKDLWYLCFA

SEQ ID NO: 277 - protein, 14 aa, synthetic construct
MDWCPIHLWHYCFN

SEQ ID NO: 278 - protein, 14 aa, synthetic construct
MDWCPRALWNVCFN

SEQ ID NO: 279 - protein, 14 aa, synthetic construct
MDWCPIDLWHLCFY

SEQ ID NO: 280 - protein, 14 aa, synthetic construct
MDWCPRDLWFLCYN

SEQ ID NO: 281 - protein, 14 aa, synthetic construct
MDWCPRHLWHECFF

SEQ ID NO: 282 - protein, 14 aa, synthetic construct
MDWCPRYLWTVCFS

SEQ ID NO: 283 - protein, 14 aa, synthetic construct
MDWCPRDLWSLCFY

SEQ ID NO: 284 - protein, 14 aa, synthetic construct
MDWCPRFLWSECFN

SEQ ID NO: 285 - protein, 14 aa, synthetic construct
MDWCPRTLWAYCFN

SEQ ID NO: 286 - protein, 14 aa, synthetic construct
MDWCPKDLWDYCFA

SEQ ID NO: 287 - protein, 14 aa, synthetic construct
MDWCPKYLWDVCFN

SEQ ID NO: 288 - protein, 14 aa, synthetic construct
MDWCPRYLWNMCFH

SEQ ID NO: 289 - protein, 14 aa, synthetic construct
MDWCPRYLWTECFN

SEQ ID NO: 290 - protein, 14 aa, synthetic construct
MDWCPRSLWHYCFA

SEQ ID NO: 291 - protein, 14 aa, synthetic construct
MDWCPRYLWAECFY

SEQ ID NO: 292 - protein, 14 aa, synthetic construct
MDWCPRDLWHECFN

SEQ ID NO: 293 - protein, 14 aa, synthetic construct
MDWCPRDLWDLCFF

SEQ ID NO: 294 - protein, 14 aa, synthetic construct
MDWCPRYLWNVCFN

SEQ ID NO: 295 - protein, 14 aa, synthetic construct
MDWCPIYLWDECFN

SEQ ID NO: 296 - protein, 14 aa, synthetic construct
MDWCPRDLWAECFN

SEQ ID NO: 297 - protein, 14 aa, synthetic construct
MDWCPRALWHECFT

SEQ ID NO: 298 - protein, 14 aa, synthetic construct
MDWCPKNLWHVCFN

SEQ ID NO: 299 - protein, 14 aa, synthetic construct
MDWCPRYLWDECFN

SEQ ID NO: 300 - protein, 14 aa, synthetic construct
MDWCPFYLWNECFY

SEQ ID NO: 301 - protein, 14 aa, synthetic construct
MHWCPRALWDVCYN

SEQ ID NO: 302 - protein, 14 aa, synthetic construct
MDWCPRDLWNVCFF

SEQ ID NO: 303 - protein, 14 aa, synthetic construct
MDWCPRYLWFECFA

SEQ ID NO: 304 - protein, 14 aa, synthetic construct
MDWCPRYLWHECFN

SEQ ID NO: 305 - protein, 14 aa, synthetic construct
MDWCPRDLWYACFA

SEQ ID NO: 306 - protein, 14 aa, synthetic construct
MDWCPRYLWFECFS

SEQ ID NO: 307 - protein, 14 aa, synthetic construct
MDWCPRHLWDECFF

SEQ ID NO: 308 - protein, 14 aa, synthetic construct
MDWCPRYLWHMCYS

SEQ ID NO: 309 - protein, 14 aa, synthetic construct
MDWCPRDLWHACFS

SEQ ID NO: 310 - protein, 14 aa, synthetic construct
MDWCPRDLWHVCFF

SEQ ID NO: 311 - protein, 14 aa, synthetic construct
MDWCPRDLWDQCYA

SEQ ID NO: 312 - protein, 14 aa, synthetic construct
MDWCPIHLWNECFA

SEQ ID NO: 313 - protein, 14 aa, synthetic construct
MDWCPRPLWDMCFF

SEQ ID NO: 314 - protein, 14 aa, synthetic construct
MDWCPVSLWHVCFY

SEQ ID NO: 315 - protein, 14 aa, synthetic construct
MDWCPRFLWNECFN

SEQ ID NO: 316 - protein, 14 aa, synthetic construct
MDWCPRALWNECFA

SEQ ID NO: 317 - protein, 14 aa, synthetic construct
MDWCPRDLWIECFF

SEQ ID NO: 318 - protein, 14 aa, synthetic construct
MDWCPSYLWTVCFA

SEQ ID NO: 319 - protein, 14 aa, synthetic construct
MDWCPRYLWDACFF

SEQ ID NO: 320 - protein, 14 aa, synthetic construct
MDWCPRSLWIYCFN

SEQ ID NO: 321 - protein, 14 aa, synthetic construct
MDWCPTYLWFECFN

SEQ ID NO: 322 - protein, 14 aa, synthetic construct
MDWCPRFLWDECFF

SEQ ID NO: 323 - protein, 14 aa, synthetic construct
MDWCPSYLWHECFA

SEQ ID NO: 324 - protein, 14 aa, synthetic construct
MDWCPKFLWHECFS

SEQ ID NO: 325 - protein, 14 aa, synthetic construct
MHWCPIYLWDECFN

SEQ ID NO: 326 - protein, 14 aa, synthetic construct
MDWCPRYLWHECFH

SEQ ID NO: 327 - protein, 14 aa, synthetic construct
MDWCPTNLWHECFA

SEQ ID NO: 328 - protein, 14 aa, synthetic construct
MDWCPRDLWDVCFA

SEQ ID NO: 329 - protein, 14 aa, synthetic construct
MDWCPMDLWDVCFN

SEQ ID NO: 330 - protein, 14 aa, synthetic construct
MDWCPRSLWNVCFF

SEQ ID NO: 331 - protein, 14 aa, synthetic construct
MDWCPVILWDFCFN

SEQ ID NO: 332 - protein, 14 aa, synthetic construct
LDWCPLNLWDLCYF

SEQ ID NO: 333 - protein, 14 aa, synthetic construct
MDWCPRHLWYACFN

SEQ ID NO: 334 - protein, 14 aa, synthetic construct
MDWCPKHLWIECFA

SEQ ID NO: 335 - protein, 14 aa, synthetic construct
MDWCPRHLWSECFY

SEQ ID NO: 336 - protein, 14 aa, synthetic construct
MHWCPRDLWVVCFN

SEQ ID NO: 337 - protein, 14 aa, synthetic construct
MHWCPEYLWNECFA

SEQ ID NO: 338 - protein, 14 aa, synthetic construct
MDWCPRDLWAVCFA

SEQ ID NO: 339 - protein, 14 aa, synthetic construct
MDWCPRHLWNVCFS

SEQ ID NO: 340 - protein, 14 aa, synthetic construct
MDFCPISLWHECFF

SEQ ID NO: 341 - protein, 14 aa, synthetic construct
MDWCPKYLWDKCFH

SEQ ID NO: 342 - protein, 14 aa, synthetic construct
MDWCPRHLWDLCFF

SEQ ID NO: 343 - protein, 14 aa, synthetic construct
MDWCPRDLWNVCFA

SEQ ID NO: 344 - protein, 14 aa, synthetic construct
MHWCPLHLWNECYH

SEQ ID NO: 345 - protein, 14 aa, synthetic construct
MDWCPKHLWHQCFH

SEQ ID NO: 346 - protein, 14 aa, synthetic construct
MDWCPRSLWSYCFH

SEQ ID NO: 347 - protein, 14 aa, synthetic construct
MDWCPRYLWTECFF

SEQ ID NO: 348 - protein, 14 aa, synthetic construct
MAWCPMNLWDQCFF

SEQ ID NO: 349 - protein, 14 aa, synthetic construct
MHWCPRALWHECFN

SEQ ID NO: 350 - protein, 14 aa, synthetic construct
MDWCPRHLWDQCFA

SEQ ID NO: 351 - protein, 14 aa, synthetic construct
MNWCPTDLWHECFN

SEQ ID NO: 352 - protein, 14 aa, synthetic construct
MFWCPRYLWHECFN

SEQ ID NO: 353 - protein, 14 aa, synthetic construct
MDWCPKFLWDLCFA

SEQ ID NO: 354 - protein, 14 aa, synthetic construct
MDWCPFYLWDECFL

SEQ ID NO: 355 - protein, 14 aa, synthetic construct
MDWCPRHLWDLCFA

SEQ ID NO: 356 - protein, 14 aa, synthetic construct
MSWCPQDLWHVCFN

SEQ ID NO: 357 - protein, 14 aa, synthetic construct
MDWCPKDLWHECFN

SEQ ID NO: 358 - protein, 14 aa, synthetic construct
MDWCPRDLWNVCFN

SEQ ID NO: 359 - protein, 14 aa, synthetic construct
MNWCPSDLWHLCFN

SEQ ID NO: 360 - protein, 14 aa, synthetic construct
MNWCPSHLWHMCYF

SEQ ID NO: 361 - protein, 14 aa, synthetic construct
MDWCPPYLWYECFA

SEQ ID NO: 362 - protein, 14 aa, synthetic construct
MDWCPMNLWSECFN

SEQ ID NO: 363 - protein, 14 aa, synthetic construct
MDWCPKHLWNECFN

SEQ ID NO: 364 - protein, 14 aa, synthetic construct
MDWCPAYLWAECFS

SEQ ID NO: 365 - protein, 14 aa, synthetic construct
MDWCPSDLWHECFH

SEQ ID NO: 366 - protein, 14 aa, synthetic construct
MDWCPVSLWDHCFN

SEQ ID NO: 367 - protein, 14 aa, synthetic construct
LDWCPRDLWHVCFF

SEQ ID NO: 368 - protein, 14 aa, synthetic construct
MDWCPWILWNECFN

SEQ ID NO: 369 - protein, 14 aa, synthetic construct
MHWCPRYLWDECFN

SEQ ID NO: 370 - protein, 14 aa, synthetic construct
MYWCPRDLWDVCFN

SEQ ID NO: 371 - protein, 14 aa, synthetic construct
MHWCPRSLWNECYF

SEQ ID NO: 372 - protein, 14 aa, synthetic construct
IDWCPRDLWALCFN

SEQ ID NO: 373 - protein, 14 aa, synthetic construct
MHWCPINLWNECFS

SEQ ID NO: 374 - protein, 14 aa, synthetic construct
MERCPRFLWNECFN

SEQ ID NO: 375 - protein, 14 aa, synthetic construct
QDWCPTYLWHHCFN

SEQ ID NO: 376 - protein, 14 aa, synthetic construct
IGKLTLCLNADLVI

SEQ ID NO: 377 - protein, 14 aa, synthetic construct
MDWCPSYLWDQCFF

SEQ ID NO: 378 - protein, 14 aa, synthetic construct
VDWCPRYLWHVCYN

SEQ ID NO: 379 - protein, 14 aa, synthetic construct
MDWCPRDMWAECFF

SEQ ID NO: 380 - protein, 14 aa, synthetic construct
MDWCPRDMWYECFN

SEQ ID NO: 381 - protein, 14 aa, synthetic construct
DVWCPKYMWSLCFN

SEQ ID NO: 382 - protein, 14 aa, synthetic construct
MDWCPMDMWVNCFN

SEQ ID NO: 383 - protein, 14 aa, synthetic construct
MDWCPSDMWHECYA

SEQ ID NO: 384 - protein, 14 aa, synthetic construct
MDWCPKHMWFMCFN

SEQ ID NO: 385 - protein, 14 aa, synthetic construct
MDWCPRYMWYQCFS

SEQ ID NO: 386 - protein, 14 aa, synthetic construct
MDWCPRHMWYECFF

SEQ ID NO: 387 - protein, 14 aa, synthetic construct
MDWCPRAMWNHCFN

SEQ ID NO: 388 - protein, 14 aa, synthetic construct
MDWCPRNMWAQCFA

SEQ ID NO: 389 - protein, 14 aa, synthetic construct
MFWCPFDMWHFCFN

SEQ ID NO: 390 - protein, 14 aa, synthetic construct
MDWCPRDMWDQCFD

SEQ ID NO: 391 - protein, 14 aa, synthetic construct
MFWCPMDMWDQCFN

SEQ ID NO: 392 - protein, 14 aa, synthetic construct
MSWCPRDMWFYCYA

SEQ ID NO: 393 - protein, 14 aa, synthetic construct
MDWCPRHMWNVCFS

SEQ ID NO: 394 - protein, 14 aa, synthetic construct
MDWCPTDMWHHCFL

SEQ ID NO: 395 - protein, 14 aa, synthetic construct
IHWCPINMWDKCYN

SEQ ID NO: 396 - protein, 14 aa, synthetic construct
MDWCPRAMWHECFY

SEQ ID NO: 397 - protein, 14 aa, synthetic construct
MDWCPTDMWIVCFA

SEQ ID NO: 398 - protein, 14 aa, synthetic construct
IDWCPQDMWFYCFN

SEQ ID NO: 399 - protein, 14 aa, synthetic construct
MDWCPRDMWFECFA

SEQ ID NO: 400 - protein, 14 aa, synthetic construct
MDWCPRNMWTVCFL

SEQ ID NO: 401 - protein, 14 aa, synthetic construct
MDWCPRAMWDKCFF

SEQ ID NO: 402 - protein, 14 aa, synthetic construct
MNWCPSYMWDQCFA

SEQ ID NO: 403 - protein, 14 aa, synthetic construct
MDWCPTYMWSECFN

SEQ ID NO: 404 - protein, 14 aa, synthetic construct
MDWCPRYMWNECFN

SEQ ID NO: 405 - protein, 14 aa, synthetic construct
MDWCPMNMWYQCFN

SEQ ID NO: 406 - protein, 14 aa, synthetic construct
MDWCPWDMWDKCFN

SEQ ID NO: 407 - protein, 14 aa, synthetic construct
TFGCPTTMWNRCFA

SEQ ID NO: 408 - protein, 14 aa, synthetic construct
NYWCPSSMWNRCLH

SEQ ID NO: 409 - protein, 14 aa, synthetic construct
FDFCPTTMWTYCQH

SEQ ID NO: 410 - protein, 14 aa, synthetic construct
TTWCPTSMWLHCFD

SEQ ID NO: 411 - protein, 14 aa, synthetic construct
MDWCPRDQWHNCFN

SEQ ID NO: 412 - protein, 14 aa, synthetic construct
MDWCPRDRWVDCFF

SEQ ID NO: 413 - protein, 14 aa, synthetic construct
MDWCPKDRWNDCYF

SEQ ID NO: 414 - protein, 14 aa, synthetic construct
MDWCPRDRWADCFF

SEQ ID NO: 415 - protein, 14 aa, synthetic construct
MDWCPRDRWIDCFN

SEQ ID NO: 416 - protein, 14 aa, synthetic construct
MDWCPRDRWSDCFN

SEQ ID NO: 417 - protein, 14 aa, synthetic construct
ITWCHVISGLECWN

SEQ ID NO: 418 - protein, 14 aa, synthetic construct
VPWCQIISGLECLT

SEQ ID NO: 419 - protein, 14 aa, synthetic construct
APWCQIISGLECLT

SEQ ID NO: 420 - protein, 14 aa, synthetic construct
VPWCLIISGLDCLN

SEQ ID NO: 421 - protein, 14 aa, synthetic construct
MDWCARFVGYGCLD

SEQ ID NO: 422 - protein, 14 aa, synthetic construct
MTWCPTSFWNRCLD

SEQ ID NO: 423 - protein, 14 aa, synthetic construct
MDWCPRALWFECFF

SEQ ID NO: 424 - protein, 14 aa, synthetic construct
MDWCPRYLWHECFS

SEQ ID NO: 425 - protein, 14 aa, synthetic construct
MDWCPRDLWNLCFF

SEQ ID NO: 426 - protein, 14 aa, synthetic construct
MDWCPSYLWHECFF

SEQ ID NO: 427 - protein, 14 aa, synthetic construct
MDWCPPYLWSECFA

SEQ ID NO: 428 - protein, 14 aa, synthetic construct
MDWCPRYLWHVCFN

SEQ ID NO: 429 - protein, 14 aa, synthetic construct
MDWCPRTLWHVCFN

SEQ ID NO: 430 - protein, 14 aa, synthetic construct
MDWCPRHLWHECYS

SEQ ID NO: 431 - protein, 14 aa, synthetic construct
MDWCPKHLWTECFA

SEQ ID NO: 432 - protein, 14 aa, synthetic construct
MDWCPRHLWYECFN

SEQ ID NO: 433 - protein, 14 aa, synthetic construct
MDWCPRYLWHECFD

SEQ ID NO: 434 - protein, 14 aa, synthetic construct
MDWCPRNLWHLCFA

SEQ ID NO: 435 - protein, 14 aa, synthetic construct
MDWCPKHLWNKCFN

SEQ ID NO: 436 - protein, 14 aa, synthetic construct
MDWCPRFMWAECFN

SEQ ID NO: 437 - protein, 14 aa, synthetic construct
MDWCPRNMWHHCFD

SEQ ID NO: 438 - protein, 14 aa, synthetic construct
MDWCPSDMWAHCYN

SEQ ID NO: 439 - protein, 14 aa, synthetic construct
MDWCPKVMWHYCFA

SEQ ID NO: 440 - protein, 14 aa, synthetic construct
MDWCPRFMWSECFS

SEQ ID NO: 441 - protein, 14 aa, synthetic construct
MDWCPRHMWINCFA

SEQ ID NO: 442 - protein, 14 aa, synthetic construct
MVGCATSMWNRCLT

SEQ ID NO: 443 - protein, 14 aa, synthetic construct
MDWCPRYLWSECFA

SEQ ID NO: 444 - protein, 14 aa, synthetic construct
MDWCPRHLWSVCFN

SEQ ID NO: 445 - protein, 14 aa, synthetic construct
MDWCPFDLWHVCYN

SEQ ID NO: 446 - protein, 14 aa, synthetic construct
MDWCPRFLWHECFN

SEQ ID NO: 447 - protein, 14 aa, synthetic construct
MDWCPRSLWHVCFN

SEQ ID NO: 448 - protein, 14 aa, synthetic construct
MDWCPRNLWHACFN

SEQ ID NO: 449 - protein, 14 aa, synthetic construct
MDWCPVFMWNECFN

SEQ ID NO: 450 - protein, 14 aa, synthetic construct
MDWCPMFMWHECFN

SEQ ID NO: 451 - protein, 14 aa, synthetic construct
MYWCATSMWNRCFV

SEQ ID NO: 452 - protein, 14 aa, synthetic construct
MDWCPRHMWHECFA

SEQ ID NO: 453 - protein, 14 aa, synthetic construct
MDWCPRHLWHVCFN

SEQ ID NO: 454 - protein, 14 aa, synthetic construct
MDWCPRFMWDLCFA

SEQ ID NO: 455 - protein, 14 aa, synthetic construct
VTSCPTTMWNRCFS

SEQ ID NO: 456 - protein, 14 aa, synthetic construct
MTLCLSVDLVHCWY

SEQ ID NO: 457 - protein, 14 aa, synthetic construct
DSFCTWSAWQECGR

SEQ ID NO: 458 - protein, 14 aa, synthetic construct
MDWCPRDFWAFCFF

SEQ ID NO: 459 - protein, 14 aa, synthetic construct
MDWCPTSFWNRCFH

SEQ ID NO: 460 - protein, 14 aa, synthetic construct
MVWCMSVGWAVCLN

SEQ ID NO: 461 - protein, 14 aa, synthetic construct
IDWCPTAGWTYCWF

SEQ ID NO: 462 - protein, 14 aa, synthetic construct
WVSCLRHHWLECFH

SEQ ID NO: 463 - protein, 14 aa, synthetic construct
MDWCPRDHWVDCYS

SEQ ID NO: 464 - protein, 14 aa, synthetic construct
IDWCPKIHWDLCYF

SEQ ID NO: 465 - protein, 14 aa, synthetic construct
MDWCPRSHWSECFS

SEQ ID NO: 466 - protein, 14 aa, synthetic construct
MDWCPRSIWYLCFN

SEQ ID NO: 467 - protein, 14 aa, synthetic construct
MDWCPRYLWAECFN

SEQ ID NO: 468 - protein, 14 aa, synthetic construct
MDWCPRYLWDYCFA

SEQ ID NO: 469 - protein, 14 aa, synthetic construct
MDWCPRSLWFLCFN

SEQ ID NO: 470 - protein, 14 aa, synthetic construct
MDWCPRDLWFACFA

SEQ ID NO: 471 - protein, 14 aa, synthetic construct
MDWCPRHLWNECFN

SEQ ID NO: 472 - protein, 14 aa, synthetic construct
MDWCPRFLWHVCFS

SEQ ID NO: 473 - protein, 14 aa, synthetic construct
MDWCPRDLWAMCFA

SEQ ID NO: 474 - protein, 14 aa, synthetic construct
MERCPRYLWHMCYS

SEQ ID NO: 475 - protein, 14 aa, synthetic construct
MDWCPIALWDFCFA

SEQ ID NO: 476 - protein, 14 aa, synthetic construct
MDWCPRYLWNVCFA

SEQ ID NO: 477 - protein, 14 aa, synthetic construct
MDWCPRDLWSVCFS

SEQ ID NO: 478 - protein, 14 aa, synthetic construct
MDWCPRYLWHVCFY

SEQ ID NO: 479 - protein, 14 aa, synthetic construct
MDWCPRALWDHCFF

SEQ ID NO: 480 - protein, 14 aa, synthetic construct
MDWCPRHLWFECFN

SEQ ID NO: 481 - protein, 14 aa, synthetic construct
MDWCPRSLWDYCFA

SEQ ID NO: 482 - protein, 14 aa, synthetic construct
MDWCPRHLWTECFS

SEQ ID NO: 483 - protein, 14 aa, synthetic construct
MDWCPLYLWAECFN

SEQ ID NO: 484 - protein, 14 aa, synthetic construct
MDWCPIYLWAECFN

SEQ ID NO: 485 - protein, 14 aa, synthetic construct
MDWCPRHLWFECFF

SEQ ID NO: 486 - protein, 14 aa, synthetic construct
MDWCPRYLWHECFF

SEQ ID NO: 487 - protein, 14 aa, synthetic construct
MDWCPRYLWHVCFD

SEQ ID NO: 488 - protein, 14 aa, synthetic construct
MDWCPRYLWTECFS

SEQ ID NO: 489 - protein, 14 aa, synthetic construct
MDWCPKFLWDECFA

SEQ ID NO: 490 - protein, 14 aa, synthetic construct
MDWCPRDLWTECFS

SEQ ID NO: 491 - protein, 14 aa, synthetic construct
MDWCPRHLWNECFA

SEQ ID NO: 492 - protein, 14 aa, synthetic construct
MDWCPRYLWPVCFH

SEQ ID NO: 493 - protein, 14 aa, synthetic construct
MDWCPKSLWAECFN

SEQ ID NO: 494 - protein, 14 aa, synthetic construct
MDWCPMFLWHECFN

SEQ ID NO: 495 - protein, 14 aa, synthetic construct
MDWCPRDLWDECFN

SEQ ID NO: 496 - protein, 14 aa, synthetic construct
MDWCPRDLWYQCFN

SEQ ID NO: 497 - protein, 14 aa, synthetic construct
MDWCPRSLWNYCFN

SEQ ID NO: 498 - protein, 14 aa, synthetic construct
MDWCPIHLWNECFN

SEQ ID NO: 499 - protein, 14 aa, synthetic construct
MDWCPTYLWHVCFS

SEQ ID NO: 500 - protein, 14 aa, synthetic construct
MDWCPRSLWNYCFA

SEQ ID NO: 501 - protein, 14 aa, synthetic construct
MDWCPRHLWHECFT

SEQ ID NO: 502 - protein, 14 aa, synthetic construct
MDWCPMFLWDECFN

SEQ ID NO: 503 - protein, 14 aa, synthetic construct
MSWCPRDLWHLCFF

SEQ ID NO: 504 - protein, 14 aa, synthetic construct
MDWCPTYLWNECFN

SEQ ID NO: 505 - protein, 14 aa, synthetic construct
MDWCPRDLWYVCFA

SEQ ID NO: 506 - protein, 14 aa, synthetic construct
MDWCPRHLWIECFN

SEQ ID NO: 507 - protein, 14 aa, synthetic construct
MDWCPTYLWNVCFA

SEQ ID NO: 508 - protein, 14 aa, synthetic construct
MDWCPRHLWYECFS

SEQ ID NO: 509 - protein, 14 aa, synthetic construct
MDWCPAYLWDECFA

SEQ ID NO: 510 - protein, 14 aa, synthetic construct
MFWCPITLWNECFN

SEQ ID NO: 511 - protein, 14 aa, synthetic construct
MHWCPRDLWHVCFN

SEQ ID NO: 512 - protein, 14 aa, synthetic construct
MDWCPRALWDHCFN

SEQ ID NO: 513 - protein, 14 aa, synthetic construct
MDWCPRALWNVCFA

SEQ ID NO: 514 - protein, 14 aa, synthetic construct
MDWCPRFLWDVCFN

SEQ ID NO: 515 - protein, 14 aa, synthetic construct
MDWCPRHLWDECFY

SEQ ID NO: 516 - protein, 14 aa, synthetic construct
IDWCPRALWDACLA

SEQ ID NO: 517 - protein, 14 aa, synthetic construct
MDWCPTDLWHECFA

SEQ ID NO: 518 - protein, 14 aa, synthetic construct
MDWCPRYLWDECFS

SEQ ID NO: 519 - protein, 14 aa, synthetic construct
MDWCPRFLWDYCFA

SEQ ID NO: 520 - protein, 14 aa, synthetic construct
MDWCPRDLWNWCFN

SEQ ID NO: 521 - protein, 14 aa, synthetic construct
MDWCPKPLWHVCFA

SEQ ID NO: 522 - protein, 14 aa, synthetic construct
MDWCPRFLWNECFY

SEQ ID NO: 523 - protein, 14 aa, synthetic construct
MDWCPRTLWDQCFF

SEQ ID NO: 524 - protein, 14 aa, synthetic construct
MSWCPIDLWSECFA

SEQ ID NO: 525 - protein, 14 aa, synthetic construct
MDWCPRHLWDECFN

SEQ ID NO: 526 - protein, 14 aa, synthetic construct
MDWCPRNLWHECFA

SEQ ID NO: 527 - protein, 14 aa, synthetic construct
MDWCPRDLWTQCFF

SEQ ID NO: 528 - protein, 14 aa, synthetic construct
MDWCPRHLWVHCFS

SEQ ID NO: 529 - protein, 14 aa, synthetic construct
MDFCPRFLWHECFN

SEQ ID NO: 530 - protein, 14 aa, synthetic construct
MDWCPRHLWHACFS

SEQ ID NO: 531 - protein, 14 aa, synthetic construct
MDWCPLFLWDQCFN

SEQ ID NO: 532 - protein, 14 aa, synthetic construct
MAWCPWYLWDECFN

SEQ ID NO: 533 - protein, 14 aa, synthetic construct
LDWCPRHLWALCFN

SEQ ID NO: 534 - protein, 14 aa, synthetic construct
MDWCPWFLWNECFN

SEQ ID NO: 535 - protein, 14 aa, synthetic construct
MDWCPMNLWHECFA

SEQ ID NO: 536 - protein, 14 aa, synthetic construct
MDWCPIHLWYECFN

SEQ ID NO: 537 - protein, 14 aa, synthetic construct
IDWCPLHLWHECYH

SEQ ID NO: 538 - protein, 14 aa, synthetic construct
MDWCPRYLWLECFN

SEQ ID NO: 539 - protein, 14 aa, synthetic construct
MDWCPMYLWDECFF

SEQ ID NO: 540 - protein, 14 aa, synthetic construct
MDWCPRLLWHECFN

SEQ ID NO: 541 - protein, 14 aa, synthetic construct
MDWCPPHLWHECFF

SEQ ID NO: 542 - protein, 14 aa, synthetic construct
MDWCPRPLWHECFA

SEQ ID NO: 543 - protein, 14 aa, synthetic construct
MDWCPMFLWHECFS

SEQ ID NO: 544 - protein, 14 aa, synthetic construct
MHWCPRHLWSECFN

SEQ ID NO: 545 - protein, 14 aa, synthetic construct
MDWCPRILWHECFS

SEQ ID NO: 546 - protein, 14 aa, synthetic construct
MNWCPMHLWAECFN

SEQ ID NO: 547 - protein, 14 aa, synthetic construct
MDWCPRSLWAQCFY

SEQ ID NO: 548 - protein, 14 aa, synthetic construct
MDWCPSYLWPVCFN

SEQ ID NO: 549 - protein, 14 aa, synthetic construct
MHWCPLYLWDECFY

SEQ ID NO: 550 - protein, 14 aa, synthetic construct
MDWCPSFLWYECFN

SEQ ID NO: 551 - protein, 14 aa, synthetic construct
MSWCPPYLWTVCYN

SEQ ID NO: 552 - protein, 14 aa, synthetic construct
MHWCPRDLWYECFA

SEQ ID NO: 553 - protein, 14 aa, synthetic construct
MTWCPAYLWHECFN

SEQ ID NO: 554 - protein, 14 aa, synthetic construct
MAWCPRYLWAECFF

SEQ ID NO: 555 - protein, 14 aa, synthetic construct
MDWCPRYLWVDCFA

SEQ ID NO: 556 - protein, 14 aa, synthetic construct
MDWCPRILWSDCFN

SEQ ID NO: 557 - protein, 14 aa, synthetic construct
MAWCPLDLWDKCFY

SEQ ID NO: 558 - protein, 14 aa, synthetic construct
MNWCPRALWHECFL

SEQ ID NO: 559 - protein, 14 aa, synthetic construct
MDWCPRHLWTYCFH

SEQ ID NO: 560 - protein, 14 aa, synthetic construct
MDWCPFDLWLECFN

SEQ ID NO: 561 - protein, 14 aa, synthetic construct
MHWCPLHLWNECFA

SEQ ID NO: 562 - protein, 14 aa, synthetic construct
MDWCPRNLWAECFS

SEQ ID NO: 563 - protein, 14 aa, synthetic construct
MDYCPSYLWHECFA

SEQ ID NO: 564 - protein, 14 aa, synthetic construct
MAWCPRILWHQCFN

SEQ ID NO: 565 - protein, 14 aa, synthetic construct
ITWCPTSLWNRCLV

SEQ ID NO: 566 - protein, 14 aa, synthetic construct
LAGCQRDLATVCVI

SEQ ID NO: 567 - protein, 14 aa, synthetic construct
IMWCPTSLWNRCVT

SEQ ID NO: 568 - protein, 14 aa, synthetic construct
ILRCQMNLQDECLN

SEQ ID NO: 569 - protein, 14 aa, synthetic construct
NHWCPTTLWNRCVA

SEQ ID NO: 570 - protein, 14 aa, synthetic construct
MDWCPRHLWLECFN

SEQ ID NO: 571 - protein, 14 aa, synthetic construct
MDWCPRFMWNFCFF

SEQ ID NO: 572 - protein, 14 aa, synthetic construct
MDWCPSDMWANCFF

SEQ ID NO: 573 - protein, 14 aa, synthetic construct
IDWCPMHMWDFCYN

SEQ ID NO: 574 - protein, 14 aa, synthetic construct
MDWCPFDMWADCFF

SEQ ID NO: 575 - protein, 14 aa, synthetic construct
LHWCPTSMWTYCYY

SEQ ID NO: 576 - protein, 14 aa, synthetic construct
MDWCPRDMWFECFN

SEQ ID NO: 577 - protein, 14 aa, synthetic construct
MDWCPRYMWSDCFF

SEQ ID NO: 578 - protein, 14 aa, synthetic construct
MDWCPKDMWAECFN

SEQ ID NO: 579 - protein, 14 aa, synthetic construct
TDWCPRDMWYLCFN

SEQ ID NO: 580 - protein, 14 aa, synthetic construct
MDWCPRAMWDYCFY

SEQ ID NO: 581 - protein, 14 aa, synthetic construct
MDWCPRNMWNECFF

SEQ ID NO: 582 - protein, 14 aa, synthetic construct
MDWCPRSMWDSCFN

SEQ ID NO: 583 - protein, 14 aa, synthetic construct
MHWCPTYMWSECFA

SEQ ID NO: 584 - protein, 14 aa, synthetic construct
MDWCPLDMWVLCFA

SEQ ID NO: 585 - protein, 14 aa, synthetic construct
MDWCPRHMWHECFH

SEQ ID NO: 586 - protein, 14 aa, synthetic construct
MSWCPWDMWNECFA

SEQ ID NO: 587 - protein, 14 aa, synthetic construct
MDWCPRDMWTSCFN

SEQ ID NO: 588 - protein, 14 aa, synthetic construct
MDWCPMSMWAFCFD

SEQ ID NO: 589 - protein, 14 aa, synthetic construct
MNWCPIDMWTECFN

SEQ ID NO: 590 - protein, 14 aa, synthetic construct
MNWCPIHMWNQCFA

SEQ ID NO: 591 - protein, 14 aa, synthetic construct
MFWCPKDMWAECFA

SEQ ID NO: 592 - protein, 14 aa, synthetic construct
LHWCPITMWTYCYY

SEQ ID NO: 593 - protein, 14 aa, synthetic construct
IDWCPRYMWHECFF

SEQ ID NO: 594 - protein, 14 aa, synthetic construct
MSWCPRFMWHECFN

SEQ ID NO: 595 - protein, 14 aa, synthetic construct
QHWCPRDMWNDCYA

SEQ ID NO: 596 - protein, 14 aa, synthetic construct
MDWCPSDMWSNCFN

SEQ ID NO: 597 - protein, 14 aa, synthetic construct
LYWCPRDMWAYCYS

SEQ ID NO: 598 - protein, 14 aa, synthetic construct
MDWCPMYMWHKCFH

SEQ ID NO: 599 - protein, 14 aa, synthetic construct
MDWCPSNMWNECFF

SEQ ID NO: 600 - protein, 14 aa, synthetic construct
IYWCPTAMWNRCSA

SEQ ID NO: 601 - protein, 14 aa, synthetic construct
MYWCPKYMWSECFA

SEQ ID NO: 602 - protein, 14 aa, synthetic construct
VDWCPAHMWNECYN

SEQ ID NO: 603 - protein, 14 aa, synthetic construct
MFWCPSTMWNRCFD

SEQ ID NO: 604 - protein, 14 aa, synthetic construct
TIFCPSTMWNRCWT

SEQ ID NO: 605 - protein, 14 aa, synthetic construct
MYWCPINMWDYCYA

SEQ ID NO: 606 - protein, 14 aa, synthetic construct
INLCPTPMWNRCWL

SEQ ID NO: 607 - protein, 14 aa, synthetic construct
ITQCPSTMWNRCSV

SEQ ID NO: 608 - protein, 14 aa, synthetic construct
MDLCPTAMWNRCFY

SEQ ID NO: 609 - protein, 14 aa, synthetic construct
MFLCPSAMWNRCFY

SEQ ID NO: 610 - protein, 14 aa, synthetic construct
VILCPTTMWNRCFH

SEQ ID NO: 611 - protein, 14 aa, synthetic construct
MNWCPSTMWNRCLT

SEQ ID NO: 612 - protein, 14 aa, synthetic construct
IHLCHWVPWIKCSH

SEQ ID NO: 613 - protein, 14 aa, synthetic construct
MAWCPADQWHECFN

SEQ ID NO: 614 - protein, 14 aa, synthetic construct
IDWCPIIQGLPCFA

SEQ ID NO: 615 - protein, 14 aa, synthetic construct
MAWCPWAQFDECLA

SEQ ID NO: 616 - protein, 14 aa, synthetic construct
IVWCPTTQWNRCAT

SEQ ID NO: 617 - protein, 14 aa, synthetic construct
MDWCPRDRWVDCYA

SEQ ID NO: 618 - protein, 14 aa, synthetic construct
MDWCPRDRWADCFN

SEQ ID NO: 619 - protein, 14 aa, synthetic construct
MDWCPVDRWADCFN

SEQ ID NO: 620 - protein, 14 aa, synthetic construct
MDWCPMSRWAFCFD

SEQ ID NO: 621 - protein, 14 aa, synthetic construct
MDWCPRDRWDVCFA

SEQ ID NO: 622 - protein, 14 aa, synthetic construct
MDWCPRDRWVDCFN

SEQ ID NO: 623 - protein, 14 aa, synthetic construct
MDWCPRDRWDDCFF

SEQ ID NO: 624 - protein, 14 aa, synthetic construct
IYWCPIDRWNDCYN

SEQ ID NO: 625 - protein, 14 aa, synthetic construct
MDWCPMTRWNDCYF

SEQ ID NO: 626 - protein, 14 aa, synthetic construct
VNQCTRYRWAECLN

SEQ ID NO: 627 - protein, 14 aa, synthetic construct
MDWCPRARWHDCFF

SEQ ID NO: 628 - protein, 14 aa, synthetic construct
MDWCPRDRWDDCFN

SEQ ID NO: 629 - protein, 14 aa, synthetic construct
MDWCPRDRWDVCYY

SEQ ID NO: 630 - protein, 14 aa, synthetic construct
MAWCPWASFDECLA

SEQ ID NO: 631 - protein, 14 aa, synthetic construct
MVDCPLISFDECLA

SEQ ID NO: 632 - protein, 14 aa, synthetic construct
EDWCPTDVWPYCFS

SEQ ID NO: 633 - protein, 14 aa, synthetic construct
MDWCPRFWWHECYA

SEQ ID NO: 634 - protein, 14 aa, synthetic construct
MDWCPRDWWHMCFN

SEQ ID NO: 635 - protein, 14 aa, synthetic construct
MDWCPSDYWYVCFA